(19) United States
(12) Patent Application Publication
(10) Pub. No.: US 2023/0105982 A1
(43) Pub. Date: Apr. 6, 2023

(54) INSULIN-INCRETIN CONJUGATES

(71) Applicant: Merck Sharp & Dohme Corp., Rahway, NJ (US)

(72) Inventors: Anandan Palani, Bridgewater, NJ (US); Chunhui Huang, Arlington, MA (US); Zhiqiang Yang, Westfield, NJ (US); Lin Yan, East Brunswick, NJ (US); Songnian Lin, Holmdel, NJ (US); Pei Huo, Millburn, NJ (US); Qiaolin Deng, Edison, NJ (US); Elisabetta Bianchi, Pomezia (IT); Federica Orvieto, Pomezia (IT)

(73) Assignee: Merck Sharp & Dohme Corp., Rahway, NJ (US)

(21) Appl. No.: 17/386,958

(22) Filed: Jul. 28, 2021

(51) Int. Cl. A61K 47/62; A61K 38/26; A61K 38/28; A61K 47/54; A61P 3/08

(52) U.S. Cl. CPC A61K 47/62; A61K 38/26; A61K 38/28; A61K 47/542; A61K 47/545; A61P 3/08

(57) ABSTRACT

Insulin-incretin conjugates comprising a peptide having agonist activity at the glucagon-like 1 (GLP-1) receptor, the glucagon (GCG) receptor, and/or the gastric inhibitory protein (GIP) receptor conjugated to an insulin molecule having agonist activity at the insulin receptor and use of the conjugates for treatment of metabolic diseases, for example, Type 2 diabetes, are described.

### CROSS REFERENCE TO RELATED APPLICATIONS

This application claims benefit of U.S. Provisional Application No. 62/310,145 filed Mar. 18, 2016, which is incorporated herein by reference in its entirety.

### BACKGROUND OF THE INVENTION

### (1) Field of the Invention

The present invention relates to insulin-incretin conjugates comprising a peptide having agonist activity at the glucagon-like 1 (GLP-1) receptor, the glucagon (GCG) receptor, and/or the gastric inhibitory protein (GIP) receptor conjugated to an insulin molecule having agonist activity at the insulin receptor and use of the conjugates for treatment of metabolic diseases, for example, Type 2 diabetes.

### (2) Description of Related Art

Insulin is an essential therapy for type 1 diabetes mellitus (T1DM) patients and many type 2 mellitus diabetics (T2DMs), prescribed to close to one third of U.S. patients among all anti-diabetic drug users in the past decade. The worldwide market for insulins is growing at a faster rate than all other anti-diabetic agents combined and is expected to reach US$32.24 billion by 2019. Type 2 diabetes mellitus (T2DM) in particular is a growing global public health problem. However, challenges of current insulin therapies, including narrow therapeutic index (TI) to hypoglycemia and body weight gain, limit their wider adoption and potential for patients to achieve ideal glycemic control, particularly for patients with T2DM.

Due to the progressive nature of diabetes, a combination(s) of two or more drugs acting on different pathophysiological processes is often necessary to achieve an early and sustained achievement of individualized glycemic targets. However, in choosing a therapy it is important that the treatment avoids hypoglycemia. Incretins are a relatively recent class of anti-diabetic drugs and have been shown to have efficacy with an acceptable safety profile. Attempts have been made to combine various incretins with a basal insulin for management of T2DM. Currently, exenatide/long acting exenatide and liraglutide have been co-administered with basal insulin like glargine and detemir, respectively. Currently, a fixed-dose or fixed-ratio combination of insulin degludec and liraglutide is under development. Fixed-dose or fixed-ratio combination or by co-administration may improve control of fasting and postprandial glucose.

Incretins are a group of gastrointestinal hormones that that are involved in a wide variety of physiological functions, including glucose homeostasis, insulin secretion, gastric emptying, and intestinal growth, as well as the regulation of food intake. Pre-proglucagon is a 158 amino acid precursor peptide that is processed in different tissues to form a number of different peptides. Incretins include a number of proglucagon-derived peptides, including glucagon (GCG), glucagon-like peptide-1 (GLP-1; amino acids 7-36 and amino acids 7-35, glucagon-like peptide-2 (GLP-2) and oxyntomodulin (OXM).

GCG is a 29-amino acid peptide that corresponds to amino acids 33 through 61 of pre-proglucagon, while GLP-1 is produced as a 37-amino acid peptide that corresponds to amino acids 72 through 108 of pre-proglucagon. GLP-1(7-36) C-terminal amide and GLP-1(7-37) acid are biologically potent forms of GLP-1 that demonstrate essentially equivalent activity at the GLP-1 receptor.

GCG is a life-saving medicine that is used in the acute treatment of severe hypoglycemia. Oxyntomodulin (OXM) has been reported to have pharmacological ability to suppress appetite and lower body weight. Clinical studies with GLP-1 receptor agonists or stabilized GLP-1 analogs have proven this family of peptides to be an effective treatment for T2DM.

Gastric inhibitory peptide or glucose-dependent insulinotropic peptide (GIP) is a member of the secretin family of hormones. GIP is derived from a 153-amino acid proprotein encoded by the GIP gene and circulates as a biologically active 42-amino acid peptide. The GIP gene is expressed in the small intestine as well as the salivary glands and is a weak inhibitor of gastric acid secretion. In addition to its inhibitory effects in the stomach, in the presence of glucose, GIP enhances insulin release by pancreatic beta islet cells when administered in physiological doses. GIP is believed to function as an enteric factor that stimulates the release of pancreatic insulin and that may play a physiological role in maintaining glucose homeostasis.

GCG-related peptide analogs and derivatives modified to have various degrees of activity at the GLP-1 receptor, the GIP receptor, and the GCG receptor have been disclosed in Published International Application Nos. WO2008/1010017, WO2009/155258, WO2011/075393, WO2012/177444, and WO2012/177443. Two independent and simultaneous papers reported the use of relatively balanced GLP-1 receptor/GCG receptor co-agonists as being of enhanced efficacy and safety relative to pure GLP1R agonists in the treatment of rodent obesity, with simultaneous improvement in glycemic control (Day et al., Nat. Chem. Biol. 5: 749-757 (2009); Pocai eta al., Diabetes 58: 2258-2266 (2009)).

While fixed-dose or fixed-ratio combination or by co-administration may lead to improved control over fasting and postprandial glucose, neither method is without limitations, e.g., pH solubility/stability incompatibility and/or limited ability to adjust the ratio between two molecules having different pharmacologies.

An improvement to simultaneous administration of insulin and an incretin to control T2DM was disclosed in WO2014158900, which disclosed conjugates formed between an insulin molecule and an incretin, including for example a GCG-related peptide, wherein the conjugate has agonist activity at both the insulin receptor and the corresponding incretin receptor(s).

### BRIEF SUMMARY OF THE INVENTION

The present invention provides insulin-incretin conjugates formed between an insulin molecule and an incretin, including for example a GCG-related peptide, wherein the conjugate has agonist activity at both the insulin receptor and the corresponding incretin receptor(s). The conjugation of a GCG-related peptide (e.g., a peptide having agonist activity at the GIP receptor, the GLP-1 receptor, the GCG receptor or combinations thereof) is anticipated to produce a beneficial therapeutic addition to the insulin molecule activity.

The present invention provides a compound comprising an insulin molecule having agonist activity at the insulin receptor conjugated via a non-peptide linking moiety to a peptide having agonist activity at the glucagon-like 1 (GLP-1) receptor, the glucagon (GCG) receptor, and/or the gastric inhibitory protein (GIP) receptor, wherein the non-peptide linking moiety comprises a 1,4-disubstituted 1, 2, 3-triazole.

In particular embodiments, the peptide comprises (a) a first linker arm having a proximal end linked to an amino acid of the peptide and a distal end linked to an azide group and the insulin molecule comprises a second linker arm having a proximal end linked to an amino acid of the insulin molecule and a distal end linked to an alkyne group, wherein the azide group and the alkyne group form the 1,4-disubstituted 1,2,3-triazole; or (b) a first linker arm having a proximal end linked to an amino acid of the peptide and a distal end linked to an alkyne group and the insulin molecule comprises a second linker arm having a proximal end linked to an amino acid of the insulin molecule and a distal end linked to an azide group, wherein the azide group and the alkyne group form the 1,4-disubstituted 1,2,3-triazole in either case.

In particular embodiments, the peptide comprises within its amino acid sequence (a) an azido-norleucine and the insulin molecule comprises a linker arm having a proximal end linked to the amino group of an amino acid of the insulin molecule and a distal end linked to an alkynyl group, wherein the azido group and the alkynyl group form the 1,4-disubstituted 1,2,3-triazole; or (b) an alkynyl-norleucine and the insulin molecule comprises a linker arm having a proximal end linked to the amino group of an amino acid of the insulin molecule and a distal end linked to an azido group, wherein the azido group and the alkynyl group form the 1,4-disubstituted 1,2,3-triazole; or (c) a propargyl-Glycine and the insulin molecule comprises a linker arm having a proximal end linked to the amino group of an amino acid of the insulin molecule and a distal end linked to an azido group, wherein the azido group and the alkynyl group form the 1,4-disubstituted 1,2,3-triazole.

In particular embodiments, the insulin molecule comprises within its amino acid sequence (a) an azido-norleucine and the peptide comprises a linker arm having a proximal end linked to the amino group of an amino acid of the peptide and a distal end linked to an alkynyl group, wherein the azido group and the alkynyl group form the 1,4-disubstituted 1,2,3-triazole; or (b) an alkynyl-norleucine and the peptide comprises a linker arm having a proximal end linked to the amino group of an amino acid of the peptide and a distal end linked to an azido group, wherein the azido group and the alkynyl group form the 1,4-disubstituted 1,2,3-triazole; or (c) a propargyl-Glycine and the insulin molecule comprises a linker arm having a proximal end linked to the amino group of an amino acid of the insulin molecule and a distal end linked to an azido group, wherein the azido group and the alkynyl group form the 1,4-disubstituted 1,2,3-triazole.

In particular embodiments, the insulin molecule comprises an A-chain peptide and a B-chain peptide.

In particular embodiments, the insulin molecule is a heterodimer molecule or a single-chain insulin molecule.

In particular embodiments, the insulin molecule is selected from the group consisting of human insulin, insulin lispro, insulin detemir, insulin glulisine, and insulin glargine.

In particular embodiments, the insulin molecule is conjugated to the peptide via the N-terminal amino acid of the A-chain peptide, the N-terminal amino acid of the B-chain peptide, or the amino acid at position 28 or 29 of the B-chain peptide.

In particular embodiments, the peptide is conjugated to the insulin molecule via an amino acid at position 20, 21, 24, 30, or 31 of the peptide.

In particular embodiments, the peptide is also conjugated to a fatty acid or fatty diacid. In further embodiments, the fatty acid or fatty diacid is conjugated to the epsilon amine of a lysine residue at amino acid position 10 or 20 of the peptide.

In particular embodiments, the conjugate comprises the formula

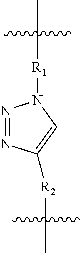

wherein R₁ and R₂ independently comprise a C₁-C₅₀ hydrocarbon chain or substituted hydrocarbon chain, a PEGₙ wherein n is 1-50, a (PEG₂)ₙ wherein n is 1-50, a (PEG₂)ₙ-(γGlu)ₚ-Cₙ wherein each n is independently 1-50 and p is 1 or 2, a (PEG₂)ₙ-Cₙ wherein each n is independently is 1-50, a (PEG)ₙ(PEG)ₙ wherein each n is independently 1-50, a PEGₙ-(Lys-(γGlu)ₚ-Cₙ)—Cₙ wherein each n is independently 1-50 and p is 1 or 2, and a C₅-Lys(γE-Cₙ)-PEGₙ wherein each n is independently 1-50, and wherein the bond between the linking moiety and the insulin molecule and the incretin peptide are indicated by the wavy lines with the proviso that if the bond adjacent to R₁ is to insulin then the bond adjacent to R₂ is to the incretin peptide or that if the bond adjacent to R₁ is to the incretin peptide then the bond adjacent to R₂ is to insulin.

In particular embodiments, the incretin peptide is a glucagon derived that comprises the amino acid sequence

(SEQ ID NO: 1)
HSQGTFTSDYSKYLDERAAQDFVQWLLDT

which further includes at least the following modifications: (i) a substitution of the amino acid at position 2 with an amino acid that renders the peptide resistant to cleavage and inactivation by dipeptidyl peptidase IV; (ii) a lipid moiety covalently linked to the peptide at a lysine residue substituted for the tyrosine residue at position 10 or the glutamine at position 20 of the peptide; (iii) an azide group or an alkyne group conjugated to an amino acid at position 20, 21, 24, 30, or 31; (iv) 0, 1, 2, 3, 4, 5, 6, 7, 8, 9, or 10 additional amino acid substitutions in addition to the substitution at position 2; and optionally, a protecting group that is joined to the C-terminal carboxy group and/or the N-terminal amino group. In embodiments in which the modified glucagon peptide has agonist activity at the GIP receptor, the Histidine at position 1 is substituted with Tyrosine, Phenylalanine, Tryptophan, or other amino acid with an aromatic group.

In particular embodiments, the peptide comprises a substitution of the Ser at position 2 with Val, Ile, Asp, Glu, Met, Trp, Asn, D-Ala, D-Ser, α-methyl-Ser, α-methyl-D-Ser or α-aminoisobutyric acid (aib or U). In particular embodiments, the Ser is substituted with D-Ser or aib. These substitutions at position 2 render the peptide resistant to DPP-4 and active at the GLP-1 receptor. Peptides with a substitution are co-agonists of the GCG and the GLP-1 receptors.

In particular embodiments, the Gln at position 3 is substituted with Glu or Asp. These substitutions increase the selectivity of the peptide for the GLP-1 receptor over the GCG receptor. Such peptides have little or no activity at the GCG receptor.

In particular embodiments, the peptide includes a substitution of the Glu at position 16 with aib, Asn, Ser, or Ala.

In particular embodiments the His at position 1 is substituted with an amino acid with a large aromatic group, for example, Tyr, Phe, or Trp. When this substitution includes the substitution of the Ser at position 2 with aib or D-Ser, the substitution of the Lys at position 12 with Ile and substitution of the Glu at position 16 with aib, the peptide has agonist activity at the GCG, GLP-1 and GIP receptors. When the peptide further includes a substitution of the Gln at position 3 with Glu or Asp, the peptide has agonist activity at the GLP-1 and GIP receptors.

In particular embodiments, the compound comprises a peptide selected from the group consisting of peptides shown in Table 1, e.g., a group of peptides consisting of PEP1, PEP2, PEP3, PEP4, PEP5, PEP6, PEP7, PEP8, PEP9, PEP10, PEP11, PEP12, PEP13, PEP14, PEP15, PEP16, PEP17, PEP18, PEP19, PEP20, PEP21, PEP22, PEP23, PEP24, PEP25, PEP26, PEP27, PEP28, PEP29, PEP30, PEP31, PEP32, PEP33, PEP34, PEP35, PEP36, PEP37, PEP38, PEP39, PEP40, PEP41, PEP42, PEP43, PEP44, PEP45, PEP46, PEP47, PEP48, PEP49, PEP50, PEP51, PEP52, PEP53, PEP54, PEP55, PEP56, PEP57, PEP58, PEP59, PEP60, PEP61, PEP62, PEP63, PEP64, PEP65, PEP66, PEP67, PEP68, PEP69, PEP70, PEP71, PEP72, PEP73, PEP74, PEP75, PEP76, PEP77, PEP78, PEP79, PEP80, PEP81, PEP82, PEP83, PEP84, PEP85, PEP86, PEP87, PEP88, PEP89, PEP90, PEP91, PEP92, PEP93, PEP94, PEP95, PEP96, PEP97, PEP98, PEP99, PEP100, PEP101, PEP102, PEP103, PEP104, PEP105, PEP106, PEP107, PEP108, PEP109, PEP110, PEP111, PEP112, PEP113, PEP114, PEP115, PEP116, PEP117, PEP118, PEP119, PEP120, PEP121, PEP122, PEP123, PEP124, PEP125, PEP126, PEP127, PEP128, PEP129, PEP130, PEP131, and PEP132.

The present invention further provides a conjugate comprising an insulin analog selected from the group consisting of INS1, INS2, INS3, INS4, INS5, INS6, INS7, INS8, INS9, INS10, INS11, INS12, INS13, INS14, INS15, INS16, INS17, INS18, INS19, INS20, INS21, INS22, INS23, INS24, INS25, INS26, INS27, INS28, INS29, INS30, and INS31 conjugated to a peptide selected from the group consisting of PEP1, PEP2, PEP3, PEP4, PEP5, PEP6, PEP7, PEP8, PEP9, PEP10, PEP11, PEP12, PEP13, PEP14, PEP15, PEP16, PEP17, PEP18, PEP19, PEP20, PEP21, PEP22, PEP23, PEP24, PEP25, PEP26, PEP27, PEP28, PEP29, PEP30, PEP31, PEP32, PEP33, PEP34, PEP35, PEP36, PEP37, PEP38, PEP39, PEP40, PEP41, PEP42, PEP43, PEP44, PEP45, PEP46, PEP47, PEP48, PEP49, PEP50, PEP51, PEP52, PEP53, PEP54, PEP55, PEP56, PEP57, PEP58, PEP59, PEP60, PEP61, PEP62, PEP63, PEP64, PEP65, PEP66, PEP67, PEP68, PEP69, PEP70, PEP71, PEP72, PEP73, PEP74, PEP75, PEP76, PEP77, PEP78, PEP79, PEP80, PEP81, PEP82, PEP83, PEP84, PEP85, PEP86, PEP87, PEP88, PEP89, PEP90, PEP91, PEP92, PEP93, PEP94, PEP95, PEP96, PEP97, PEP98, PEP99, PEP100, PEP101, PEP102, PEP103, PEP104, PEP105, PEP106, PEP107, PEP108, PEP109, PEP110, PEP111, PEP112, PEP113, PEP114, PEP115, PEP116, PEP117, PEP118, PEP119, PEP120, PEP121, PEP122, PEP123, PEP124, PEP125, PEP126, PEP127, PEP128, PEP129, PEP130, PEP131, and PEP132.

The present invention further provides a conjugate selected from group of conjugates shown in Table 4, e.g., a conjugate selected from the group consisting of CON1, CON2, CON3, CON4, CON5, CON6, CON7, CON8, CON9, CON10, CON11, CON12, CON13, CON14, CON15, CON16, CON17, CON19, CON20, CON21, CON22, CON23, CON24, CON25, CON26, CON27, CON28, CON29, CON30, CON31, CON32, CON33, CON34, CON35, CON36, CON37, CON38, CON39, CON40, CON41, CON42, CON43, CON44, CON45, CON46, CON47, CON48, CON49, CON50, CON51, CON52, CON53, CON55, CON56, CON57, CON58, CON59, CON60, CON61, CON62, CON63, CON64, CON65, CON66, CON67, CON68, CON69, CON70, CON71, CON72, CON73, CON74, CON75, CON76, CON77, CON78, CON79, CON80, CON81, CON82, CON83, CON84, CON85, CON86, CON87, CON88, CON89, CON90, CON91, CON92, CON93, CON94, CON95, CON96, CON97, CON98, CON99, CON101, CON102, CON103, CON104, CON105, CON106, CON107, CON108, CON109, CON110, CON111, CON112, CON113, CON114, CON115, CON116, CON117, CON118, CON119, CON120, CON121, CON122, CON123, CON124, CON125, CON126, CON127, CON128, CON129, CON130, CON131, CON132, CON133, CON134, CON135, CON136, CON137, CON138, CON139, CON140, CON141, CON142, CON143, CON144, CON145, CON146, CON147, CON148, CON149, CON150, CON151, CON152, CON153, CON154, CON155, CON156, CON157, CON158, CON159, CON160, and CON161.

The present invention further provides pharmaceutical formulation comprising a compound of the above and a pharmaceutically acceptable carrier.

The present invention further provides for the use of a compound of the above in a treatment for a metabolic disease.

The present invention further provides for the use of a compound of the above for the manufacture of a medicament for the treatment of a metabolic disease.

The present invention further provides a method for treating a metabolic disease, comprising administering to an individual in need an effective amount of a compound of any one of the preceding claims to treat the metabolic disease. In particular embodiments, the metabolic disease is diabetes.

### Definitions

The term “about” as used herein means greater or lesser than the value or range of values stated by 10 percent, but is not intended to designate any value or range of values to only this broader definition. Each value or range of values preceded by the term “about” is also intended to encompass the embodiment of the stated absolute value or range of values.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the U.S. Federal government or listed in the U.S. Pharmacopeia for use in animals, including humans.

As used herein the term “pharmaceutically acceptable salt” refers to salts of compounds that retain the biological activity of the parent compound, and which are not biologically or otherwise undesirable. Many of the compounds disclosed herein are capable of forming acid and/or base salts by virtue of the presence of amino and/or carboxyl groups or groups similar thereto.

Pharmaceutically acceptable base addition salts can be prepared from inorganic and organic bases. Salts derived from inorganic bases, include by way of example only, sodium, potassium, lithium, ammonium, calcium and magnesium salts. Salts derived from organic bases include, but are not limited to, salts of primary, secondary and tertiary amines.

As used herein, the term “treating” includes prophylaxis of the specific disorder or condition, or alleviation of the symptoms associated with a specific disorder or condition and/or preventing or eliminating said symptoms. For example, as used herein the term “treating diabetes” will refer in general to altering glucose blood levels in the direction of normal levels and may include increasing or decreasing blood glucose levels depending on a given situation.

As used herein an “effective” amount or a “therapeutically effective amount” of a glucagon peptide refers to a nontoxic but sufficient amount of the peptide to provide the desired effect or a meaningful patient benefit. For example one desired effect would be the prevention or treatment of hyperglycemia, e.g., as measured by a change in blood glucose level closer to normal, or inducing weight loss/preventing weight gain, e.g., as measured by reduction in body weight, or preventing or reducing an increase in body weight, or normalizing body fat distribution. The amount that is “effective” will vary from subject to subject, depending on the age and general condition of the individual, mode of administration, and the like. Thus, it is not always possible to specify an exact “effective amount.” However, an appropriate “effective” amount in any individual case may be determined by one of ordinary skill in the art using routine experimentation.

The term, “parenteral” means not through the alimentary canal but by some other route, e.g., subcutaneous, intramuscular, intraspinal, or intravenous.

As used herein, the term “peptide” encompasses a chain of 11 or more amino acids and typically less than 50 amino acids, wherein the amino acids are naturally occurring or coded or non-naturally occurring or non-coded amino acids. Non-naturally occurring amino acids refer to amino acids that do not naturally occur in vivo but which, nevertheless, can be incorporated into the peptide structures described herein. “Non-coded” as used herein refers to an amino acid that is not an L-isomer of any of the following 20 amino acids: Ala, Cys, Asp, Glu, Phe, Gly, His, Ile, Lys, Leu, Met, Asn, Pro, Gln, Arg, Ser, Thr, Val, Trp, Tyr. “Coded” as used herein refers to an amino acid that is an L-isomer of any of the following 20 amino acids: Ala, Cys, Asp, Glu, Phe, Gly, His, Ile, Lys, Leu, Met, Asn, Pro, Gln, Arg, Ser, Thr, Val, Trp, Tyr. In some embodiments, the peptides and variant peptides described herein are about the same length as SEQ ID NO: 1 (which is 29 amino acids in length), e.g. 25-35 amino acids in length. Exemplary lengths include 29, 30, 31, 32, 33, 34, 35, 36, 37, 38, 39, 40, 41, 42, 43, 44, 45, 46, 47, 48, 49, or 50 amino acids in length. The term further includes peptides wherein one or more amino acids is conjugated to a second molecule via a linker.

Amino acid “modification” refers to an insertion, deletion or substitution of one amino acid with another. In some embodiments, the amino acid substitution or replacement is a conservative amino acid substitution, e.g., a conservative substitution of the amino acid at one or more of positions 2, 5, 7, 10, 11, 12, 13, 14, 16, 17, 18, 19, 20, 21, 24, 27, 28 or 29. As used herein, the term “conservative amino acid substitution” is the replacement of one amino acid with another amino acid having similar properties, e.g., size, charge, hydrophobicity, hydrophilicity, and/or aromaticity, and includes exchanges within one of the following five groups:

I. Small aliphatic, nonpolar or slightly polar residues:

### Ala, Ser, Thr, Pro, Gly;

II. Polar, negative-charged residues and their amides and esters:
Asp, Asn, Glu, Gln, cysteic acid and homocysteic acid;
III. Polar, positive-charged residues:

### His, Arg, Lys; Omithine (Orn)

IV. Large, aliphatic, nonpolar residues:
Met, Leu, Ile, Val, Cys, Norleucine (Nle), homocysteine
V. Large, aromatic residues:
Phe, Tyr, Trp, acetyl phenylalanine

In some embodiments, the amino acid substitution is not a conservative amino acid substitution, e.g., is a non-conservative amino acid substitution.

As used herein the term “charged amino acid” or “charged residue” refers to an amino acid that comprises a side chain that is negative-charged (i.e., de-protonated) or positive-charged (i.e., protonated) in aqueous solution at physiological pH. For example negative-charged amino acids include aspartic acid, glutamic acid, cysteic acid, homocysteic acid, and homoglutamic acid, whereas positive-charged amino acids include arginine, lysine and histidine. Charged amino acids include the charged amino acids among the 20 coded amino acids, as well as atypical or non-naturally occurring or non-coded amino acids.

As used herein the term “acidic amino acid” refers to an amino acid that comprises a second acidic moiety (other than the carboxylic acid of the amino acid), including for example, a carboxylic acid or sulfonic acid group.

As used herein, the term “acylated amino acid” refers to an amino acid comprising an acyl group which is non-native to a naturally-occurring amino acid, regardless of the means by which it is produced (e.g. acylation prior to incorporating the amino acid into a peptide, or acylation after incorporation into a peptide).

As used herein the term “alkylated amino acid” refers to an amino acid comprising an alkyl group which is non-native to a naturally-occurring amino acid, regardless of the means by which it is produced. Accordingly, the acylated amino acids and alkylated amino acids of the present disclosures are non-coded amino acids.

As used herein, the term “selectivity” of a molecule for a first receptor relative to a second receptor refers to the following ratio: EC₅₀ of the molecule at the second receptor divided by the EC₅₀ of the molecule at the first receptor. For example, a molecule that has an EC50 of 1 nM at a first receptor and an EC₅₀ of 100 nM at a second receptor has 100-fold selectivity for the first receptor relative to the second receptor.

As used herein, “glucagon potency” or “potency compared to native glucagon” of a molecule refers to the inverse ratio of the EC₅₀ of the molecule at the glucagon receptor divided by the EC₅₀ of native glucagon at glucagon receptor.

As used herein, “GLP-1 potency” or “potency compared to native GLP-1” of a molecule refers to the inverse ratio of the EC₅₀ of the molecule at GLP-1 receptor divided by the EC₅₀ of native GLP-1 at GLP-1 receptor.

As used herein, “PEG” refers to a polyethylene glycol molecule and “PEGₙ” refers to a polyethylene glycol molecule have n number of ethylene groups.

### BRIEF DESCRIPTION OF THE DRAWINGS

FIG. 1A shows a 3D schematic representation of an insulin-incretin conjugate in which a 1,4-disubstituted 1, 2, 3,-triazole linking moiety links the B1 amino acid to an Nle amino acid in an incretin peptide.

FIG. 1B shows in the upper panel a schematic representation of a Cu(I)-catalyzed Azide-Alkyne Cycloaddition (CuAAC) process that may be used to construct the insulin-incretin conjugates disclosed herein and in the lower panel a Cu-free AAC process that may be used to construct insulin-incretin conjugates wherein one linker comprises a terminal azide and the other linker comprises a strained cyclooctyne.

FIG. 2A, FIG. 2B, and FIG. 2C show various exemplary insulin-incretin conjugates in which the Norleucine (Nle) amino acid of particular incretins is conjugated to the epsilon amino group of Lysine (Lys) at position B29 of the B-chain peptide. The “*” indicates that the incretin has the amino acid sequence and structure of the indicated incretin but wherein the azide group of the linker has formed a 1,2-disubstituted 1,2,3-triazole with the alkyne group on the insulin.

FIG. 3 shows various exemplary insulin-incretin conjugates in which the Nle amino acid of particular incretins is conjugated to the amino group of the Glycine (Gly) at position A1 of the A-chain peptide. The “*” indicates that the incretin has the amino acid sequence and structure of the indicated incretin but wherein the azide group of the linker has formed a 1,2-disubstituted 1,2,3-triazole with the alkyne group on the insulin.

FIG. 4A, FIG. 4B, FIG. 4C, FIG. 4D, FIG. 4E, FIG. 4F, FIG. 4G, FIG. 4H, FIG. 4I, FIG. 4J, FIG. 4K, FIG. 4L, FIG. 4M, FIG. 4N, FIG. 4O, and FIG. 4P show exemplary insulin-incretin conjugates in which the Nle amino acid of particular incretins is conjugated to the amino group of the Phenylalanine (Phe) at position B1 of the B-chain peptide. The “*” indicates that the incretin has the amino acid sequence and structure of the indicated incretin but wherein the azide group of the linker has formed a 1,2-disubstituted 1,2,3-triazole with the alkyne group on the insulin.

FIG. 5 shows an exemplary insulin-incretin conjugate in which the Nle amino acid of PEP74* is conjugated to the amino group of the Phe at position B1 of the B-chain peptide. The “*” indicates that the incretin has the amino acid sequence and structure of the indicated incretin but wherein the azide group of the linker has formed a 1,4,5-trisubstituted 1,2,3-triazole with the alkyne group on the insulin.

FIG. 6A, FIG. 6B, FIG. 6C, and FIG. 6D show exemplary insulin-incretin conjugates in which the epsilon amino group of Lys of the incretin is conjugated to the amino group of the Phe at position B1 of the B-chain peptide. The “*” indicates that the incretin has the amino acid sequence and structure of the indicated incretin but wherein the azide group of the linker has formed a 1,2-disubstituted 1,2,3-triazole with the alkyne group on the insulin.

FIG. 7 shows that the length of the linking moiety at B1 has an effect on the ratio of glucagon (GCG) activity to GLP-1 activity.

FIG. 8 shows that the length of the linking moiety at B29 has an effect on the activity of the insulin at the insulin receptor ratio of GCG activity to GLP-1 activity.

FIG. 9A, FIG. 9B, FIG. 9C, FIG. 9D, FIG. 9E, FIG. 9F, FIG. 9G, FIG. 9H, FIG. 9I, FIG. 9J, FIG. 9K, and FIG. 9L show exemplary insulin-incretin conjugates in which the Norleucine (Nle) amino acid of the incretin is conjugated to the amino group of the Phe at position B1 of the B-chain peptide. The “*” indicates that the incretin has the amino acid sequence and structure of the indicated incretin but wherein the azide group of the linker has formed a 1,2-disubstituted 1,2,3-triazole with the alkyne group on the insulin.

FIG. 10A, FIG. 10B, FIG. 10C, FIG. 10D, FIG. 10E, FIG. 10F, and FIG. 10G show exemplary insulin-incretin conjugates in which the epsilon amino group of Lys of the incretin is conjugated to the amino group of the Phenylalanine (Phe) at position B1 of the B-chain peptide. The “*” indicates that the incretin has the amino acid sequence and structure of the indicated incretin but wherein the azide group of the linker has formed a 1,2-disubstituted 1,2,3-triazole with the alkyne group on the insulin.

FIG. 11 shows exemplary insulin-incretin conjugates CON106 and CON107.

### DETAILED DESCRIPTION OF THE INVENTION

The present invention provides insulin-incretin conjugates that are anticipated to impart a beneficial addition to insulin therapies for diabetes. For example, linking an incretin peptide having agonist activity at the GCG receptor to an insulin molecule may enhance targeting of the conjugate to the liver since the GCG receptor is predominately located in the liver. Targeting the conjugate to the liver may be desirable since the liver is primarily involved in glucose production not glucose utilization. Thus, targeting the liver may provide a safer approach to shutting off glucose production than would occur when the insulin contacts other tissues such as muscle or fat, where in addition to turning off glucose production it also stimulates glucose use leading to a higher risk of hypoglycemia. Also, there are GCG receptors present on the alpha cells of the pancreas. Delivering the conjugate to the alpha cells may suppress additional glucagon production or make the alpha cell more sensitive to hypoglycemia. It is also anticipated that the presence of GCG in the conjugates may serve as a buffer on the activity of the insulin to provide a more baseline activity and thus avoid spikes in blood glucose levels. Furthermore, whereas insulin stimulates lipogenesis in fat cells and weight gain, GCG increases lipolysis and energy expenditure and effects a decrease in weight gain, which may be beneficial in countering the weight gain that may occur during insulin therapies.

Similarly, it is anticipated that conjugates of insulin with other GCG-related peptides including the incretins GLP-1 and GIP and other related peptides having activity at the GLP-1 and/or GIP receptors may produce conjugates having beneficial properties. For example, GLP-1 receptor agonist-insulin conjugate may be targeted to the hypothalamus, to decrease appetite as well as reduce blood glucose. Alternatively or additionally, the GLP-1 receptor agonist-insulin conjugate may be targeted to the beta cells to drive anabolic response (increase islet beta cells production of insulin).

The incretin-insulin conjugates herein are also suitable for further structural enhancements that are envisioned to yield improved therapeutic index, through the use of prodrug chemistry; extended duration of action, by linkage of plasma proteins such as albumin, or other modifications, including pegylation and acylation; and enhanced physical stability, by glycosylation. The preparation of single chain insulin analogs using a C-peptide or peptide linker also provides a novel structural location for where many of these chemical modifications can be successfully deployed. The primary use of the conjugates disclosed herein would be in the treatment of insulin-dependent diabetes, including for example, T1DM, T2DM, and gestational diabetes.

### Insulin-Incretin Conjugates

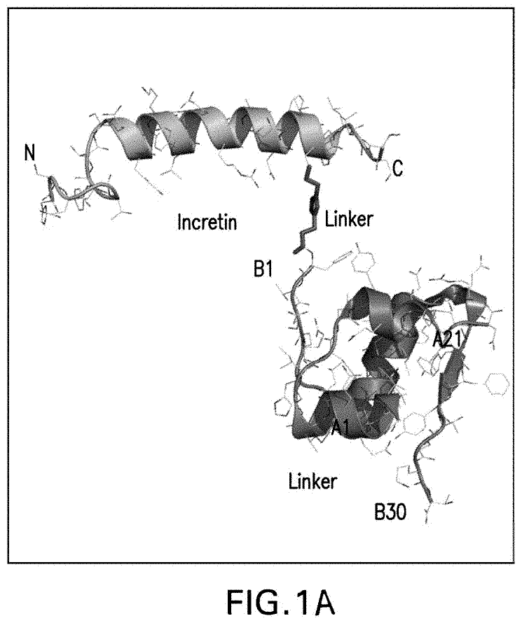

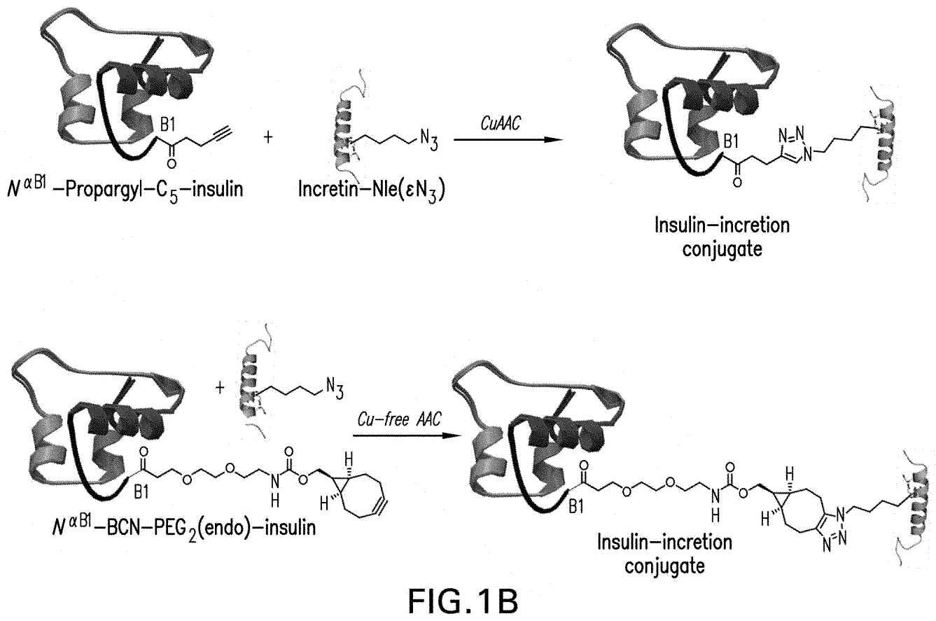

The insulin-incretin conjugate of the present invention comprises an insulin molecule having agonist activity at the insulin receptor conjugated via a non-peptide linking moiety to a peptide having incretin activity, wherein the non-peptide linking moiety comprises a 1,4-disubstituted 1, 2, 3-triazole and the incretin is a peptide having agonist activity at the glucagon-like 1 (GLP-1) receptor, the glucagon (GCG) receptor, the gastric inhibitory protein (GIP) receptor, or both the GLP-1 receptor and the GCG receptor or both the GLP-1 receptor and the GIP receptor. FIG. 1A provides a schematic representation of an insulin-incretin conjugate.

In specific embodiments, the insulin may have an EC₅₀ at the insulin receptor of about 20 nM or less, 10 nM or less, 5 nM or less, or between 1 to nM. In specific embodiments, the incretin may have an EC₅₀ at the GLP-1 receptor of about 10 nM or less, 5 nM or less, 1 nM or less, or between 0.01 nM and 1 nM. In specific embodiments, the incretin may have an EC₅₀ at the GIP receptor of about 10 nM or less, 5 nM or less, 1 nM or less, or between 0.01 nM and 1 nM. In specific embodiments, the incretin may have an EC₅₀ at the GCG1 receptor of about 10 nM or less, 5 nM or less, 1 nM or less, or between 0.01 nM and 1 nM. In specific embodiments, the incretin is a co-agonist in which the activity at the GLP-1 receptor and the GCG receptor are relatively balanced.

In particular embodiments, the insulin-incretin conjugate comprises (i) an insulin molecule having insulin activity and comprising an alkyne group conjugated to a peptide having incretin activity and comprising an azide group under conditions wherein the alkyne group and the azide group form a linking moiety comprising a 1,4-disubstituted 1, 2, 3-triazole or (ii) an insulin molecule comprising an azide group conjugated to an incretin peptide comprising an alkyne group under conditions wherein the azide group and the alkyne group form a 1,4-disubstituted 1, 2, 3-triazole.

In particular embodiments, the peptide comprises (i) a first linker arm having a proximal end linked to an amino acid of the peptide and a distal end linked to an azide group and the insulin molecule comprises a second linker arm having a proximal end linked to an amino acid of the insulin molecule and a distal end linked to an alkyne group; or (ii) the compound of claim 1, wherein the peptide comprises a first linker arm having a proximal end linked to an amino acid of the peptide and a distal end linked to an alkyne group and the insulin molecule comprises a second linker arm having a proximal end linked to an amino acid of the insulin molecule and a distal end linked to an azide group; or (c) a propargyl-Glycine and the insulin molecule comprises a linker arm having a proximal end linked to the amino group of an amino acid of the insulin molecule and a distal end linked to an azido group, wherein the azido group and the alkynyl group form the 1,4-disubstituted 1,2,3-triazole.

In a particular embodiment, the insulin-incretin conjugate comprises an insulin molecule having agonist activity at the insulin receptor conjugated via a non-peptide linking moiety to a peptide having agonist activity at the glucagon-like 1 (GLP-1) receptor, the glucagon (GCG) receptor, or both the GLP-1 receptor and the GCG receptor, wherein the non-peptide linking moiety comprises a 1,4-disubstituted 1, 2, 3-triazole; or in a particular embodiment, the insulin-incretin conjugate comprises an insulin molecule having agonist activity at the insulin receptor conjugated via a non-peptide linking moiety to a peptide having agonist activity at the gastric inhibitory protein (GIP) receptor or both the GLP-1 receptor and the GIP receptor, wherein the non-peptide linking moiety comprises a 1,4-disubstituted 1, 2, 3-triazole.

In particular embodiments of the insulin-incretin conjugates herein, the insulin-incretin conjugate comprises (i) an insulin molecule comprising an alkyne group conjugated to an incretin peptide comprising an azide group under conditions wherein the alkyne group and the azide group form a linking moiety comprising a 1,4-disubstituted 1, 2, 3-triazole or (ii) an insulin molecule comprising an azide group conjugated to an incretin peptide comprising an alkyne group under conditions wherein the azide group and the alkyne group form a 1,4-disubstituted 1, 2, 3-triazole; or (iii) a propargyl-Glycine and the insulin molecule comprises a linker arm having a proximal end linked to the amino group of an amino acid of the insulin molecule and a distal end linked to an azido group, wherein the azido group and the alkynyl group form the 1,4-disubstituted 1,2,3-triazole.

In particular embodiments, the peptide comprises (a) a first linker arm having a proximal end linked to an amino acid of the peptide and a distal end linked to an azide group and the insulin molecule comprises a second linker arm having a proximal end linked to an amino acid of the insulin molecule and a distal end linked to an alkyne group; or (b) the compound of claim 1, wherein the peptide comprises a first linker arm having a proximal end linked to an amino acid of the peptide and a distal end linked to an alkyne group and the insulin molecule comprises a second linker arm having a proximal end linked to an amino acid of the insulin molecule and a distal end linked to an azide group.

In particular embodiments, the linking moiety comprises the formula

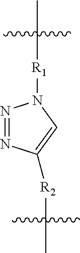

wherein R₁ and R₂ independently comprise a C₁-C₅₀ hydrocarbon chain or substituted hydrocarbon chain, a PEGₙ wherein n is 1-50, a (PEG₂)ₙ wherein n is 1-50, a (PEG₂)ₙ-(γGlu)ₚ-Cₙ wherein each n is independently 1-50 and p is 1 or 2, a (PEG₂)ₙ-Cₙ wherein each n is independently is 1-50, a (PEG)ₙ(PEG)ₙ wherein each n is independently 1-50, a PEGₙ-(Lys-(γGlu)ₚ-Cₙ)—Cₙ wherein each n is independently 1-50 and p is 1 or 2, and a C₅-Lys(γE-Cₙ)-PEGₙ wherein each n is independently 1-50, and wherein the bond between the linking moiety and the insulin molecule and the incretin peptide are indicated by the wavy lines with the proviso that if the bond adjacent to R₁ is to insulin then the bond adjacent to R₂ is to the incretin peptide or that if the bond adjacent to R₁ is to the incretin peptide then the bond adjacent to R₂ is to insulin.

In particular aspects, the peptide optionally includes a protecting group covalently joined to the N-terminal amino group. A protecting group covalently joined to the N-terminal amino group of the peptide reduces the reactivity of the amino terminus under in vivo conditions. Amino protecting groups include —C₁₋₁₀ alkyl, —C₁₋₁₀ substituted alkyl, —C₂₋₁₀ alkenyl, —C₂₋₁₀ substituted alkenyl, aryl, —C₁₋₆ alkyl aryl, —C(O)—(CH₂)₁₋₆—COOH, —C(O)—C₁₋₆ alkyl, —C(O)-aryl, —C(O)—O—C₁₋₆ alkyl, or —C(O)—O-aryl. In particular embodiments, the amino terminus protecting group is selected from the group consisting of acetyl, propyl, succinyl, benzyl, benzyloxycarbonyl, and t-butyloxycarbonyl. Deamination of the N-terminal amino acid is another modification that is contemplated for reducing the reactivity of the amino terminus under in vivo conditions.

In particular aspects, the peptide may be modified to have a protecting group covalently joined to the C-terminal carboxy group, which reduces the reactivity of the carboxy terminus under in vivo conditions. For example, carboxylic acid groups of the peptide, whether carboxyl-terminal or side chain, may be provided in the form of a salt of a pharmacologically-acceptable cation or esterified to form a C₁₋₆ ester, or converted to an amide of formula NRR₂ wherein R and R₂ are each independently H or C₁₋₆ alkyl, or combined to form a heterocyclic ring, such as a 5- or 6-membered ring. The carboxy terminus protecting group is preferably attached to the α-carbonyl group of the last amino acid. Carboxy terminus protecting groups include, but are not limited to, amide, methylamide, and ethylamide. Amino groups of the peptide, whether N-terminal or side chain, may be in the form of a pharmacologically-acceptable acid addition salt, such as the HCl, HBr, acetic, benzoic, toluene sulfonic, maleic, tartaric, and other organic salts, or may be modified to C₁₋₆ alkyl or dialkyl amino or further converted to an amide.

The present invention further provides a conjugate comprising the formula

A-LM-B

wherein A is human insulin molecule or human insulin analog molecule; B is an incretin peptide (e.g., glucagon, GLP-1, or GIP) or a glucagon peptide modified to have agonist activity at the GLP1 receptor, agonist activity at the GIP receptor, agonist the GLP1 and GCG receptors, or agonist activity at the GLP-1 and GIP receptors; LM is a linking moiety comprising a cyclic or acyclic bisamide, a heterocycle and a substituted heterocycle, a C₁-C₅₀ hydrocarbon chain or substituted hydrocarbon chain, a PEGₙ wherein n is 1-50, a (PEG₂)ₙ wherein n is 1-50, a (PEG₂)ₙ-(γGlu)ₚ-Cₙ wherein each n is independently 1-50 and p is 1 or 2, a (PEG₂)ₙ-Cₙ wherein each n is independently is 1-50, a (PEG)ₙ(PEG)ₙ wherein each n is independently 1-50, a PEGₙ-(Lys-(γGlu)ₚ-Cₙ)—Cₙ wherein each n is independently 1-50 and p is 1 or 2, or a C₅-Lys(γE-Cₙ)-PEGₙ wherein each n is independently 1-50.

In particular embodiments, LM is selected from a straight or branched, saturated or unsaturated, optionally substituted C₁₋₃₀ hydrocarbon chain wherein one or more methylene units of Y are optionally and independently replaced by —O—, —S—, —N(R)—, —C(O)—, C(O)O—, OC(O)—, —N(R)C(O)—, —C(O)N(R)—, —S(O)—, —S(O)₂—, —N(R)SO₂—, SO₂N(R)—, a heterocyclic group, an aryl group, or a heteroaryl group, wherein each occurrence of R is independently hydrogen, a suitable protecting group, an acyl moiety, arylalkyl moiety, aliphatic moiety, aryl moiety, heteroaryl moiety, or heteroaliphatic moiety.

In particular embodiments, the human insulin molecule or human insulin analog molecule may be a heterodimer comprising an A-chain peptide and a B-chain peptide connected by disulfide linkages characteristic of human insulin or a single-chain insulin molecule comprising the disulfide linkages characteristic of human insulin wherein the C-terminal amino acid of the B-chain is conjugated to the N-terminal amino acid of the A-chain peptide by a peptide or non-peptide linker.

In particular embodiments, the N-terminal amino acid of the A-chain peptide of the human insulin or human insulin analog molecule is conjugated via LM to an amino acid in the incretin peptide or modified glucagon peptide; or, the N-terminal amino acid of the B-chain peptide of the human insulin or human insulin analog molecule is conjugated via LM to an amino acid in the incretin peptide or modified glucagon peptide; or, the epsilon amino group of a Lysine in the human insulin or human insulin analog molecule is conjugated via LM to an amino acid in the incretin peptide or modified glucagon peptide.

In particular embodiments, the N-terminal amino acid of the A-chain peptide of the human insulin or human insulin analog molecule is conjugated via LM to a Lysine or Norleucine in the incretin peptide or modified glucagon peptide; or, the N-terminal amino acid of the B-chain peptide of the human insulin or human insulin analog molecule is conjugated via LM to a Lysine or Norleucine in the incretin peptide or modified glucagon peptide; or, the epsilon amino group of a Lysine in the human insulin or human insulin analog molecule is conjugated via LM to a Lysine or Norleucine in the incretin peptide or modified glucagon peptide.

### Incretin

The incretin comprising the insulin-incretin conjugate may be any peptide having agonist activity at the glucagon-like 1 (GLP-1) receptor, the glucagon (GCG) receptor, the gastric inhibitory protein (GIP) receptor, or both the GLP-1 receptor and the GCG receptor or both the GLP-1 receptor and the GIP receptor. In particular embodiments, the incretin is a glucagon peptide modified to have agonist activity at the glucagon-like 1 (GLP-1) receptor, the glucagon (GCG) receptor, the gastric inhibitory protein (GIP) receptor, or both the GLP-1 receptor and the GCG receptor or both the GLP-1 receptor and the GIP receptor.

In particular embodiments, the peptide comprises a modified glucagon peptide comprising the amino acid sequence

(SEQ ID NO: 1)
HSQGTFTSDYSKYLDERAAQDFVQWLLDT,

which further includes at least the following modifications: (i) a substitution of the amino acid at position 2 with an amino acid that renders the peptide resistant to cleavage and inactivation by dipeptidyl peptidase IV; (ii) a lipid moiety covalently linked to the peptide at a lysine residue substituted for the tyrosine residue at position 10 or the glutamine at position 20 of the peptide; (iii) an azide group or an alkyne group conjugated to an amino acid at position 20, 21, 24, 30, or 31; (iv) 0, 1, 2, 3, 4, 5, 6, 7, 8, 9, or 10 additional amino acid substitutions in addition to the substitution at position 2; and optionally, a protecting group that is joined to the C-terminal carboxy group and/or the N-terminal amino group. In embodiments in which the modified glucagon peptide has agonist activity at the GIP receptor, the Histidine at position 1 is substituted Tyrosine, Phenylalanine, Tryptophan, or other amino acid with an aromatic group.

In general, the peptide comprises a substitution of the Ser at position 2 with Val, Ile, Asp, Glu, Met, Trp, Asn, D-Ala, D-Ser, α-methyl-Ser, α-methyl-D-Ser or α-aminoisobutyric acid (aib or U). In particular embodiments, the Ser is substituted with D-Ser or aib. These substitutions at position 2 render the peptide resistant to DPP-4 and active at the GLP-1 receptor. Peptides with a substitution are co-agonists of the GCG and the GLP-1 receptors.

In particular embodiments, the Gln at position 3 is substituted with Glu or Asp. These substitutions increase the selectivity of the peptide for the GLP-1 receptor over the GCG receptor. Such peptides have little or no activity at the GCG receptor.

In particular embodiments, the peptide includes a substitution of the Glu at position 16 with aib, Asn, Ser, or Ala.

In particular embodiments the His at position 1 is substituted with an amino acid with a large aromatic group, for example, Tyr, Phe, or Trp. When this substitution includes the substitution of the Ser at position 2 with aib or D-Ser, the substitution of the Lys at position 12 with Ile and substitution of the Glu at position 16 with aib, the peptide has agonist activity at the GCG, GLP-1 and GIP receptors. When the peptide further includes a substitution of the Gln at position 3 with Glu or Asp, the peptide has agonist activity at the GLP-1 and GIP receptors.

In particular embodiments, the insulin molecule comprises an alkyne group and the peptide agonist activity is selective for the GLP-1 receptor and comprises the structure

(SEQ ID NO: 2)
HX²X³GTFTSX⁹X¹⁰SX¹²YX¹⁴DX¹⁶X¹⁷X¹⁸AX²⁰X²¹X²²VX²⁴X²⁵X²⁶X²⁷
X²⁸X²⁹X³⁰X³¹,

wherein X² is amionisobutyric acid (Aib), Gly, D-Serine (s), alpha-methyl Serine (αMS), or alpha-methyl D-Serine (αMs);

### X³ is Val, Glu or Asp;

X⁹ is Asp or alpha-methyl Aspartic acid (αMD);
X¹⁰ is Tyr or Lys conjugated to a lipid moiety;

### X¹² is Lysine, Leucine, or Serine;

X¹⁴ is Leu of alpha-methyl Leucine (αML);
X¹⁶ is Glutamic acid, Asparagine, Serine, Alanine, or Aib;

### X¹⁷ is Arginine or Lysine;

### X¹⁸ is Alanine or Arginine;

X²⁰ is Glutamine or Lysine conjugated via its epsilon amino group to a non-peptide linker comprising a terminal azide group or to a fatty acid or fatty diacid;
X²¹ is Aspartic acid, αMD, Azidonorleucine (Norleucine conjugated via its epsilon carbon to an azide group (Nle(εN₃))), or Lysine conjugated via its epsilon amino group to a non-peptide linker comprising a terminal azide group;

### X²² is Phe or αMF;

X²⁴ is Glutamine, Nle(εN₃), or Lysine conjugated via its epsilon amino group to a non-peptide linker comprising a terminal azide group;
X²⁵ is Tryptophan or alpha-methyl Tryptophan (αMW);

### X²⁶ is Leucine or αML;

X²⁷ is Methionine, Leucine, Methionine sulfoxide, or L-methionine sulphone (2);
X²⁸ is Aspartic acid, Alanine, Lysine, Asparagine, γGlu, Glutamine, or αMD;

### X²⁹ is Threonine or Glycine;

X³⁰ is Arginine, Lysine, or Nle(εN₃), or X³⁰ is absent; and
X³¹ is Glycine, γGlu, Nle(εN₃), or Lysine conjugated via its epsilon amino group to a non-peptide linker comprising a terminal azide group, or X³¹ is absent; and

wherein the C-terminal amino acid optionally is amidated, and with the proviso that either X¹⁰ is a Lysine residue conjugated via its epsilon amino acid to the lipid moiety and one of X²⁰, X²¹, X²⁴, X³⁰, or X³¹ comprises the azide group or X²⁰ is a Lysine residue conjugated via its epsilon amino acid to the lipid moiety and one of X²¹, X²⁴, X³⁰, or X³¹ comprises the azide group.

In particular embodiments, the insulin molecule comprises an azide group and the peptide agonist activity is selective for the GLP-1 receptor and comprises the structure

(SEQ ID NO: 3)
HX²X³GTFTSX⁹X¹⁰SX¹²YX¹⁴DX¹⁶X¹⁷X¹⁸AX²⁰X²¹X²²V-
X²⁴X²⁵X²⁶X²⁷X²⁸X²⁹X³⁰X³¹,

wherein X² is aminoisobutyric acid (Aib), D-Serine (s), or alpha-methyl Serine (αMS);

### X³ is Val, Glu, or Asp;

X⁹ is Asp or alpha-methyl Aspartic acid (αMD);
X¹⁰ is Tyr or Lys conjugated to a lipid moiety;

### X¹² is Lysine, Leucine, or Serine;

X¹⁴ is Leu of alpha-methyl Leucine (αML);
X¹⁶ is Glutamic acid, Asparagine, Serine, Alanine, or Aib;

### X¹⁷ is Arginine or Lysine;

### X¹⁸ is Alanine or Arginine;

X²⁰ is Glutamine or Lysine conjugated via its epsilon amino group to a non-peptide linker comprising a terminal alkyne group or to a fatty acid or fatty diacid;
X²¹ is Aspartic acid, αMD, Alkynylnorleucine (Norleucine conjugated via its epsilon carbon to an alkyne group (Nle(ε-alkyne))), or Lysine conjugated via its epsilon amino group to a non-peptide linker comprising a terminal alkyne group;
X²² is Phe or alpha-methyl Phenylalanine (αMF);
X²⁴ is Glutamine, Nle(ε-alkyne), or Lysine conjugated via its epsilon amino group to a non-peptide linker comprising a terminal alkyne group;
X²⁵ is Tryptophan or alpha-methyl Tryptophan (αMW);

### X²⁶ is Leucine or αML;

X²⁷ is Methionine, Leucine, methionine sulfoxide, or L-methionine sulphone (2);
X²⁸ is Aspartic acid, Alanine, Lysine, Asparagine, γGlu, Glutamine, or αMD;

### X²⁹ is Threonine or Glycine;

X³⁰ is Arginine, Lysine, or Nle(ε-alkyne), or X³⁰ is absent; and
X³¹ is Glycine, γGlu, Nle(ε-alkyne), or Lysine conjugated via its epsilon amino group to a non-peptide linker comprising a terminal alkyne group, or X³¹ is absent; and

wherein the C-terminal amino acid optionally is amidated, and with the proviso that either X¹⁰ is a Lysine residue conjugated via its epsilon amino acid to the lipid moiety and one of X²⁰, X²¹, X²⁴, X³⁰, or X³¹ comprises the alkyne group or X²⁰ is a Lysine residue conjugated via its epsilon amino acid to the lipid moiety and one of X²¹, X²⁴, X³⁰, or X³¹ comprises the alkyne group.

In particular embodiments, the insulin molecule comprises an alkyne group and the peptide has agonist activity at the GLP-1 and GCG receptors and comprises the structure

(SEQ ID NO: 4)
HX²QGTFTSX⁹X¹⁰SX¹²YX¹⁴DX¹⁶X¹⁷X¹⁸AX²⁰X²¹X²²V-
X²⁴X²⁵X²⁶X²⁷X²⁸X²⁹X³⁰X³¹,

wherein X² is aminoisobutyric acid (Aib), D-Serine (s), or alpha-methyl Serine (αMS);
X⁹ is Asp or alpha-methyl Aspartic acid (αMD);
X¹⁰ is Tyr or Lys conjugated to a lipid moiety;

### X¹² is Lysine, Leucine, or Serine;

X¹⁴ is Leu of alpha-methyl Leucine (αML);
X¹⁶ is Glutamic acid, Asparagine, Serine, Alanine, or Aib;

### X¹⁷ is Arginine or Lysine;

### X¹⁸ is Alanine or Arginine;

X²⁰ is Glutamine or Lysine conjugated via its epsilon amino group to a non-peptide linker comprising a terminal azide group or to a fatty acid or fatty diacid;
X²¹ is Aspartic acid, αMD, Azidonorleucine (Norleucine conjugated via its epsilon carbon to an azide group (Nle(εN₃))), or Lysine conjugated via its epsilon amino group to a non-peptide linker comprising a terminal azide group;
X²² is Phe or alpha-methyl Phenylalanine (αMF);
X²⁴ is Glutamine, Nle(εN₃), or Lysine conjugated via its epsilon amino group to a non-peptide linker comprising a terminal azide group;
X²⁵ is Tryptophan or alpha-methyl Tryptophan (αMW);

### X²⁶ is Leucine or αML;

X²⁷ is Methionine, Leucine, methionine sulfoxide, or L-methionine sulphone (2);
X²⁸ is Aspartic acid, Alanine, Lysine, Asparagine, γGlu, Glutamine, or αMD;

### X²⁹ is Threonine or Glycine;

X³⁰ is Arginine, Lysine, or Nle(εN₃), or X³⁰ is absent; and
X³¹ is Glycine, γGlu, Nle(εN₃), or Lysine conjugated via its epsilon amino group to a non-peptide linker comprising a terminal azide group, or X³¹ is absent; and

wherein the C-terminal amino acid optionally is amidated, and with the proviso that either X¹⁰ is a Lysine residue conjugated via its epsilon amino acid to the lipid moiety and one of X²⁰, X²¹, X²⁴, X³⁰, or X³¹ comprises the azide group or X²⁰ is a Lysine residue conjugated via its epsilon amino acid to the lipid moiety and one of X²¹, X²⁴, X³⁰, or X³¹ comprises the azide group.

In particular embodiments, the insulin molecule comprises an azide group and the peptide has agonist activity at the GLP-1 and GCG receptors and comprises the structure

(SEQ ID NO: 5)
HX²QGTFTSX⁹X¹⁰SX¹²YX¹⁴DX¹⁶X¹⁷X¹⁸AX²⁰X²¹X²²V-
X²⁴X²⁵X²⁶X²⁷X²⁸X²⁹X³⁰X³¹,

wherein X² is aminoisobutyric acid (Aib), D-Serine (s), or alpha-methyl Serine (αMS);
X⁹ is Asp or alpha-methyl Aspartic acid (αMD);
X¹⁰ is Tyr or Lys conjugated to a lipid moiety;

### X¹² is Lysine, Leucine, or Serine;

X¹⁴ is Leu of alpha-methyl Leucine (αML);
X¹⁶ is Glutamic acid, Asparagine, Serine, Alanine, or Aib;

### X¹⁷ is Arginine or Lysine;

### X¹⁸ is Alanine or Arginine;

X²⁰ is Glutamine or Lysine conjugated via its epsilon amino group to a non-peptide linker comprising a terminal alkyne group or to a fatty acid or fatty diacid;
X²¹ is Aspartic acid, αMD, Alkynylnorleucine (Norleucine conjugated via its epsilon carbon to an alkyne group (Nle(ε-alkyne))), or Lysine conjugated via its epsilon amino group to a non-peptide linker comprising a terminal alkyne group;
X²² is Phe or alpha-methyl Phenylalanine (αMF);
X²⁴ is Glutamine, Nle(ε-alkyne), or Lysine conjugated via its epsilon amino group to a non-peptide linker comprising a terminal alkyne group;
X²⁵ is Tryptophan or alpha-methyl Tryptophan (αMW);

### X²⁶ is Leucine or αML;

X²⁷ is Methionine, Leucine, methionine sulfoxide, or L-methionine sulphone (2);
X²⁸ is Aspartic acid, Alanine, Lysine, Asparagine, γGlu, Glutamine, or αMD;

### X²⁹ is Threonine or Glycine;

X³⁰ is Arginine, Lysine, or Nle(ε-alkyne), or X³⁰ is absent; and
X³¹ is Glycine, γGlu, Nle(ε-alkyne), or Lysine conjugated via its epsilon amino group to a non-peptide linker comprising a terminal alkyne group, or X³¹ is absent; and

wherein the C-terminal amino acid optionally is amidated, and with the proviso that either X¹⁰ is a Lysine residue conjugated via its epsilon amino acid to the lipid moiety and one of X²⁰, X²¹, X²⁴, X³⁰, or X³¹ comprises the alkyne group or X²⁰ is a Lysine residue conjugated via its epsilon amino acid to the lipid moiety and one of X²¹, X²⁴, X³⁰, or X³¹ comprises the alkyne group.

In particular embodiments, the insulin molecule comprises an alkyne group and the peptide has agonist activity predominantly at the GLP-1 receptor and GIP receptor and comprises the structure

(SEQ ID NO: 6)
X¹X²X³GTFTSX⁹X¹⁰SX¹²YX¹⁴DX¹⁶X¹⁷X¹⁸AX²⁰X²¹X²²V-
X²⁴X²⁵X²⁶X²⁷X²⁸X²⁹X³⁰X³¹,

wherein X¹ is Tyrosine, Phenylalanine, Tryptophan, or other amino acid with an aromatic group;
X² is amionisobutyric acid (Aib), Gly, D-Serine (s), or alpha-methyl Serine (αMS);

### X³ is Val, Glu, or Asp;

X⁹ is Asp or alpha-methyl Aspartic acid (αMD);
X¹⁰ is Tyr or Lys conjugated to a lipid moiety;

### X¹² is Isoleucine, Lysine, Leucine, or Serine;

X¹⁴ is Leu of alpha-methyl Leucine (αML);
X¹⁶ is Glutamic acid, Asparagine, Serine, Alanine, or Aib;

### X¹⁷ is Arginine or Lysine;

### X¹⁸ is Alanine or Arginine;

X²⁰ is Glutamine or Lysine conjugated via its epsilon amino group to a non-peptide linker comprising a terminal azide group or to a fatty acid or fatty diacid;
X²¹ is Aspartic acid, αMD, Azidonorleucine (Norleucine conjugated via its epsilon carbon to an azide group (Nle(εN₃))), or Lysine conjugated via its epsilon amino group to a non-peptide linker comprising a terminal azide group;
X²² is Phe or alpha-methyl Phenylalanine (αMF),
X²⁴ is Glutamine, Nle(εN₃), or Lysine conjugated via its epsilon amino group to a non-peptide linker comprising a terminal azide group;
X²⁵ is Tryptophan or alpha-methyl Tryptophan (αMW);

### X²⁶ is Leucine or αML;

X²⁷ is Met, Leucine, methionine sulfoxide, or L-methionine sulphone (2);
X²⁸ is Aspartic acid, Alanine, Lysine, Asparagine, γGlu, Glutamine, or αMD;

### X²⁹ is Threonine or Glycine;

X³⁰ is Arginine, Lysine, or Nle(εN₃), or X³⁰ is absent; and
X³¹ is Glycine, γGlu, Nle(εN₃), or Lysine conjugated via its epsilon amino group to a non-peptide linker comprising a terminal azide group, or X³¹ is absent; and

wherein the C-terminal amino acid optionally is amidated, and with the proviso that either X¹⁰ is a Lysine residue conjugated via its epsilon amino acid to the lipid moiety and one of X²⁰, X²¹, X²⁴, X³⁰, or X³¹ comprises the azide group or X²⁰ is a Lysine residue conjugated via its epsilon amino acid to the lipid moiety and one of X²¹, X²⁴, X³⁰, or X³¹ comprises the azide group.

In particular embodiments, the insulin molecule comprises an azide group and the peptide has agonist activity at the GLP-1 receptor and GIP receptor and comprises the structure

(SEQ ID NO: ⁷)
X¹X²X³GTFTSX⁹X¹⁰SX¹²YX¹⁴DX¹⁶X¹⁷X¹⁸AX²⁰X²¹X²²V-
X²⁴X²⁵X²⁶X²⁷X²⁸X²⁹X³⁰X³¹,

wherein X¹ is Tyrosine, Phenylalanine, Tryptophan, or other amino acid with an aromatic group;
X² is aminoisobutyric acid (Aib), D-Serine (s), or alpha-methyl Serine (αMS);

### X³ is Val, Glu, or Asp;

X⁹ is Asp or alpha-methyl Aspartic acid (αMD);
X¹⁰ is Tyr or Lys conjugated to a lipid moiety;

### X¹² is Isoleucine, Lysine, Leucine, or Serine;

X¹⁴ is Leu of alpha-methyl Leucine (αML);
X¹⁶ is Glutamic acid, Asparagine, Serine, Alanine, or Aib;

### X¹⁷ is Arginine or Lysine;

### X¹⁸ is Alanine or Arginine;

X²⁰ is Glutamine or Lysine conjugated via its epsilon amino group to a non-peptide linker comprising a terminal alkyne group or to a fatty acid or fatty diacid;
X²¹ is Aspartic acid, αMD, Alkynylnorleucine (Norleucine conjugated via its epsilon carbon to an alkyne group (Nle(ε-alkyne))), or Lysine conjugated via its epsilon amino group to a non-peptide linker comprising a terminal alkyne group;
X²² is Phe or alpha-methyl Phenylalanine (αMF), X²⁴ is Glutamine, Nle(ε-alkyne), or Lysine conjugated via its epsilon amino group to a non-peptide linker comprising a terminal alkyne group;
X²⁵ is Tryptophan or alpha-methyl Tryptophan (αMW);

### X²⁶ is Leucine or αML;

X²⁷ is Methionine, Leucine, methionine sulfoxide, or L-methionine sulphone (2);
X²⁸ is Aspartic acid, Alanine, Lysine, Asparagine, γGlu, Glutamine, or αMD;

### X²⁹ is Threonine or Glycine;

X³⁰ is Arginine, Lysine, or Nle(ε-alkyne), or X³⁰ is absent; and
X³¹ is Glycine, γGlu, Nle(ε-alkyne), or Lysine conjugated via its epsilon amino group to a non-peptide linker comprising a terminal alkyne group, or X³¹ is absent; and

wherein the C-terminal amino acid optionally is amidated, and with the proviso that either X¹⁰ is a Lysine residue conjugated via its epsilon amino acid to the lipid moiety and one of X²⁰, X²¹, X²⁴, X³⁰, or X³¹ comprises the alkyne group or X²⁰ is a Lysine residue conjugated via its epsilon amino acid to the lipid moiety and one of X²¹, X²⁴, X³⁰, or X³¹ comprises the alkyne group.

In particular embodiments, the insulin molecule comprises an alkyne group and the peptide has agonist activity at the GLP-1, GIP, and GCG receptors and comprises the structure

(SEQ ID NO: 8)
X¹X²QGTFTSX⁹X¹⁰SX¹²YX¹⁴DX¹⁶X¹⁷X¹⁸AX²⁰X²¹X²²V-
X²⁴X²⁵X²⁶X²⁷X²⁸X²⁹X³⁰X³¹,

wherein X¹ is Tyrosine, Phenylalanine, Tryptophan, or other amino acid with an aromatic group;
X² is amionisobutyric acid (Aib), Gly, D-Serine (s), or alpha-methyl Serine (αMS);
X⁹ is Asp or alpha-methyl Aspartic acid (αMD);
X¹⁰ is Tyr or Lys conjugated to a lipid moiety;

### X¹² is Isoleucine, Lysine, Leucine, or Serine;

X¹⁴ is Leu of alpha-methyl Leucine (αML);
X¹⁶ is Glutamic acid, Asparagine, Serine, Alanine, or Aib;

### X¹⁷ is Arginine or Lysine;

### X¹⁸ is Alanine or Arginine;

X²⁰ is Glutamine or Lysine conjugated via its epsilon amino group to a non-peptide linker comprising a terminal azide group or to a fatty acid or fatty diacid;
X²¹ is Aspartic acid, αMD, Azidonorleucine (Norleucine conjugated via its epsilon carbon to an azide group (Nle(εN₃))), or Lysine conjugated via its epsilon amino group to a non-peptide linker comprising a terminal azide group;
X²² is Phe or alpha-methyl Phenylalanine (αMF);
X²⁴ is Glutamine, Nle(εN₃), or Lysine conjugated via its epsilon amino group to a non-peptide linker comprising a terminal azide group;
X²⁵ is Tryptophan or alpha-methyl Tryptophan (αMW);

### X²⁶ is Leucine or αML;

X²⁷ is methionine, Leucine, methionine sulfoxide, or L-methionine sulphone (2);
X²⁸ is Aspartic acid, Alanine, Lysine, Asparagine, γGlu, Glutamine, or αMD;

### X²⁹ is Threonine or Glycine;

X³⁰ is Arginine, Lysine, or Nle(εN₃), or X³⁰ is absent; and
X³¹ is Glycine, γGlu, Nle(εN₃), or Lysine conjugated via its epsilon amino group to a non-peptide linker comprising a terminal azide group, or X³¹ is absent; and

wherein the C-terminal amino acid optionally is amidated, and with the proviso that either X¹⁰ is a Lysine residue conjugated via its epsilon amino acid to the lipid moiety and one of X²⁰, X²¹, X²⁴, X³⁰, or X³¹ comprises the azide group or X²⁰ is a Lysine residue conjugated via its epsilon amino acid to the lipid moiety and one of X²¹, X²⁴, X³⁰, or X³¹ comprises the azide group.

In particular embodiments, the insulin molecule comprises an azide group and the peptide has agonist activity at the GLP-1, GIP, and GCG receptors and comprises the structure

(SEQ ID NO: 9)
X¹X²QGTFTSX⁹X¹⁰SX¹²YX¹⁴DX¹⁶X¹⁷X¹⁸AX²⁰X²¹FV-
X²⁴X²⁵X²⁶X²⁷X²⁸X²⁹X³⁰X³¹,

wherein X¹ is Tyrosine, Phenylalanine, Tryptophan, or other amino acid with an aromatic group;
X² is aminoisobutyric acid (Aib), D-Serine (s), or alpha-methyl Serine (αMS);
X⁹ is Asp or alpha-methyl Aspartic acid (αMD);
X¹⁰ is Tyr or Lys conjugated to a lipid moiety;

### X¹² is Isoleucine, Lysine, Leucine, or Serine;

X¹⁴ is Leu of alpha-methyl Leucine (αML);
X¹⁶ is Glutamic acid, Asparagine, Serine, Alanine, or Aib;

### X¹⁷ is Arginine or Lysine;

### X¹⁸ is Alanine or Arginine;

X²⁰ is Glutamine or Lysine conjugated via its epsilon amino group to a non-peptide linker comprising a terminal alkyne group or to a fatty acid or fatty diacid;
X²¹ is Aspartic acid, αMD, Alkynylnorleucine (Norleucine conjugated via its epsilon carbon to an alkyne group (Nle(ε-alkyne))), or Lysine conjugated via its epsilon amino group to a non-peptide linker comprising a terminal alkyne group;
X²² is Phe or alpha-methyl Phenylalanine (αMF);
X²⁴ is Glutamine, Nle(ε-alkyne), or Lysine conjugated via its epsilon amino group to a non-peptide linker comprising a terminal alkyne group;
X²⁵ is Tryptophan or alpha-methyl Tryptophan (αMW);

### X²⁶ is Leucine or αML;

X²⁷ is methionine, Leucine, methionine sulfoxide, or L-methionine sulphone (2);
X²⁸ is Aspartic acid, Alanine, Lysine, Asparagine, γGlu, Glutamine, or αMD;

### X²⁹ is Threonine or Glycine;

X³⁰ is Arginine, Lysine, or Nle(ε-alkyne), or X³⁰ is absent; and
X³¹ is Glycine, γGlu, Nle(ε-alkyne), or Lysine conjugated via its epsilon amino group to a non-peptide linker comprising a terminal alkyne group, or X³¹ is absent; and

wherein the C-terminal amino acid optionally is amidated, and with the proviso that either X¹⁰ is a Lysine residue conjugated via its epsilon amino acid to the lipid moiety and one of X²⁰, X²¹, X²⁴, X³⁰, or X³¹ comprises the alkyne group or X²⁰ is a Lysine residue conjugated via its epsilon amino acid to the lipid moiety and one of X²¹, X²⁴, X³⁰, or X³¹ comprises the alkyne group.

The lipid moiety may be a monocarboxylic acid comprising an aliphatic chain of 13 to 20 methylene groups (fatty acid) wherein one end of the molecule is the proximal end and the other end is the distal end and only one of the proximal end and the distal end has a carboxyl (COOH) group. The fatty acid may be represented by the structure HO₂C(CH₂)ₙCH₃, wherein n is 11, 12, 13, 14, 15, 16, 17, or 18. The fatty acid may have one of the following structures

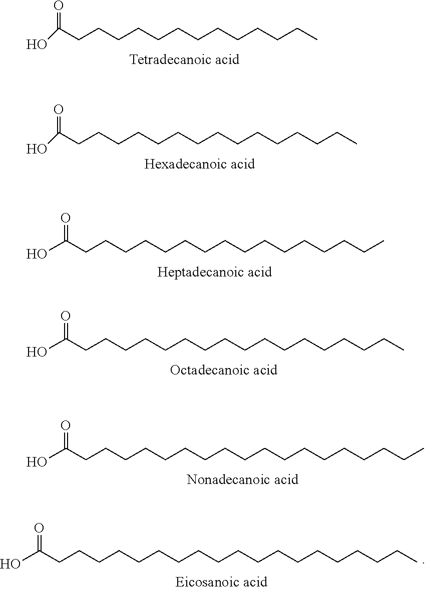 [SMILES: CCCCCCCCCCCCCC(=O)O ; CCCCCCCCCCCCCCCC(=O)O ; CCCCCCCCCCCCCCCCC(=O)O ; CCCCCCCCCCCCCCCCCC(=O)O ; CCCCCCCCCCCCCCCCCCC(=O)O ; CCCCCCCCCCCCCCCCCCCC(=O)O]

The lipid moiety may be an α,ω-dicarboxylic acid comprising an aliphatic chain of 13 to 20 methylene groups (fatty diacid) wherein one end of the molecule is the proximal end and the other end is the distal end and wherein the proximal end and the distal end both have a carboxyl (COOH) group. The fatty diacid may be represented by the structure HO₂C(CH₂)ₙCO₂H, wherein n is 11, 12, 13, 14, 15, 16, 17, or 18. The fatty diacid may have one of the following structures

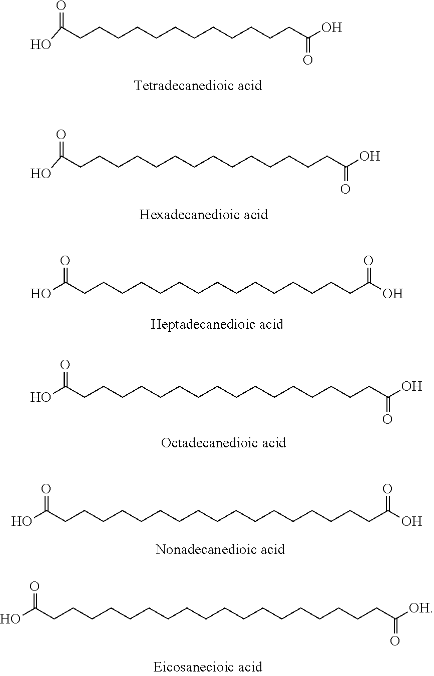 [SMILES: O=C(O)CCCCCCCCCCCCC(=O)O ; O=C(O)CCCCCCCCCCCCCCC(=O)O ; O=C(O)CCCCCCCCCCCCCCCC(=O)O ; O=C(O)CCCCCCCCCCCCCCCCC(=O)O ; O=C(O)CCCCCCCCCCCCCCCCCC(=O)O ; O=C(O)CCCCCCCCCCCCCCCCCCC(=O)O]

As a component of the peptide, the acid functionality at the proximal end of the fatty diacid is conjugated to the amino group of a linker in a C(O)—NH linkage and the acid functionality at the distal end of the fatty diacid is a free carboxyl group (COOH). The COOH group at the distal end helps confer a longer half-life to the peptide by its ability to non-covalently bind to serum albumin, a known carrier for fatty acids in serum. The COOH group enhances duration of action as it provides a better non-covalent interaction with serum albumin than peptides that have been acylated using a fatty acid, which bind serum albumin less efficiently and form a less stable non-covalent interaction with the serum albumin.

When the fatty acid or diacid is conjugated to a linking moiety or linker, it is subsequently referred to as a fatty acid component. The linker may be PEG₂ (8-amino-3,6-dioxaoctanoic acid) linked to Gamma-Glutamic acid (gamma-Glu, γGlu, or γE), which has the structure

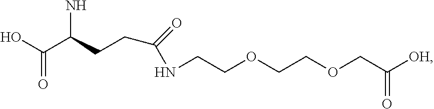 [SMILES: N[C@@H](CCC(=O)NCCOCCOCC(=O)O)C(=O)O]

or the linker may be Gamma-Glutamic acid-gamma glutamic acid (gamma-Glu-gamma-Glu, or γGlu-γGlu, or γEγE), which has the structure

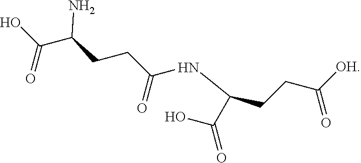 [SMILES: N[C@@H](CCC(=O)N[C@@H](CCC(=O)O)C(=O)O)C(=O)O]

The structure of K(PEG₂PEG₂γE-fatty acid) wherein the linker is PEG₂PEG₂γE and the fatty acid component comprises C14, C16, C17, C18, C19, or C20 fatty acid is represented by

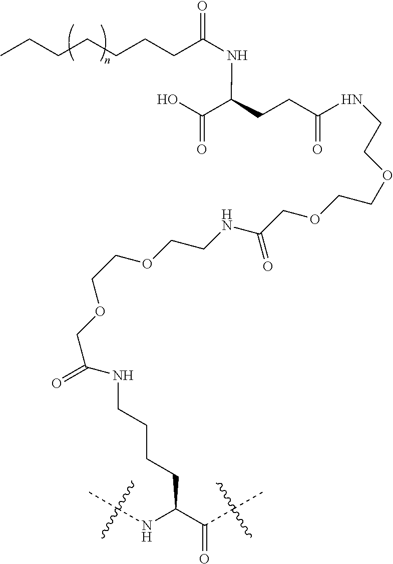

wherein n is 7, 9, 10, 11, 12, 13, or 14 respectively, and the wavy lines represent the bonds between adjacent amino acids in the peptide sequence.

The structure of K(γEγE-fatty acid) wherein the linker is γEγE and the fatty acid component comprises C14, C16, C17, C18, C19, or C20 fatty acid is represented by

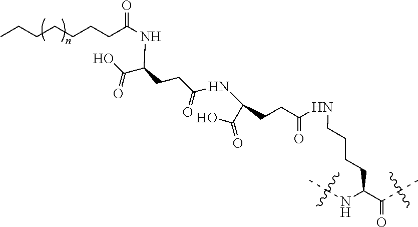

wherein n is 7, 9, 10, 11, 12, 13, or 14, respectively, and the wavy lines represent the bonds between adjacent amino acids in the peptide sequence.

The structure of K(PEG₂PEG₂γE-fatty acid) wherein the linker is PEG₂PEG₂γE and the fatty acid component comprises C14, C16, C17, C18, C19, or C20 fatty diacid is represented by

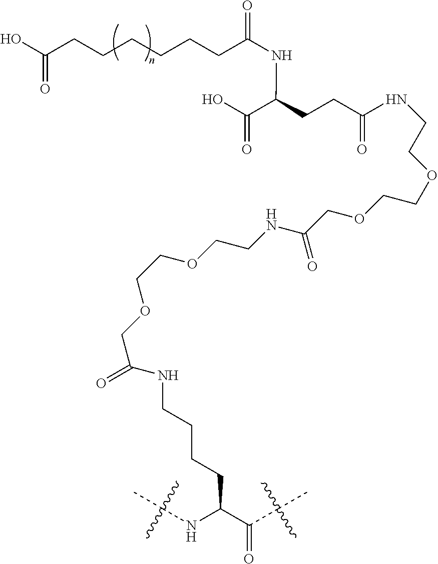

wherein n is 7, 9, 10, 11, 12, 13, or 14 respectively, and the wavy lines represent the bonds between adjacent amino acids in the peptide sequence.

The structure of K(γEγE-fatty acid) wherein the linker is γEγE and the fatty acid component comprises C14, C16, C17, C18, C19, or C20 fatty diacid is represented by

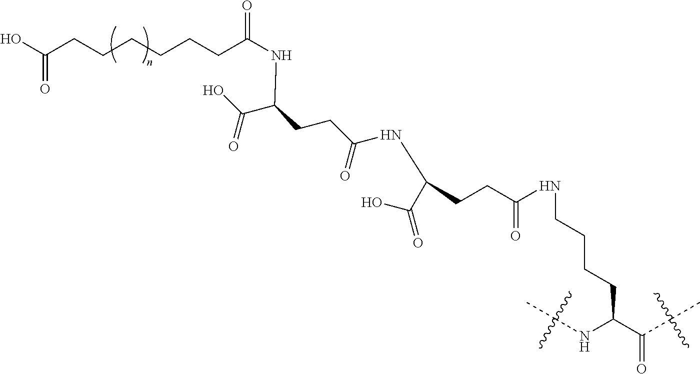

wherein n is 7, 9, 10, 11, 12, 13, or 14, respectively, and the wavy lines represent the bonds between adjacent amino acids in the peptide sequence.

In particular aspects, the peptide may comprise a lysine residue at the C-terminus that is conjugated to a γE residue to provide a KγE at position 30 in the peptide, which is represented by

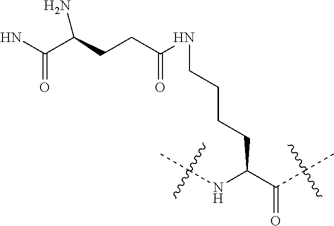

wherein the wavy lines represent the bonds between adjacent amino acids in the peptide sequence.

In a further embodiment, the insulin molecule comprises an alkyne group and the peptide has agonist activity at the GLP-1 and GCG receptors and comprises the structure

(SEQ ID NO: 10)
HX²QGTFTSX⁹X¹⁰SX¹²YXL⁴DX¹⁶X¹⁷X¹⁸AX²⁰X²¹FV-
X²⁴X²⁵X²⁶X²⁷X²⁸X²⁹X³⁰X³¹,

wherein X² is aminoisobutyric acid (Aib), D-Serine (s), or alpha-methyl Serine (αMS);
X⁹ is Serine or alpha-methyl Aspartic acid (αMD);
X¹⁰ is Tyr or Lys conjugated to γGlu-γGlu-C16;

### X¹² is Lysine, Leucine, or Serine;

X¹⁴ is Leu of alpha-methyl Leucine (αML);
X¹⁶ is Glutamic acid, Asparagine, Serine, Alanine, or Aib;

### X¹⁷ is Arginine or Lysine;

### X¹⁸ is Alanine or Arginine;

X²⁰ is Glutamine or Lysine conjugated via its epsilon amine group to PEG₂-C₅N₃, PEG₂PEG₂-C₅N₃, PEG₂PEG₂-γGlu-C₁₆N₃, PEG₂PEG₂-γGlu-C₁₈—OH, or PEG₂PEG₂γE-C₂₀—OH;
X²¹ is Aspartic acid, alpha-methyl Phenylalanine (αMF), αMD, Azidonorleucine (Norleucine conjugated via its epsilon carbon to an azide group (Nle(εN₃))), or Lys conjugated via its epsilon amine group to PEG₂-C₅N₃, PEG₂PEG₂-C₅N₃, or PEG₂PEG₂-γGlu-C₁₆N₃;
X²⁴ is Glutamine, Nle(εN₃), or Lysine conjugated to PEG₂PEG₂-γGlu-C₁₆N₃;
X²⁵ is Tryptophan or alpha-methyl Tryptophan (αMW);

### X²⁶ is Leucine or αML;

X²⁷ is Leucine or L-methionine sulphone (2);
X²⁸ is Aspartic acid, Alanine, Lysine, Asparagine, γGlu, Glutamine, or αMD;

### X²⁹ is Threonine or Glycine;

X³⁰ is Arginine, Lysine, or Nle(εN₃), or X³⁰ is absent; and
X³¹ is Glycine, γGlu, Nle(εN₃), or Lysine conjugated via its epsilon amine group to PEG₂PEG₂-γGlu-C₁₆N₃, PEG₂-C₅N₃ or PEG₂PEG₂-C₅N₃, or X³¹ is absent; and

wherein the C-terminal amino acid optionally is amidated, and with the proviso that either X¹⁰ is a Lysine residue conjugated via its epsilon amino group to a γGlu-γGlu-C₁₆ and one of X²⁰, X²¹, X²⁴, X³⁰, or X³¹ comprises the azide or N₃ group or X²⁰ is a Lysine residue conjugated via its epsilon amino group to the PEG₂PEG₂-γGlu-C₁₈—OH or the PEG₂PEG₂γE-C₂₀—OH and one of X²¹, X²⁴, X³⁰, or X³¹ comprises the azide or N₃ group.

In a further embodiment, the insulin molecule comprises an alkyne group and the peptide has agonist activity at the GLP-1 and GCG receptors and comprises the structure

(SEQ ID NO: 11)
HX²QGTFTSX⁹X¹⁰SX¹²YXL⁴DX¹⁶X¹⁷X¹⁸AX²⁰X²¹FV-
X²⁴X²⁵X²⁶X²⁷X²⁸X²⁹X³⁰X³¹,

wherein X² is aminoisobutyric acid (Aib), D-Serine (s), or alpha-methyl Serine (αMS);
X⁹ is Serine or alpha-methyl Aspartic acid (αMD);
X¹⁰ is Tyr or Lys conjugated to γGlu-γGlu-C₁₆;

### X¹² is Lysine, Leucine, or Serine;

X¹⁴ is Leu of alpha-methyl Leucine (αML);
X¹⁶ is Glutamic acid, Asparagine, Serine, Alanine, or Aib;

### X¹⁷ is Arginine or Lysine;

### X¹⁸ is Alanine or Arginine;

X²⁰ is Glutamine or Lysine conjugated via its epsilon amine group to PEG₂-C₅N₃, PEG₂PEG₂-C₅-alkyne, PEG₂PEG₂-γGlu-C₁₆-alkyne, PEG₂PEG₂-γGlu-C₁₈—OH, or PEG₂PEG₂γE-C₂₀—OH;
X²¹ is Aspartic acid, alpha-methyl Phenylalanine (αMF), αMD, Alkynylnorleucine (Norleucine conjugated via its epsilon carbon to an alkyne group (Nle(ε-alkyne))), or Lys conjugated via its epsilon amine group to PEG₂-C₅-alkyne, PEG₂PEG₂-C₅-alkyne, or PEG₂PEG₂-γGlu-C₁₆-alkyne;
X²⁴ is Glutamine, Nle(ε-alkyne), or Lysine conjugated to PEG₂PEG₂-γGlu-C₁₆-alkyne;
X²⁵ is Tryptophan or alpha-methyl Tryptophan (αMW);

### X²⁶ is Leucine or αML;

X²⁷ is Leucine or L-methionine sulphone (2);
X²⁸ is Aspartic acid, Alanine, Lysine, Asparagine, γGlu, Glutamine, or αMD;

### X²⁹ is Threonine or Glycine;

X³⁰ is Arginine, Lysine, or Nle(ε-alkyne), or X³⁰ is absent; and
X³¹ is Glycine, γGlu, Nle(ε-alkyne), or Lysine conjugated via its epsilon amine group to PEG₂PEG₂-γGlu-C₁₆-alkyne, PEG₂-C₅-alkyne, PEG₂PEG₂-C₅-alkyne, or X³¹ is absent; and

wherein the C-terminal amino acid optionally is amidated, and with the proviso that either X¹⁰ is a Lysine residue conjugated via its epsilon amino group to a γGlu-γGlu-C₁₆ and one of X²⁰, X²¹, X²⁴, X³⁰, or X³¹ comprises the -alkyne or X²⁰ is a Lysine residue conjugated via its epsilon amino group to the PEG₂PEG₂-γGlu-C₁₈—OH or the PEG₂PEG₂γE-C₂₀—OH and one of X²¹, X²⁴, X³⁰, or X³¹ comprises the -alkyne.

In a further embodiment, the insulin molecule comprises an alkyne group and the peptide has agonist activity selective for the GLP-1 receptor and comprises the structure

(SEQ ID NO: 12)
HX²X³GTFTSX⁹X¹⁰SX¹²YX¹⁴DX¹⁶X¹⁷X¹⁸AX²⁰X²¹FV-
X²⁴X²⁵X²⁶X²⁷X²⁸X²⁹X³⁰X³¹,

wherein X² is aminoisobutyric acid (Aib), D-Serine (s), or alpha-methyl Serine (αMS);

### X³ is Glu or Asp;

X⁹ is Serine or alpha-methyl Aspartic acid (αMD);
X¹⁰ is Tyr or Lys conjugated to γGlu-γGlu-C₁₆;

### X¹² is Lysine, Leucine, or Serine;

X¹⁴ is Leu of alpha-methyl Leucine (αML);
X¹⁶ is Glutamic acid, Asparagine, Serine, Alanine, or Aib;

### X¹⁷ is Arginine or Lysine;

### X¹⁸ is Alanine or Arginine;

X²⁰ is Glutamine or Lysine conjugated via its epsilon amine group to PEG₂-C₅N₃, PEG₂PEG₂-C₅N₃, PEG₂PEG₂-γGlu-C₁₆N₃, PEG₂PEG₂-γGlu-C₁₈—OH, or PEG₂PEG₂γE-C₂₀—OH;
X²¹ is Aspartic acid, alpha-methyl Phenylalanine (αMF), αMD, Azidonorleucine (Norleucine conjugated via its epsilon carbon to an azide group (Nle(εN₃))), or Lys conjugated via its epsilon amine group to PEG₂-C₅N₃, PEG₂PEG₂-C₅N₃, or PEG₂PEG₂-γGlu-C₁₆N₃;
X²⁴ is Glutamine, Nle(εN₃), or Lysine conjugated to PEG₂PEG₂-γGlu-C₁₆N₃;
X²⁵ is Tryptophan or alpha-methyl Tryptophan (αMW);

### X²⁶ is Leucine or αML;

X²⁷ is Leucine or L-methionine sulphone (2);
X²⁸ is Aspartic acid, Alanine, Lysine, Asparagine, γGlu, Glutamine, or αMD;

### X²⁹ is Threonine or Glycine;

X³⁰ is Arginine, Lysine, or Nle(εN₃), or X³⁰ is absent; and
X³¹ is Glycine, γGlu, Nle(εN₃), or Lysine conjugated via its epsilon amine group to PEG₂PEG₂-γGlu-C₁₆N₃, PEG₂-C₅N₃ or PEG₂PEG₂-C₅N₃, or X³¹ is absent; and

wherein the C-terminal amino acid optionally is amidated, and with the proviso that either X¹⁰ is a Lysine residue conjugated via its epsilon amino group to a γGlu-γGlu-C₁₆ and one of X²⁰, X²¹, X²⁴, X³⁰, or X³¹ comprises the azide or N₃ group or X²⁰ is a Lysine residue conjugated via its epsilon amino group to the PEG₂PEG₂-γGlu-C₁₈—OH or the PEG₂PEG₂γE-C₂₀—OH and one of X²¹, X²⁴, X³⁰, or X³¹ comprises the azide or N₃ group.

In a further embodiment, the insulin molecule comprises an alkyne group and the peptide has agonist activity selective for the GLP-1 receptor and comprises the structure

(SEQ ID NO: 13)
HX²X³GTFTSX⁹X¹⁰SX¹²YX¹⁴DX¹⁶X¹⁷X¹⁸AX²⁰X²¹FV-
X²⁴X²⁵X²⁶X²⁷X²⁸X²⁹X³⁰X³¹,

wherein X² is aminoisobutyric acid (Aib), D-Serine (s), or alpha-methyl Serine (αMS);

### X³ is Glu or Asp;

X⁹ is Serine or alpha-methyl Aspartic acid (αMD);
X¹⁰ is Tyr or Lys conjugated to γGlu-γGlu-C₁₆;

### X¹² is Lysine, Leucine, or Serine;

X¹⁴ is Leu of alpha-methyl Leucine (αML);
X¹⁶ is Glutamic acid, Asparagine, Serine, Alanine, or Aib;

### X¹⁷ is Arginine or Lysine;

### X¹⁸ is Alanine or Arginine;

X²⁰ is Glutamine or Lysine conjugated via its epsilon amine group to PEG₂-C₅N₃, PEG₂PEG₂-C₅-alkyne, PEG₂PEG₂-γGlu-C₁₆-alkyne, PEG₂PEG₂-γGlu-C₁₈—OH, or PEG₂PEG₂γE-C₂₀—OH;
X²¹ is Aspartic acid, alpha-methyl Phenylalanine (αMF), αMD, Alkynylnorleucine (Norleucine conjugated via its epsilon carbon to an alkyne group (Nle(ε-alkyne))), or Lys conjugated via its epsilon amine group to PEG₂-C₅-alkyne, PEG₂PEG₂-C₅-alkyne, or PEG₂PEG₂-γGlu-C₁₆-alkyne;
X²⁴ is Glutamine, Nle(ε-alkyne), or Lysine conjugated to PEG₂PEG₂-γGlu-C₁₆-alkyne;
X²⁵ is Tryptophan or alpha-methyl Tryptophan (αMW);

### X²⁶ is Leucine or αML;

X²⁷ is Leucine or L-methionine sulphone (2);
X²⁸ is Aspartic acid, Alanine, Lysine, Asparagine, γGlu, Glutamine, or αMD;

### X²⁹ is Threonine or Glycine;

X³⁰ is Arginine, Lysine, or Nle(ε-alkyne), or X³⁰ is absent; and
X³¹ is Glycine, γGlu, Nle(ε-alkyne), or Lysine conjugated via its epsilon amine group to PEG₂PEG₂-γGlu-C₁₆-alkyne, PEG₂-C₅-alkyne, PEG₂PEG₂-C₅-alkyne, or X³¹ is absent; and

wherein the C-terminal amino acid optionally is amidated, and with the proviso that either X¹⁰ is a Lysine residue conjugated via its epsilon amino group to a γGlu-γGlu-C₁₆ and one of X²⁰, X²¹, X²⁴, X³⁰, or X³¹ comprises the -alkyne or X²⁰ is a Lysine residue conjugated via its epsilon amino group to the PEG₂PEG₂-γGlu-C₁₈—OH or the PEG₂PEG₂γE-C₂₀—OH and one of X²¹, X²⁴, X³⁰, or X³¹ comprises the -alkyne.

In a further embodiment, the insulin molecule comprises an alkyne group and the peptide has agonist activity at the GLP-1 and GIP receptors and comprises the structure

(SEQ ID NO: 14)
X¹X²X³GTFTSX⁹X¹⁰SX¹²YX¹⁴DX¹⁶X¹⁷X¹⁸AX²⁰X²¹FV-
x²⁴x²⁵X²⁶x²⁷X²⁸x²⁹x³⁰x³¹,

wherein X¹ is Tyrosine, Phenylalanine, Tryptophan, or other amino acid with an aromatic group;
X² is aminoisobutyric acid (Aib), D-Serine (s), or alpha-methyl Serine (αMS);

### X³ is Glu or Asp;

X⁹ is Serine or alpha-methyl Aspartic acid (αMD);
X¹⁰ is Tyr or Lys conjugated to γGlu-γGlu-C₁₆;

### X¹² is Isoleucine, Lysine, Leucine, or Serine;

X¹⁴ is Leu of alpha-methyl Leucine (αML);
X¹⁶ is Glutamic acid, Asparagine, Serine, Alanine, or Aib;

### X¹⁷ is Arginine or Lysine;

### X¹⁸ is Alanine or Arginine;

X²⁰ is Glutamine or Lysine conjugated via its epsilon amine group to PEG₂-C₅N₃, PEG₂PEG₂-C₅N₃, PEG₂PEG₂-γGlu-C₁₆N₃, PEG₂PEG₂-γGlu-C₁₈—OH, or PEG₂PEG₂γE-C₂₀—OH;
X²¹ is Aspartic acid, alpha-methyl Phenylalanine (αMF), αMD, Azidonorleucine (Norleucine conjugated via its epsilon carbon to an azide group (Nle(εN₃))), or Lys conjugated via its epsilon amine group to PEG₂-C₅N₃, PEG₂PEG₂-C₅N₃, or PEG₂PEG₂-γGlu-C₁₆N₃;
X²⁴ is Glutamine, Nle(εN₃), or Lysine conjugated to PEG₂PEG₂-γGlu-C₁₆N₃;
X²⁵ is Tryptophan or alpha-methyl Tryptophan (αMW);

### X²⁶ is Leucine or αML;

X²⁷ is Leucine or L-methionine sulphone (2);
X²⁸ is Aspartic acid, Alanine, Lysine, Asparagine, γGlu, Glutamine, or αMD;

### X²⁹ is Threonine or Glycine;

X³⁰ is Arginine, Lysine, or Nle(εN₃), or X³⁰ is absent; and
X³¹ is Glycine, γGlu, Nle(εN₃), or Lysine conjugated via its epsilon amine group to PEG₂PEG₂-γGlu-C₁₆N₃, PEG₂-C₅N₃ or PEG₂PEG₂-C₅N₃, or X³¹ is absent; and

wherein the C-terminal amino acid optionally is amidated, and with the proviso that either X¹⁰ is a Lysine residue conjugated via its epsilon amino group to a γGlu-γGlu-C₁₆ and one of X²⁰, X²¹, X²⁴, X³⁰, or X³¹ comprises the azide or N₃ group or X²⁰ is a Lysine residue conjugated via its epsilon amino group to the PEG₂PEG₂-γGlu-C₁₈—OH or the PEG₂PEG₂γE-C₂₀—OH and one of X²¹, X²⁴, X³⁰, or X³¹ comprises the azide or N₃ group.

In a further embodiment, the insulin molecule comprises an alkyne group and the peptide has agonist activity at the GLP-1 and GIP receptors and comprises the structure

(SEQ ID NO: 15)
X¹X²X³GTFTSX⁹X¹⁰SX¹²YX¹⁴DX¹⁶X¹⁷X¹⁸AX²⁰X²¹FV-
X²⁴X²⁵X²⁶X²⁷X²⁸X²⁹X³⁰X³¹,

wherein X¹ is Tyrosine, Phenylalanine, Tryptophan, or other amino acid with an aromatic group;
X² is aminoisobutyric acid (Aib), D-Serine (s), or alpha-methyl Serine (αMS);

### X³ is Glu or Asp;

X⁹ is Serine or alpha-methyl Aspartic acid (αMD);
X¹⁰ is Tyr or Lys conjugated to γGlu-γGlu-C₁₆;

### X¹² is Isoleucine, Lysine, Leucine, or Serine;

X¹⁴ is Leu of alpha-methyl Leucine (αML);
X¹⁶ is Glutamic acid, Asparagine, Serine, Alanine, or Aib;

### X¹⁷ is Arginine or Lysine;

### X¹⁸ is Alanine or Arginine;

X²⁰ is Glutamine or Lysine conjugated via its epsilon amine group to PEG₂-C₅N₃, PEG₂PEG₂-C₅-alkyne, PEG₂PEG₂-γGlu-C₁₆-alkyne, PEG₂PEG₂-γGlu-C₁₈—OH, or PEG₂PEG₂γE-C₂₀—OH;
X²¹ is Aspartic acid, alpha-methyl Phenylalanine (αMF), αMD, Alkynylnorleucine (Norleucine conjugated via its epsilon carbon to an alkyne group (Nle(ε-alkyne))), or Lys conjugated via its epsilon amine group to PEG₂-C₅-alkyne, PEG₂PEG₂-C₅-alkyne, or PEG₂PEG₂-γGlu-C16-alkyne;
X²⁴ is Glutamine, Nle(ε-alkyne), or Lysine conjugated to PEG₂PEG₂-γGlu-C₁₆-alkyne;
X²⁵ is Tryptophan or alpha-methyl Tryptophan (αMW);

### X²⁶ is Leucine or αML;

X²⁷ is Leucine or L-methionine sulphone (2);
X²⁸ is Aspartic acid, Alanine, Lysine, Asparagine, γGlu, Glutamine, or αMD;

### X²⁹ is Threonine or Glycine;

X³⁰ is Arginine, Lysine, or Nle(ε-alkyne), or X³⁰ is absent; and
X³¹ is Glycine, γGlu, Nle(ε-alkyne), or Lysine conjugated via its epsilon amine group to PEG₂PEG₂-γGlu-C₁₆-alkyne, PEG₂-C₅-alkyne, PEG₂PEG₂-C₅-alkyne, or X³¹ is absent; and

wherein the C-terminal amino acid optionally is amidated, and with the proviso that either X¹⁰ is a Lysine residue conjugated via its epsilon amino group to a γGlu-γGlu-C₁₆ and one of X²⁰, X²¹, X²⁴, X³⁰, or X³¹ comprises the -alkyne or X²⁰ is a Lysine residue conjugated via its epsilon amino group to the PEG₂PEG₂-γGlu-C₁₈—OH or the PEG₂PEG₂γE-C₂₀—OH and one of X²¹, X²⁴, X³⁰, or X³¹ comprises the -alkyne.

In a further embodiment, the insulin molecule comprises an alkyne group and the peptide has agonist activity at the GLP-1, GIP, and GCG receptors and comprises the structure

(SEQ ID NO: 16)
X¹X²QGTFTSX⁹X¹⁰SX¹²YX¹⁴DX¹⁶X¹⁷X¹⁸AX²⁰X²¹FV-
X²⁴X²⁵X²⁶X²⁷X²⁸X²⁹X³⁰X³¹,

wherein X¹ is Tyrosine, Phenylalanine, Tryptophan, or other amino acid with an aromatic group;
X² is aminoisobutyric acid (Aib), D-Serine (s), or alpha-methyl Serine (αMS);
X⁹ is Serine or alpha-methyl Aspartic acid (αMD);
X¹⁰ is Tyr or Lys conjugated to γGlu-γGlu-C₁₆;

### X¹² is Isoleucine, Lysine, Leucine, or Serine;

X¹⁴ is Leu of alpha-methyl Leucine (αML);
X¹⁶ is Glutamic acid, Asparagine, Serine, Alanine, or Aib;

### X¹⁷ is Arginine or Lysine;

### X¹⁸ is Alanine or Arginine;

X²⁰ is Glutamine or Lysine conjugated via its epsilon amine group to PEG₂-C₅N₃, PEG₂PEG₂-C₅N₃, PEG₂PEG₂-γGlu-C₁₆N₃, PEG₂PEG₂-γGlu-C₁₈—OH, or PEG₂PEG₂γE-C₂₀—OH;
X²¹ is Aspartic acid, alpha-methyl Phenylalanine (αMF), αMD, Azidonorleucine (Norleucine conjugated via its epsilon carbon to an azide group (Nle(εN₃))), or Lys conjugated via its epsilon amine group to PEG₂-C₅N₃, PEG₂PEG₂-C₅N₃, or PEG₂PEG₂-γGlu-C₁₆N₃;
X²⁴ is Glutamine, Nle(εN₃), or Lysine conjugated to PEG₂PEG₂-γGlu-C₁₆N₃;
X²⁵ is Tryptophan or alpha-methyl Tryptophan (αMW);

### X²⁶ is Leucine or αML;

X²⁷ is Leucine or L-methionine sulphone (2);
X²⁸ is Aspartic acid, Alanine, Lysine, Asparagine, γGlu, Glutamine, or αMD;

### X²⁹ is Threonine or Glycine;

X³⁰ is Arginine, Lysine, or Nle(εN₃), or X³⁰ is absent; and
X³¹ is Glycine, γGlu, Nle(εN₃), or Lysine conjugated via its epsilon amine group to PEG₂PEG₂-γGlu-C₁₆N₃, PEG₂-C₅N₃ or PEG₂PEG₂-C₅N₃, or X³¹ is absent; and

wherein the C-terminal amino acid optionally is amidated, and with the proviso that either X¹⁰ is a Lysine residue conjugated via its epsilon amino group to a γGlu-γGlu-C₁₆ and one of X²⁰, X²¹, X²⁴, X³⁰, or X³¹ comprises the azide or N₃ group or X²⁰ is a Lysine residue conjugated via its epsilon amino group to the PEG₂PEG₂-γGlu-C₁₈—OH or the PEG₂PEG₂γE-C₂₀—OH and one of X²¹, X²⁴, X³⁰, or X³¹ comprises the azide or N₃ group.

In a further embodiment, the insulin molecule comprises an alkyne group and the peptide has agonist activity at the GLP-1, GIP, and GCG receptors and comprises the structure

(SEQ ID NO: 17)
X¹X²QGTFTSX⁹X¹⁰SX¹²YX¹⁴DX¹⁶X¹⁷X¹⁸AX²⁰X²¹FV-
X²⁴X²⁵X²⁶X²⁷X²⁸X²⁹X³⁰X³¹,

wherein X¹ is Tyrosine, Phenylalanine, Tryptophan, or other amino acid with an aromatic group;
X² is aminoisobutyric acid (Aib), D-Serine (s), or alpha-methyl Serine (αMS);
X⁹ is Serine or alpha-methyl Aspartic acid (αMD);
X¹⁰ is Tyr or Lys conjugated to γGlu-γGlu-C₁₆;

### X¹² is Isoleucine, Lysine, Leucine, or Serine;

X¹⁴ is Leu of alpha-methyl Leucine (αML);
X¹⁶ is Glutamic acid, Asparagine, Serine, Alanine, or Aib;

### X¹⁷ is Arginine or Lysine;

### X¹⁸ is Alanine or Arginine;

X²⁰ is Glutamine or Lysine conjugated via its epsilon amine group to PEG₂-C₅N₃, PEG₂PEG₂-C₅-alkyne, PEG₂PEG₂-γGlu-C₁₆-alkyne, PEG₂PEG₂-γGlu-C₁₈—OH, or PEG₂PEG₂γE-C₂₀—OH;
X²¹ is Aspartic acid, alpha-methyl Phenylalanine (αMF), αMD, Alkynylnorleucine (Norleucine conjugated via its epsilon carbon to an alkyne group (Nle(ε-alkyne))), or Lys conjugated via its epsilon amine group to PEG₂-C₅-alkyne, PEG₂PEG₂-C₅-alkyne, or PEG₂PEG₂-γGlu-C₁₆-alkyne;
X²⁴ is Glutamine, Nle(ε-alkyne), or Lysine conjugated to PEG₂PEG₂-γGlu-C₁₆-alkyne;
X²⁵ is Tryptophan or alpha-methyl Tryptophan (αMW);

### X²⁶ is Leucine or αML;

X²⁷ is Leucine or L-methionine sulphone (2);
X²⁸ is Aspartic acid, Alanine, Lysine, Asparagine, γGlu, Glutamine, or αMD;

### X²⁹ is Threonine or Glycine;

X³⁰ is Arginine, Lysine, or Nle(ε-alkyne), or X³⁰ is absent; and
X³¹ is Glycine, γGlu, Nle(ε-alkyne), or Lysine conjugated via its epsilon amine group to PEG₂PEG₂-γGlu-C₁₆-alkyne, PEG₂-C₅-alkyne, PEG₂PEG₂-C₅-alkyne, or X³¹ is absent; and

wherein the C-terminal amino acid optionally is amidated, and with the proviso that either X¹⁰ is a Lysine residue conjugated via its epsilon amino group to a γGlu-γGlu-C₁₆ and one of X²⁰, X²¹, X²⁴, X³⁰, or X³¹ comprises the -alkyne or X²⁰ is a Lysine residue conjugated via its epsilon amino group to the PEG₂PEG₂-γGlu-C₁₈—OH or the PEG₂PEG₂γE-C₂₀—OH and one of X²¹, X²⁴, X³⁰, or X³¹ comprises the -alkyne.

In particular embodiments, the peptide is a GLP-1 analog having the amino acid sequence

(SEQ ID NO: 18)
HGEGTFTSDX¹⁰SSYLEEQAAX²⁰X²¹FIAWLVX²⁸GGGX²⁹,

wherein X¹⁰ is Valine, Nle(ε—N₃), or Lysine conjugated via its epsilon amine group to PEG₂PEG₂-γGlu-C₁₆—N₃, PEG₂-C₅—N₃, or PEG₂PEG₂-C₅—N₃;
X²⁰ is Lysine, Nle(ε—N₃), or Lysine conjugated via its epsilon amine group to PEG₂PEG₂-γGlu-C₁₆—N₃, PEG₂-C₅—N₃, or PEG₂PEG₂-C₅—N₃;
X²¹ is glutamic acid, Nle(ε—N₃), or Lysine conjugated via its epsilon amine group to PEG₂PEG₂-γGlu-C₁₆—N₃, PEG₂-C₅—N₃, or PEG₂PEG₂-C₅—N₃;
X²⁸ is Lysine, Nle(ε—N₃), or Lysine conjugated via its epsilon amine group to PEG₂PEG₂-γGlu-C₁₆—N₃, PEG₂-C₅—N₃, or PEG₂PEG₂-C₅—N₃;
X²⁹ Nle(ε—N₃), or Lysine conjugated via its epsilon amine group to PEG₂PEG₂-γGlu-C₁₆—N₃, PEG₂-C₅—N₃, PEG₂PEG₂-C₅—N₃ or absent;

with the proviso that only one of X¹⁰, X²⁰, X²¹, or X²⁸, or X²⁹ is Nle(ε—N₃) or Lysine conjugated via its epsilon amine group to PEG₂PEG₂-γGlu-C₁₆—N₃, PEG₂-C₅—N₃, PEG₂PEG₂-C₅—N₃.

The peptide may be a GLP-1 analog, for example a GLP(7-37) molecule and analogs thereof comprising 1, 2, 3, 4, 5, or 6 amino acid substitutions or deletions.

In particular embodiments, the peptide is aGLP-1 analog having the amino acid sequence

(SEQ ID NO: 19)
HGEGTFTSDX¹⁰SSYLEEQAAX²⁰X²¹FIAWLVX²⁸GGGX²⁹,

wherein X¹⁰ is Valine, Nle(ε-alkyne), or Lysine conjugated via its epsilon amine group to PEG₂PEG₂-γGlu-C₁₆-alkyne, PEG₂-C₅-alkyne, or PEG₂PEG₂-C₅-alkyne;
X²⁰ is Lysine, Nle(ε—N₃), or Lysine conjugated via its epsilon amine group to PEG₂PEG₂-γGlu-C₁₆-alkyne, PEG₂-C₅-alkyne, or PEG₂PEG₂-C₅-alkyne;
X²¹ is glutamic acid, Nle(ε-alkyne), or Lysine conjugated via its epsilon amine group to PEG₂PEG₂-γGlu-C₁₆-alkyne, PEG₂-C₅-alkyne, or PEG₂PEG₂-C₅-alkyne;
X²⁸ is Lysine, Nle(ε-alkyne), or Lysine conjugated via its epsilon amine group to PEG₂PEG₂-γGlu-C₁₆-alkyne, PEG₂-C₅-alkyne, or PEG₂PEG₂-C₅-alkyne;
X²⁹ Nle(ε-alkyne), or Lysine conjugated via its epsilon amine group to PEG₂PEG₂-γGlu-C₁₆-alkyne, PEG₂-C₅-alkyne, PEG₂PEG₂-C₅-alkyne or absent;

with the proviso that only one of X¹⁰, X²⁰, X²¹, or X²⁸, or X²⁹ is Nle(ε—N₃) or Lysine conjugated via its epsilon amine group to PEG₂PEG₂-γGlu-C₁₆-alkyne, PEG₂-C₅-alkyne, PEG₂PEG₂-C₅-alkyne.

Table 1 shows exemplary peptides comprising an azide group that may be conjugated to an insulin molecule comprising an alkyne group under conditions suitable for the azide group and the alkyne group to form a 1,4-disubstituted 1, 2, 3-triazole.

TABLE 1

| | SEQ | |
|---|---|---|
| | ID | |
| Peptide | NO: | Sequence |
| PEP1 | 35 | HsQGTFTSD—K(γEγEC₁₆)SKYLDERAAQ—Nle(εN₃)FVQWLLDT—NH₂ |
| PEP2 | 36 | HUQGTFTSD—K(γEγEC₁₆)SKYLDERAAQDFV—Nle(εN₃)WLLDGRG—NH₂ |
| PEP3 | 37 | HUQGTFTSD—K(γEγEC₁₆)SKYLDURAAQDFV—Nle(εN₃)WL2KGRG—NH₂ |
| PEP4 | 38 | HsQGTFTSD—K(γEγEC₁₆)—SLYLDURAAQDFV—Nle(εN₃)WLLNT—K(γE)—NH₂ |
| PEP5 | 39 | HsQGTFTSD—K(γEγEC₁₆)—SLYLDURAAQDFVQWLLNT—Nle(εN₃)—γE—NH₂ |
| PEP6 | 40 | HUQGTFTSD—K(γEγEC₁₆)SKYLDURRAQDFVQWLLDT—Nle(εN₃)γE—NH₂ |
| PEP7 | 41 | HsQGTFTSD—K(γEγEC₁₆)SKYLDURAAQDFV—Nle(εN₃)—WLLDT—NH₂ |
| PEP8 | 42 | HsQGTFTSD—K(γEγEC₁₆)SSYLDURAAQDFVQWLLNT—Nle(εN₃)—γE—NH₂ |
| PEP9 | 43 | HsQGTFTSD—K(γEγEC₁₆)SSYLDURAAQDFV—Nle(εN₃)—WLLNTKγE—NH₂ |
| PEP10 | 44 | HUQGTFTSD—K(γEγEC₁₆)SKYLDURAAQDFV—Nle(εN₃)—WL2DT—NH₂ |
| PEP11 | 45 | HsQGTFTSD—K(γEγEC₁₆)SKYLDERAAQDFV—Nle(εN₃)—WLLγET—NH₂ |
| PEP12 | 46 | HsQGTFTSD—K(γEγEC₁₆)SKYLDNKRAQDFV—Nle(εN₃)—WLLQT—NH₂ |
| PEP13 | 47 | HsQGTFTSD—K(γEγEC₁₆)SKYLDSRRAHDFV—Nle(εN₃)—WLLNT—NH₂ |
| PEP14 | 48 | HsQGTFTSD—K(γEγEC₁₆)SKYLDERAAQDFV—K(PEG₂—C₅N₃)WLLDT—NH₂ |
| PEP15 | 49 | HsQGTFTSD—K(γEγEC₁₆)SKYLDERAAQDFV—K(PEG₂PEG₂—C₅N₃)—WLLDT—NH₂ |
| PEP16 | 50 | HsQGTFTSD—K(γEγEC₁₆)SKYLDERAA—K(PEG₂—C₅N₃)—DFVQWLLDT—NH₂ |
| PEP17 | 51 | HsQGTFTSD—K(γEγEC₁₆)SKYLDERAA—K(PEG₂PEG₂—C₅N₃)—DFVQWLLDT—NH₂ |
| PEP18 | 52 | HUQGTFTSDYSKYLDURAAQDFVQWLLDTK—Nle(εN₃)—NH₂ |
| PEP19 | 53 | HUQGTFTSD—K(γEγEC₁₆)SKYLDURAAQDFVQWLLDTK—Nle(εN₃)—NH₂ |
| PEP20 | 54 | HUQGTFTSD—K(γEγEC₁₆)SKYLDURAAQDFVQWLLDTK—K(PEG₂—C₅N₃)—NH2 |
| PEP21 | 55 | HUQGTFTSD—K(γEγEC₁₆)SKYLDURAAQDFVQWLLDTK—K(PEG₂PEG₂—C₅N₃)—NH₂ |
| PEP22 | 56 | HUQGTFTSD—K(γEγEC₁₆)SKYLDURAAQDFV—Nle(εN₃)—WLLDTKγE—NH₂ |
| PEP23 | 57 | HsQGTFTSDK(γEγEC₁₆)SKYLDERAAQNle(εN₃)FVQWL2DT—NH₂ |
| PEP24 | 58 | HsQGTFTSDK(γEγEC₁₆)SKYLDERAAQNle(εN₃)FVQWL2αMDT—NH₂ |
| PEP25 | 59 | HsQGTFTSDYSKYLDERAAQDFV—K(PEG₂PEG₂γE—C₁₆N₃)—WLLDT—NH₂ |
| PEP26 | 60 | HsQGTFTSDYSKYLDERAA—K(PEG₂PEG₂γE—C₁₆N₃)—DFVQWLLDT—NH₂ |
| PEP27 | 61 | HsQGTFTSDYSKYLDERAAQ—K(PEG₂PEG₂γE—C₁₆N₃)—FVQWLLDT—NH₂ |
| PEP28 | 62 | HUQGTFTSDYSKYLDURAAQDFVQWLLDTK—K(PEG₂PEG₂γE—C₁₆N₃)—NH₂ |
| PEP29 | 63 | HsQGTFTSDK(PEG₂PEG₂γEC₁₈—OH)SKYLDERAAQDFVNle(εN₃)WLLDT—NH₂ |
| PEP30 | 64 | HUQGTFTSDYSKYLDARAAK(PEG₂PEG₂γEC18—OH)DFVNle(εN₃)WL2DT—NH₂ |
| PEP31 | 65 | HUQGTFTSDYSKYLDARAAK(PEG₂PEG₂γEC20—OH)DFVNle(εN₃)WL2DT—NH₂ |
| PEP32 | 66 | HsQGTFTSαMDK(γEγEC₁₆)SKYLeuDERAAQDFVNle(εN₃)WL2DT—NH₂ |
| PEP33 | 67 | HsQGTFTSDK(γEγEC₁₆)SKYαMLDERAAQDFVNIe(εN₃)WL2DT—NH₂ |
| PEP34 | 68 | HsQGTFTSDK(γEγEC₁₆)SKYLDERAAQαMDFVNle(εN₃)WL2DT—NH₂ |
| PEP35 | 69 | HsQGTFTSDK(γEγEC₁₆)SKYLDERAAQDαMFVNle(εN₃)WL2DT—NH₂ |
| PEP36 | 70 | HsQGTFTSDK(γEγEC₁₆)SKYLDERAAQDFVNle(εN₃)αMWL2DT—NH₂ |
| PEP37 | 71 | HsQGTFTSDK(γEγEC₁₆)SKYLDERAAQDFVNle(εN₃)WαML2DT—NH₂ |
| PEP38 | 72 | HsQGTFTSDK(γEγEC₁₆)SKYLDERAAQDFVNle(εN₃)WL2αMDT—NH₂ |
| PEP39 | 73 | HαMSQGTFTSDK(γEγEC₁₆)SKYLDURAAQDFV—Nle(εN₃)—WLLATKγE—NH₂ |
| PEP40 | 74 | HαMsQGTFTSDK(γEγEC₁₆)SKYLDURAAQDFV—Nle(εN₃)—WLLATKγE—NH₂ |
| PEP41 | 75 | HαMSQGTFTSDK(γEγEC₁₆)SKYLDARAAQDFV—Nle(εN₃)—WLLDT—NH₂ |
| PEP42 | 76 | HαMsQGTFTSDK(γEγEC₁₆)SKYLDARAAQDFV—Nle(εN₃)—WLLDT—NH₂ |
| PEP43 | 77 | HαMsVGTFTSDK(γEγEC₁₆)SKYLDURAAQDFV—Nle(εN₃)—WL2DT—NH₂ |
| PEP44 | 78 | HUQGTFTSDK(γEγEC₁₆)SKYLDERAAQDFVNle(εN₃)WL2DGRG—NH₂ |
| PEP45 | 79 | HUQGTFTSDK(γEγEC₁₆)SKYLDERAAQDFVNle(εN₃)WL2αMDGRG—NH₂ |
| PEP46 | 80 | HUQGTFTSDK(γEγEC₁₆)SKYLDURAAQDFVQWLLDTNle(εN₃)γE—NH₂ |
| PEP47 | 81 | HUQGTFTSDK(γEγEC₁₆)SKYLDURAAQDFVQWL2DTNle(εN₃)γE—NH₂ |
| PEP48 | 82 | HUQGTFTSDK(γEγEC₁₆)SKYLDURAAQDFVQWL2αMDTNle(εN₃)γE—NH₂ |
| PEP49 | 83 | HUQGTFTSDK(γEγEC₁₆)SKYLDURAAQDFVNle(εN₃)WL2αMDT—NH₂ |
| PEP50 | 84 | HsEGTFTSDK(γEγEC₁₆)SKYLDERAAQDFVNle(εN₃)WLLDT—NH₂ |
| PEP51 | 85 | HsQGTFTSDK(γEγEC₁₆)SKYLDERAAQDFVK(PEG₂PEG₂—C₅N₃)WL2DT—NH₂ |
| PEP52 | 86 | HsQGTFTSDK(γEγEC₁₆)SKYLDERAAQDFVK(PEG₂PEG₂—C₅N₃)WL2αMDT—NH₂ |
| PEP53 | 87 | HsQGTFTSDK(γEγEC₁₆)SKYLDERAAQDFVK(PEG₂—C₁₀N₃)WLLDT—NH₂ |
| PEP54 | 88 | HsQGTFTSDK(γEγEC₁₆)SKYLDERAAQDFVK(PEG₂—C₁₆N₃)WLLDT—NH₂ |
| PEP55 | 89 | HUQGTFTSDK(γEγEC₁₆)SKYLDURAAQDFVK(PEG₂—C₅N₃)WL2DT—NH₂ |
| PEP56 | 90 | HUQGTFTSDK(γEγEC₁₆)SKYLDURAAQDFVK(PEG₂PEG₂—C₅N₃)WL2DT—NH₂ |
| PEP57 | 91 | HsQGTFTSDK(γEγEC₁₆)SKYLDERAAK(PEG₂PEG₂γE—C₁₀N₃)DFVQWLLDT—NH₂ |
| PEP58 | 92 | HsQGTFTSDK(γEγEC₁₆)SKYLDERAAK(PEG₂PEG₂γE—C₁₆N₃)DFVQWLLDT—NH₂ |
| PEP59 | 93 | HsQGTFTSDK(γEγEC₁₆)SKYLDURAAQDFVQWLLDTKNle(εN₃)—NH₂ |
| PEP60 | 94 | HsQGTFTSDK(γEγEC₁₆)SKYLDERAAQDFVQWLLDTKNle(εN₃)—NH₂ |
| PEP61 | 95 | HsQGTFTSDK(γEγEC₁₆)SKYLDURAAQDFVQWLLDTKK(PEG₂PEG₂—C₅N₃)NH₂ |
| PEP62 | 96 | HsQGTFTSDK(γEγEC₁₆)SKYLDERAAQDFVQWLLDTKK(PEG₂PEG₂—C₅N₃)NH₂ |
| PEP63 | 97 | HsQGTFTSDK(γEγEC₁₆)SKYLDURAAQDFVNle(εN₃)WLLDTKγE—NH₂ |
| PEP64 | 98 | HUQGTFTSDK(γEγEC₁₆)SKYLDURAAQDFVK(PEG₂—C₅N₃)WLLDTKγE—NH₂ |
| PEP65 | 99 | HUQGTFTSDK(γEγEC₁₆)SKYLDURAAQDFVK(PEG₂PEG₂—C5N₃)WLLDTKγE—NH₂ |
| PEP66 | 100 | HsQGTFTSDK(γEγEC₁₆)SKYLDERAAQK(PEG₂PEG₂γEC₁₆N₃)FVQWLLDT—NH₂ |
| PEP67 | 101 | HsQGTFTSDK(γEγEC₁₆)SKYLDERAAQDFVK(PEG₂PEG₂γEC₁₆N₃)WLLDT—NH₂ |
| PEP68 | 102 | HUQGTFTSDKSKYLDURAAQDFVNle(εN₃)WLMNTKQ—COOH |
| PEP69 | 103 | HsQGTFTSDKSKYLDERAAQDFVNle(εN₃)WLLDT—NH₂ |
| PEP70 | 104 | HUQGTFTSDKSKYLDURAANle(εN₃)DFVQWLMNTKQ—COOH |
| PEP71 | 105 | HsQGTFTSDKSKYLDERAANle(εN₃)DFVQWLLDT—NH₂ |
| PEP72 | 106 | HUQGTFTSDKSKYLDURAAQDFVQWLMNTKQ Nle(εN₃)—NH₂ |
| PEP73 | 107 | HsQGTFTSDKSKYLDERAAQDFVQWLLDT Nle(εN₃)—NH₂ |
| PEP74 | 108 | HsQGTFTSDK(γEγEC₁₆)SKYLDERAAQDFVNle(εN₃)WLLDT—NH₂ |
| PEP75 | 109 | HsEGTFTSDK(γEγEC₁₆)SKYLDERAAQDFVNle(εN₃)WLLDT—NH₂ |
| PEP76 | 110 | HUQGTFTSDK(γEC₁₆)SKYLDERAAQDFVNle(εN₃)WLLDGGPSSGAPPPS—NH₂ |
| PEP77 | 111 | HGEGTFTSDLSKQMEEEAVRLFI Nle(εN₃)WLKNGGPSSGAPPPS—NH₂ |
| PEP78 | 112 | HsQGTFTSDK(γEγEC₁₆)SKYLDERAAQDFVKWLLDT—NH₂ |
| PEP79 | 113 | HsQGTFTSDK(γEγEC₁₆)SKYαMLDERAAQDFVNle(εN₃)WL2DT—NH₂ |
| PEP80 | 114 | HsQGTFTSDK(γEγEC₁₆)SKYLDERAAQDFVNle(εN₃)αMWL2DT—NH₂ |
| PEP81 | 115 | HsQGTFTSDK(γEγEC₁₆)SKYLDERAAQDFVNIe(εN₃)WL2αMDT—NH₂ |
| PEP82 | 116 | HαMSQGTFTSDK(γEγEC₁₆)SKYLDURAAQDFV—Nle(εN₃)WLLATKγE—NH₂ |
| PEP83 | 117 | HαMsQGTFTSDK(γEγEC₁₆)SKYLDURAAQDFVNle(εN₃)WLLATKγE—NH₂ |
| PEP84 | 118 | HαMsVGTFTSDK(γEγEC₁₆)SKYLDURAAQDFVNle(εN₃)WL2DT—NH₂ |
| PEP85 | 119 | HαMsQGTFTSDK(γEγEC₁₆)SKYLDARAAQDFVNle(εN₃)WLLDT—NH₂ |
| PEP86 | 120 | HαMSQGTFTSDK(γEγEC₁₆)SKYLDARAAQDFVNle(εN₃)WLLDT—NH₂ |
| PEP87 | 121 | HsEGTFTSDK(γEγEC₁₆)SKYLDERAAQDFVNle(εN₃)WLLDT—NH₂ |
| PEP88 | 122 | HsQGTFTSD—K(γEγEC₁₆)SKYLDERAAQNle(εN₃)FVQWL2DT—NH₂ |
| PEP89 | 123 | HsQGTFTSD—K(γEγEC₁₆)SKYLDERAAQNle(εN₃)FVQWL2αMDT—NH₂ |
| PEP90 | 124 | HUQGTFTSDK(γEγEC₁₆)SKYLDURAAQDFVQWL2αMDTNle(εN₃)γE—NH₂ |
| PEP91 | 125 | HUQGTFTSDK(γEγEC₁₆)SKYLDURAAQDFVNle(εN₃)WL2αMDT—NH₂ |
| PEP92 | 126 | HUQGTFTSDK(γEγEC₁₆)SKYLDERAAQDFVNle(εN₃)WL2DGRG—NH₂ |
| PEP93 | 127 | HUQGTFTSDK(γEγEC₁₆)SKYLDURAAQDFVQWLLDTNle(εN₃)γE—NH₂ |
| PEP94 | 128 | HUQGTFTSDK(γEγEC₁₆)SKYLDURAAQDFVQWL2DTNle(εN₃)γE—NH₂ |
| PEP95 | 129 | HsQGTFTSDK(γEγEC₁₆)SKYLDERAAQDFVK(PEG₂PEG₂—C₅N₃)WL2DT—NH₂ |
| PEP96 | 130 | HsQGTFTSDK(γEγEC₁₆)SKYLDERAAQDFVK(PEG₂PEG₂—C₅N₃)WL2αMDT—NH₂ |
| PEP97 | 131 | HsQGTFTSDK(γEγEC₁₆)SKYLDERAAQDFVK(PEG₂—C₁₀N₃)WLLDT—NH₂ |
| PEP98 | 132 | HsQGTFTSDK(γEγEC₁₆)SKYLDERAAQDFVK(PEG₂—C₁₆N₃)WLLDT—NH₂ |
| PEP99 | 133 | HsQGTFTSDK(γEγEC₁₆)SKYLDERAAK(PEG₂PEG₂γE—C₁₀N₃)DFVQWLLDT—NH₂ |
| PEP100 | 134 | HsQGTFTSDK(γEγEC₁₆)SKYLDERAAK(PEG₂PEG₂γE—C₁₆N₃)DFVQWLLDT—NH₂ |
| PEP101 | 135 | HsQGTFTSDK(γEγEC₁₆)SKYLDERAAQDFVpraWLLDT—NH₂ |
| PEP102 | 136 | HsQGTFTSDK(γEγEC₁₆)SKYLDURAAQDFVQWLLDTKNle(εN₃)—NH₂ |
| PEP103 | 137 | HsQGTFTSDK(γEγEC₁₆)SKYLDURAAQDFVQWLLDTKK(PEG₂PEG₂—C₅N₃)—NH₂ |
| PEP104 | 138 | HsQGTFTSDK(γEγEC₁₆)SKYLDERAAQK(PEG₂PEG₂γEC₁₆N₃)FVQWLLDT—NH₂ |
| PEP105 | 139 | HUQGTFTSDK(γEγEC₁₆)SKYLDURAAQDFVK(PEG₂PEG₂—C₅N₃)WL2DT—NH₂ |
| PEP106 | 140 | HsQGTFTSDK(γEγEC₁₆)SKYLDERAAQDFVQWLLDTKNle(εN₃)—CONH₂ |
| PEP107 | 141 | HsQGTFTSDK(γEγEC₁₆)SKYLDERAAQDFVK(PEG₂PEG₂γEC₁₆N₃)WLLDT—NH₂ |
| PEP108 | 142 | HUQGTFTSDK(γEγEC₁₆)SKYLDURAAQDFVK(PEG₂—C₅N₃)WL2DT—NH₂ |
| PEP109 | 143 | HsQGTFTSDK(γEγEC₁₆)SKYLDERAAQDFVQWLLDTKK(PEG₂PEG₂—C₅N₃)CONH₂ |
| PEP110 | 144 | HsQGTFTSDK(γEγEC₁₆)SKYLDURAAQDFVNle(εN₃)WLLDTKγE—NH₂ |
| PEP111 | 145 | HUQGTFTSDK(γEγEC₁₆)SKYLDURAAQDFVK(PEG₂PEG₂—C₅N₃)WLLDTKγE—NH₂ |
| PEP112 | 146 | HUQGTFTSDK(γEγEC₁₆)SKYLDURAAQDFVK(PEG₂—C₅N₃)WLLDTKγE—NH₂ |
| PEP113 | 147 | HsQGTFTSDK(γEγEC₁₆)SKYLDSRAAQDFVNle(εN₃)WLLDT—NH₂ |
| PEP114 | 148 | HsQGTFTSDK(γEγEC₁₆)SKYLDERAAQDFVNle(εN₃)WLLγET—NH₂ |
| PEP115 | 149 | HsQGTFTSDK(γEγEC₁₆)SKYLDNKRAQDFVNle(εN₃)WLMQT—NH₂ |
| PEP116 | 150 | HUQGTFTSDK(γEγEC₁₆)SKYLDURAAQDFVNle(εN₃)WL2γET—NH₂ |
| PEP117 | 151 | HUQGTFVSDK(γEγEC₁₆)SKYLDURAAQDFVNle(εN₃)WL2ET—NH₂ |
| PEP118 | 152 | HUQGTFTSDK(γEγEC₁₆)SKYLDURAAQDFVNle(εN₃)WLLdTKγE—NH₂ |
| PEP119 | 153 | HUQGTFTSDK(γEγEC₁₆)SKYLDERAAQDFVNle(εN₃)αMWL2DT—NH₂ |
| PEP120 | 154 | HUQGTFTSDK(γEγEC₁₆)SKYLDERAAQDFVNle(εN₃)WL2αMDT—NH₂ |
| PEP121 | 155 | HUQGTFTSDK(γEγEC₁₆)SKYLDURAAQDFVNle(εN₃)αMWL2DT—NH₂ |
| PEP122 | 156 | HUQGTFTSDK(γEγEC₁₆)SKYLDURAAQDFVNle(εN₃)WL2αMDT—NH₂ |
| PEP123 | 157 | HsQGTFTSDK(γEγEC₁₆)SKYLDERAAQDFVQWLZαMDTKNle(εN₃)—NH₂ |
| PEP124 | 158 | HsQGTFTSDK(γEγEC₁₆)SKYLDERAAQDFVQαMWL2DTKNle(εN₃)—NH₂ |
| PEP125 | 159 | HsQGTFTSDK(γEγEC₁₆)SKYLDERAAQDFVQαMWL2DTKK(PEG₂PEG₂C₅N₃)—NH₂ |
| PEP126 | 160 | HsQGTFTSDK(γEγEC₁₆)SKYLDERAAQDFVQWL2αMDTKK(PEG₂PEG2C₅N₃)—NH₂ |
| PEP127 | 161 | HUQGTFTSDYSKYLDARAAK(PEG₂PEG₂γEC₁₈OH)DFVQWL2DTKNle(εN3)—NH₂ |
| PEP128 | 162 | HsQGTFTSDK(PEG₂PEG_{2γ}EC₁₈OH)SKYLDERAAQDFVQWLLDTKNle(εN₃)—NH₂ |
| PEP129 | 163 | HUQGTFTSDYSKYLDARAAK(PEG₂PEG₂γEC₁₈OH)DFVNle(εN₃)WL2αMDT—NH₂ |
| PEP130 | 164 | HUQGTFTSDYSKYLDARAAK(PEG₂PEG₂γEC₁₈OH)DFVK(PEG₂PEG₂C₁₆N₃)WL2αMDT—NH₂ |
| PEP131 | 165 | HUQGTFTSαMDK(γEγEC₁₆)SKYLDERAAQDFVNle(εN₃)WL2DT—NH₂ |
| PEP132 | 166 | HUQGTFTSαMDK(γEγEC₁₆)SKYLDERAAQDFVK(PEG₂PEG₂C₁₆N₃)WL2DT—NH₂ |

Table legend:
U = aminoisobutyric acid;
γE = γ-glutamic acid;
2 = L-methionine sulphone;
αMD = alpha-Methyl-L-Aspartic acid;
αML = alpha-Methyl-L-leucine;
αMF = alpha-Methyl-L-phenylalanine;
αMW = alpha-Methyl-L-tryptophan;
s = D-serine;
αMS = alpha-Methyl-L-Serine;
αMs = alpha-Methyl-D-Serine;
Nle(εN₃) = ε-azidonorleucine;
PEG₂ = 8-amino-3,6-dioxaoctanoic acid;
C₅N₃ = 5-azido pentinoic acid;
C₁₀N₃ = 10-azido-decanoic acid;
C₁₆N₃ = 16-azido-hexadecanoic acid;
C₁₈—OH = —CO—(CH₂)₁₆—COOH;
C₂₀—OH = —CO—(CH₂)₁₈—COOH;
NH₂ = C-terminal amide;
pra = propargyl glycine

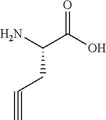

The peptide disclosed herein may have anywhere from at least about 1% (including at least about 1.5%, 2%, 5%, 7%, 10%, 20%, 30%, 40%, 50%, 60%, 75%, 100%, 125%, 150%, 175%) to about 200% or higher activity at the GLP-1 receptor relative to native GLP-1 and anywhere from 5 at least about 1% (including about 1.5%, 2%, 5%, 7%, 10%, 20%, 30%, 40%, 50%, 60%, 75%, 100%, 125%, 150%, 175%, 200%, 250%, 300%, 350%, 400%, 450%) to about 500% or higher activity at the glucagon receptor relative to native glucagon. In some embodiments, the peptides described herein exhibit no more than about 100%, 1000%, 10,000%, 100,000%, or 1,000,000% of the activity of native glucagon at the glucagon receptor. In some embodiments, the peptides described herein exhibit no more than about 100%, 1000%, 10,000%, 100,000%, or 1,000,000% of the activity of native GLP-1 at the GLP-1 receptor. In exemplary embodiments, a peptide may exhibit at least 10% of the activity of native glucagon at the glucagon receptor and at least 50% of the activity of native GLP-1 at the GLP-1 receptor, or at least 40% of the activity of native glucagon at the glucagon receptor and at least 40% of the activity of native GLP-1 at the GLP-1 receptor, or at least 60% of the activity of native glucagon at the glucagon receptor and at least 60% of the activity of native GLP-1 at the GLP-1 receptor.

Selectivity of a peptide for the glucagon receptor versus the GLP-1 receptor can be described as the relative ratio of glucagon/GLP-1 activity (the peptide analog's activity at the glucagon receptor relative to native glucagon, divided by the peptide's activity at the GLP-1 receptor relative to native GLP-1). For example, a peptide that exhibits 60% of the activity of native glucagon at the glucagon receptor and 60% of the activity of native GLP-1 at the GLP-1 receptor has a 1:1 ratio of glucagon/GLP-1 activity. Exemplary ratios of glucagon/GLP-1 activity include about 1:1, 1.5:1, 2:1, 3:1, 4:1, 5:1, 6:1, 7:1, 8:1, 9:1 or 10:1, or about 1:10, 1:9, 1:8, 1:7, 1:6, 1:5, 1:4, 1:3, 1:2, or 1:1.5. As an example, a glucagon/GLP-1 activity ratio of 10:1 indicates a 10-fold selectivity for the glucagon receptor versus the GLP-1 receptor. Similarly, a GLP-1/glucagon activity ratio of 10:1 indicates a 10-fold selectivity for the GLP-1 receptor versus the glucagon receptor.

### Insulin Molecules

The insulin molecule comprising the conjugates disclosed herein encompasses all salt and non-salt forms of the insulin molecule. It will be appreciated that the salt form may be anionic or cationic depending on the insulin molecule. The term “insulin” or “an insulin molecule” is intended to encompass both wild-type insulin and modified forms of insulin as long as they are bioactive (i.e., capable of causing a detectable reduction in glucose when administered in vivo). Wild-type insulin includes insulin from any species whether in purified, synthetic or recombinant form (e.g., human insulin, porcine insulin, bovine insulin, rabbit insulin, sheep insulin, etc.). A number of these are available commercially, e.g., from Sigma-Aldrich (St. Louis, Mo.). A variety of modified forms of insulin are known in the art (e.g. see Crotty and Reynolds, Pediatr. Emerg. Care. 23:903-905, 2007 and Gerich, Am. J Med. 113:308-16, 2002 and references cited therein). Modified forms of insulin (insulin analogs) may be chemically modified (e.g., by addition of a chemical moiety such as a PEG group or a fatty acyl chain as described below) and/or mutated (i.e., by addition, deletion or substitution of one or more amino acids).

In particular embodiments, an insulin molecule comprising the conjugate may be wild-type human recombinant insulin or may differ from a wild-type insulin by 1-10 (e.g., 1-9, 1-8, 1-7, 1-6, 1-5, 1-4, 1-3, 1-2, 2-9, 2-8, 2-7, 2-6, 2-5, 2-4, 2-3, 3-9, 3-8, 3-7, 3-6, 3-5, 3- 4, 4-9, 4-8, 4-7, 4-6, 4-5, 5-9, 5-8, 5-7, 5-6, 6-9, 6-8, 6-7, 7-9, 7-8, 8-9, 9, 8, 7, 6, 5, 4, 3, 2 or 1) amino acid substitutions, additions and/or deletions. In particular embodiments, an insulin molecule of the present disclosure will differ from wild-type insulin by amino acid substitutions only. In particular embodiments, an insulin molecule of the present disclosure will differ from wild-type insulin by amino acid additions only. In particular embodiments, an insulin molecule of the present disclosure will differ from wild-type insulin by both amino acid substitutions and additions. In particular embodiments, an insulin molecule of the present disclosure will differ from a wild-type insulin by both amino acid substitutions and deletions.

In particular embodiments, amino acid substitutions may be made on the basis of similarity in polarity, charge, solubility, hydrophobicity, hydrophilicity, and/or the amphipathic nature of the residues involved. In particular embodiments, a substitution may be conservative, that is, one amino acid is replaced with one of similar shape and charge. Conservative substitutions are well known in the art and typically include substitutions within the following groups: glycine, alanine; valine, isoleucine, leucine; aspartic acid, glutamic acid; asparagine, glutamine; serine, threonine; lysine, arginine; and tyrosine, phenylalanine. In particular embodiments, the hydrophobic index of amino acids may be considered in choosing suitable mutations. The importance of the hydrophobic amino acid index in conferring interactive biological function on a peptide is generally understood in the art. Alternatively, the substitution of like amino acids can be made effectively on the basis of hydrophilicity. The importance of hydrophilicity in conferring interactive biological function of a peptide or peptide is generally understood in the art. The use of the hydrophobic index or hydrophilicity in designing peptides is further discussed in U.S. Pat. No. 5,691,198.

The wild-type sequence of recombinant human insulin (A-chain and B-chain) is shown in Table 2 below. In various embodiments, an insulin molecule of the present disclosure is mutated at the B28 and/or B29 positions of the B-peptide sequence. For example, insulin lispro (HUMALOG®) is a rapid acting insulin mutant in which the penultimate lysine and proline residues on the C-terminal end of the B-peptide have been reversed (Lys^{B28}Pro^{B29}-human insulin) (SEQ ID NO:22). This modification blocks the formation of insulin multimers. Insulin aspart (NOVOLOG®) is another rapid acting insulin mutant in which proline at position B28 has been substituted with aspartic acid (AspB²⁸-human insulin) (SEQ ID NO:23). This mutant also prevents the formation of multimers. In some embodiments, mutation at positions B28 and/or B29 is accompanied by one or more mutations elsewhere in the insulin peptide. For example, insulin glulisine (APIDRA®) is yet another rapid acting insulin mutant in which aspartic acid at position B3 has been replaced by a lysine residue and lysine at position B29 has been replaced with a glutamic acid residue (Lys^{B3}Glu^{B29}-human insulin) (SEQ ID NO:24).

TABLE 2

| SEQ ID | | |
|---|---|---|
| NO: | Description | Sequence |
| 20 | Human insulin A chain | GIVEQCCTSICSLYQLENYCN |
| 21 | Human insulin B chain | FVNQHLCGSHLVEALYLVCGERGFFYTPKT |
| 22 | Insulin lispro B chain | FVNQHLCGSHLVEALYLVCGERGFFYTKPT |
| 23 | Insulin aspart B chain | FVNQHLCGSHLVEALYLVCGERGFFYTDKT |
| 24 | Insulin glusiline B | FVKQHLCGSHLVEALYLVCGERGFFYTPET |
| | chain | |
| 25 | Insulin glargine A chain | GIVEQCCTSICSLYQLENYCG |
| 26 | Insulin glargine B chain | FVNQHLCGSHLVEALYLVCGERGFFYTPKT |
| | | RR |

In various embodiments, the insulin molecule comprising the conjugate may have an isoelectric point that is shifted relative to human insulin. In some embodiments, the shift in isoelectric point is achieved by adding one or more arginine residues to the N-terminus of the insulin A-peptide and/or the C-terminus of the insulin B-peptide. Examples of such insulin peptides include Arg^{A0}-human insulin, Arg^{B31}Arg^{B32}-human insulin, Gly^{A21}Arg^{B31}Arg^{B32}-human insulin, Arg^{A0}Arg^{B31}Arg^{B32}-human insulin, and Arg^{A0}Gly^{A2}1Arg^{B31}Arg^{B32}-human insulin. By way of further example, insulin glargine (LANTUS®) is an exemplary long acting insulin mutant in which Asp^{A21} has been replaced by glycine (SEQ ID NO:25), and two arginine residues have been added to the C-terminus of the B-peptide (SEQ ID NO:26). The effect of these changes is to shift the isoelectric point, producing a solution that is completely soluble at pH 4. Thus, in some embodiments, an insulin molecule of the present disclosure comprises an A-peptide sequence wherein A21 is Gly and B-peptide sequence wherein B31 and B32 are Arg-Arg. It is to be understood that the present disclosure encompasses all single and multiple combinations of these mutations and any other mutations that are described herein (e.g., Gly^{A2}1-human insulin, Gly^{A2}1Arg^{B31}-human insulin, Arg^{B31}Arg^{B32}-human insulin, Arg^{B31}-human insulin).

In various embodiments, the insulin molecule comprising the conjugate may be truncated. For example, in particular embodiments, a B-peptide sequence of an insulin peptide of the present disclosure is missing B1, B2, B3, B26, B27, B28, B29 and/or B30. In particular embodiments, combinations of residues are missing from the B-peptide sequence of an insulin peptide of the present disclosure. For example, the B-peptide sequence may be missing residues B(1-2), B(1-3), B(29-30), B(28-30), B(27-30) and/or B(26-30). In some embodiments, these deletions and/or truncations apply to any of the aforementioned insulin molecules (e.g., without limitation to produce des(B30)-insulin lispro, des(B30)-insulin aspart, des(B30)-insulin glulisine, des(B30)-insulin glargine, etc.).

In some embodiments, the insulin molecule may comprise additional amino acid residues on the N- or C-terminus of the A or B-peptide sequences. In some embodiments, one or more amino acid residues are located at positions A0, A21, B0 and/or B31. In some embodiments, one or more amino acid residues are located at position A0. In some embodiments, one or more amino acid residues are located at position A21. In some embodiments, one or more amino acid residues are located at position B0. In some embodiments, one or more amino acid residues are located at position B31. In particular embodiments, an insulin molecule does not include any additional amino acid residues at positions A0, A21, B0 or B31.

In particular embodiments, the insulin molecule comprising the conjugate may be mutated such that one or more amidated amino acids are replaced with acidic forms. For example, asparagine may be replaced with aspartic acid or glutamic acid. Likewise, glutamine may be replaced with aspartic acid or glutamic acid. In particular, Asn^{A18}, Asn^{A21}, or Asn^{B3}, or any combination of those residues, may be replaced by aspartic acid or glutamic acid. Gln^{A15} or Gln^{B4}, or both, may be replaced by aspartic acid or glutamic acid. In particular embodiments, an insulin molecule has aspartic acid at position A21 or aspartic acid at position B3, or both.

One skilled in the art will recognize that it is possible to mutate yet other amino acids in the insulin molecule while retaining biological activity. For example, without limitation, the following modifications are also widely accepted in the art: replacement of the histidine residue of position B10 with aspartic acid (His^{B10}→Asp^{B10}); replacement of the phenylalanine residue at position B1 with aspartic acid (Phe^{B1}→Asp^{B1}); replacement of the threonine residue at position B30 with alanine (Thr^{B30}→Ala^{B30}); replacement of the tyrosine residue at position B26 with alanine (Tyr^{B26}→Ala^{B26}); and replacement of the serine residue at position B9 with aspartic acid (Ser^{B9}→Asp^{B9}).

In various embodiments, the insulin molecule comprising the conjugate may have a protracted profile of action. Thus, in particular embodiments, an insulin molecule of the present disclosure may be acylated with a fatty acid. That is, an amide bond is formed between an amino group on the insulin molecule and the carboxylic acid group of the fatty acid. The amino group may be the alpha-amino group of an N-terminal amino acid of the insulin molecule, or may be the epsilon-amino group of a lysine residue of the insulin molecule. An insulin molecule of the present disclosure may be acylated at one or more of the three amino groups that are present in wild-type human insulin or may be acylated on lysine residue that has been introduced into the wild-type human insulin sequence. In particular embodiments, an insulin molecule may be acylated at position B1. In particular embodiments, an insulin molecule may be acylated at position B29. In particular embodiments, the fatty acid is selected from myristic acid (C₁₄), pentadecylic acid (C₁₅), palmitic acid (C₁₆), heptadecylic acid (C₁₇) and stearic acid (C₁₈). For example, insulin detemir (LEVEMIR®) is a long acting insulin mutant in which Thr^{B30} has been deleted, and a C₁₄ fatty acid chain (myristic acid) has been attached to Lys^{B29}.

In some embodiments, the N-terminus of the A-peptide, the N-terminus of the B-peptide, the epsilon-amino group of Lys at position B29 or any other available amino group in an insulin molecule of the present disclosure is covalently linked to a fatty acid moiety of general formula:

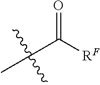

wherein R^{F} is hydrogen or a C₁₋₃₀ alkyl group. In some embodiments, R^{F} is a C₁₋₂₀ alkyl group, a C₃₋₁₉ alkyl group, a C₅₋₁₈ alkyl group, a C₆₋₁₇ alkyl group, a C₈₋₁₆ alkyl group, a C₁₀₋₁₅ alkyl group, or a C₁₂₋₁₄ alkyl group. In particular embodiments, the insulin molecule is conjugated to the moiety at the A1 position. In particular embodiments, the insulin molecule is conjugated to the moiety at the B1 position. In particular embodiments, the insulin molecule is conjugated to the moiety at the epsilon-amino group of Lys at position B29. In particular embodiments, position B28 of the insulin molecule is Lys and the epsilon-amino group of Lys^{B28} is conjugated to the fatty acid moiety. In particular embodiments, position B3 of the insulin molecule is Lys and the epsilon-amino group of Lys^{B3} is conjugated to the fatty acid moiety. In some embodiments, the fatty acid chain is 8-20 carbons long. In some embodiments, the fatty acid is octanoic acid (C8), nonanoic acid (C9), decanoic acid (C10), undecanoic acid (C11), dodecanoic acid (C12), or tridecanoic acid (C13). In particular embodiments, the fatty acid is myristic acid (C14), pentadecanoic acid (C15), palmitic acid (C16), heptadecanoic acid (C17), stearic acid (C18), nonadecanoic acid (C19), or arachidic acid (C20).

In various embodiments, the insulin molecule comprising the conjugate may have the three wild-type disulfide bridges (i.e., one between position 7 of the A-chain peptide and position 7 of the B-chain peptide, a second between position 20 of the A-chain peptide and position 19 of the B-chain peptide, and a third between positions 6 and 11 of the A-chain peptide). In particular embodiments, an insulin molecule is mutated such that the site of mutation is used as a conjugation point, and conjugation at the mutated site reduces binding to the insulin receptor (e.g., Lys^{A3}). In particular other embodiments, conjugation at an existing wild-type amino acid or terminus reduces binding to the insulin receptor (e.g., Gly^{A1}). In some embodiments, an insulin molecule is conjugated at position A4, A5, A8, A9, or B30. In particular embodiments, the conjugation at position A4, A5, A8, A9, or B30 takes place via a wild-type amino acid side chain (e.g., Glu^{A4}). In particular other embodiments, an insulin molecule is mutated at position A4, A5, A8, A9, or B30 to provide a site for conjugation (e.g., Lys^{A4}, Lys^{A5}, Lys^{A8}, Lys^{A9}, or Lys^{B3}30).

In particular embodiments, the insulin molecule comprising the conjugate may have an A chain sequence comprising a sequence of GIVEQCCXISICSLYQLENYCX₂ (SEQ ID NO: 27); and a B chain sequence comprising a sequence of X₃LCGX₄X₅LVEALYLVCG ERGFF (SEQ ID NO: 28) or X₈VNQX₃LCGX₄X₅LVEALYLVCGERGFFYTX₆ X₇(SEQ ID NO: 29) wherein

X₁ is selected from the group consisting of threonine and histidine;

X₂ is asparagine or glycine;

X₃ is selected from the group consisting of histidine and threonine;

X₄ is selected from the group consisting of alanine, glycine and serine;

X₅ is selected from the group consisting of histidine, aspartic acid, glutamic acid, homocysteic acid and cysteic acid;

X₆ is aspartate-lysine dipeptide, a lysine-proline dipeptide, or a proline-lysine dipeptide;

X₇ is threonine, alanine, or a threonine-arginine-arginine tripeptide; and

X₈ is selected from the group consisting of phenylalanine and desamino-phenylalanine. In particular embodiments, the insulin is conjugated to an alkyne-C₂-C₁₆ acyl, alkyne-PEGₙ wherein n is 1-50, or alkyne-(PEG₂)ₙ wherein n is 1-20 or the one Lysine is conjugated to an azide-C₂-C₁₆ acyl, azide-PEGₙ wherein n is 1-50, or azide-(PEG₂)ₙ wherein n is 1-20 with the proviso that one of R1, R2, or R3 is alkyne —C₂-C₁₆ acyl, azide-PEGₙ wherein n is 1-50, or azide-(PEG₂)ₙ wherein n is 1-20.

In particular embodiments, the A-chain may have the amino acid sequence set forth in SEQ ID NO:20 or SEQ ID NO:25 and the B-chain may have the amino acid sequence set forth in SEQ ID NO:21, SEQ ID NO:22, SEQ ID NO:23, or SEQ ID NO:24. In particular embodiments, the A-chain may have the amino acid sequence set forth in SEQ ID NO:25 and the B-chain may have the amino acid sequence set forth in SEQ ID NO:26. In particular embodiments, the insulin analog is a desB30 insulin analog, a des B29-B30 insulin analog, a des B28-B30 insulin analog, a des B27-B30 insulin analog or a des B26-B30 insulin analog.

In any one of the above embodiments, at least one amino group of the insulin molecule is conjugated to a linker comprising a terminal azide group or alkyne group. The amino group may be at the A1 position, the B1 position, or an epsilon amino group of a lysine residue on the A chain or the B chain. In particular embodiments, the lysine residue is at the B29 position of the B chain. In particular embodiments, the lysine residue is at the B28 position of the B chain, for example, insulin lispro has a lysine at the B28 position. In particular embodiments, the lysine residue is at the B3 position of the B chain, for example, insulin glulisine has a lysine at the B3 position. In particular embodiments, the epsilon amine of the lysine residue is converted to an azide group, which provides a norleucine with an epsilon azide group.

In particular embodiments, the insulin molecule comprising the conjugate has an A chain peptide sequence comprising a sequence of X₁I X₂E X₃CCX₄ X₅ X₆CS X₇ X₈ X₉LE X₁₀YC X₁₁ X₁₂ (SEQ ID NO:30); and a B chain peptide sequence comprising a sequence of X₁₃VX₁₄X₁₅HLCGSHLVEALX₁₆X₁₇VCGERGFX₁₈YTX₁₉X₂₀X₂₁X₂₂X₂₃X₂₄X₂₅X₂₆ (SEQ ID NO:31) wherein

X₁ is glycine (G) or lysine (K);

X₂ is valine (V), glycine (G), or lysine (K);

X₃ is glutamine (Q) or lysine (K);

X₄ is threonine (T) or histidine (H);

X₅ is serine (S) or lysine (K);

X₆ is isoleucine (I) or lysine;

X₇ is leucine (L) or lysine (K);

X₈ is tyrosine (Y) or lysine (K);

X₉ is glutamine (Q) or lysine (K);

X₁₀ is asparagine (N) or lysine (K);

X₁₁ is asparagine (N) or glycine (G);

X₁₂ is arginine (R), lysine (K) or absent;

X₁₃ is phenylalanine (F) or lysine (K);

X₁₄ is asparagine (N) or lysine (K);

X₁₅ is glutamine (Q) or lysine (K);

X₁₆ is tyrosine (Y) or lysine (K);

X₁₇ is leucine (L) or lysine (K);

X₁₈ is phenylalanine (F) or lysine (K);

X₁₉ is proline (P) or lysine (K);

X₂₀ is lysine (K) or proline (P);

X₂₁ is threonine (T) or absent;

X₂₂ is arginine (R) if X₂₁ is threonine (T), or absent;

X₂₃ is proline (P) if X₂₂ is arginine (R), or absent;

X₂₄ is arginine (R) if X₂₃ is proline (P), or absent;

X₂₅ is proline (P) if X₂₄ is arginine (R), or absent; and

X₂₆ is arginine (R) if X₂₅ is proline (P), or absent,

With the proviso that at least one of X₁, X₂, X₃, X₅, X₆, X₇, X₈, X₉, X₁₀, X₁₂, X₁₃, X₁₄, X₁₅, X₁₆, X₁₇, X₁₈, X₁₉, or X₂₀ is a lysine (K) wherein when X₂₀ is a lysine (K) then X₂₁ is absent or if X₂₁ is present then at least one of X₁, X₂, X₃, X₄, X₅, X₆, X₇, X₈, X₉, X₁₀, X₁₁, X₁₂, X₁₃, X₁₄, X₁₅, X₁₆, X₁₇ is lysine (K), or X₄ is histidine (H), or X₁₁ is glycine (G); or at least one of X₁₂ or X₂₂ is present. In particular aspects, if X₁, X₂, X₃, X₅, X₆, X₇, X₈, X₉, X₁₀, X₁₂, X₁₃, X₁₄, X₁₅, X₁₆, X₁₇, X₁₈, or X₁₉ is a lysine (K) then X₂₀ is not a lysine (K); and with the proviso that at least one Lysine (K) is conjugated to a linker having a terminal alkyne or azide group or the epsilon amine of at least one lysine residue is converted to an azide group, which provides a norleucine with an epsilon azide group.

In particular embodiments, the insulin molecule comprising the conjugate may be a desB30 human insulin analog, which may comprise an A chain peptide sequence comprising a sequence of X₁I X₂E X₃CCX₄ X₅ X₆CS X₇ X₈ X₉LE X₁₀YC X₁₁X₁₂ (SEQ ID NO:30); and a B chain peptide sequence comprising a sequence of X₁₃VX₁₄X₁₅HLCGSHLVEALX₁₆X₁₇VCGERGFX₁₈YTX₁₉X₂₀ (SEQ ID NO:32) wherein

X₁ is glycine (G) or lysine (K);

X₂ is valine (V), glycine (G), or lysine (K);

X₃ is glutamine (Q) or lysine (K);

X₄ is threonine (T) or histidine (H);

X₅ is serine (S) or lysine (K);

X₆ is isoleucine (I) or lysine;

X₇ is leucine (L) or lysine (K);

X₈ is tyrosine (Y) or lysine (K);

X₉ is glutamine (Q) or lysine (K);

X₁₀ is asparagine (N) or lysine (K);

X₁₁ is asparagine (N) or glycine (G);

X₁₂ is arginine (R), lysine (K) or absent;

X₁₃ is phenylalanine (F) or lysine (K);

X₁₄ is asparagine (N) or lysine (K);

X₁₅ is glutamine (Q) or lysine (K);

X₁₆ is tyrosine (Y) or lysine (K);

X₁₇ is leucine (L) or lysine (K);

X₁₈ is phenylalanine (F) or lysine (K);

X₁₉ is proline (P) or lysine (K);

X₂₀ is lysine (K) or proline (P);

With the proviso that at least one of X₁, X₂, X₃, X₅, X₆, X₇, X₈, X₉, X₁₀, X₁₂, X₁₃, X₁₄, X₁₅, X₁₆, X₁₇, X₁₈, X₁₉, or X₂₀ is a lysine (K); and with the proviso that at least one Lysine (K) is conjugated to a linker having a terminal alkyne or azide group or the epsilon amine of at least one lysine residue is converted to an azide group, which provides a norleucine with an epsilon azide group.

In particular embodiments, the insulin molecule comprising the conjugate may comprise an A chain peptide sequence comprising a sequence of X₁I X₂E X₃CCX₄ X₅ X₆CS X₇ X₈ X₉LE X₁₀YC X₁₁ X₁₂ (SEQ ID NO:30); and a B chain peptide sequence comprising a sequence of X₁₃VX₁₄X₁₅HLCGSHLVEALX₁₆X₁₇VCGERGFX₁₈YTX₁₉X₂₀TRPRPR (SEQ ID NO:33) wherein

X₁ is glycine (G) or lysine (K);

X₂ is valine (V), glycine (G), or lysine (K);

X₃ is glutamine (Q) or lysine (K);

X₄ is threonine (T) or histidine (H);

X₅ is serine (S) or lysine (K);

X₆ is isoleucine (I) or lysine;

X₇ is leucine (L) or lysine (K);

X₈ is tyrosine (Y) or lysine (K);

X₉ is glutamine (Q) or lysine (K);

X₁₀ is asparagine (N) or lysine (K);

X₁₁ is asparagine (N) or glycine (G);

X₁₂ is arginine (R), lysine (K) or absent;

X₁₃ is phenylalanine (F) or lysine (K);

X₁₄ is asparagine (N) or lysine (K);

X₁₅ is glutamine (Q) or lysine (K);

X₁₆ is tyrosine (Y) or lysine (K);

X₁₇ is leucine (L) or lysine (K);

X₁₈ is phenylalanine (F) or lysine (K);

X¹⁹ is proline (P) or lysine (K);

X₂₀ is lysine (K) or proline (P);

with the proviso that at least one of X₁, X₂, X₃, X₅, X₆, X₇, X₈, X₉, X₁₀, X₁₂, X₁₃, X₁₄, X₁₅, X₁₆, X₁₇, X₁₈, X₁₉, or X₂₀ is a lysine (K); and with the proviso that at least one Lysine (K) is conjugated to a linker having a terminal alkyne or azide group or the epsilon amine of at least one lysine residue is converted to an azide group, which provides a norleucine with an epsilon azide group.

In particular embodiments, the insulin molecule comprising the conjugate may have an A chain peptide sequence comprising a sequence of X₁I X₂E X₃CCX₄ X₅ X₆CS X₇ X₈ X₉LE X₁₀YC X₁₁ X₁₂ (SEQ ID NO: 30); and a B chain peptide sequence comprising a sequence of X₁₃VX₁₄X₁₅HLCGSHLVEALX₁₆X₁₇VCGERGFX₁₈YTX₁₉X₂₀TRPR (SEQ ID NO: 34) wherein

X₁ is glycine (G) or lysine (K);

X₂ is valine (V), glycine (G), or lysine (K);

X₃ is glutamine (Q) or lysine (K);

X₄ is threonine (T) or histidine (H);

X₅ is seine (S) or lysine (K);

X₆ is isoleucine (I) or lysine;

X₇ is leucine (L) or lysine (K);

X₈ is tyrosine (Y) or lysine (K);

X₉ is glutamine (Q) or lysine (K);

X₁₀ is asparagine (N) or lysine (K);

X₁₁ is asparagine (N) or glycine (G);

X₁₂ is arginine (R), lysine (K) or absent;

X₁₃ is phenylalanine (F) or lysine (K);

X₁₄ is asparagine (N) or lysine (K);

X₁₅ is glutamine (Q) or lysine (K);

X₁₆ is tyrosine (Y) or lysine (K);

X₁₇ is leucine (L) or lysine (K);

X₁₈ is phenylalanine (F) or lysine (K);

X¹⁹ is proline (P) or lysine (K);

X₂₀ is lysine (K) or proline (P);

with the proviso that at least one of X₁, X₂, X₃, X₅, X₆, X₇, X₈, X₉, X₁₀, X₁₂, X₁₃, X₁₄, X₁₅, X₁₆, X₁₇, X₁₈, X₁₉, or X₂₀ is a lysine (K); and with the proviso that at least one Lysine (K) is conjugated to a linker having a terminal alkyne or azide group or the epsilon amine of at least one lysine residue is converted to an azide group, which provides a norleucine with an epsilon azide group.

The following structure represents human insulin (e.g., recombinant human insulin (RHI)) or any insulin analog disclosed herein

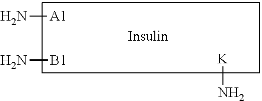

wherein the insulin is a heterodimer in which the cysteine residues a positions 6 and 11 of the A chain are linked in a disulfide bond, the cysteine residues at position 7 of the A chain and position 7 of the B chain are linked in a disulfide bond, and the cysteine residues at position 20 of the A chain and 19 of the B chain are linked in a disulfide bond and wherein A1 is the amino acid at position 1 of the A chain peptide, B1 is the amino acid at position 1 of the B chain peptide, and K is a lysine, which may be in any position in the insulin or insulin analog. In particular embodiments, the lysine is at position B29, B28, or B3. In particular embodiments, the lysine is at position B29 and is represented by the structure

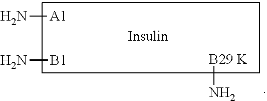

In particular embodiments, the lysine is at position B28 and is represented by the structure.

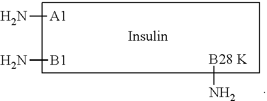

In particular embodiments, the lysine is at position B3 and is represented by the structure

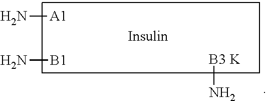

Unless otherwise indicated, the term “insulin” is used to indicate the insulin is a human insulin in which the A chain has amino acid sequence GIVEQCCTSICSLYQLENYCN (SEQ ID NO:20) and the B chain has amino acid sequence FVNQHLCGSHLVEALYLVCGERGFFYTPKT (SEQ ID NO:21).

The following structure represents a single-chain insulin analog (SCI) in which the C-terminal amino acid of the B chain is covalently linked the N-terminal amino acid of the A chain by a non-peptide linker or a peptide linker comprising three to 35 amino acids

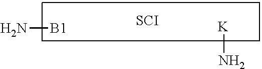

wherein the cysteine residues a positions 6 and 11 of the A chain are linked in a disulfide bond, the cysteine residues at position 7 of the A chain and position 7 of the B chain are linked in a disulfide bond, and the cysteine residues at position 20 of the A chain and 19 of the B chain are linked in a disulfide bond and wherein B1 is the amino acid at position 1 of the B chain peptide and K is a lysine, which may be in any position in the insulin or insulin analog. In particular embodiments, the lysine is at position B29, B28, or B3. In particular embodiments, the lysine is at position B29 and is represented by the structure

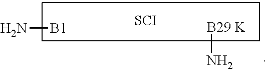

In particular embodiments, the lysine is at position B28 and is represented by the structure

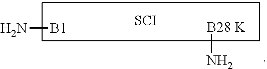

In particular embodiments, the lysine is at position B3 and is represented by the structure

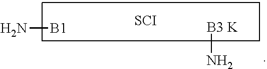

Unless otherwise indicated, the term “SCI” is used to indicate the single-chain insulin an A chain having an amino acid sequence GIVEQCCTSICSLYQLENYCN (SEQ ID NO:20) and a B chain having the amino acid sequence FVNQHLCGSHLVEALYLVCGERGFFYTPKT (SEQ ID NO:21).

Single-chain insulins and analogs thereof have been disclosed in U.S. Pat. No. 8,940,860, which is incorporated herein by reference in its entirety and U.S. Publications US 20150374795 and US 20150299285, which are incorporated herein by reference in their entirety.

### Linkers

The linker conjugated to the insulin or the insulin may be any non-peptide linker comprising a terminal azide group or a terminal alkyne group with the proviso that when the incretin is conjugated to a linker comprising an azide group then the insulin is conjugated to a linker comprising an alkyne group and when the incretin is conjugated to a linker comprising an azide group then the insulin is conjugated to a linker comprising an alkyne group.

The non-peptide linker may comprise a C₁-C₅₀ hydrocarbon chain or substituted hydrocarbon chain, a PEGₙ wherein n is 1-50, a (PEG₂)ₙ wherein n is 1-50, a (PEG₂)ₙ-(γGlu)ₚ-Cₙ wherein each n is independently 1-50 and p is 1 or 2, a (PEG₂)ₙ-Cₙ wherein each n is independently is 1-50, a (PEG)ₙ(PEG)ₙ wherein each n is independently 1-50, a (PEG)ₙ(PEG)ₙ(PEG)ₙ wherein each n is independently 1-50a PEGₙ-(Lys-(γGlu)ₚ-Cₙ)—Cₙ wherein each n is independently 1-50 and p is 1 or 2, and a C₅-Lys(γE-Cₙ)-PEGₙ wherein each n is independently 1-50.

In particular embodiments, the linker may be a propargyl-polyethylene glycol (PEG) linker having the general formula

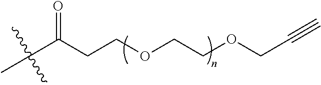

wherein n is 0-50 and the wavy line indicates the bond between the linker and an amino group on the insulin molecule or analog or an amino group on the incretin peptide. In particular embodiments n is 1-25. In particular embodiments, the linker may be selected from the group

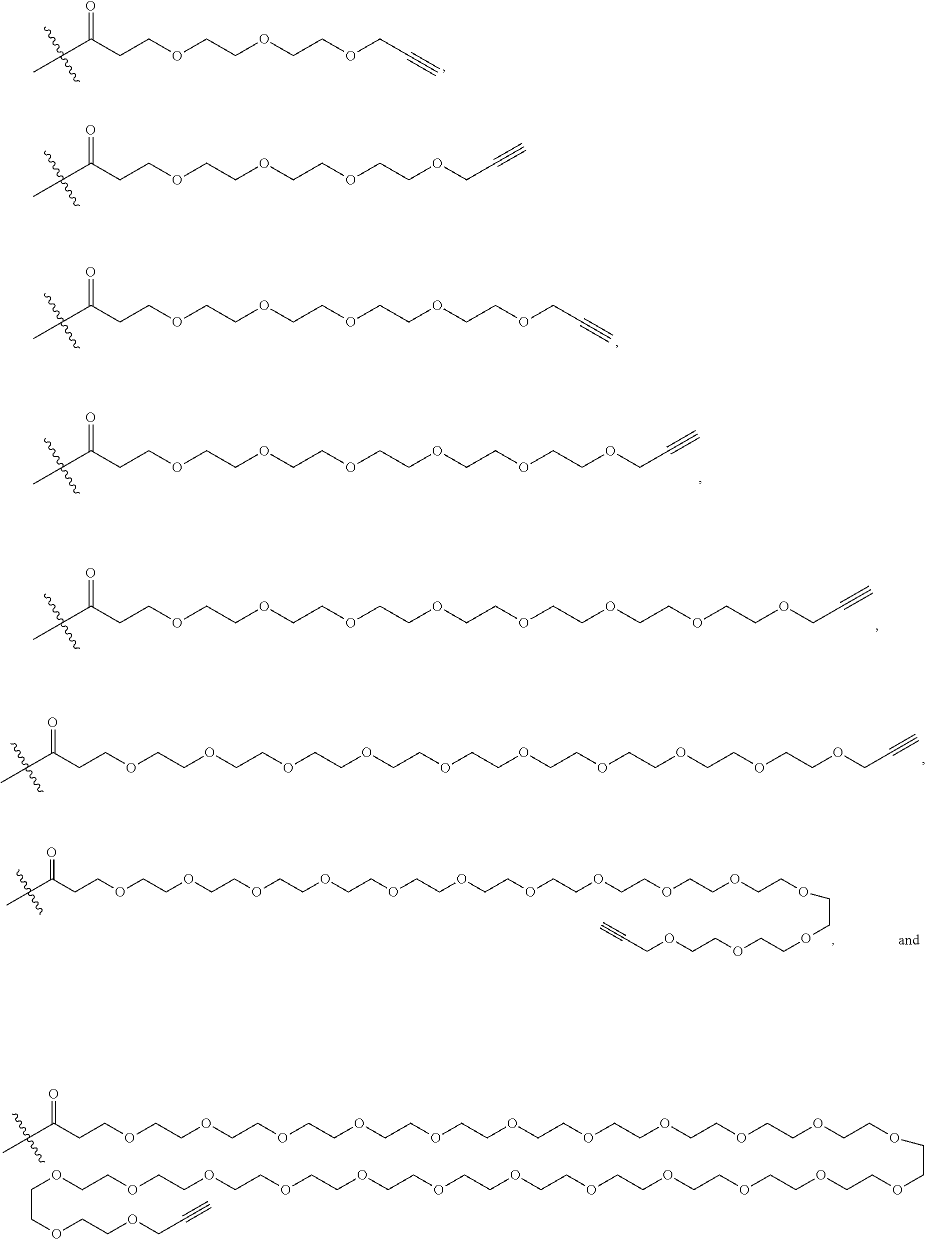

In particular embodiments, the linker may be a propargyl-C₅-(polyethylene glycol 2)ₙ ((PEG₂)ₙ) linker having the general formula

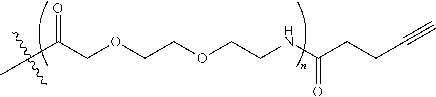

wherein n is 1-50 and the wavy line indicates the bond between the linker and an amino group on the insulin molecule or analog or an amino group on the incretin peptide. In particular embodiments n is 1-5. In particular embodiments, the linker may be selected from the group

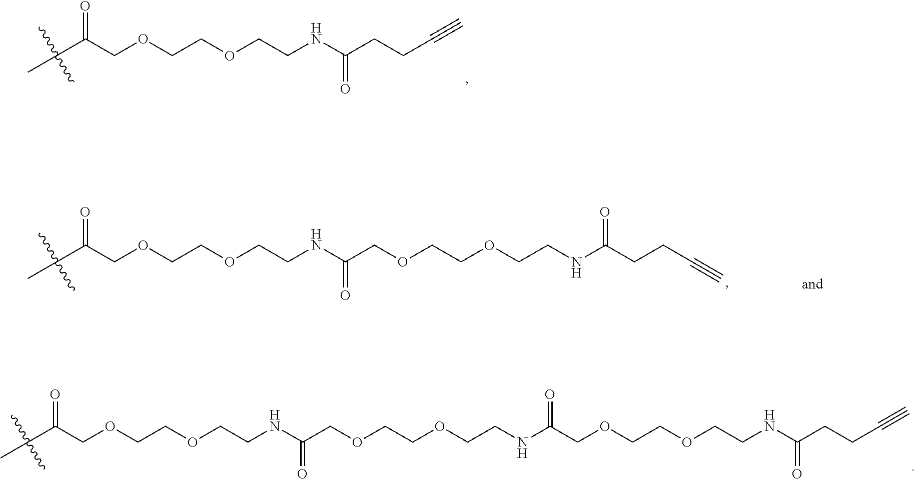 [SMILES: *C(=O)COCCOCCNC(=O)CCC#C ; *C(=O)COCCOCCNC(=O)COCCOCCNC(=O)CCC#C ; *C(=O)COCCOCCNC(=O)COCCOCCNC(=O)COCCOCCNC(=O)CCC#C]

In particular embodiments, the linker may be a propargyl-C₅-Lys(γE-Cₙ)-PEGₙ linker having the general formula

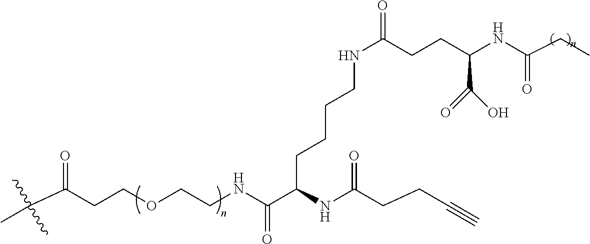

wherein each n is independently 1-50 and the wavy line indicates the bond between the linker and an amino group on the insulin molecule or analog or an amino group on the incretin peptide. In particular embodiments, the linker has the formula

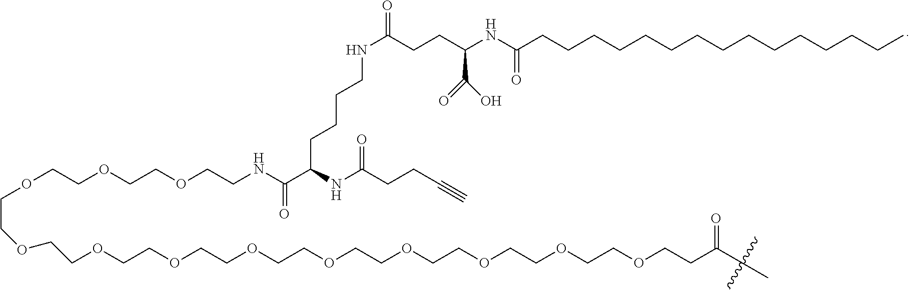 [SMILES: *C(=O)CCOCCOCCOCCOCCOCCOCCOCCOCCOCCOCCOCCOCCNC(=O)[C@@H](CCCCNC(=O)CC[C@@H](NC(=O)CCCCCCCCCCCCCCC)C(=O)O)NC(=O)CCC#C]

In particular embodiments, the linker may be a propargyl-(PEGₙ)(PEGₙ) linker having the general formula

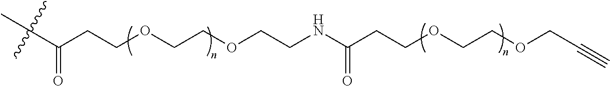

wherein each n is independently 0-50 and the wavy line indicates the bond between the linker and an amino group on the insulin molecule or analog or an amino group on the incretin peptide. In particular embodiments, the linker is selected from

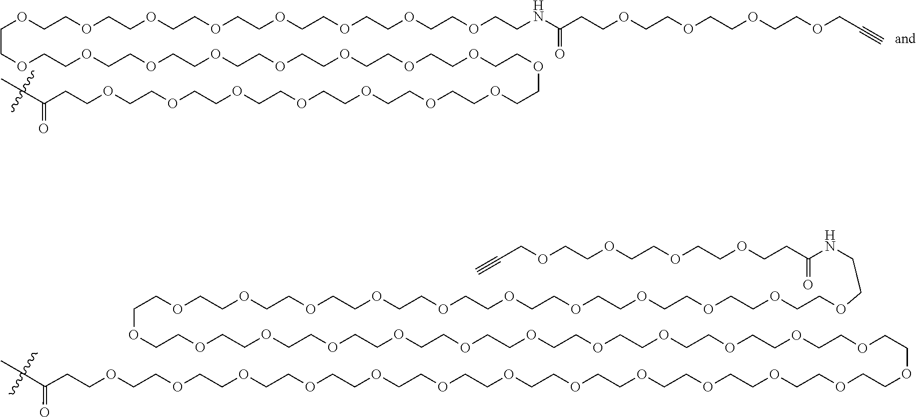 [SMILES: *C(=O)CCOCCOCCOCCOCCOCCOCCOCCOCCOCCOCCOCCOCCOCCOCCOCCOCCOCCOCCOCCOCCOCCOCCOCCOCCNC(=O)CCOCCOCCOCCOCC#C ; *C(=O)CCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCNC(=O)CCOCCOCCOCCOCC#C]

In particular embodiments, the linker may be an propargyl-(PEGₙ)(PEGₙ)(PEGₙ) linker having the general formula

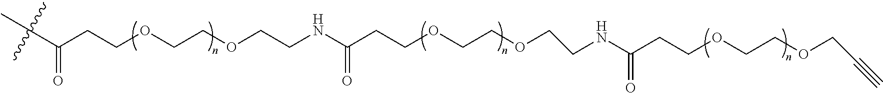

wherein each n is independently 0-50 and the wavy line indicates the bond between the linker and an amino group on the insulin molecule or analog or an amino group on the incretin peptide. In particular embodiments, the linker may be

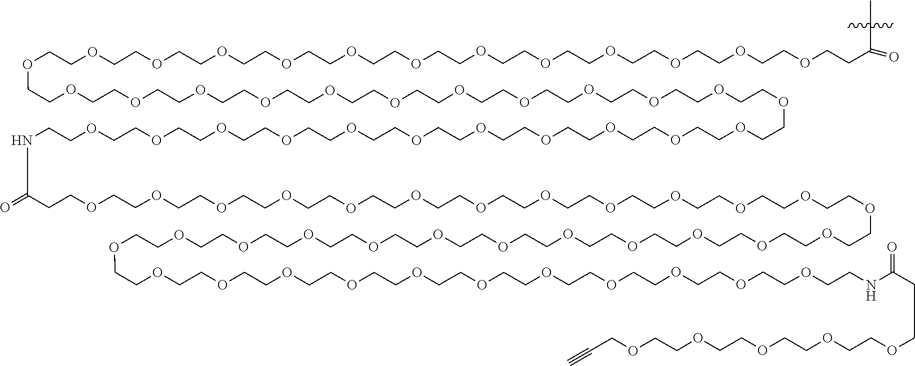 [SMILES: *C(=O)CCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCNC(=O)CCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCNC(=O)CCOCCOCCOCCOCCOCC#C]

In particular embodiments, the linker may be a propargyl-Cₙ having the general formula

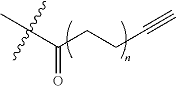

wherein n is 1-50 and the wavy line indicates the bond between the linker and an amino group on the insulin molecule or analog or an amino group on the incretin peptide. In particular embodiments, the linker is

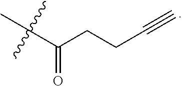 [SMILES: *C(=O)CCC#C]

In particular embodiments, the linker may be a BCN-PEG₄(endo) linker having the general formula

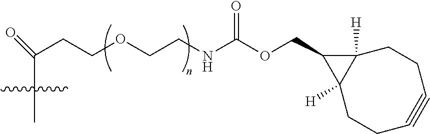

wherein n is 1-50 and the wavy line indicates the bond between the linker and an amino group on the insulin molecule or analog or an amino group on the incretin peptide. In particular embodiments, the linker is

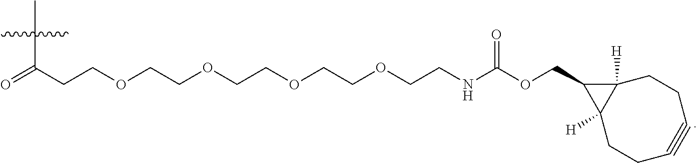 [SMILES: *C(=O)CCOCCOCCOCCOCCNC(=O)OC[C@@H]1[C@@H]2CCC#CCC[C@@H]21]

In particular embodiments, the linker may be a propargyl-phenylacetate linker having the general formula

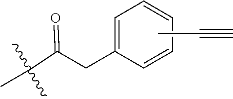

wherein the wavy line indicates the bond between the linker and an amino group on the insulin molecule or analog or an amino group on the incretin peptide. In particular embodiments, the linker is selected from

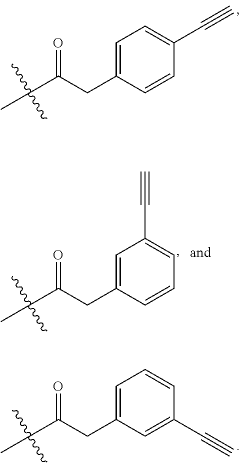 [SMILES: *C(=O)Cc1ccc(C#C)cc1 ; *C(=O)Cc1cccc(C#C)c1 ; *C(=O)Cc1cccc(C#C)c1]

In particular embodiments, the linker may be an azido-polyethylene glycol (PEG) linker having the general formula

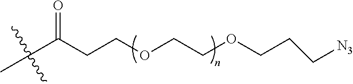

wherein n is 0-50 and the wavy line indicates the bond between the linker and an amino group on the insulin molecule or analog or an amino group on the incretin peptide. In particular embodiments n is 1-25. In particular embodiments, the linker may be selected from the group

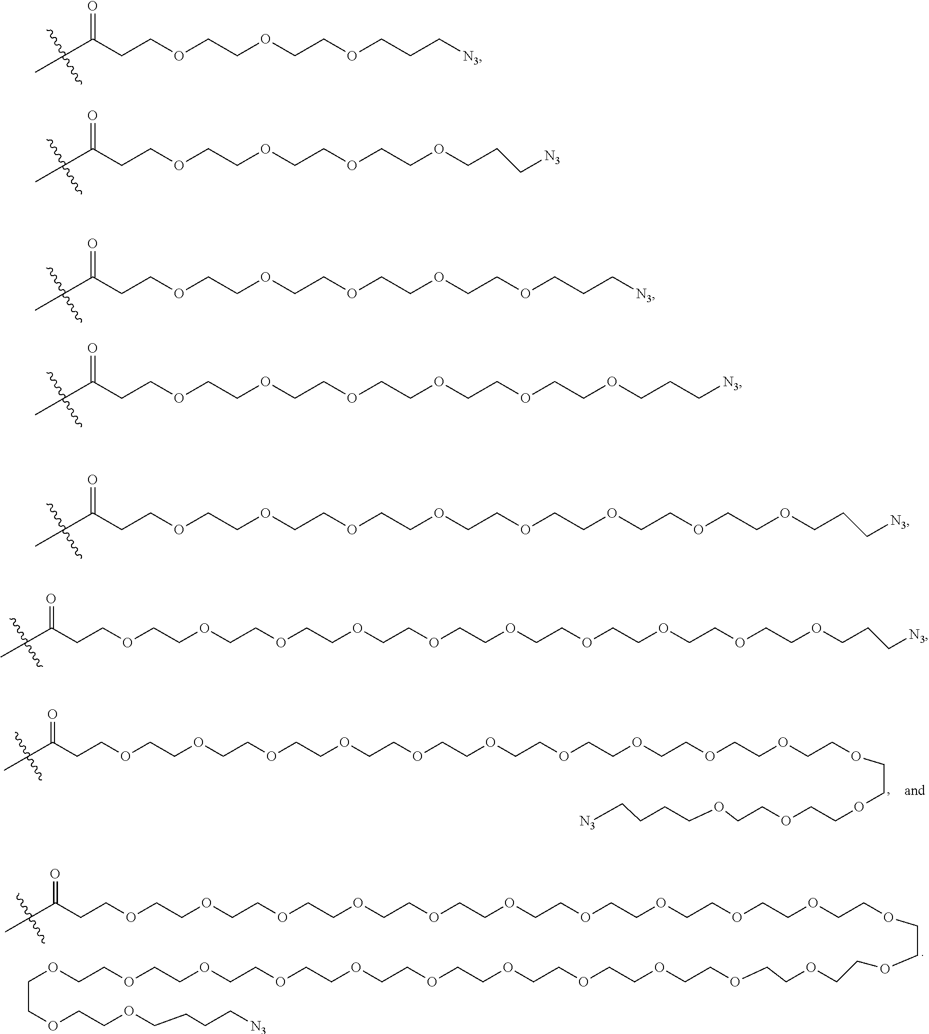

In particular embodiments, the linker may be an azido-C₅-Lys(γE-Cₙ)-PEGₙ linker having the general formula

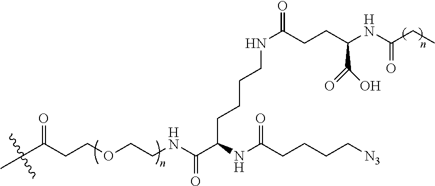

wherein each n is independently 1-50 and the wavy line indicates the bond between the linker and an amino group on the insulin molecule or analog or an amino group on the incretin peptide. In particular embodiments, the linker has the formula

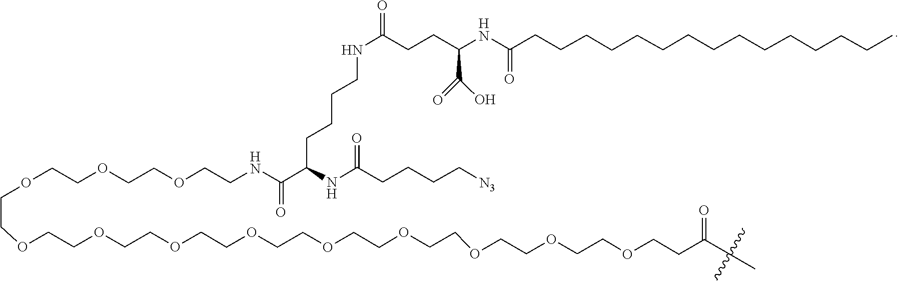 [SMILES: *C(=O)CCOCCOCCOCCOCCOCCOCCOCCOCCOCCOCCOCCOCCNC(=O)[C@@H](CCCCNC(=O)CC[C@@H](NC(=O)CCCCCCCCCCCCCCC)C(=O)O)NC(=O)CCCCN=[N+]=[N-]]

In particular embodiments, the linker may be an azido-(PEGₙ)(PEGₙ) linker having the general formula

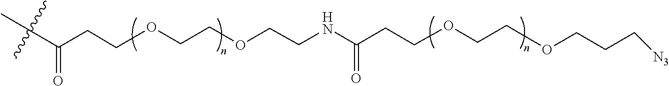

wherein each n is independently 0-50 and the wavy line indicates the bond between the linker and an amino group on the insulin molecule or analog or an amino group on the incretin peptide. In particular embodiments, the linker is selected from

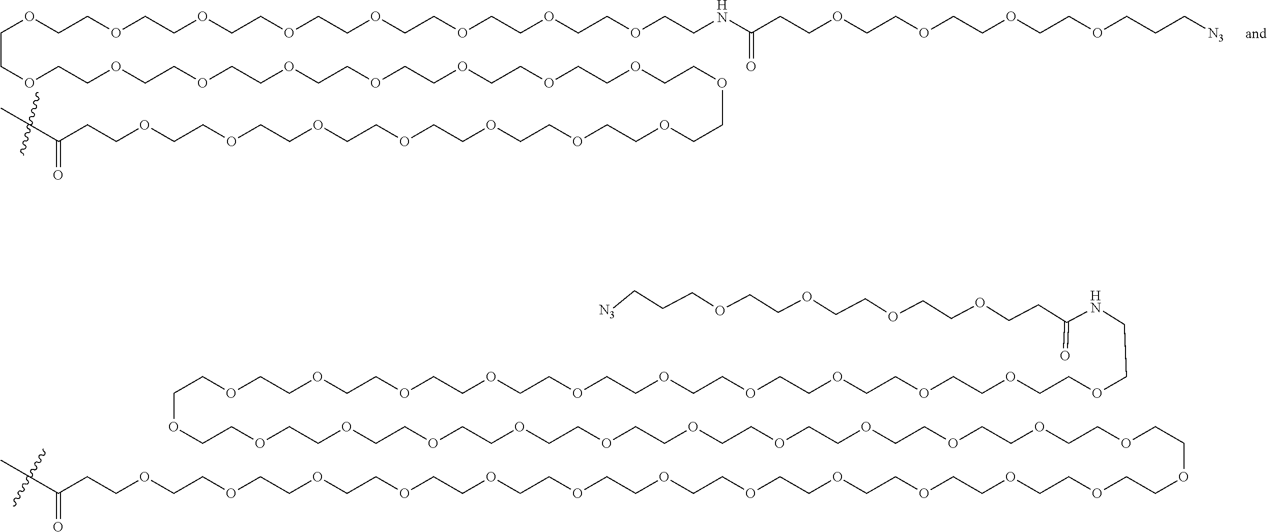 [SMILES: *C(=O)CCOCCOCCOCCOCCOCCOCCOCCOCCOCCOCCOCCOCCOCCOCCOCCOCCOCCOCCOCCOCCOCCOCCOCCOCCNC(=O)CCOCCOCCOCCOCCCN=[N+]=[N-] ; *C(=O)CCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCNC(=O)CCOCCOCCOCCOCCCN=[N+]=[N-]]

In particular embodiments, the linker may be an azido-(PEGₙ)(PEGₙ)(PEGₙ) linker having the general formula

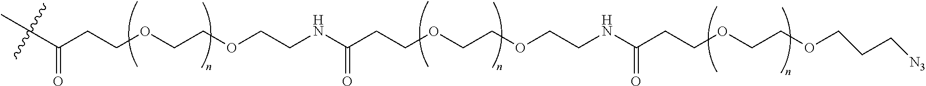

wherein each n is independently 0-50 and the wavy line indicates the bond between the linker and an amino group on the insulin molecule or analog or an amino group on the incretin peptide. In particular embodiments, the linker may be

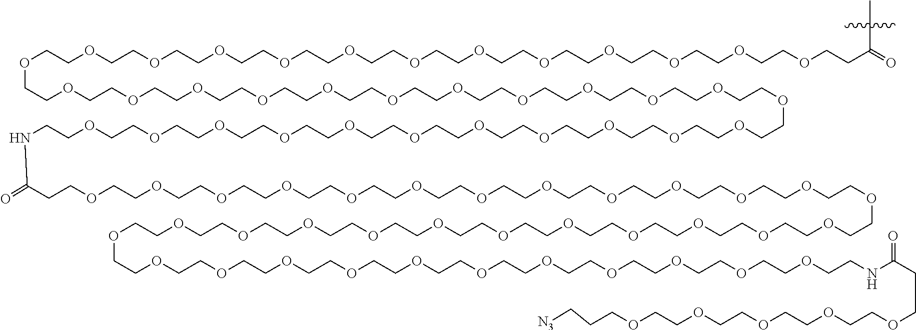 [SMILES: *C(=O)CCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCNC(=O)CCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCNC(=O)CCOCCOCCOCCOCCOCCCN=[N+]=[N-]]

In particular embodiments, the linker may be an azido-Cₙ having the general formula

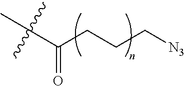

wherein n is 1-50 and the wavy line indicates the bond between the linker and an amino group on the insulin molecule or analog or an amino group on the incretin peptide. In particular embodiments, the linker is

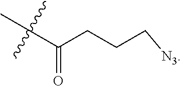 [SMILES: *C(=O)CCCN=[N+]=[N-]]

In particular embodiments, the linker may be an azido-phenylacetate linker having the general formula

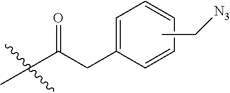

wherein the wavy line indicates the bond between the linker and an amino group on the insulin molecule or analog or an amino group on the incretin peptide. In particular embodiments, the linker is selected from

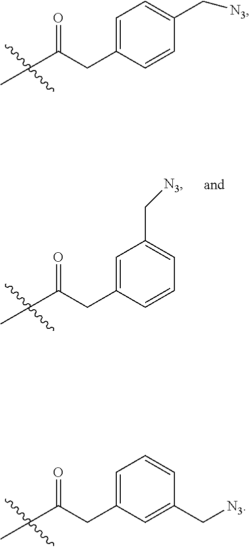 [SMILES: *C(=O)Cc1ccc(CN=[N+]=[N-])cc1 ; *C(=O)Cc1cccc(CN=[N+]=[N-])c1 ; *C(=O)Cc1cccc(CN=[N+]=[N-])c1]

In particular embodiments, the linker may be an azido-Cₙ-(PEG₂)ₙ linker having the general formula

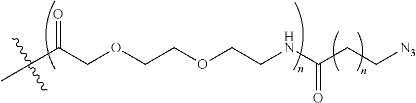

wherein each n is independently 1-10 and the wavy line indicates the bond between the linker and an amino group on the insulin molecule or analog or an amino group on the incretin peptide.

In particular embodiments, Cₙ is C₅ and the linker may be selected from the group

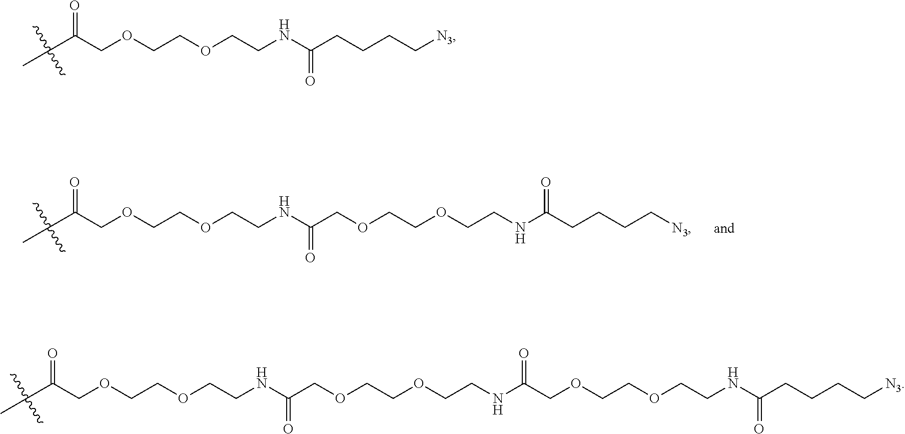 [SMILES: *C(=O)COCCOCCNC(=O)CCCCN=[N+]=[N-] ; *C(=O)COCCOCCNC(=O)COCCOCCNC(=O)CCCCN=[N+]=[N-] ; *C(=O)COCCOCCNC(=O)COCCOCCNC(=O)COCCOCCNC(=O)CCCCN=[N+]=[N-]]

In particular embodiments, Cₙ is C₁₀ and the linker may be selected from the group

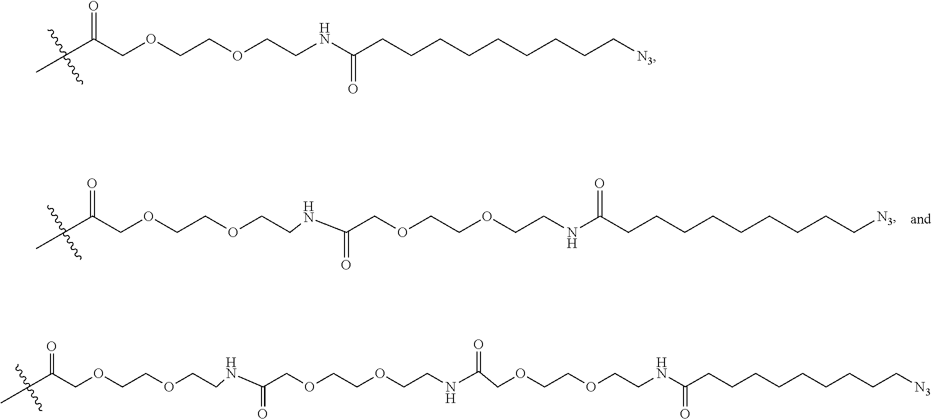 [SMILES: *C(=O)COCCOCCNC(=O)CCCCCCCCCN=[N+]=[N-] ; *C(=O)COCCOCCNC(=O)COCCOCCNC(=O)CCCCCCCCCN=[N+]=[N-] ; *C(=O)COCCOCCNC(=O)COCCOCCNC(=O)COCCOCCNC(=O)CCCCCCCCCN=[N+]=[N-]]

In particular embodiments, Cₙ is C₁₆ and the linker may be selected from the group

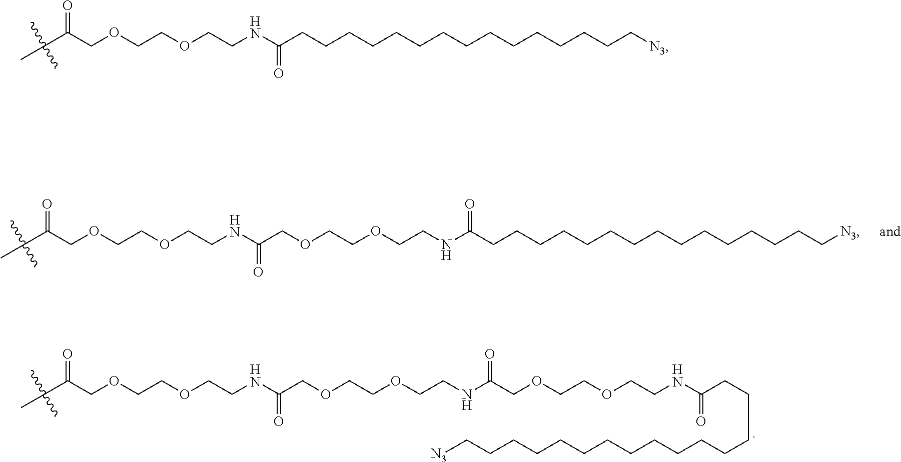 [SMILES: *C(=O)COCCOCCNC(=O)CCCCCCCCCCCCCCCN=[N+]=[N-] ; *C(=O)COCCOCCNC(=O)COCCOCCNC(=O)CCCCCCCCCCCCCCCN=[N+]=[N-] ; *C(=O)COCCOCCNC(=O)COCCOCCNC(=O)COCCOCCNC(=O)CCCCCCCCCCCCCCCN=[N+]=[N-]]

In particular embodiments, the linker may be an azido-Cₙ-γE-(PEG₂)ₙ-linker having the general formula

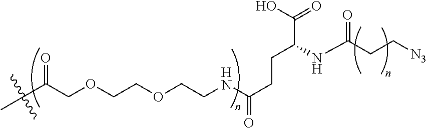

wherein each n is independently 1-10 and the wavy line indicates the bond between the linker and an amino group on the insulin molecule or analog or an amino group on the incretin peptide. In particular embodiments, Cₙ is C₁₆ and the linker may be selected from the group

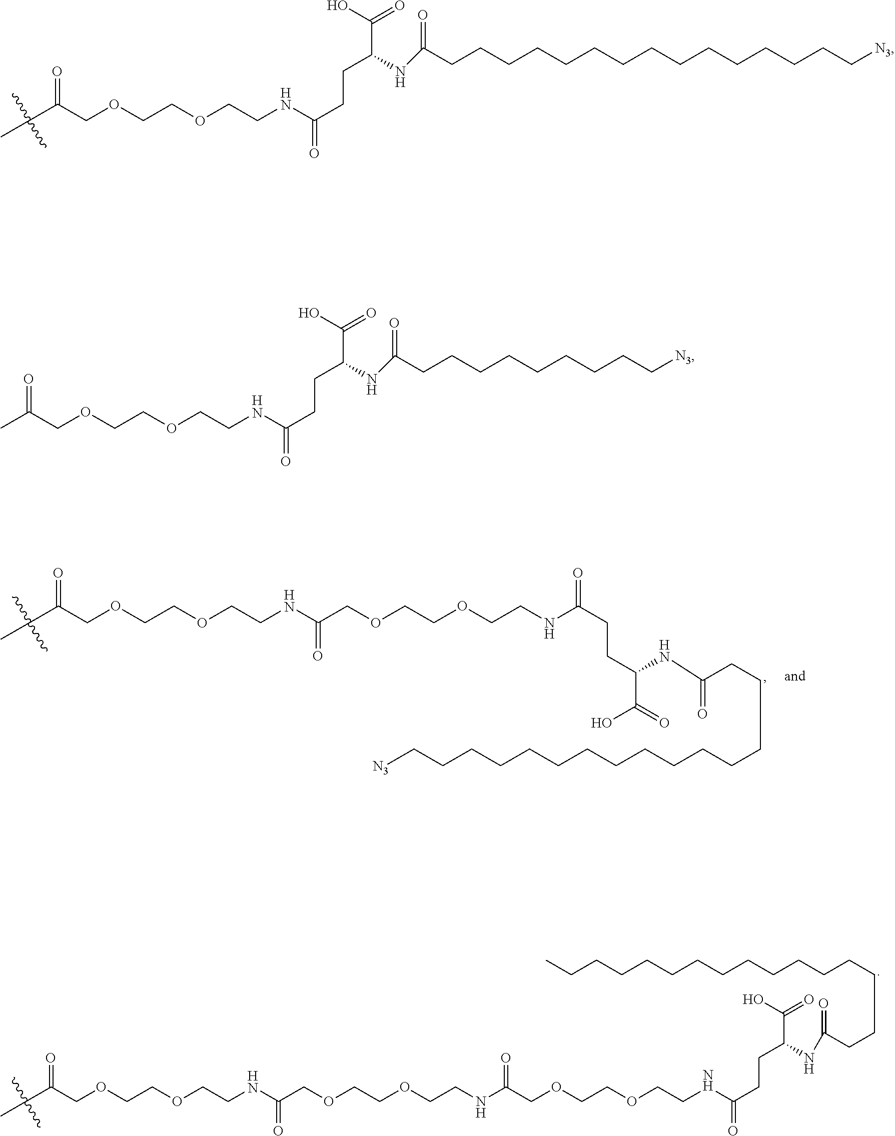 [SMILES: *C(=O)COCCOCCNC(=O)CC[C@@H](NC(=O)CCCCCCCCCCCCCCCN=[N+]=[N-])C(=O)O ; CC(=O)COCCOCCNC(=O)CC[C@@H](NC(=O)CCCCCCCCCN=[N+]=[N-])C(=O)O ; *C(=O)COCCOCCNC(=O)COCCOCCNC(=O)CC[C@H](NC(=O)CCCCCCCCCCCCCCCN=[N+]=[N-])C(=O)O ; *C(=O)COCCOCCNC(=O)COCCOCCNC(=O)COCCOCCNC(=O)CC[C@@H](NC(=O)CCCCCCCCCCCCCCCC)C(=O)O]

In particular embodiments, Cₙ is C₁₀ and the linker may be selected from the group

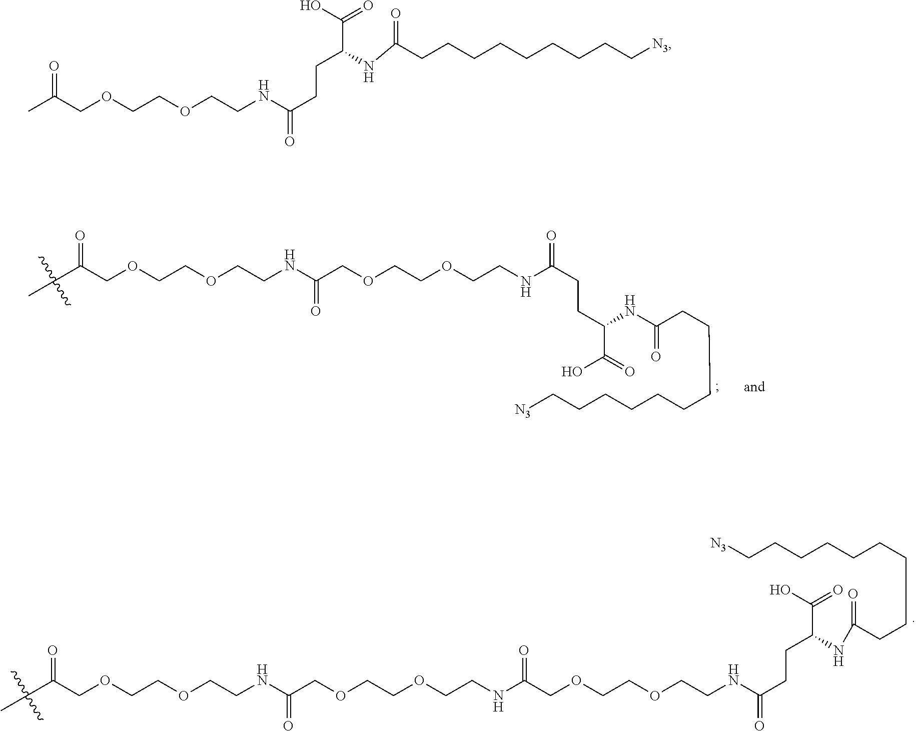 [SMILES: CC(=O)COCCOCCNC(=O)CC[C@@H](NC(=O)CCCCCCCCCN=[N+]=[N-])C(=O)O ; *C(=O)COCCOCCNC(=O)COCCOCCNC(=O)CC[C@H](NC(=O)CCCCCCCCCN=[N+]=[N-])C(=O)O ; *C(=O)COCCOCCNC(=O)COCCOCCNC(=O)COCCOCCNC(=O)CC[C@@H](NC(=O)CCCCCCCCCN=[N+]=[N-])C(=O)O]

In particular embodiments, the linker may be azido-norleucine having the structure

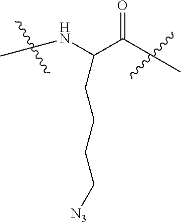 [SMILES: *NC(CCCCN=[N+]=[N-])C(*)=O]

wherein the wavy lines indicate the bonds between the azido-norleucine and adjacent amino acids in either the insulin molecule or the incretin peptide.

In particular embodiments, the linking moiety conjugating the insulin molecule to the incretin peptide comprises the formula

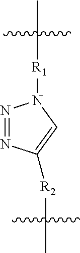

wherein R₁ and R₂ independently comprise a C₁-C₅₀ hydrocarbon chain or substituted hydrocarbon chain, a PEGₙ wherein n is 1-50, a (PEG₂)ₙ wherein n is 1-50, a (PEG₂)ₙ-(γGlu)ₚ-Cₙ wherein each n is independently 1-50 and p is 1 or 2, a (PEG₂)ₙ-Cₙ wherein each n is independently is 1-50, a (PEG)ₙ(PEG)ₙ wherein each n is independently 1-50, a PEGₙ-(Lys-(γGlu)ₚ-Cₙ)—Cₙ wherein each n is independently 1-50 and p is 1 or 2, and a C₅-Lys(γE-Cₙ)-PEGₙ wherein each n is independently 1-50, and wherein the bond between the linking moiety and the insulin molecule and the incretin peptide are indicated by the wavy lines with the proviso that if the bond adjacent to R₁ is to insulin then the bond adjacent to R₂ is to the incretin peptide or that if the bond adjacent to R₁ is to the incretin peptide then the bond adjacent to R₂ is to insulin.

Exemplary linking moieties are shown in Table 3. For structures 1-25, the wavy line on the left indicates the bond between alpha and beta carbons of Norleucine (Nle) or Lysine (Lys or K) and the wavy line on the right indicates the bond between the CO and an amino group of an amino acid. For 26, the wavy line on the left is a bound between an amino acid having an alkyne group and the wavy line on the right is a bound between an amino acid having an azide group.

TABLE 3

| # | Name | Structure |
|---|---|---|
| 1 | Nle(εN₃) x C₅ | 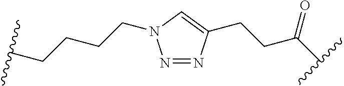 [SMILES: *CCCCn1cc(CCC(*)=O)nn1] |
| 2 | Nle(εN₃ x PEG₃ | 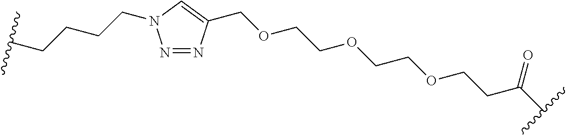 [SMILES: *CCCCn1cc(COCCOCCOCCC(*)=O)nn1] |
| 3 | Nle(εN₃) x PEG₄ | 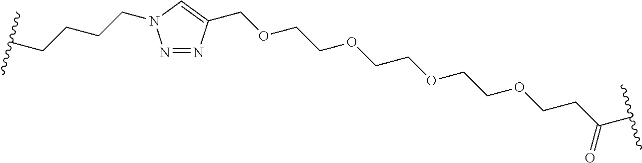 [SMILES: *CCCCn1cc(COCCOCCOCCOCCC(*)=O)nn1] |
| 4 | Nle(εN₃) x PEG₅ | 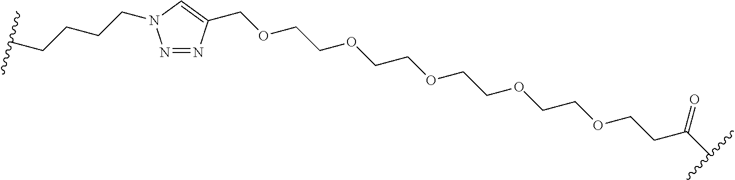 [SMILES: *CCCCn1cc(COCCOCCOCCOCCOCCC(*)=O)nn1] |
| 5 | Nle(εN₃) x PEG₆ | 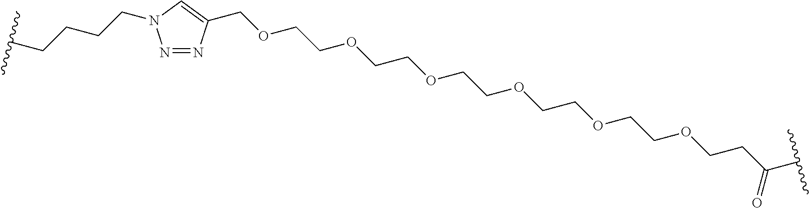 [SMILES: *CCCCn1cc(COCCOCCOCCOCCOCCOCCC(*)=O)nn1] |
| 6 | Nle(εN₃) x PEG₈ | 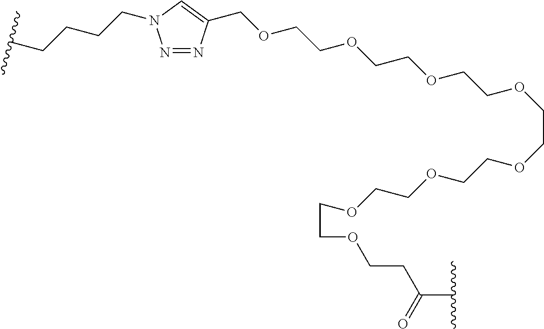 [SMILES: *CCCCn1cc(COCCOCCOCCOCCOCCOCCOCCOCCC(*)=O)nn1] |
| 7 | Nle(εN₃) x PEG₁₀ | 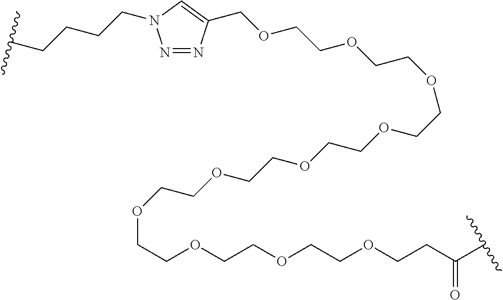 [SMILES: *CCCCn1cc(COCCOCCOCCOCCOCCOCCOCCOCCOCCOCCC(*)=O)nn1] |
| 8 | Nle(εN₃) x PEG₁₃ | 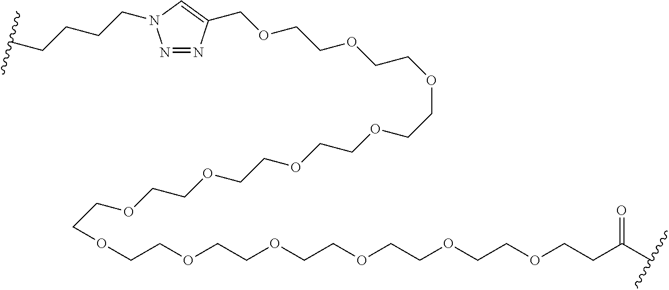 [SMILES: *CCCCn1cc(COCCOCCOCCOCCOCCOCCOCCOCCOCCOCCOCCOCCOCCC(*)=O)nn1] |
| 9 | Nle(εN₃) x PEG₁₄ | 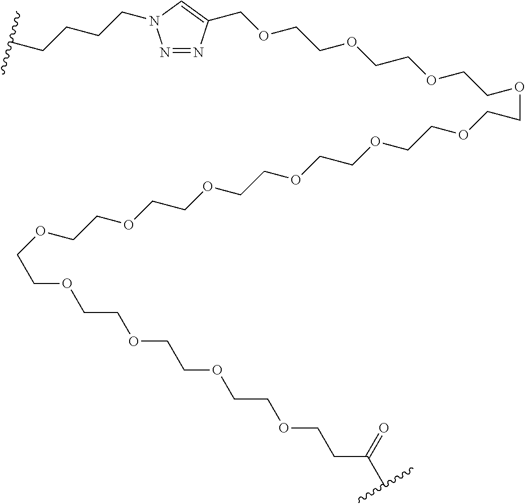 [SMILES: *CCCCn1cc(COCCOCCOCCOCCOCCOCCOCCOCCOCCOCCOCCOCCOCCOCCC(*)=O)nn1] |
| 10 | Nle(εN₃) x PEG₂₅ | 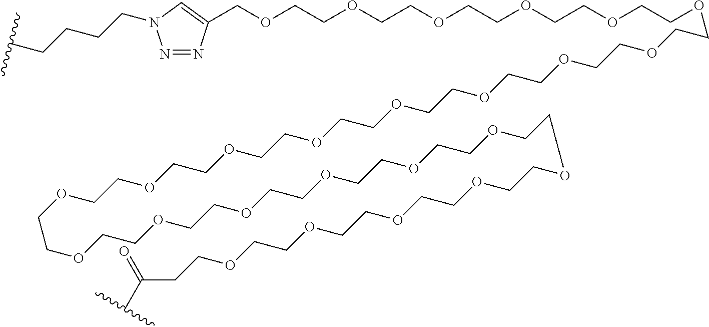 [SMILES: *CCCCn1cc(COCCOCCOCCOCCOCCOCCOCCOCCOCCOCCOCCOCCOCCOCCOCCOCCOCCOCCOCCOCCOCCOCCOCCOCCOCCC(*)=O)nn1] |
| 11 | Nle(εN₃) x (PEG₂)C₅ | 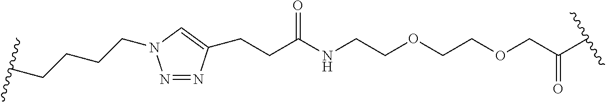 [SMILES: *CCCCn1cc(CCC(=O)NCCOCCOCC(*)=O)nn1] |
| 12 | Nle(εN₃) x (PEG₂)₃ C₅ | 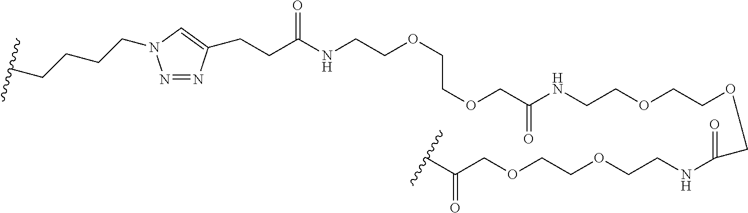 [SMILES: *CCCCn1cc(CCC(=O)NCCOCCOCC(=O)NCCOCCOCC(=O)NCCOCCOCC(*)=O)nn1] |
| 13 | Nle(εN₃) x (PEG₂₄)(PEG₄) | 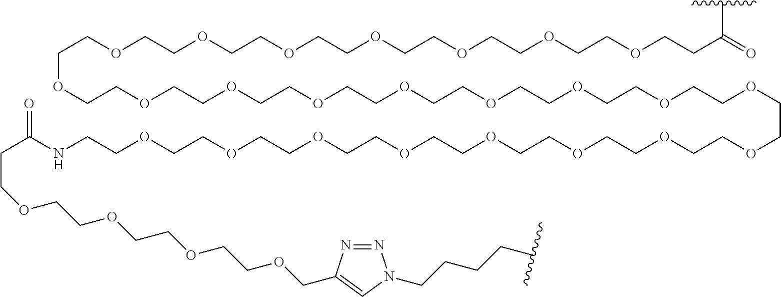 [SMILES: *CCCCn1cc(COCCOCCOCCOCCC(=O)NCCOCCOCCOCCOCCOCCOCCOCCOCCOCCOCCOCCOCCOCCOCCOCCOCCOCCOCCOCCOCCOCCOCCOCCOCCC(*)=O)nn1] |
| 14 | Nle(εN₃) x (PEG₃₆)(PEG₄) | 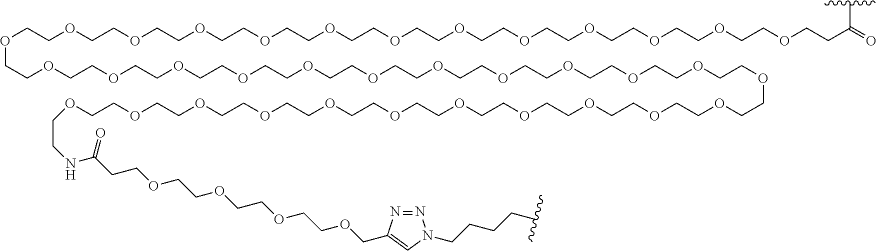 [SMILES: *CCCCn1cc(COCCOCCOCCOCCC(=O)NCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCC(*)=O)nn1] |
| 15 | Nle(εN₃) x PEG₁₂(LysγE-C₁₆) C₅ | 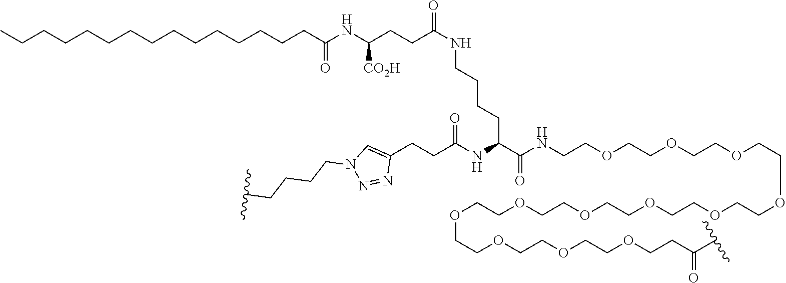 [SMILES: *CCCCn1cc(CCC(=O)N[C@@H](CCCCNC(=O)CC[C@H](NC(=O)CCCCCCCCCCCCCCC)C(=O)O)C(=O)NCCOCCOCCOCCOCCOCCOCCOCCOCCOCCOCCOCCOCCC(*)=O)nn1] |
| 16 | Nle(εN₃) x BCN | 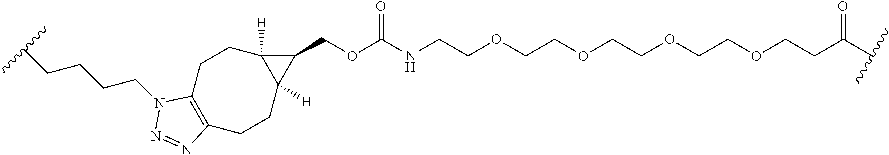 [SMILES: *CCCCn1nnc2c1CC[C@@H]1[C@H](CC2)[C@@H]1COC(=O)NCCOCCOCCOCCOCCC(*)=O] |
| 17 | Lys(PEG₂)C₅N₃ x C₅) | 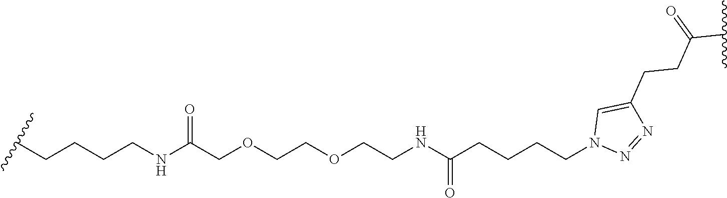 [SMILES: *CCCCNC(=O)COCCOCCNC(=O)CCCCn1cc(CCC(*)=O)nn1] |
| 18 | Lys(PEG₂)(PEG₂)C₅N₃ x C₅) | 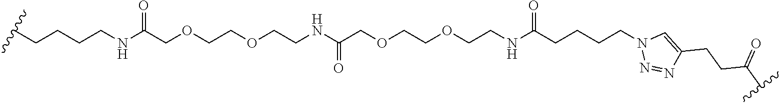 [SMILES: *CCCCNC(=O)COCCOCCNC(=O)COCCOCCNC(=O)CCCCn1cc(CCC(*)=O)nn1] |
| 19 | Lys(PEG₂)₂γEC₁₆N₃ x C₅) | 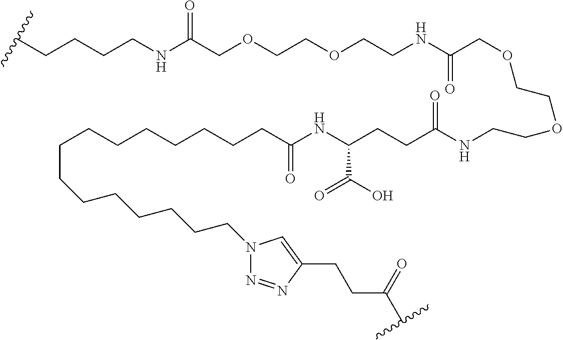 [SMILES: *CCCCNC(=O)COCCOCCNC(=O)COCCOCCNC(=O)CC[C@@H](NC(=O)CCCCCCCCCCCCCCCn1cc(CCC(*)=O)nn1)C(=O)O] |
| 20 | Nle(εN₃) x (PEG₃₆)₂(PEG₅) | 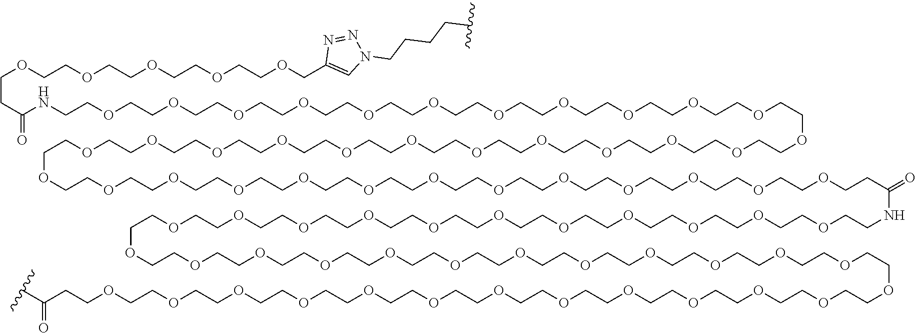 [SMILES: *CCCCn1cc(COCCOCCOCCOCCOCCC(=O)NCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCC(=O)NCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCC(*)=O)nn1] |
| 21 | Nle(εN₃) x propargyl-C5- Lys(γE-C₁₆)—PEG₂₄ | 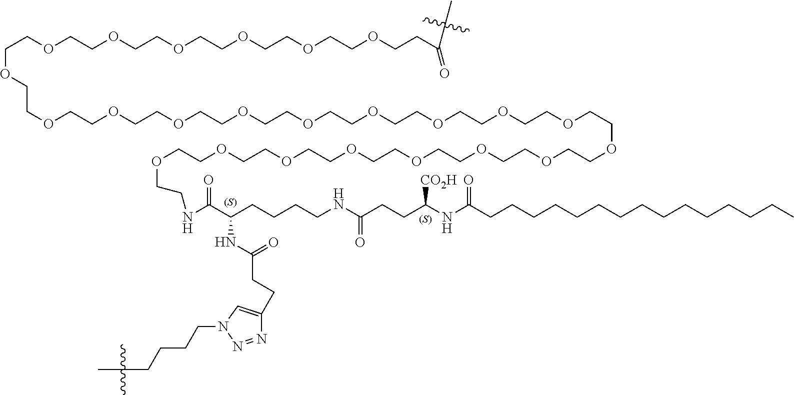 |
| 22 | Nle(εN₃) x propargyl-C5- Lys(γE-C₁₂)—PEG₁₂ | 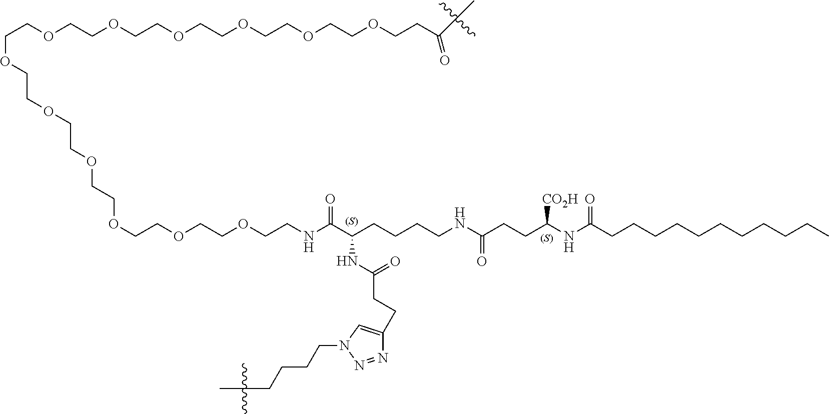 |
| 23 | Nle(εN₃) x propargyl-C5- Lys(γE-C₁₆)—PEG₄ | 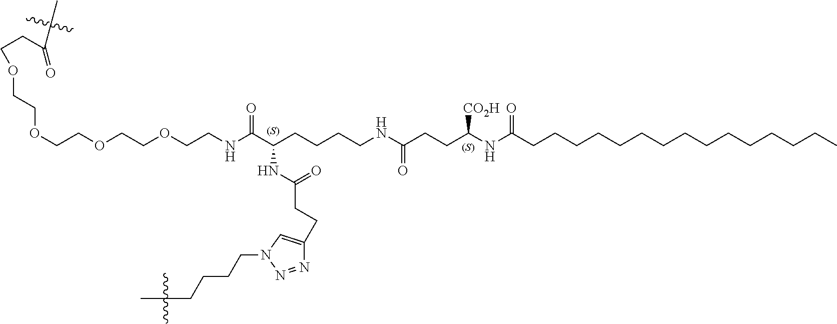 |
| 24 | Nle(εN₃) x propargyl-C5- Lys(γE-C₁₈—OH)—PEG₄ | 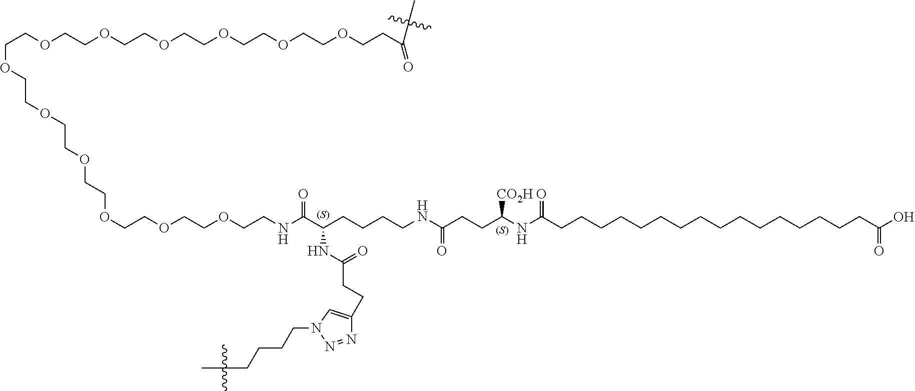 |
| 25 | C8 hydrocarbon linker | 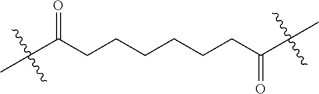 [SMILES: *C(=O)CCCCCCC(*)=O] |
| 26 | pra x azide | 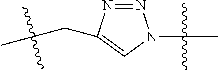 [SMILES: *Cc1cn(*)nn1] |

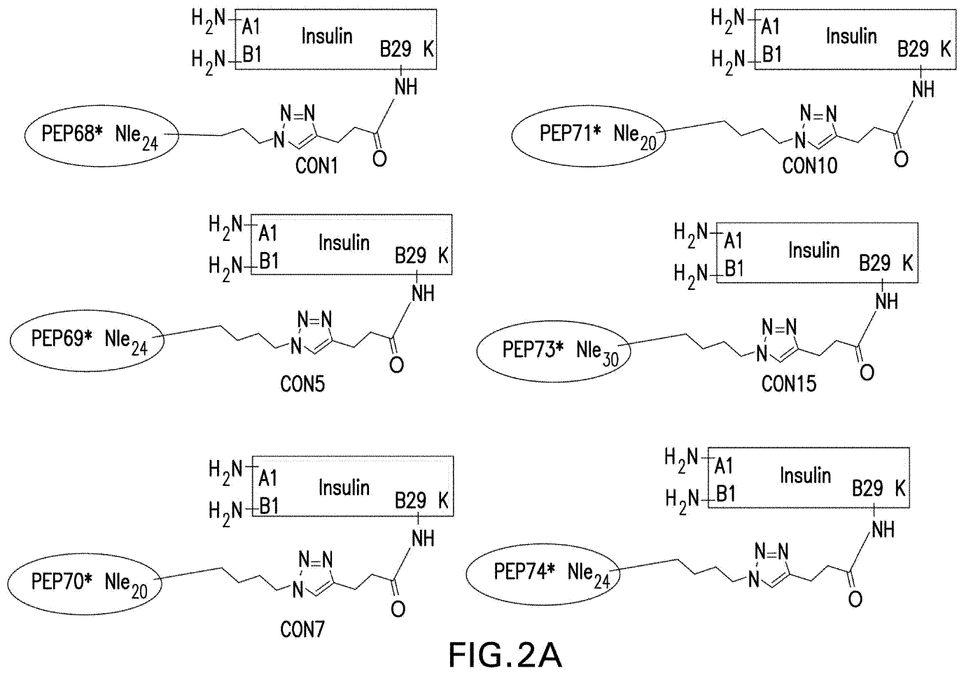

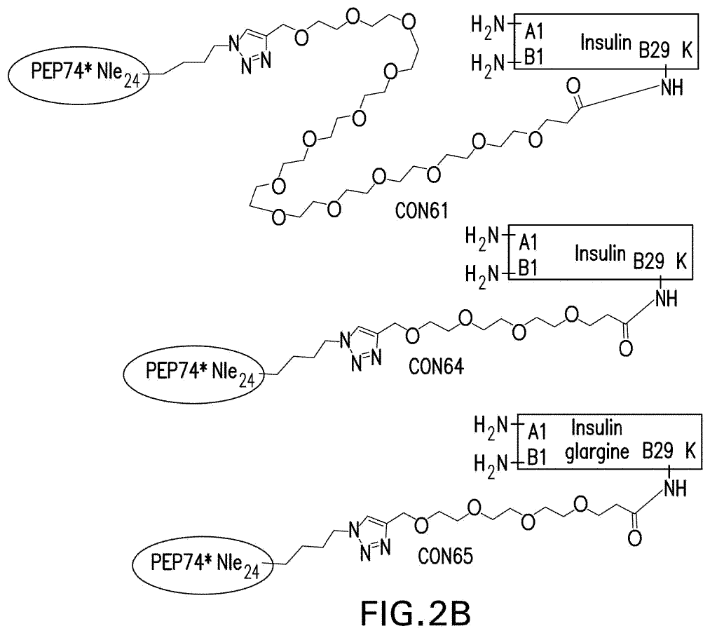

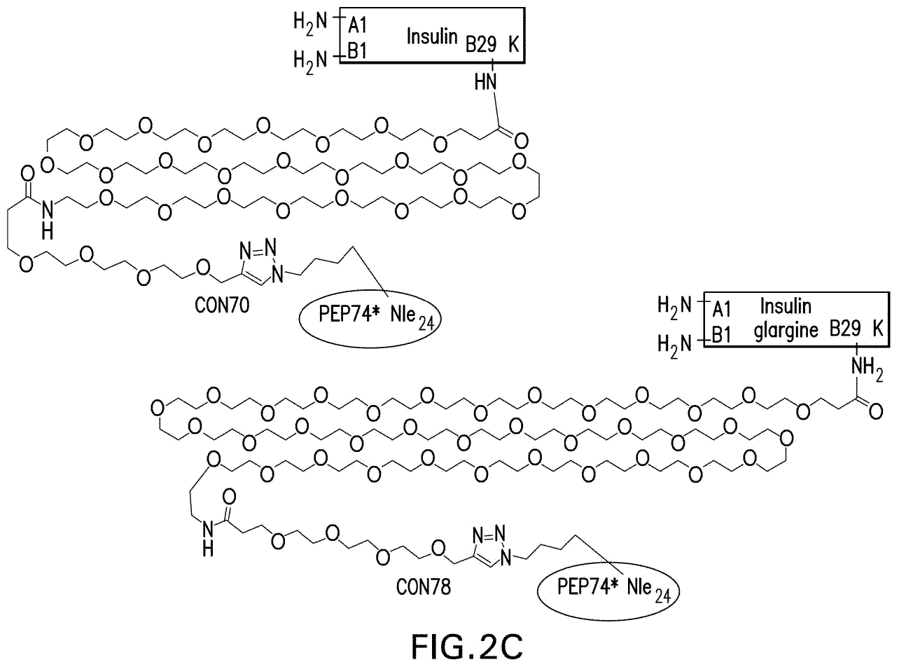

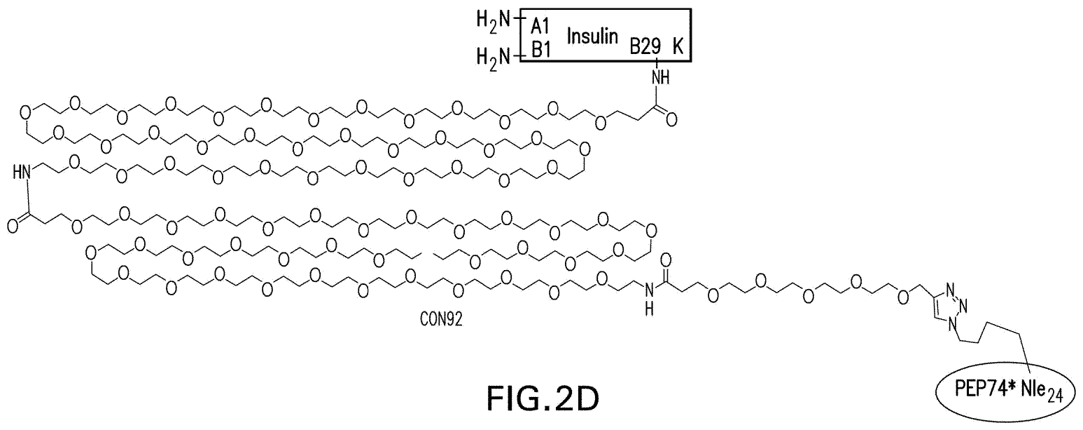

FIG. 2A, FIG. 2B, and FIG. 2C show various exemplary insulin-incretin conjugates in which the Norleucine (Nle) amino acid of particular incretins is conjugated to the epsilon amino group of Lysine (Lys) at position B29 of the B-chain peptide.

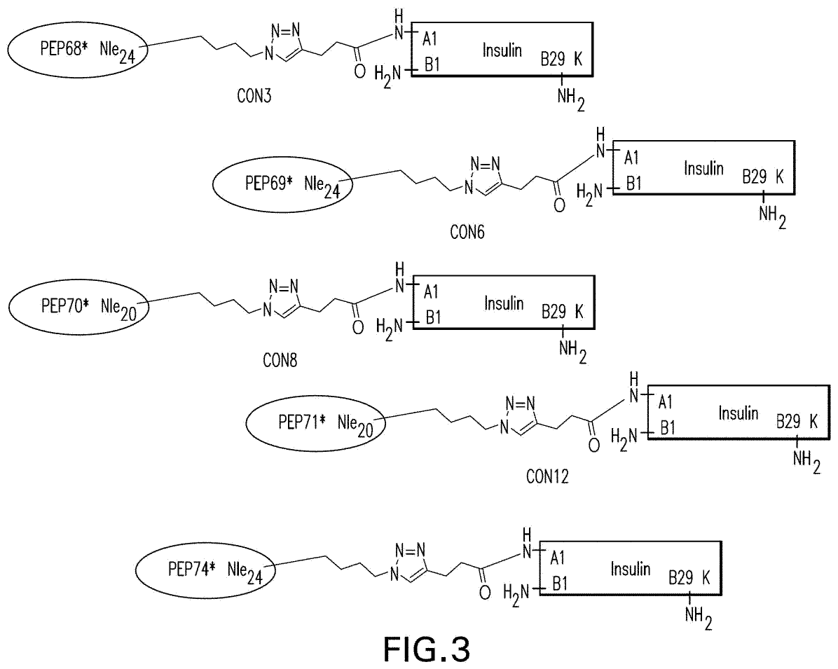

FIG. 3 shows various exemplary insulin-incretin conjugates in which the Nle amino acid of particular incretins is conjugated to the amino group of the Glycine (Gly) at position A1 of the A-chain peptide.

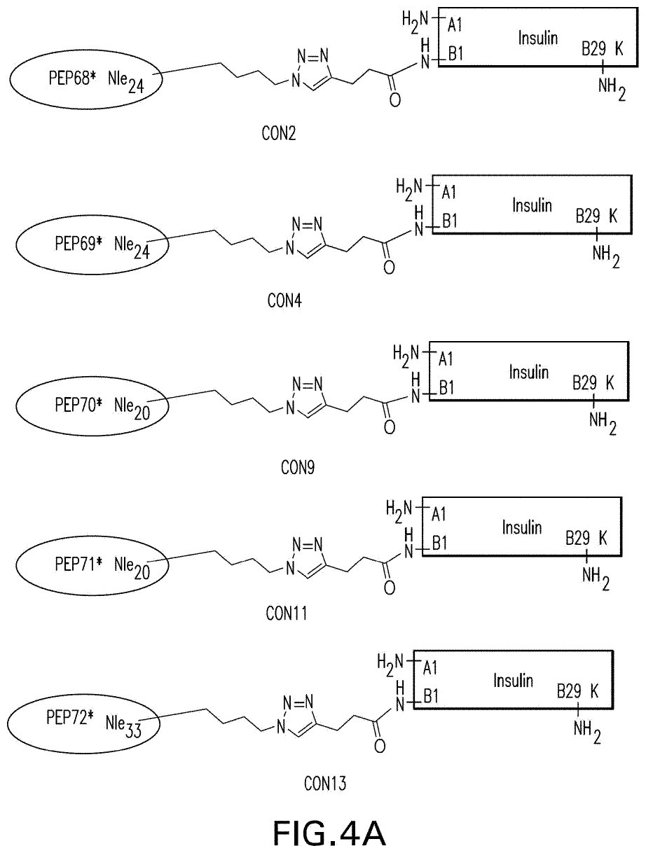

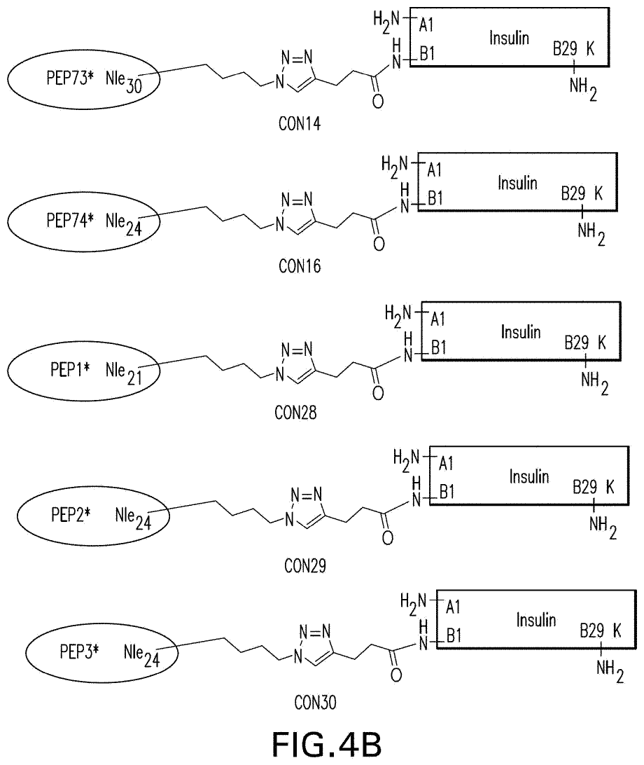

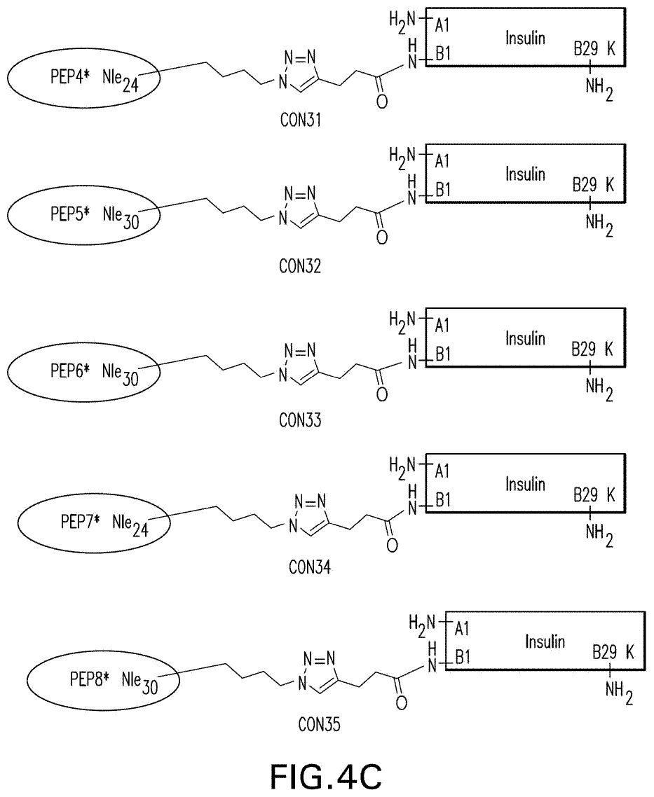

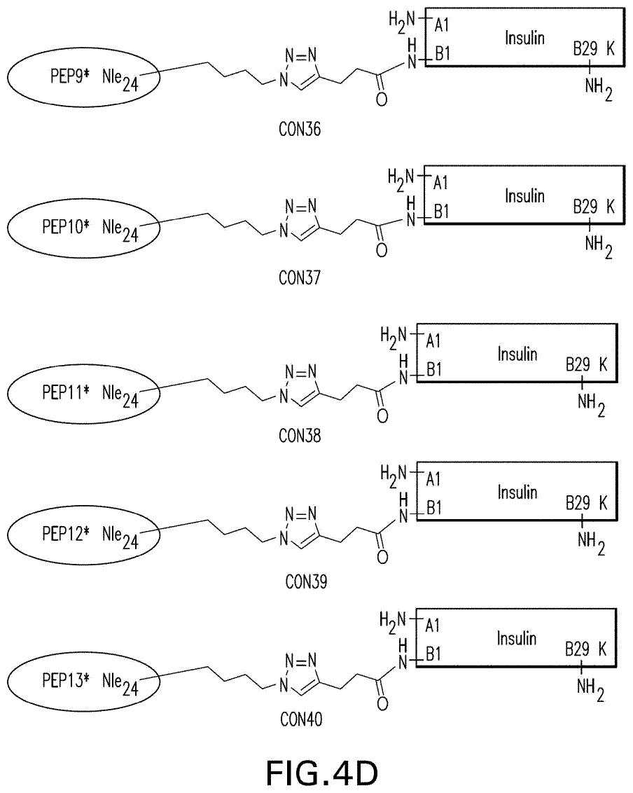

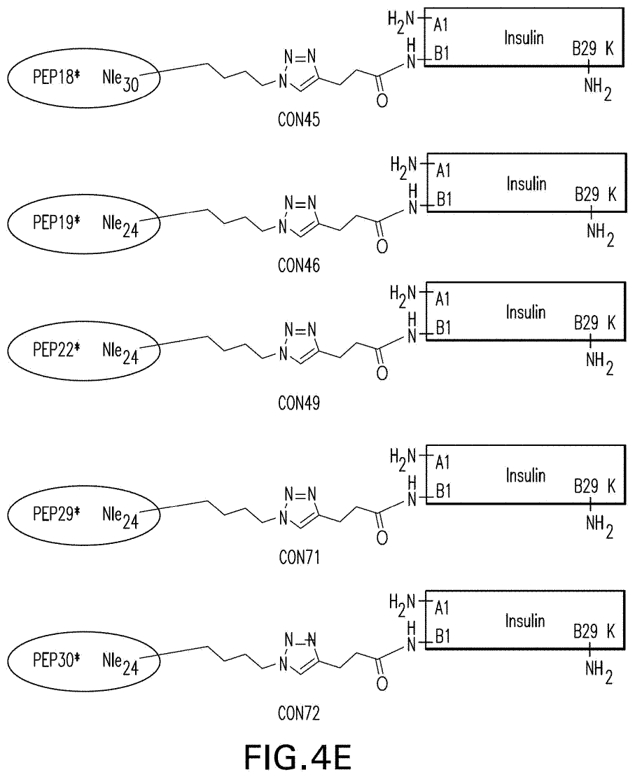

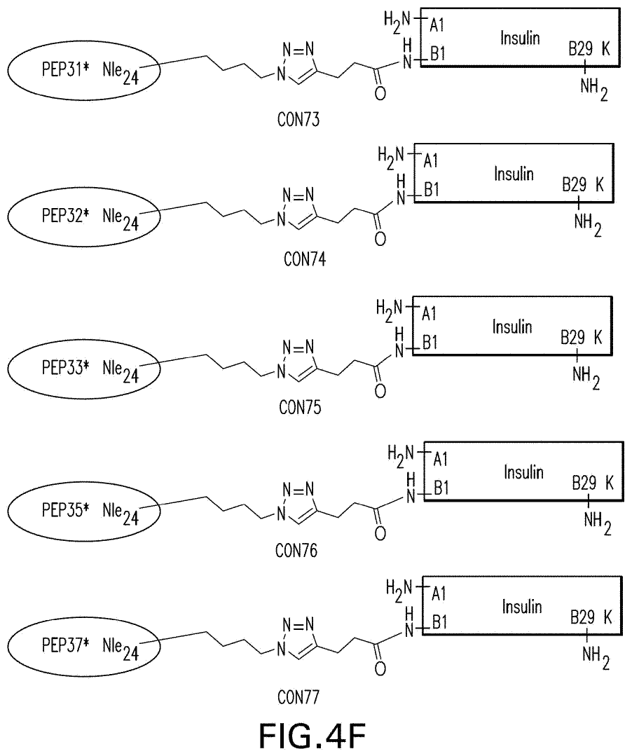

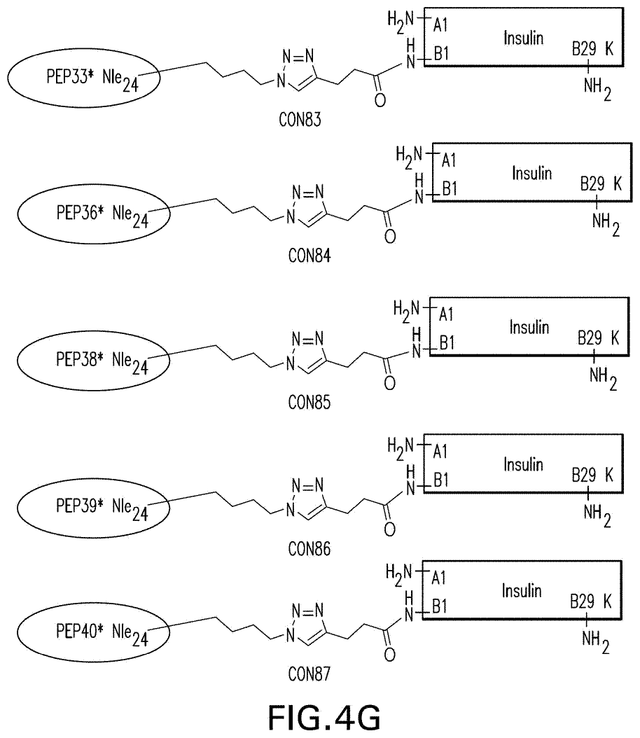

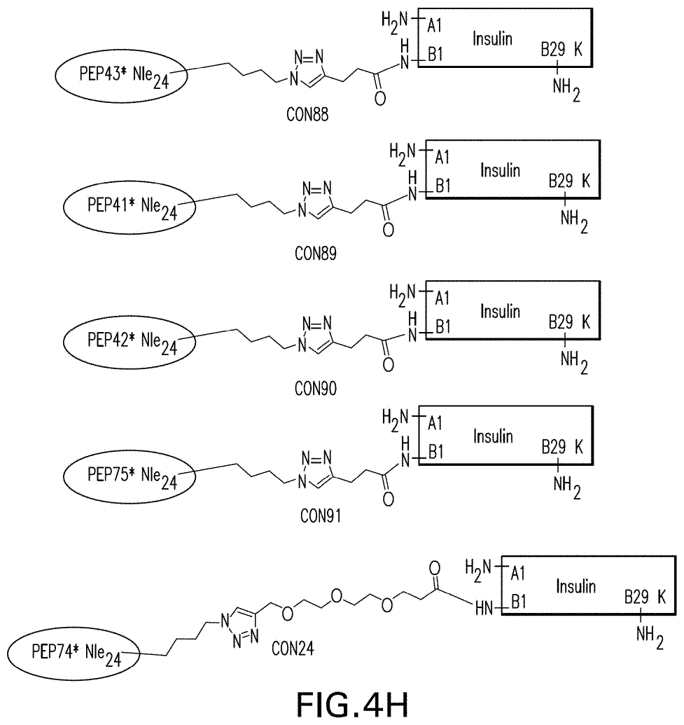

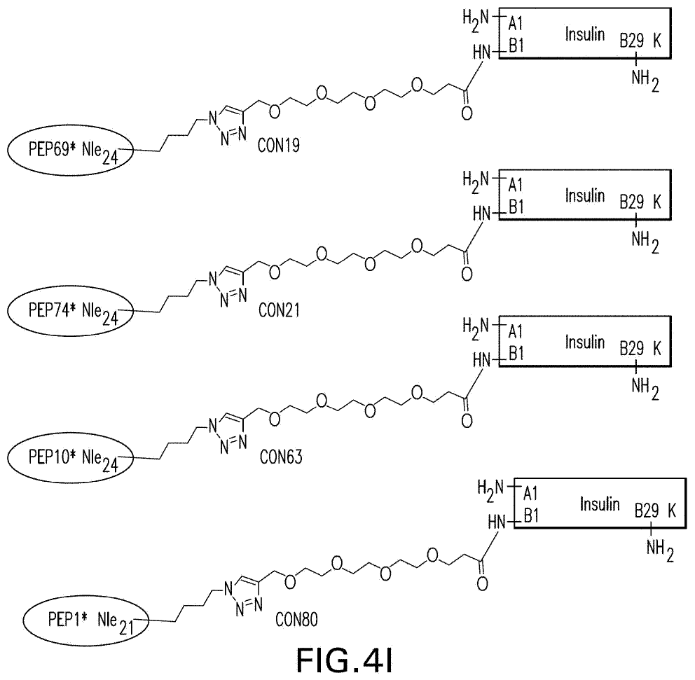

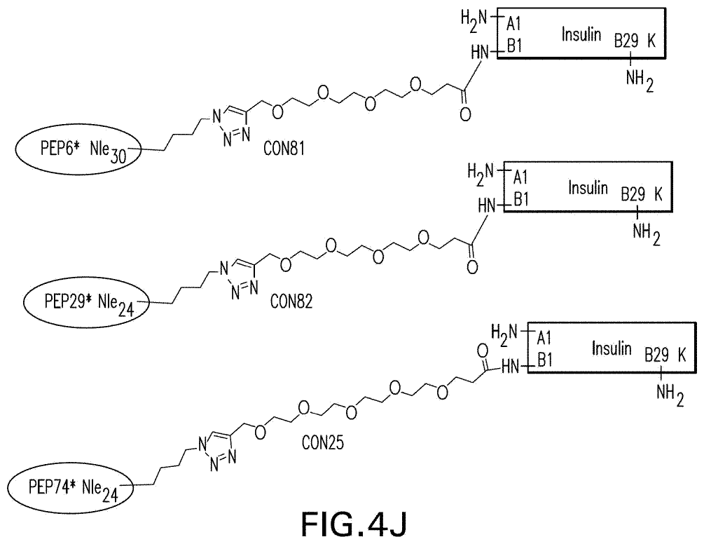

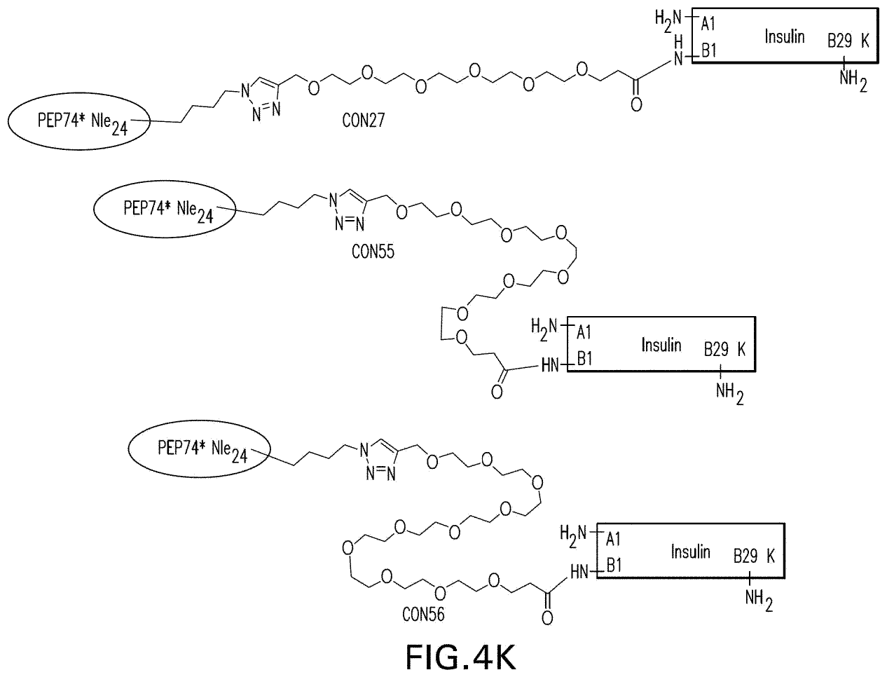

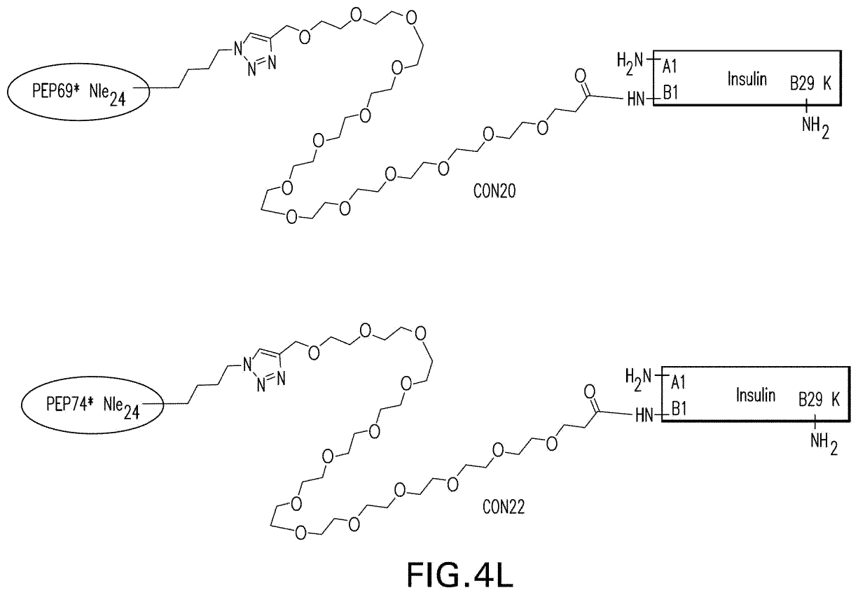

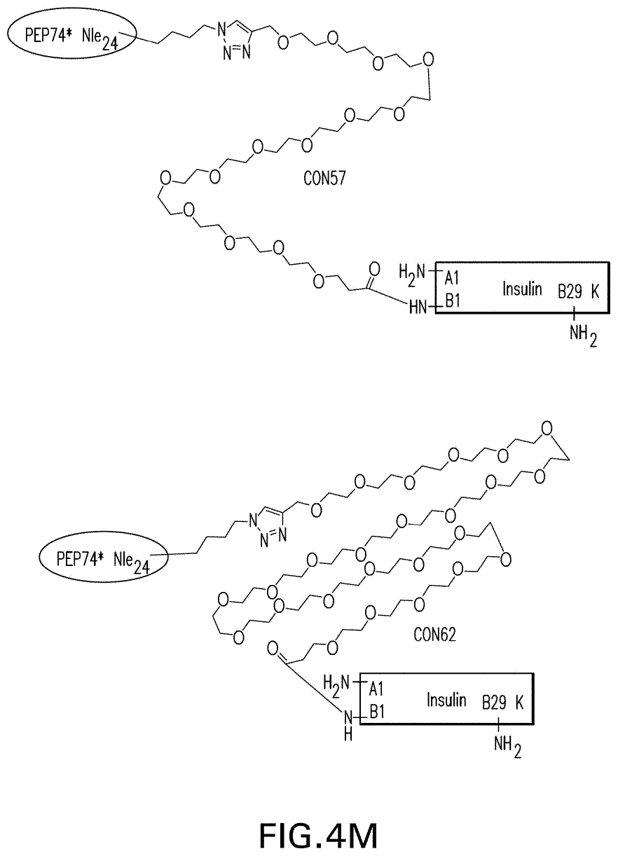

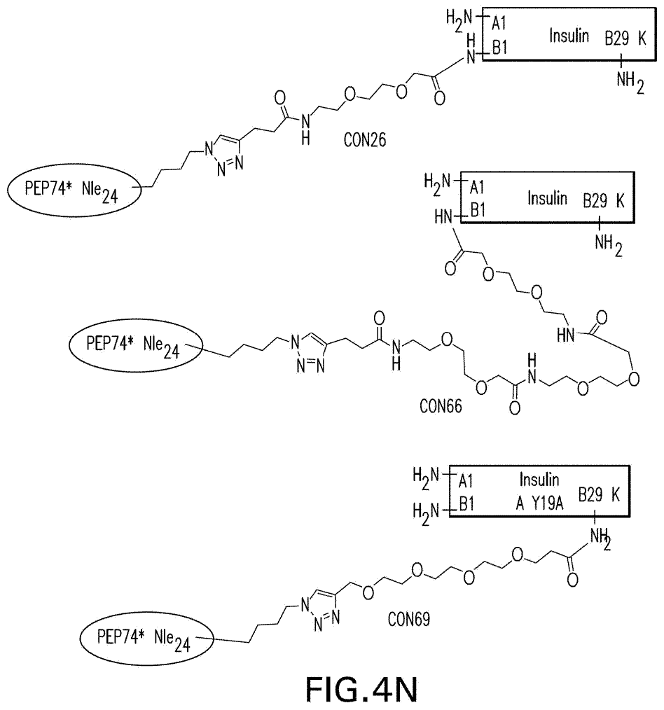

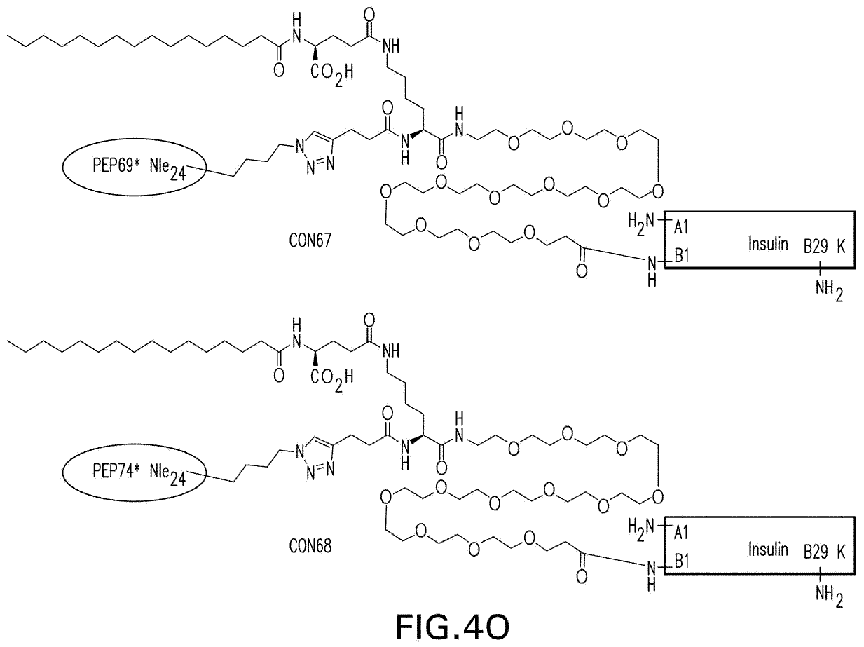

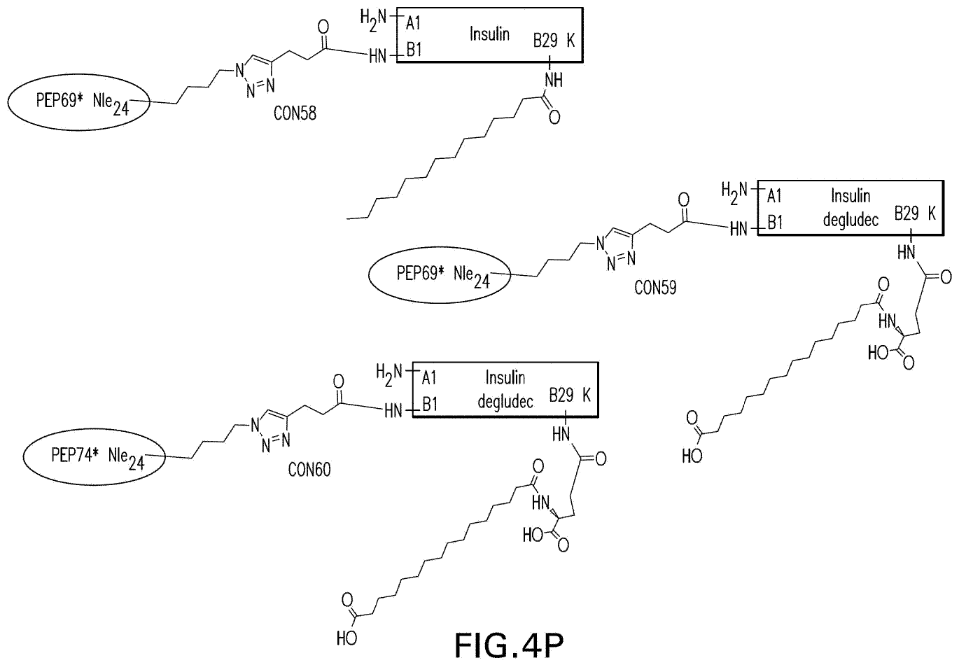

FIG. 4A, FIG. 4B, FIG. 4C, FIG. 4D, FIG. 4E, FIG. 4F, FIG. 4G, FIG. 4H, FIG. 4I, FIG. 4J, FIG. 4K, FIG. 4L, FIG. 4M, FIG. 4N, FIG. 4O, and FIG. 4P show exemplary insulin-incretin conjugates in which the Nle amino acid of particular incretins is conjugated to the amino group of the Phenylalanine (Phe) at position B1 of the B-chain peptide.

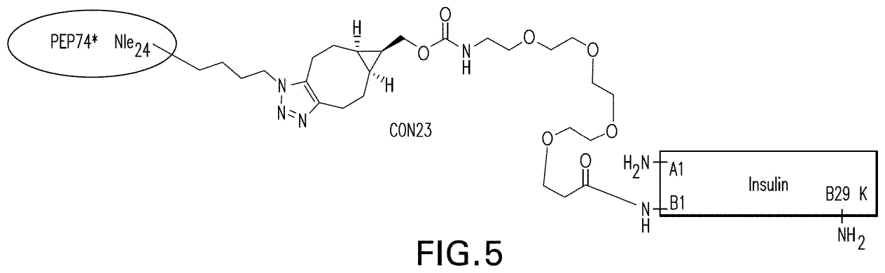

FIG. 5 shows an exemplary insulin-incretin conjugate in which the Nle amino acid of PEP74* is conjugated to the amino group of the Phe at position B1 of the B-chain peptide.

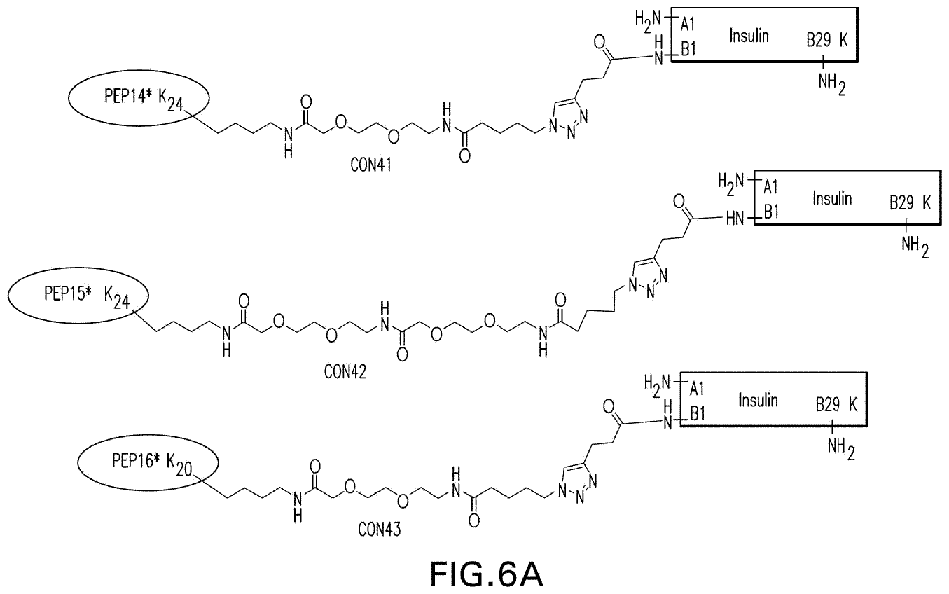

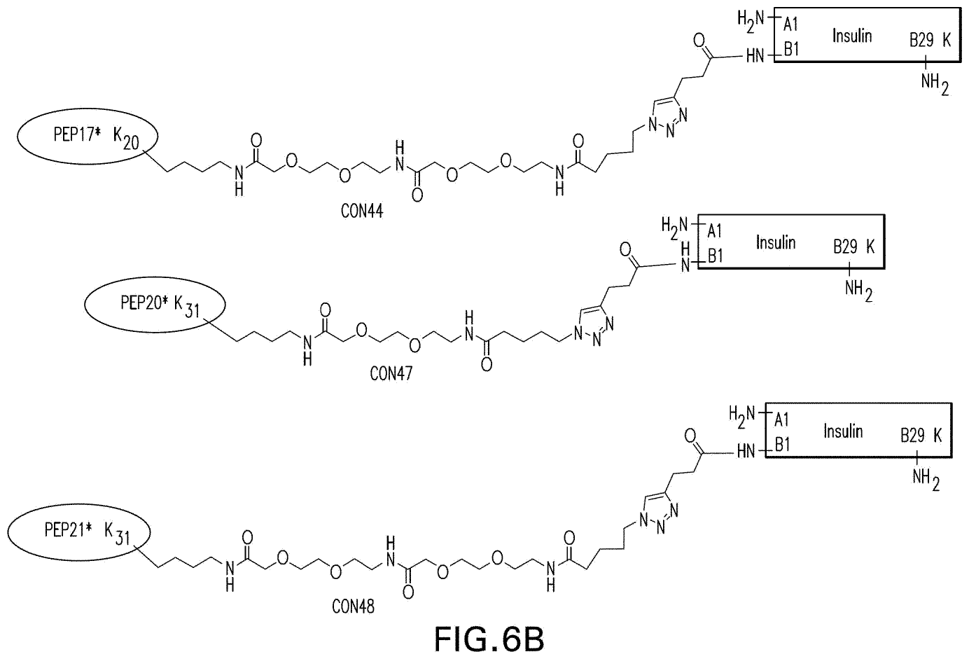

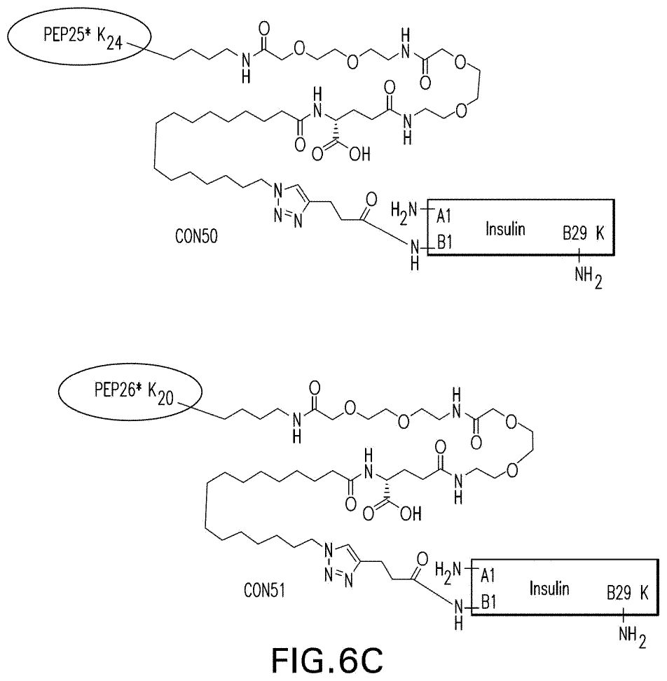

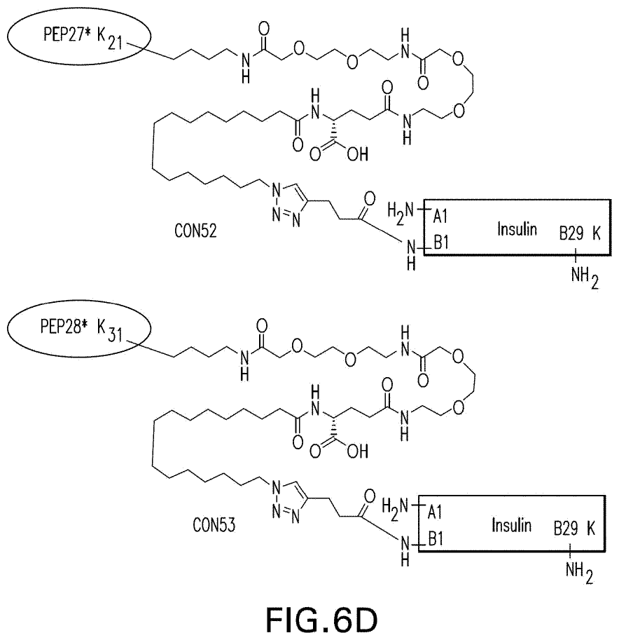

FIG. 6A, FIG. 6B, FIG. 6C, and FIG. 6D show exemplary insulin-incretin conjugates in which the epsilon amino group of Lys of the incretin is conjugated to the amino group of the Phe at position B1 of the B-chain peptide.

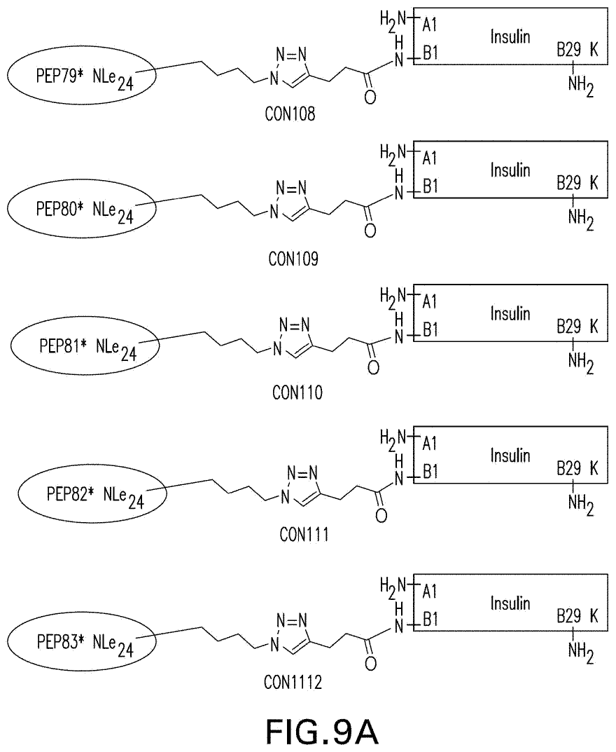

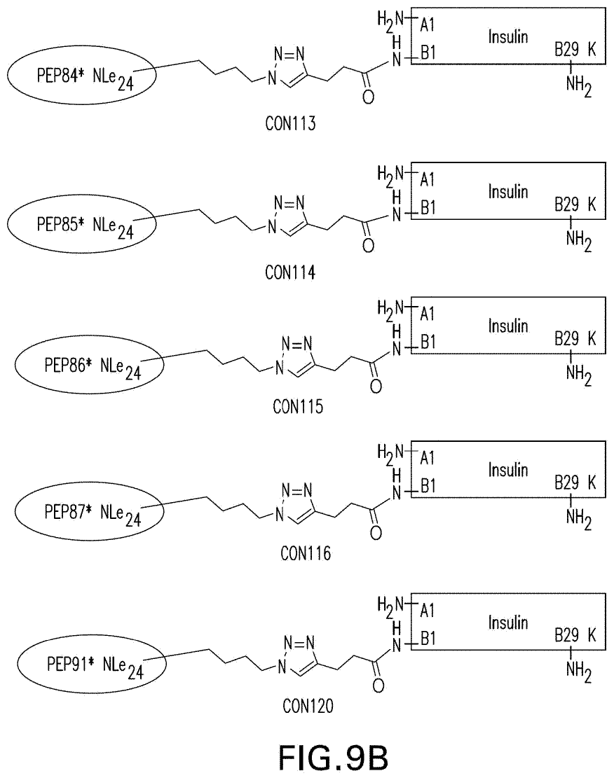

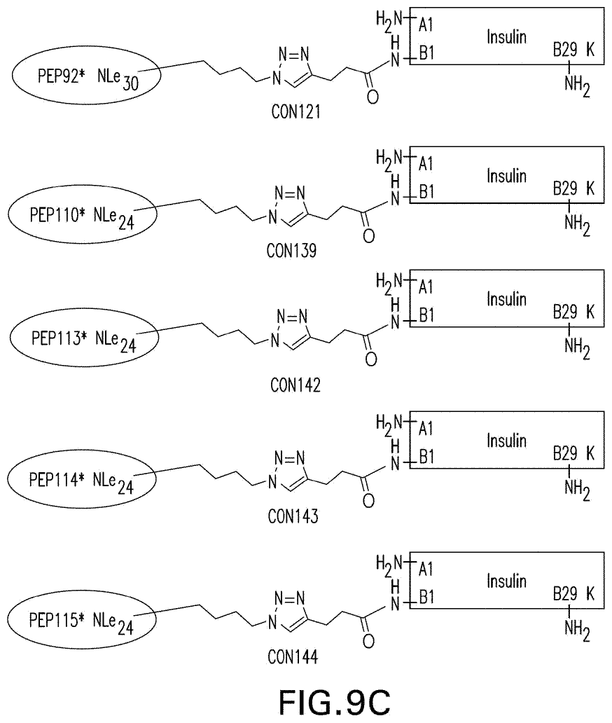

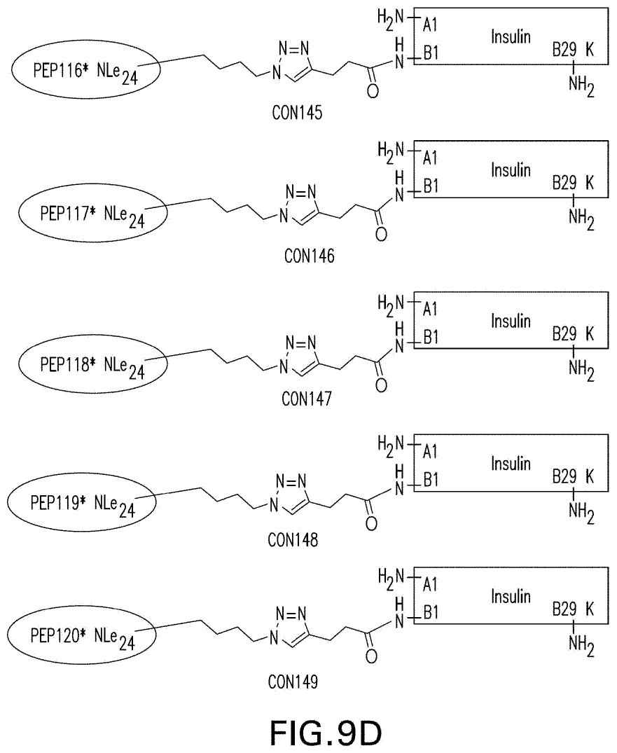

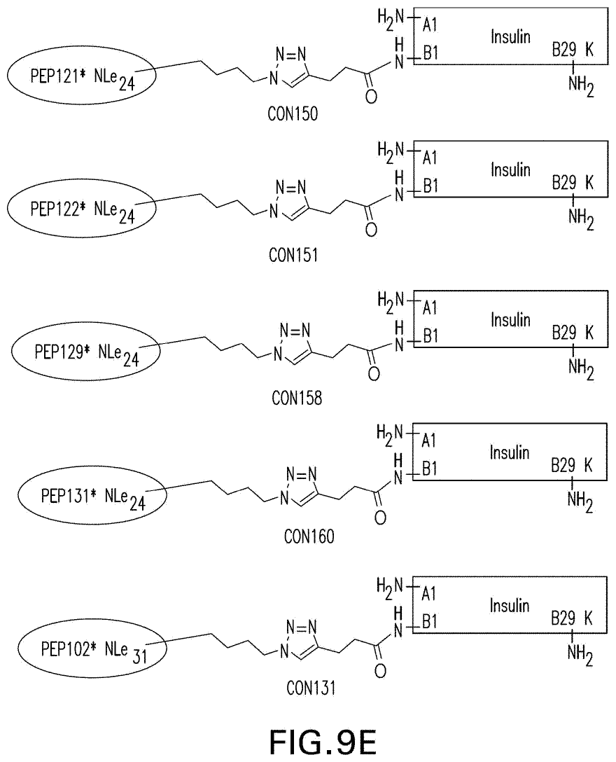

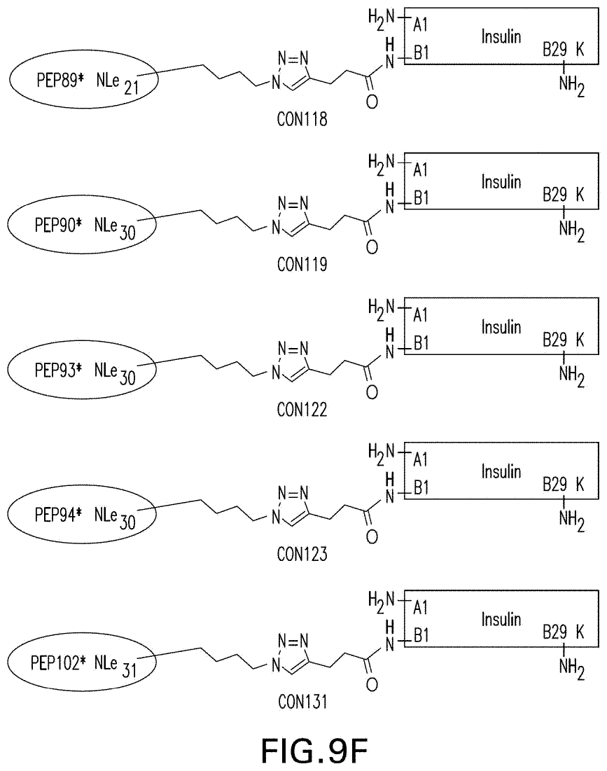

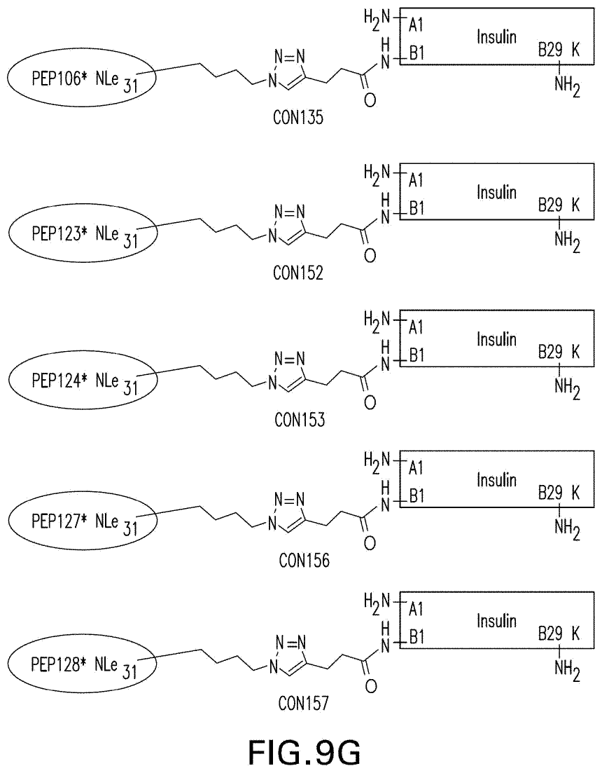

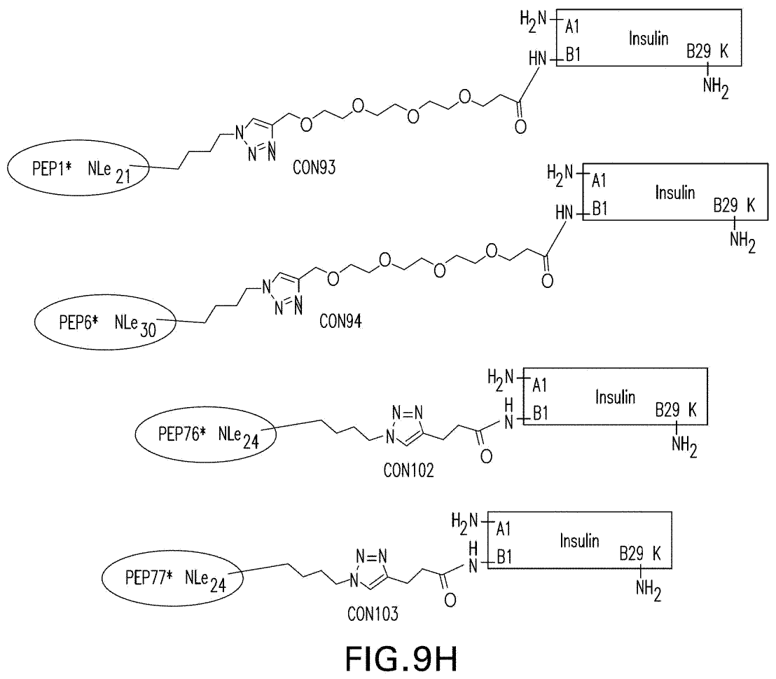

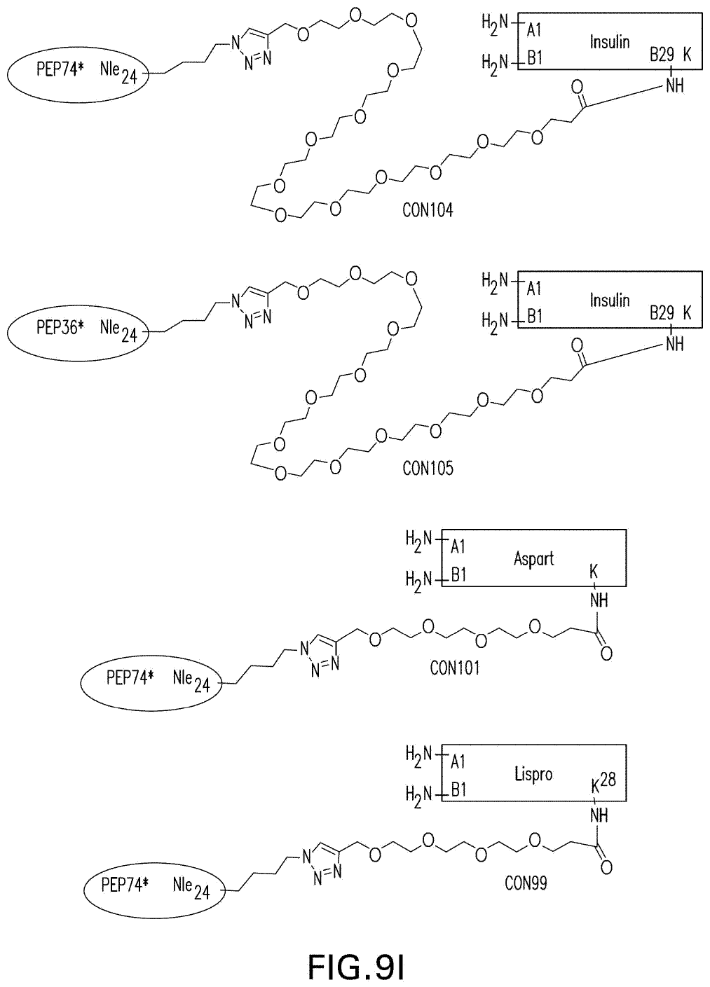

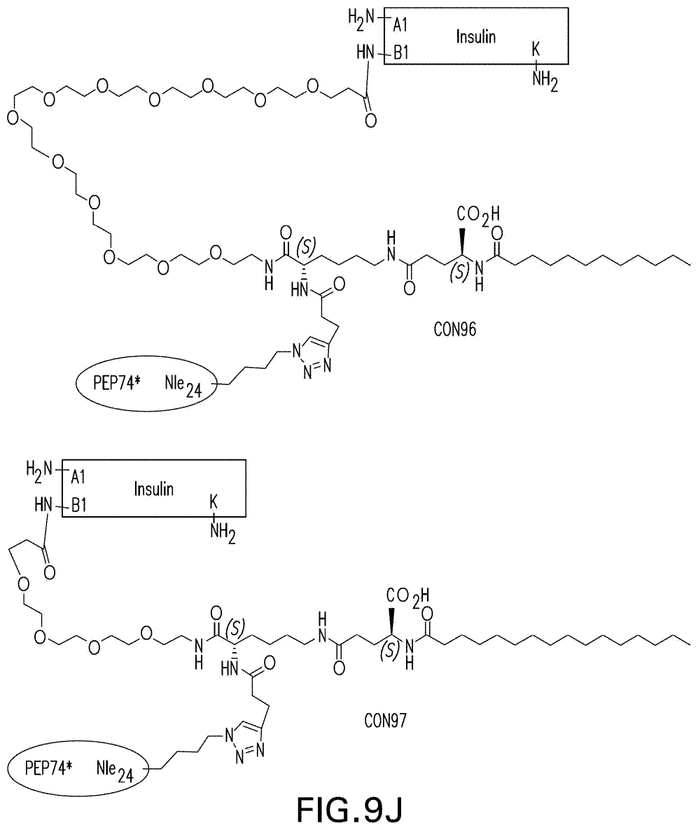

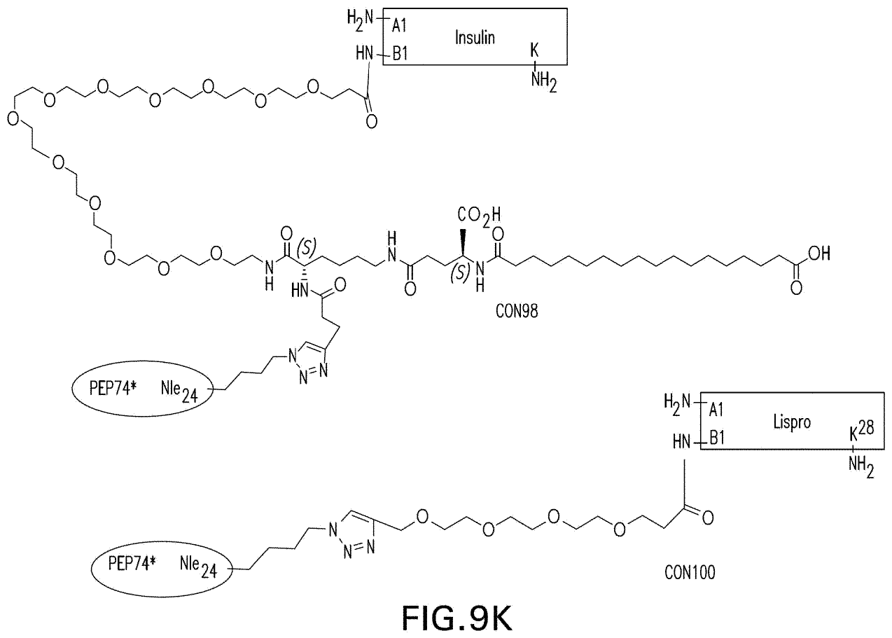

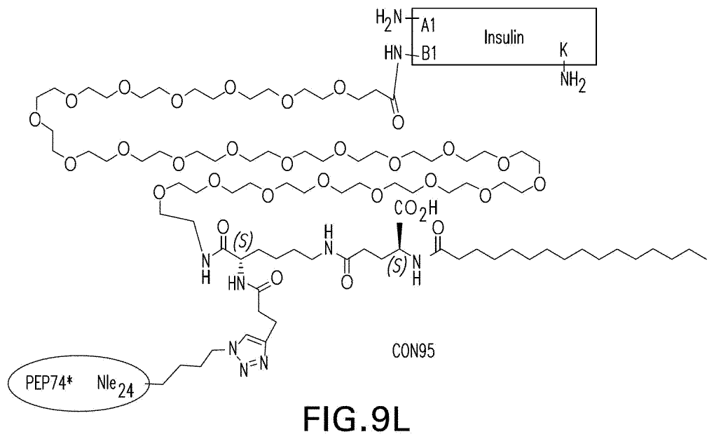

FIG. 9A, FIG. 9B, FIG. 9C, FIG. 9D, FIG. 9E, FIG. 9F, FIG. 9G, FIG. 9H, FIG. 9I, FIG. 9J, FIG. 9K, and FIG. 9L, which show exemplary insulin-incretin conjugates in which the Norleucine (Nle) amino acid of the incretin is conjugated to the amino group of the Phe at position B1 of the B-chain peptide.

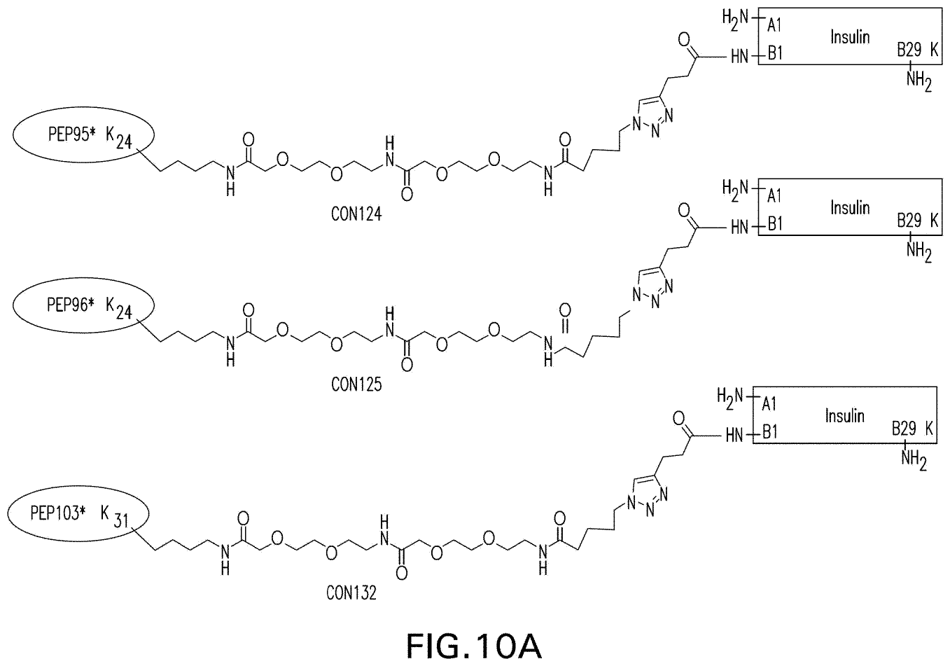

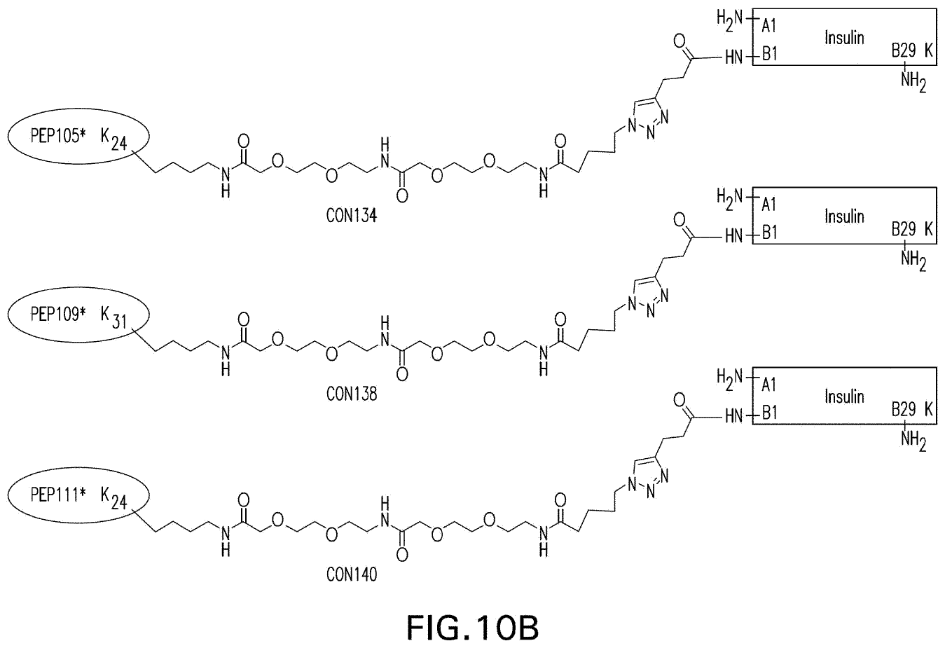

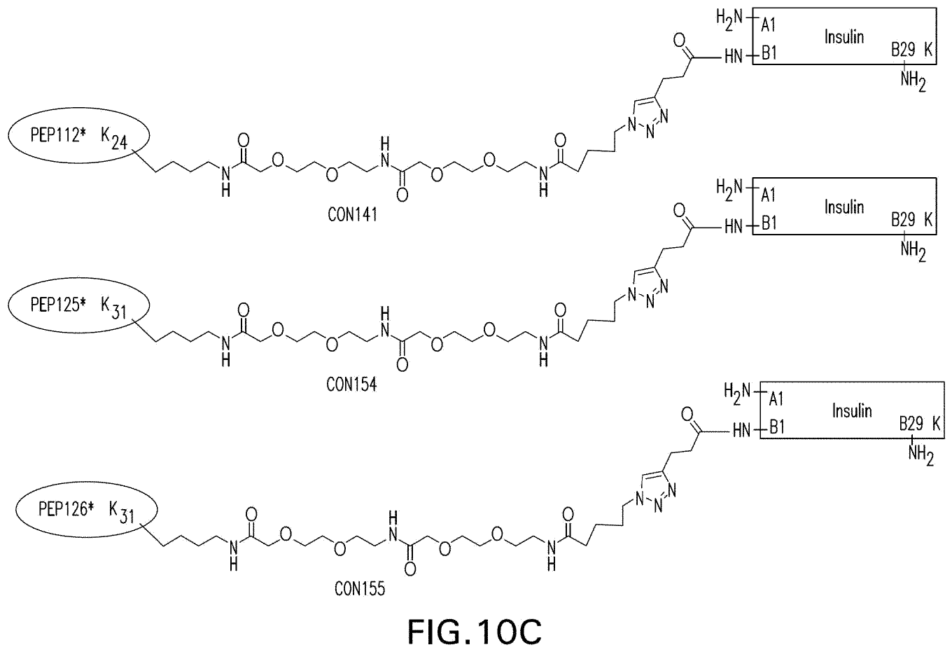

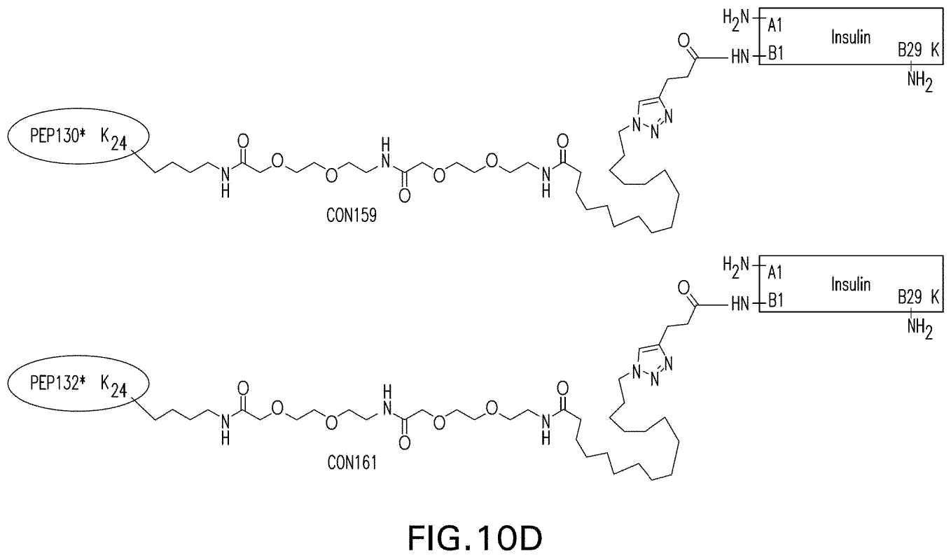

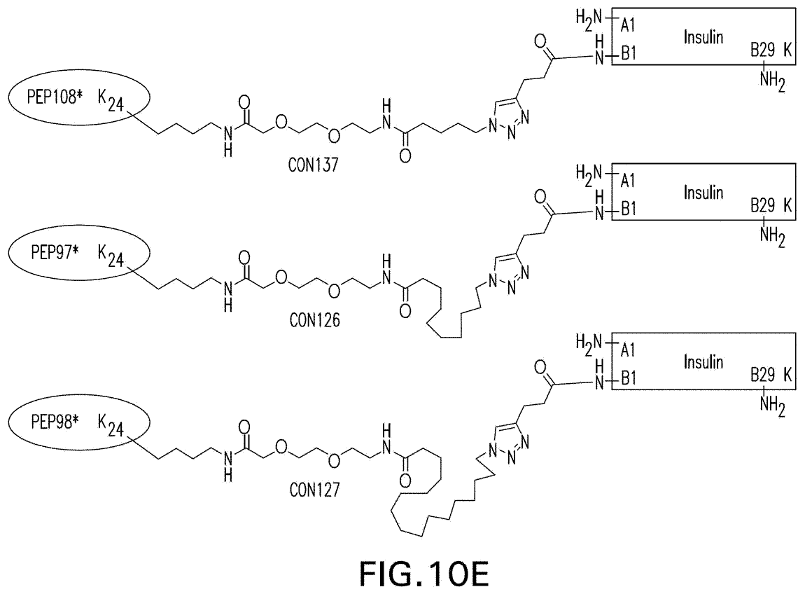

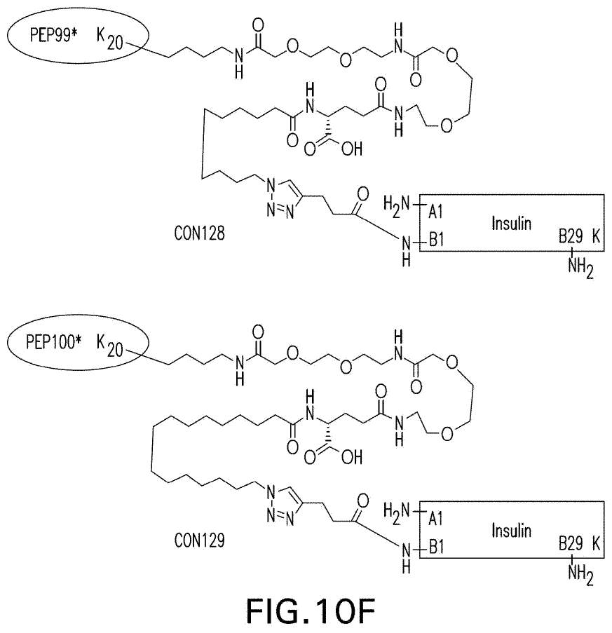

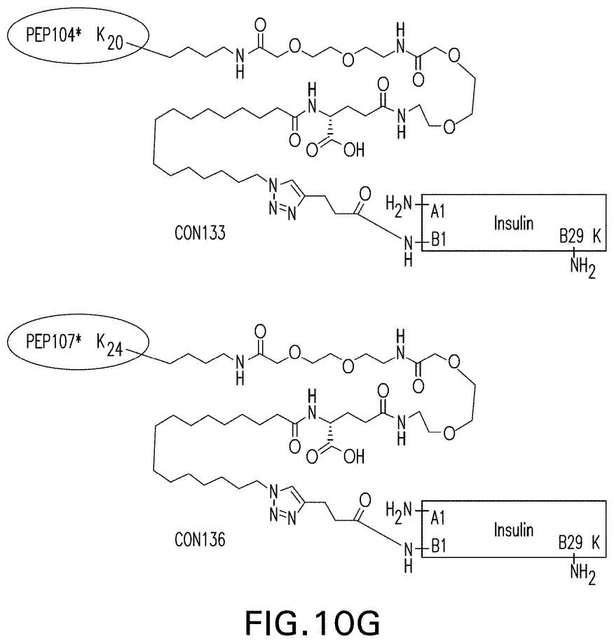

FIG. 10A, FIG. 10B, FIG. 10C, FIG. 10D, FIG. 10E, FIG. 10F, and FIG. 10G, which show exemplary insulin-incretin conjugates in which the epsilon amino group of Lys of the incretin is conjugated to the amino group of the Phenylalanine (Phe) at position B1 of the B-chain peptide.

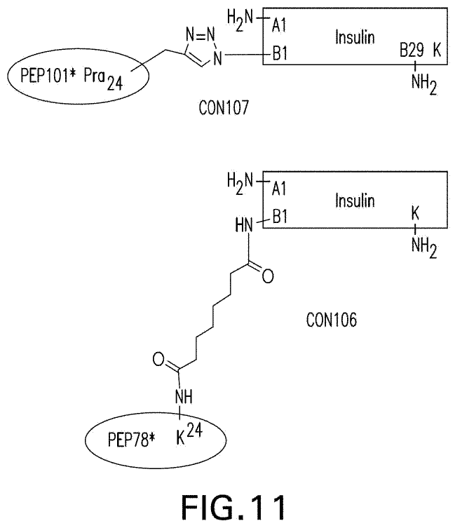

FIG. 11, which shows exemplary insulin-incretin conjugates CON106 and CON107.

### Pharmaceutical Compositions

Further provided are pharmaceutical compositions comprising a therapeutically effective amount of one or more of the peptides disclosed herein for the treatment of a metabolic disorder in an individual. Such disorders include, but are not limited to, obesity, metabolic syndrome or syndrome X, type II diabetes, complications of diabetes such as retinopathy, hypertension, dyslipidemias, cardiovascular disease, gallstones, osteoarthritis, and certain forms of cancers. The obesity-related disorders herein are associated with, caused by, or result from obesity.

“Obesity” is a condition in which there is an excess of body fat. The operational definition of obesity is based on the Body Mass Index (BMI), calculated as body weight per height in meters squared (kg/m2). “Obesity” refers to a condition whereby an otherwise healthy subject has a Body Mass Index (BMI) greater than or equal to 30 kg/m2, or a condition whereby a subject with at least one co-morbidity has a BMI greater than or equal to 27 kg/m2. An “obese subject” is an otherwise healthy subject with a Body Mass Index (BMI) greater than or equal to 30 kg/m2 or a subject with at least one co-morbidity with a BMI greater than or equal to 27 kg/m2. A “subject at risk for obesity” is an otherwise healthy subject with a BMI of 25 kg/m2 to less than 30 kg/m2 or a subject with at least one co-morbidity with a BMI of 25 kg/m2 to less than 27 kg/m2.

The increased risks associated with obesity occur at a lower Body Mass Index (BMI) in Asians. In Asian countries, including Japan, “obesity” refers to a condition whereby a subject with at least one obesity-induced or obesity-related co-morbidity that requires weight reduction or that would be improved by weight reduction, has a BMI greater than or equal to 25 kg/m2. In Asian countries, including Japan, an “obese subject” refers to a subject with at least one obesity-induced or obesity-related co-morbidity that requires weight reduction or that would be improved by weight reduction, with a BMI greater than or equal to 25 kg/m2. In Asian countries, a “subject at risk of obesity” is a subject with a BMI of greater than 23 kg/m2 to less than 25 kg/m2.

As used herein, the term “obesity” is meant to encompass all of the above definitions of obesity.

Obesity-induced or obesity-related co-morbidities include, but are not limited to, diabetes, non-insulin dependent diabetes mellitus—type 2, impaired glucose tolerance, impaired fasting glucose, insulin resistance syndrome, dyslipidemia, hypertension, hyperuricacidemia, gout, coronary artery disease, myocardial infarction, angina pectoris, sleep apnea syndrome, Pickwickian syndrome, non-alcoholic fatty liver disease (NAFLD), non-alcoholic steatohepatitis (NASH), fatty liver; cerebral infarction, cerebral thrombosis, transient ischemic attack, orthopedic disorders, arthritis deformans, lumbodynia, emmeniopathy, and infertility. In particular, co-morbidities include: hypertension, hyperlipidemia, dyslipidemia, glucose intolerance, cardiovascular disease, sleep apnea, diabetes mellitus, and other obesity-related conditions.

“Treatment” (of obesity and obesity-related disorders) refers to the administration of the compounds of the present invention to reduce or maintain the body weight of an obese subject. One outcome of treatment may be reducing the body weight of an obese subject relative to that subject's body weight immediately before the administration of the compounds of the present invention. Another outcome of treatment may be preventing body weight regain of body weight previously lost as a result of diet, exercise, or pharmacotherapy. Another outcome of treatment may be decreasing the occurrence of and/or the severity of obesity-related diseases. The treatment may suitably result in a reduction in food or calorie intake by the subject, including a reduction in total food intake, or a reduction of intake of specific components of the diet such as carbohydrates or fats; and/or the inhibition of nutrient absorption; and/or the inhibition of the reduction of metabolic rate; and in weight reduction in patients in need thereof. The treatment may also result in an alteration of metabolic rate, such as an increase in metabolic rate, rather than or in addition to an inhibition of the reduction of metabolic rate; and/or in minimization of the metabolic resistance that normally results from weight loss.

“Prevention” (of obesity and obesity-related disorders) refers to the administration of the compounds of the present invention to reduce or maintain the body weight of a subject at risk of obesity. One outcome of prevention may be reducing the body weight of a subject at risk of obesity relative to that subject's body weight immediately before the administration of the compounds of the present invention. Another outcome of prevention may be preventing body weight regain of body weight previously lost as a result of diet, exercise, or pharmacotherapy. Another outcome of prevention may be preventing obesity from occurring if the treatment is administered prior to the onset of obesity in a subject at risk of obesity. Another outcome of prevention may be decreasing the occurrence and/or severity of obesity-related disorders if the treatment is administered prior to the onset of obesity in a subject at risk of obesity. Moreover, if treatment is commenced in already obese subjects, such treatment may prevent the occurrence, progression or severity of obesity-related disorders, such as, but not limited to, arteriosclerosis, Type II diabetes, polycystic ovarian disease, cardiovascular diseases, osteoarthritis, dermatological disorders, hypertension, insulin resistance, hypercholesterolemia, hypertriglyceridemia, and cholelithiasis.

The obesity-related disorders herein are associated with, caused by, or result from obesity. Examples of obesity-related disorders include overeating and bulimia, hypertension, diabetes, elevated plasma insulin concentrations and insulin resistance, dyslipidemias, hyperlipidemia, endometrial, breast, prostate and colon cancer, osteoarthritis, obstructive sleep apnea, cholelithiasis, gallstones, heart disease, abnormal heart rhythms and arrythmias, myocardial infarction, congestive heart failure, coronary heart disease, sudden death, stroke, polycystic ovarian disease, craniopharyngioma, the Prader-Willi Syndrome, Frohlich's syndrome, GH-deficient subjects, normal variant short stature, Turner's syndrome, and other pathological conditions showing reduced metabolic activity or a decrease in resting energy expenditure as a percentage of total fat-free mass, e.g, children with acute lymphoblastic leukemia. Further examples of obesity-related disorders are metabolic syndrome, also known as syndrome X, insulin resistance syndrome, sexual and reproductive dysfunction, such as infertility, hypogonadism in males and hirsutism in females, gastrointestinal motility disorders, such as obesity-related gastro-esophageal reflux, respiratory disorders, such as obesity-hypoventilation syndrome (Pickwickian syndrome), cardiovascular disorders, inflammation, such as systemic inflammation of the vasculature, arteriosclerosis, hypercholesterolemia, hyperuricaemia, lower back pain, gallbladder disease, gout, and kidney cancer. The compounds of the present invention are also useful for reducing the risk of secondary outcomes of obesity, such as reducing the risk of left ventricular hypertrophy.

The term “diabetes,” as used herein, includes both insulin-dependent diabetes mellitus (IDDM, also known as type I diabetes) and non-insulin-dependent diabetes mellitus (NIDDM, also known as Type II diabetes). Type I diabetes, or insulin-dependent diabetes, is the result of an absolute deficiency of insulin, the hormone which regulates glucose utilization. Type II diabetes, or insulin-independent diabetes (i.e., non-insulin-dependent diabetes mellitus), often occurs in the face of normal, or even elevated levels of insulin and appears to be the result of the inability of tissues to respond appropriately to insulin. Most of the Type II diabetics are also obese. The compounds of the present invention are useful for treating both Type I and Type II diabetes. The compounds are especially effective for treating Type II diabetes. The compounds of the present invention are also useful for treating and/or preventing gestational diabetes mellitus.

U.S. Pat. No. 6,852,690, which is incorporated herein in its entirety, discloses methods for enhancing metabolism of nutrients comprising administering to a non-diabetic patient a formulation comprising a nutritively effective amount of one or more nutrients or any combination thereof and one or more insulinotropic peptides. The peptides disclosed herein are insulinotropic and can be administered to patients with a disturbed glucose metabolism such as insulin resistance but no overt diabetes, as well as patients who for any reason cannot receive nutrition through the alimentary canal. Such patients include surgery patients, comatose patients, patients in shock, patients with gastrointestinal disease, patients with digestive hormone disease, and the like. In particular, obese patients, atherosclerotic patients, vascular disease patients, patients with gestational diabetes, patients with liver disease such as liver cirrhosis, patients with acromegaly, patients with glucorticoid excess such as cortisol treatment or Cushings disease, patients with activated counterregulatory hormones such as would occur after trauma, accidents and surgery and the like, patients with hypertriglyceridemia and patients with chronic pancreatitis can be readily and suitably nourished according to the invention without subjecting the patient to hypo- or hyperglycemia. In particular, the administration to such a patient aims to provide a therapy to as rapidly as possible deliver the nutritional and caloric requirements to the patient while maintaining his plasma glucose below the so-called renal threshold of about 160 to 180 milligrams per deciliter of glucose in the blood. Although normal patients not having glucose levels just below the renal threshold can also be treated according to the invention as described above, patients with disturbed glucose metabolism such as hyperglycemic patients whose plasma glucose level is just above the renal threshold also find the therapy suitable for their condition. In particular, such patients who have a degree of hyperglycemia below the renal threshold at intermittent intervals can receive a combination treatment of nutrients plus insulinotropic peptides according to any of the following regimens. Normal patients not suffering from such hyperglycemia can also be treated using the peptides disclosed herein.

The peptides disclosed herein may be used in a pharmaceutical composition when combined with a pharmaceutically acceptable carrier. Such compositions comprise a therapeutically-effective amount of one or more of the peptides disclosed herein and a pharmaceutically acceptable carrier. Such a composition may also be comprised of (in addition to the peptides disclosed herein and a carrier) diluents, fillers, salts, buffers, stabilizers, solubilizers, and other materials well known in the art. Compositions comprising the peptides disclosed herein can be administered, if desired, in the form of salts provided the salts are pharmaceutically acceptable. Salts may be prepared using standard procedures known to those skilled in the art of synthetic organic chemistry.

The term “individual” is meant to include humans and companion or domesticated animals such as dogs, cats, horses, and the like. Therefore, the compositions comprising a compound as disclosed herein are also useful for treating or preventing obesity and obesity-related disorders in cats and dogs. As such, the term “mammal” includes companion animals such as cats and dogs.

The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like. The term “pharmaceutically acceptable salt” further includes all acceptable salts such as acetate, lactobionate, benzenesulfonate, laurate, benzoate, malate, bicarbonate, maleate, bisulfate, mandelate, bitartrate, mesylate, borate, methylbromide, bromide, methylnitrate, calcium edetate, methylsulfate, camsylate, mucate, carbonate, napsylate, chloride, nitrate, clavulanate, N-methylglucamine, citrate, ammonium salt, dihydrochloride, oleate, edetate, oxalate, edisylate, pamoate (embonate), estolate, palmitate, esylate, pantothenate, fumarate, phosphate/diphosphate, gluceptate, polygalacturonate, gluconate, salicylate, glutamate, stearate, glycollylarsanilate, sulfate, hexylresorcinate, subacetate, hydrabamine, succinate, hydrobromide, tannate, hydrochloride, tartrate, hydroxynaphthoate, teoclate, iodide, tosylate, isothionate, triethiodide, lactate, panoate, valerate, and the like which can be used as a dosage form for modifying the solubility or hydrolysis characteristics or can be used in sustained release or pro-drug formulations. It will be understood that, as used herein, references to the OXM analogs disclosed herein are meant to also include the pharmaceutically acceptable salts.

The following examples are intended to promote a further understanding of the present invention.

### Example 1

A general procedure for synthesizing the peptides shown in Table 1 may be performed as follows.

The peptides may be synthesized by solid phase synthesis using Fmoc/t-Bu chemistry on a peptide multisynthesizer Symphony (Protein Technologies Inc.) on a 150 μmol scale, using either a Rink-amide PEG-PS resin (Champion, Biosearch Technologies, loading 0.28 mmol/g) or a Rink-amide PS resin (ChemImpex loading 0.47 mmol/g).

All the amino acids are dissolved at a 0.3 M concentration in DMF. The amino acids are activated with equimolar amounts of HATU (O-(7-azabenzotriazol-1-yl)-N,N,N′,N′-tetramethyluronium hexafluorophosphate) solution 0.3 M in DMF, and a 2-fold molar excess of DIEA (N,N-diisopropylethylamine), solution 2M in NMP. The acylation reactions are performed in general for 1 hour with a 5-fold excess of activated amino acid over the resin free amino groups with double 45 minutes acylation reactions performed from His¹ to Thr⁷, from D¹⁵ to U¹⁶ and from F²² to V²³.

The side chain protecting groups may be: tert-butyl for Glu, Ser, D-Ser, Thr and Tyr; trityl for Asn, Gln and His; tert-butoxy-carbonyl for Lys, Trp; and, 2,2,4,6,7-pentamethyldihydrobenzofuran-5-sulfonyl for Arg; His may be introduced as Boc-His(Trt)-OH at the end of the sequence assembly. Amino acid 2 (L-methionine-sulphone) may be introduced by acylation of Fmoc-L-methionine-sulphone-COOH; Nle(εN₃) (ε-azidonorleucine) was introduced by acylation of Fmoc-ε-azidonorleucine-COOH.

The lysine that may be used for linker-lipid derivatization, may be incorporated with a Dde [1-(4,4-dimethyl-2,6-dioxocyclohex-1-ylidene)ethyl] protecting group or Alloc (allyloxycarbonyl) protecting group on the side chain amino group. For those sequences comprising an alpha methyl amino acid and the corresponding following residue, the incorporation may be performed by manual coupling with HOAt (Hydroxybenzoazatriazole) and DIC (N,N′-diisopropylcarbodiimide).

At the end of the assembly, the Dde protecting group of Lys(Dde) is removed by treatment with 2% hydrazine in DMF and the Alloc protecting group of Lys(Alloc) is removed by treatment with Pd(PPh₃)₄ and PhSiH₃. The side chains of Lys are derivatized with different linkers and fatty acids by incorporation of Fmoc-Glu-OtBu (γ-glutamic acid), Fmoc-PEG₂ [8-(9-Fluorenylmethyloxycarbonyl-amino)-3,6-dioxaoctanoic acid], the lipid diacids (Octadecanedioic acid; Eicosanedioic acid) and Azido acids (C₅N₃=5-azido pentinoic acid; C₁₀N₃=10-azido-decanoic acid; C₁₆N₃=16-azido-hexadecanoic acid) using HOAt and DIC as activators.

At the end of the synthesis, the dry peptide-resins are individually treated with 25 mL of the cleavage mixture, 88% TFA, 5% phenol, 2% triisopropylsilane and 5% water for 1.5 hours at room temperature. Each resin is then filtered and then added to cold methyl-t-butyl ether in order to precipitate the peptide. After centrifugation, the peptide pellets are washed with fresh cold methyl-t-butyl ether to remove the organic scavengers. The process may be repeated twice. Final pellets are dried, resuspended in H₂O, 20% acetonitrile, and lyophilized.

The crude peptides (140 mg in 3 mL of DMSO) are purified by reverse-phase HPLC using preparative Waters Deltapak C4 (40×200 mm, 15 μm, 300 Å) and using as eluents (A) 0.1% TFA in water and (B) 0.1% TFA in acetonitrile.

Analytical HPLC may be performed on an Acquity UPLC Waters Chromatograph with a BEH300 C4 Acquity Waters column 2.1×100 mm, 1.7 μm, at 45° C., using H₂O, 0.1% TFA (A) and CH₃CN, 0.1% TFA (B) as solvents. The peptides may be characterized by electrospray mass spectrometry on an Acquity SQ Detector.

### Example 2

Synthesis of Linker 16-Azido esadecanoic acid may be performed as follows.

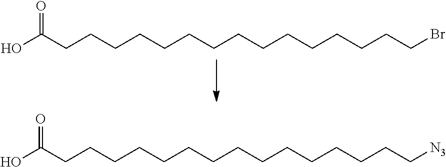 [SMILES: O=C(O)CCCCCCCCCCCCCCCBr ; [N-]=[N+]=NCCCCCCCCCCCCCCCC(=O)O]

To a solution of 16-bromo hexadecanoic acid in DMF, sodium azide (2 eq) is added. After 12 hours at 85 C°, the reaction mixture is cooled to room temperature. DCM is added and the organic phase is washed with HCl 0.1N, brine and dried over Na₂SO₄. The solvents are removed under reduced pressure and 16-azido hexadecanoic acid is obtained. ¹H NMR (400 MHz, CDCl₃-d₆, 300K) δ 12.35 (s, 1H), 3.30-3.22 (m, 2H), 2.40-2.32 (m, 2H), 1.69-1.56 (m, 4H), 1.4-1.2 (m, 20H).

### Example 3

Synthesis of Linker Propargyl-PEG₂₅-acid may be as follows.

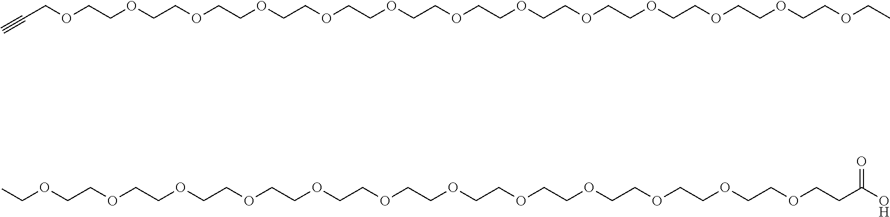

Step 1: To a suspension of sodium hydride, 60% dispersion in mineral oil (18 mg, 0.450 mmol) in THF (1 mL) cooled in an ice bath is added a solution of hydroxy-PEG₂₄-t-butyl ester (250 mg, 0.208 mmol) in Tetrahydrofuran (THF) (1.5 mL). The reaction mixture is stirred for 15 minutes and propargyl bromide, 80% in toluene (26.9 μl, 0.249 mmol) is added. The ice bath is removed and the reaction is allowed to warm to room temperature (RT) and stirred overnight. The reaction is quenched with water (50 μL), diluted with EtOAc, dried over sodium sulfate, filtered and concentrated to give the crude product propargyl-PEG25-t-butyl ester.

Step 2: TFA (1 mL, 12.98 mmol) is added to the crude propargyl-PEG₂₅-t-butyl ester and the reaction is stirred at RT for one hour. The volatiles are evaporated and the residue is purified by mass-directed RP-HPLC (ACN/water with 0.1% ammonium hydroxide as modifier) to give propargyl-PEG₂₅-acid. MS: 1186 (M+1).

### Example 4

Synthesis of Linker 2-(2-(2-(Pent-4-ynamido)ethoxy)ethoxy)acetic acid (Propargyl-C₅-PEG₂-acid) may be as follows.

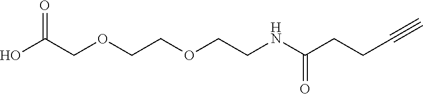 [SMILES: C#CCCC(=O)NCCOCCOCC(=O)O]

To a solution of 2-(2-(2-aminoethoxy)ethoxy)acetic acid (1.0 g, 6.13 mmol) and 2,5-dioxopyrrolidin-1-yl pent-4-ynoate (1.2 g, 6.13 mmol) in dimethylformamide (DMF) (10 mL) is added N,N-diisopropylethylamine (DIPEA) (1.28 ml, 7.35 mmol) at RT. The mixture is stirred at RT overnight. The reaction is quenched with water and lyophilized to dryness. The residue is purified by mass-directed RP-HPLC (ACN/water with 0.1% TFA as modifier) to give 2-(2-(2-(pent-4-ynamido)ethoxy)ethoxy)acetic acid. MS: 266 (M+23).

### Example 5

Synthesis of Linker 10,19,28-Trioxo-3,6,12,15,21,24-hexaoxa-9,18,27-triazadotriacont-31-ynoic acid (Propargyl-C₅-(PEG₂)₃-acid).

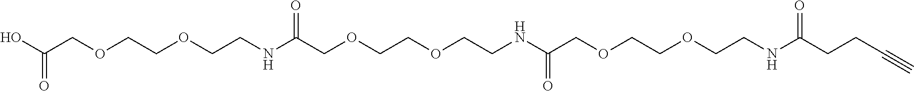 [SMILES: C#CCCC(=O)NCCOCCOCC(=O)NCCOCCOCC(=O)NCCOCCOCC(=O)O]

Step 1: In a 20 mL vial is added 2-(2-(2-(pent-4-ynamido)ethoxy)ethoxy)acetic acid (500 mg, 2.055 mmol) and DMSO (2 mL). TSTU (dimethylamino-(2,5-dioxopyrrolidin-1-yl)oxymethylidene]-dimethylazanium; tetrafluoroborate; 681 mg, 2.261 mmol) and triethylamine (573 μl, 4.11 mmol) are added. The mixture is stirred at RT for two hours. The freshly prepared N-Hydroxysuccinimide (NHS) ester is then added to a solution of 2-(2-aminoethoxy)ethoxy)acetic acid (419 mg, 2.57 mmol) and triethylamine (2.86 ml, 20.55 mmol) in DMSO (1 mL). The reaction is stirred at RT for 48 hours and triethylamine is removed under reduced pressure. Two drops of trifluoroacetate (TFA) are added to neutralize the reaction. The mixture is filtered through a syringe filter. The residue is purified by mass-directed RP-HPLC (ACN/water with 0.1% TFA as modifier) to give 10,19-dioxo-3,6,12,15-tetraoxa-9,18-diazatricos-22-ynoic acid.

Step 2: In a 20 ml vial is added 10,19-dioxo-3,6,12,15-tetraoxa-9,18-diazatricos-22-ynoic acid (132 mg, 0.340 mmol) and DMSO (1 mL). TSTU (113 mg, 0.374 mmol) and triethylamine (95 μl, 0.680 mmol) are added. The mixture is stirred at RT for two hours. The freshly prepared NHS ester is then added to a solution of 2-(2-(2-aminoethoxy)ethoxy)acetic acid (111 mg, 0.680 mmol) and triethylamine (474 μl, 3.40 mmol) in DMSO (1 mL). The reaction is quenched with TFA aqueous solution to slightly acidic. Water is added and the mixture is lyophilized to dryness. The residue purified by mass-directed RP-HPLC (ACN/water with 0.1% TFA as modifier) to give 10,19,28-trioxo-3,6,12,15,21,24-hexaoxa-9,18,27-triazadotriacont-31-ynoic acid. MS: 534 (M+1).

### Example 6

Synthesis of Linker Propargyl-C₅-Lys(γE-^{t}BuC₁₆)-PEG₁₂-acid.

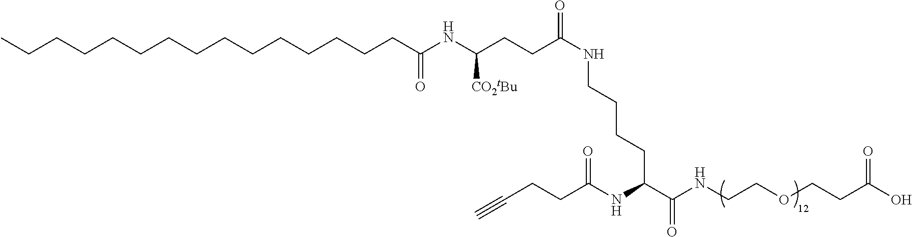

Step 1: To a mixture of L-Glu-O^{t}Bu (580 mg, 2.85 mmol) and NaHCO₃ (527 mg, 6.28 mmol) in water (10 mL) and THF (5 ml) at RT is added a solution of 2,5-dioxopyrrolidin-1-yl palmitate (1.01 g, 2.85 mmol) in THF (10 ml). The reaction mixture is stirred at RT overnight. THF is evaporated under reduced pressure. The reaction mixture is neutralized with TN HCl (7 mL), diluted with water, extracted with EtOAc, washed with brine, dried over sodium sulfate, filtered and concentrated to give the crude product (S)-5-(tert-butoxy)-5-oxo-4-palmitamidopentanoic acid.

Step 2: To a mixture of H-Lys(Boc)-OH (600 mg, 2.436 mmol) and NaHCO₃ (450 mg, 5.36 mmol) in water (10 mL) and THF (5 mL) at RT is added a solution of 2,5-dioxopyrrolidin-1-yl pent-4-ynoate (475 mg, 2.436 mmol) in THF (10 mL). The reaction mixture is stirred at RT overnight. THF is evaporated under reduced pressure. The reaction mixture is neutralized with TN HCl (7 mL), diluted with water, extracted with EtOAc, washed with brine, dried over sodium sulfate, filtered and concentrated to give the product (S)-6-((tert-butoxycarbonyl)amino)-2-(pent-4-ynamido)hexanoic acid as light yellow oil. TFA (3 mL, 38.9 mmol) is added to the crude (S)-6-((tert-butoxycarbonyl)amino)-2-(pent-4-ynamido)hexanoic acid (0.795 g, 2.436 mmol) at room temperature and the reaction mixture is stirred for one hour. TFA is evaporated under reduced pressure and the residue is quenched with water and lyophilized to give the product (S)-6-amino-2-(pent-4-ynamido)hexanoic acid, 2TFA.

Step 3: In a 20 mL vial is added (S)-5-(tert-butoxy)-5-oxo-4-palmitamidopentanoic acid (500 mg, 1.13 mmol) and DMSO (1 mL). TSTU (375 mg, 1.245 mmol) and triethylamine (316 μl, 2.264 mmol) are added. The mixture is stirred at RT for two hours. The freshly prepared NHS ester is then added to a solution of (S)-6-amino-2-(pent-4-ynamido)hexanoic acid, 2TFA (643 mg, 1.415 mmol) and triethylamine (1.58 mL, 11.32 mmol) in DMSO (1 mL). The reaction mixture is stirred at RT overnight. The reaction is diluted with water, acidified with 1N HCl, extracted with EtOAc, washed with water and brine, dried over sodium sulfate, filtered and concentrated. The residue is purified by mass-directed RP-HPLC (ACN/water with 0.1% TFA as modifier) to give N⁶—((S)-5-(tert-butoxy)-5-oxo-4-palmitamidopentanoyl)-N²-(pent-4-ynoyl)-L-lysine.

Step 4: In a 20 mL vial is added N⁶—((S)-5-(tert-butoxy)-5-oxo-4-palmitamidopentanoyl)-N²-(pent-4-ynoyl)-L-lysine (100 mg, 0.154 mmol) and DMSO (0.8 mL). TSTU (51 mg, 0.169 mmol) and triethylamine (43 μl, 0.308 mmol) is added. The mixture is stirred at RT for two hours. The freshly prepared NHS ester is then added to a solution of amino-PEG₁₂-acid (124 mg, 0.192 mmol) and triethylamine (214 μl, 1.539 mmol) in DMSO (0.5 mL). The reaction is stirred at RT overnight. The reaction is diluted with water and lyophilized to dryness. The residue is purified by mass-directed RP-HPLC (ACN/water with 0.1% TFA as modifier) to give propargyl-C₅-Lys(γE-^{t}Bu₁₆)-PEG₁₂-acid. MS: 1250 (M+1).

### Example 7

Synthesis of Propargyl-(PEG₄)(PEG₂₄)-acid.

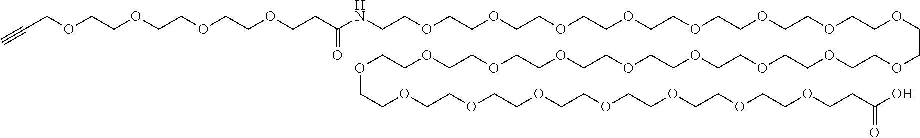 [SMILES: C#CCOCCOCCOCCOCCC(=O)NCCOCCOCCOCCOCCOCCOCCOCCOCCOCCOCCOCCOCCOCCOCCOCCOCCOCCOCCOCCOCCOCCOCCOCCOCCC(=O)O]

Amino-PEG₂₄-acid (100 mg, 0.087 mmol) and NaHCO₃ (16.12 mg, 0.192 mmol) are suspended in water (2 mL) and THF (1 ml) at room temperature. To the mixture is added a solution of propargyl-PEG₄-NHS (32.7 mg, 0.092 mmol) in THF (2 mL). The reaction is stirred at RT overnight. The reaction is neutralized by 0.1 M HCl (2 mL) and lyophilized to dryness. The residue is purified by mass-directed RP-HPLC (ACN/water with 0.1% TFA as modifier) to give propargyl-(PEG₄)(PEG₂₄)-acid. MS: 695 (M+2)/2.

### Example 8

Synthesis of Propargyl-(PEG₄)(PEG₃₆)-acid.

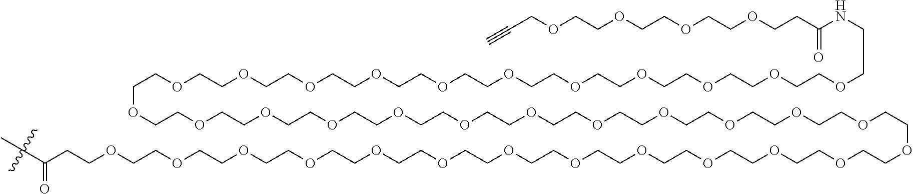 [SMILES: *C(=O)CCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCNC(=O)CCOCCOCCOCCOCC#C]

Amino-PEG₃₆-acid (100 mg, 0.060 mmol) and NaHCO₃ (11.03 mg, 0.131 mmol) are suspended in water (2 mL) and THF (1 mL) at room temperature. To the mixture is added a solution of propargyl-PEG₄-NHS (22.4 mg, 0.063 mmol) in THF (2 mL). The reaction is stirred at RT overnight. The reaction is neutralized by 0.1 M HCl (2 mL) and lyophilized to dryness. The residue is purified by mass-directed RP-HPLC (ACN/water with 0.1% TFA as modifier) to give propargyl-(PEG₄)(PEG₃₆)-acid. MS: 640 (M+3)/3.

### Example 9

Synthesis of Propargyl-(PEG₅(PEG₃₆)₂-acid.

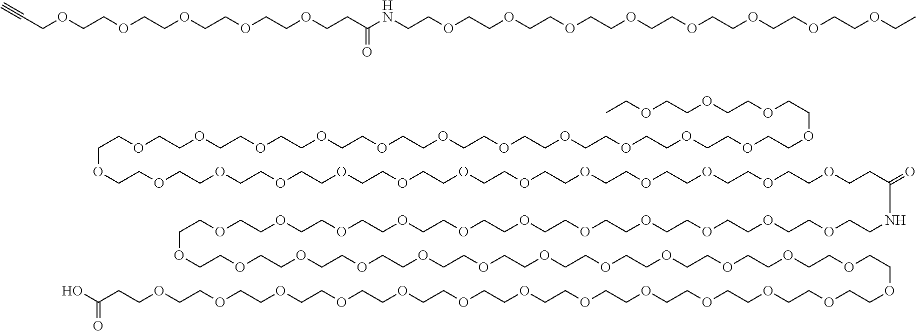 [SMILES: C#CCOCCOCCOCCOCCOCCC(=O)NCCOCCOCCOCCOCCOCCOCCOCCOCC ; CCOCCOCCOCCOCCOCCOCCOCCOCCOCCOCCOCCOCCOCCOCCOCCOCCOCCOCCOCCOCCOCCOCCOCCOCCOCCOCCOCCOCCC(=O)NCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCC(=O)O]

Step 1: Amino-PEG₃₆-acid (1.04 g, 0.623 mmol) and NaHCO₃ (115 mg, 1.37 mmol) are suspended in water (20 mL) and THF (10 mL) at room temperature. To the mixture is added a solution of propargyl-PEG₅-NHS (262 mg, 0.65 mmol) in THF (20 mL). The reaction is stirred at RT overnight. The reaction is neutralized by 0.1 M HCl (20 mL) and lyophilized to dryness. The residue is purified by mass-directed RP-HPLC (ACN/water with 0.1% TFA as modifier) to give propargyl-(PEG₅)(PEG₃₆)-acid as white solids.

Step 2: In a 20 ml vial was added propargyl-(PEG₅)(PEG₃₆)-acid (100 mg, 0.051 mmol) and DMSO (0.8 mL). TSTU (16.88 mg, 0.056 mmol) and triethylamine (14.21 μL, 0.102 mmol) was added. The mixture was stirred at RT for two hours. The freshly prepared NHS ester is then added to a solution of amino-PEG₃₆-acid (107 mg, 0.064 mmol) and triethylamine (71.1 μL, 0.510 mmol) in DMSO (0.5 mL). The reaction is stirred at RT overnight. The reaction is diluted with water and lyophilized to dryness. The residue is purified by mass-directed RP-HPLC (ACN/water with 0.1% TFA as modifier) to give proparyl-(PEG₅)(PEG₃₆)₂-acid as white solids. MS: 517.85 (M+7)/7.

### Example 10

Synthesis of N^{αA1}N^{ε29} bis-Boc RHI.

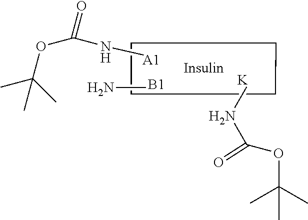

RHI (10 g) is dissolved in DMSO (200 mL) at RT the mixture is stirred at RT until homogeneous. To the solution is added 2,2,6,6-tetramethylpiperidine (5.8 mL) followed slow addition of a solution of tert-butyl (2,5-dioxopyrrolidin-1-yl) carbonate (Boc-OSu, 0.8 g) in DMSO (10 mL). The reaction is agitated for four hours and the mixture is transferred to IPAc (1 L) over 20 minutes. The slurry is centrifuged for two hours and the solid is filtered, washed with IPAc (20 ml×3) and dried under vacuum to give N^{αA1}N^{εB29} bis-Boc RHI.

### Example 11

Synthesis of N^{αA1}-propargyl-C₅ RHI (INS1).

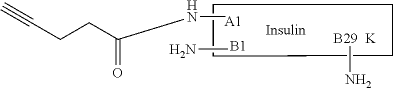

Insulin (655 mg, 0.113 mmol) is first dissolved in pH 2.5 water (12 mL) and then adjusted to pH 8.5 with 1N NaOH. A stock solution of 2,5-dioxopyrrolidin-1-yl pent-4-ynoate (35 mg, 0.174 mmol) in DMSO (200 μL) is added in four portions to the above insulin solution over one hour. The pH is maintained at 8.0-8.5. The reaction is quenched with ethanolamine (17 μL) after five hours and adjusted to pH 7.0. The reaction mixture is first purified by IEC HPLC with the following conditions: PolySULFOETHYL ATM column, 250×21.0 mm, 5 um, 1000 A, eluent (A: 1 L ACN, 3 L H₂O, 4 ml H₃PO₄; B: 1 L ACN, 2.6 L H₂O, 400 ml 5M NaCl, 4 ml H₃PO₄), FR=15 mL/minute, wavelength 210 nm, B % from 10% to 40%. The major fractions are concentrated down to 15 mL by 8×10K Amicon centrifuge tube. The concentrated crude is further purified by RP-HPLC, using the separation condition (28-33%) 0.05% TFA in ACN/H₂O on Kromasil 100-10-C8 from AkzoNobel, 100 A and 10 uM 50×250 mm column; FR=85 ml/min, ramp 25 minutes, wavelength=210 nm. The desired fractions are collected and lyophilized to N^{αA1}-propargyl-C₅ RHI.

### Example 12

Synthesis of N^{αB1}-propargyl-C5 RHI (INS2).

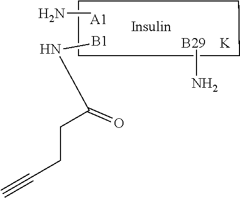

Step 1: RHI (2.74 g, 0.472 mmol) is dissolved in DMSO (50 mL) and the mixture is stirred at RT until homogeneous (about 45 minutes). To the solution is added 1,1,3,3-tetramethylguanidine (0.19 mL, 1.179 mmol) and followed by slow addition of a solution of Boc-OSu (0.216 g, 1.005 mmol) DMSO (2.1 mL, plus 0.4 mL wash) over 30 minutes. The reaction is further stirred for 1.5 hours for N^{αA1}N^{εB29} bis-Boc RHI to be formed. To the reaction mixture is added a solution of 2,5-dioxopyrrolidin-1-yl pent-4-ynoate in DMSO (1 mL) using a syringe pump over 30 minutes. After stirring for two hours, the reaction mixture is dropwise added to a stirred 275 mL of IPAc to precipitate the product. The white solids are filtered, washed with IPAc and dried under N₂ and vacuum for one hour.

Step 2: TFA (15 mL, 195 mmol) is added to the above crude material (3 g, 0.493 mmol) and the reaction is stirred and sonicated until solids fully dissolved (about 30 minutes). The reaction mixture is dropwise added to water (75 mL) and the precipitated solids are filtered and dried under N₂ and vacuum. The crude material is purified using RP-HPLC to give the N^{αB1}-propargyl-C₅ RHI.

### Example 13

Synthesis of N^{αB1}-propargyl-PEG₄ RHI (INS3).

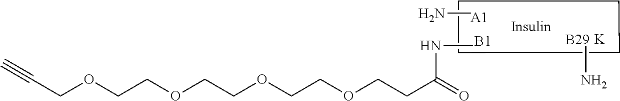

Step 1: To a solution of N^{αA1}N^{εB29} bis-Boc RHI (317 mg, 0.053 mmol) in DMSO (2 mLl) is added Et₃N (37 μL, 0.264 mmol) and followed by slow addition of alkyne-PEG₄-NHS (2,5-dioxopyrrolidin-1-yl 4,7,10,13-tetraoxahexadec-15-ynoate, 37.7 mg, 0.106 mmol) in DMSO (800 μL) via syringe pump over one hour. The reaction mixture is stirred at RT overnight. The reaction mixture is dropwise added to stirred isopropyl acetate (IPAc, 50 mL) to precipitate the product. The white precipitate is collected by filtration and dried under nitrogen gas and vacuum for one hour to give the crude product N^{αA1}N^{εB29} bis-Boc, N^{αB1}-propargyl-PEG₄ RHI.

Step 2: TFA (1.5 mL, 19.47 mmol) is added to the crude N^{αA1}N^{εB29} bis-Boc, N^{αB1}-PEG₄-alkyne RHI. The reaction is stirred and sonicated at RT until solids fully dissolved (about 30 minutes). The reaction mixture is dropwise added to tert-butyl methyl ether (TBME, 20 mL) and solids precipitated out. The reaction is filtered, washed with TBME and IPAc. The white solid is dried under nitrogen gas and vacuum for one hour to give crude product as white solids. The solids are dissolved in water (15 mL). It is purified by RP-HPLC column using the separation condition (28-35%) 0.05% TFA in AcCN/H₂O on Kromasil 100-10-C8 from AkzoNobel, 100 A and 10 uM 50×250 mm column; FR=85 mL/minute, ramp 25 minutes, wavelength=210 nm. The fractions are collected and lyophilized to give N^{αB1}-propargyl-PEG₄-RHI.

INS4-INS6 shown below may be prepared using the methodology herein and the general procedure described for INS3.

| | |
|---|---|
| INS4 | N^{αB1}-propargyl-PEG₃ RHI 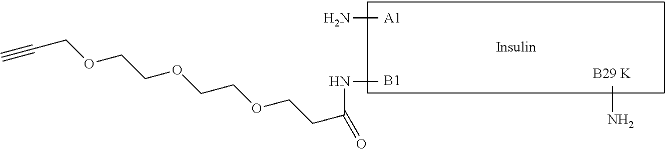 |
| INS5 | N^{αB1}-propargyl-PEG₅ RHI 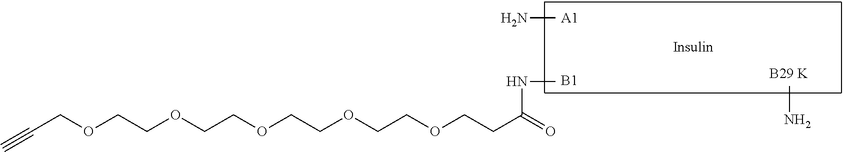 |
| INS6 | N^{αB1}-BCN—PEG₄(endo) RHI 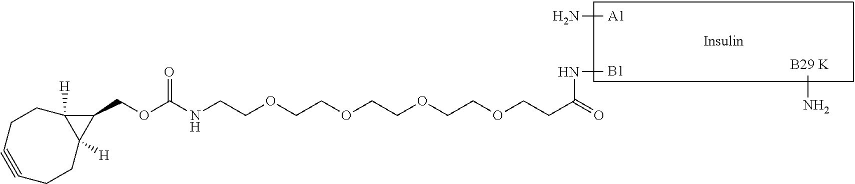 BCN = bicyclononyne |

### Example 14

Synthesis of N^{αB1}-propargyl-PEG₆ RHI (INS7).

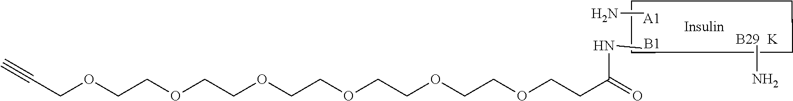

In a small vial, propargyl-PEG₆-acid (4,7,10,13,16,19-hexaoxadocos-21-yn-1-oic acid, 11.6 mg, 0.033 mmol) is dissolved in DMSO (500 μL). To the solution is added TSTU (O—(N-Succinimidyl)-N,N,N′,N′-tetramethyluronium tetrafluoroborate, 10.0 mg, 0.033 mmol) and Et₃N (4.6 μl, 0.033 mmol). The mixture is stirred at RT for two hours to give the crude propargyl-PEG₆-NHS. The freshly made propargyl-PEG₆-NHS solution is added to a solution of N^{αA1}N^{εB29} bis-Boc RHI (100 mg, 0.017 mmol) and Et₃N (12 μL, 0.086 mmol) in DMSO (1 mL) via syringe pump over 30 minutes. The reaction mixture is stirred at RT for two hours. The reaction mixture is dropwise added to a stirred IPAc (20 mL) to precipitate the product. The white precipitate is collected by filtration and dried under nitrogen gas and vacuum for one hour to give the crude product N^{αA1}N^{εB29} bis-Boc, N^{αB1}-propargyl-PEG₆ RHI.

TFA (0.5 ml, 6.49 mmol) is added to the crude N^{αA1}N^{εB29} bis-Boc, N^{αB1}-PEG₆-alkyne RHI. The reaction is stirred and sonicated at RT until solids fully dissolved (about 30 minutes). The reaction mixture is dropwise added to tert-butyl methyl ether (TBME, 10 mL) and solids precipitated out. The reaction is filtered, washed with TBME and IPAc. The white solid is dried under nitrogen gas and vacuum for one hour to give crude product as white solids. The solids are dissolved in water (8 mL). It is purified by RP-HPLC column using the separation condition (28-35%) 0.05% TFA in AcCN/H₂O on Kromasil 100-10-C8 from AkzoNobel, 100 A and 10 μM 50×250 mm column; FR=85 mL/minute, ramp 25 minutes, wavelength=210 nm. The fractions are collected and lyophilized to give the N^{αB1}-propargyl PEG₆ RHI.

INS 8-INS15 shown below may be prepared using the methodology herein and the general procedure described for INS7.

| | |
|---|---|
| INS8 | N^{αB1}-propargyl-PEG₈ RHI 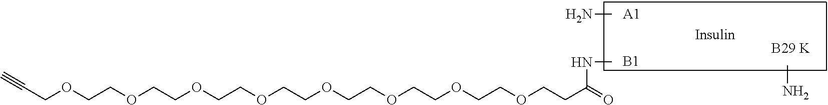 |
| INS9 | N^{αB1}-propargyl-PEG₁₀ RHI 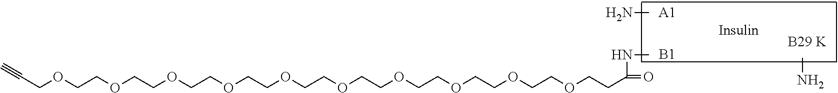 |
| INS10 | N^{αB1}-propargyl-PEG₁₃ RHI 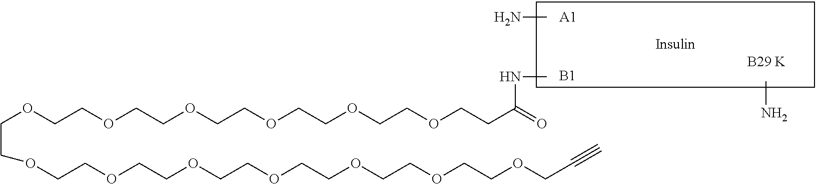 |
| INS11 | N^{αB1}-propargyl-PEG₁₄ RHI 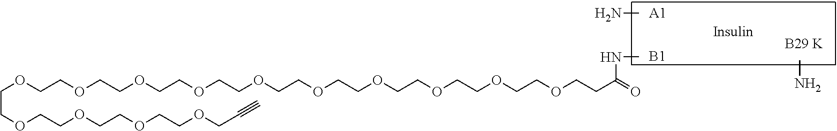 |
| INS12 | N^{αB1}-propargyl-PEG₂₅ RHI 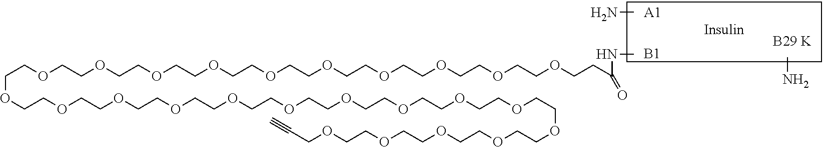 |
| INS13 | N^{αB1}-propargyl-PEG₂—C₅ RHI 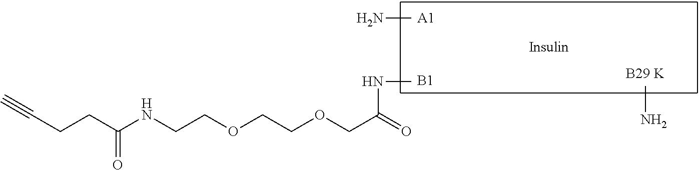 |
| INS14 | N^{αB1}-propargyl-C5—(PEG₂)₃ RHI 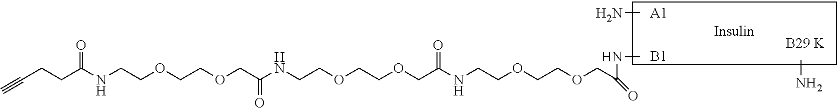 |
| INS15 | N^{αB1}-propargyl-C5-Lys(γE-C₁₆)—PEG₁₂— RHI 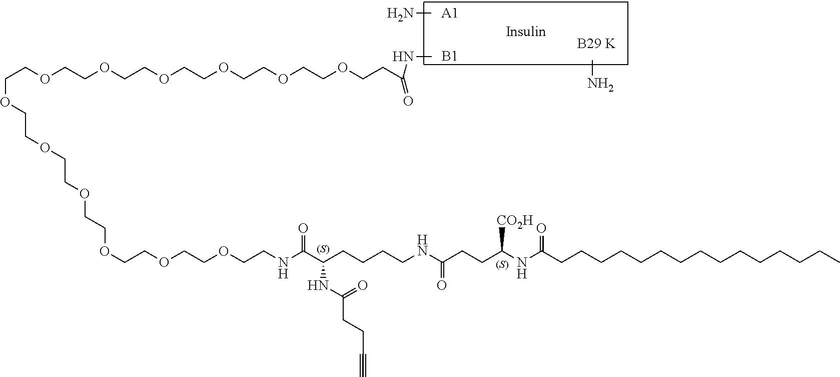 |

### Example 15

Synthesis of N^{εB29}-propargyl-C₅ RHI (INS16).

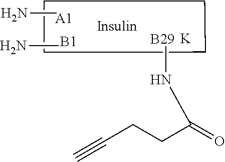

General Method: In an appropriate sized container, RHI is dissolved, with gentle stirring, at room temperature in a mixed solvent: 2:3 v/v 0.1 M Na₂CO₃:AcCN. After the mixture cleared, the pH is adjusted to the value of 10.5-10.8 using alkaline solution, e.g., 0.1 N NaOH. In a separate vial, 2,5-dioxopyrrolidin-1-yl pent-4-ynoate is dissolved in an organic solvent, e.g., DMSO, at room temperature. Aliquots of the solution of the activated ester is added over a period of time to the solution containing insulin until UPLC chromatogram showed that most of the unmodified insulin had been reacted and that a substantial portion of the reaction mixture had been converted into B29-conjugated insulin. The reaction is quenched by the addition of an amine nucleophile, e.g., 2-aminoethanol. The reaction solution is stirred at room temperature for 30 minutes. The resulting solution is carefully diluted with cold H₂O (20×) at 0° C. and its pH is adjusted to a final pH of 2.5 using 1 N HCl (and 0.1 N NaOH if needed). The solution is first concentrated by ultrafiltration, either through a tangential flow filtration (TFF) system or using Amicon Ultra-15 Centrifugal Units, with 1K, 3K or 10K MWCO membrane. The concentrated solution may be first subjected to ion exchange chromatography (PolySULFOETHYL A column, PolyLC Inc., 250×21 mm, 5 m, 1000 Å; Buffer A: 0.1% (v/v)H₃PO₄/25% AcCN; Buffer B: 0.1% (v/v)H₃PO₄/25% AcCN/0.5 M NaCl). Fractions containing B29-conjugate with desired purity are combined and concentrated using TFF system or Amicon Ultra-15. The resulting solution is then further purified by reverse phase HPLC (Waters C4 250×50 mm column, 10 m, 1000 Å column or Kromasil C8 250×50 mm, 10 m, 100 Å column; Buffer A: 0.05-0.1% TFA in water; Buffer B: 0.05-0.1% TFA in AcCN). Fractions containing the title conjugate are combined and freeze-dried or buffer exchanged using TFF system and/or Amicon Ultra-15 to give the N^{εB29}-propargyl-C₅ RHI. MS: 1472.56 (M+4)/4.

### Example 16

Synthesis of N^{εB29}-propargyl-PEG₄ RHI (INS17).

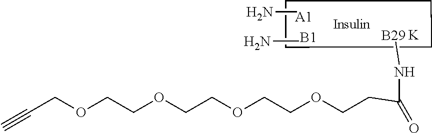

In a 20 mL vial, RHI (200 mg, 0.034 mmol) is dissolved in DMSO (1 mL) and aged at RT until homogeneous. 1,1,3,3-tetramethylguanidine (86 μl, 0.689 mmol) is added and followed by slow addition of alkyne-PEG₄-NHS (2,5-dioxopyrrolidin-1-yl 4,7,10,13-tetraoxahexadec-15-ynoate, 13.5 mg, 0.038 mmol) solution in DMSO (0.5 ml) via syringe pump over 30 min. The reaction is stirred at RT for one hour. The mixture is added dropwise to a mixed solvent IPAc/TBME(20 mL, 4:1) to precipitate out the product. HOAc (200 μl, 3.49 mmol) is added. The solids are filtered and dried under nitrogen gas and vacuum for 30 minutes. The solids are dissolved in 15 mL mix solvent (80% water, 20% ACN, pH=3.0), adjusted pH to 3. The residue is purified by RP-HPLC column, using the separation condition (28-35%) 0.05% TFA in AcCN/H₂O on Kromasil 100-10-C8 from AkzoNobel, 100 A and 10 μM 50×250 mm column; FR=85 mL/minute, ramp 25 minutes, wavelength=210 nm. The fractions are collected and lyophilized to give N^{εB29}-propargyl-PEG₄ RHI.

### Example 17

N^{εB29}-propargyl-PEG₄ insulin glargine (INS18).

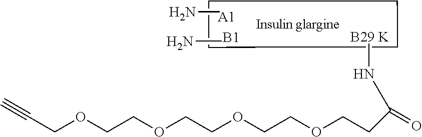

N^{εB29}-propargyl-PEG₄ insulin glargine may be prepared using the methodology herein and the general procedure described for INS16.

### Example 18

Synthesis of N^{εB29}-propargyl-PEG₁₃ RHI (INS19).

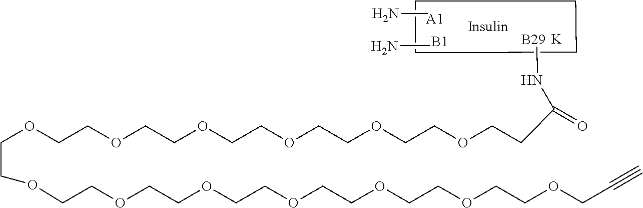

In a small vial, propargyl-PEG₁₃-acid (4,7,10,13,16,19,22,25,28,31,34,37,40-tridecaoxatritetracont-42-ynoic acid, 24.9 mg, 0.038 mmol) is dissolved in DMSO (500 μL). To the solution is added TSTU (11.4 mg, 0.038 mmol) and Et₃N (7.2 μl, 0.052 mmol). The mixture is stirred at RT for two hours. The freshly made propargyl-PEG6-NHS solution is added to a solution of RHI (200 mg, 0.034 mmol) and 1,1,3,3-tetramethylguanidine (0.086 ml, 0.689 mmol) in DMSO two mL via syringe pump over 60 minutes. The reaction mixture is stirred at RT for 30 minutes. The reaction is quenched with 2-aminoethanol (10.4 μl, 0.172 mmol) for 10 minutes. The reaction mixture is dropwise added to a stirred 20 mL of IPAc/TBME (4:1) to precipitate the product. The solids are filtered, washed with IPAc, and dried under nitrogen gas and vacuum for one hour. The residue is purified by RP-HPLC (ACN/water with 0.1% TFA as modifier) to give N^{εB29}-propargyl-PEG₁₃ RHI.

INS20-INS21 shown below may be prepared using the methodology herein and the general procedure described for INS19.

| | |
|---|---|
| INS20 | N^{εB29}-propargyl-(PEG₄)—(PEG₂₄) RHI 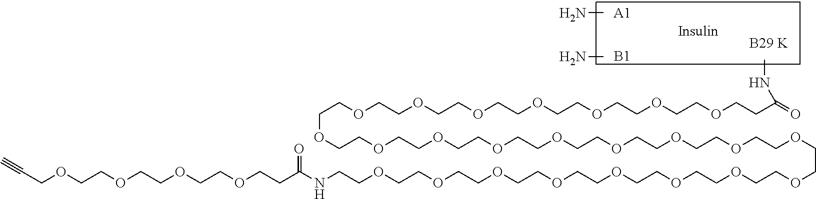 |
| INS21 | N^{εB29}-propargyl-(PEG₄)—(PEG₃₆) RHI 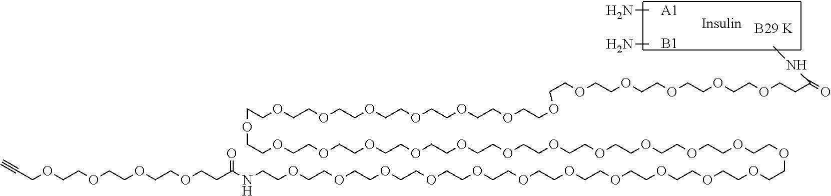 |
| INS25 | N^{εB29}-propargyl-(PEG₅)—(PEG₃₆)₂ RHI 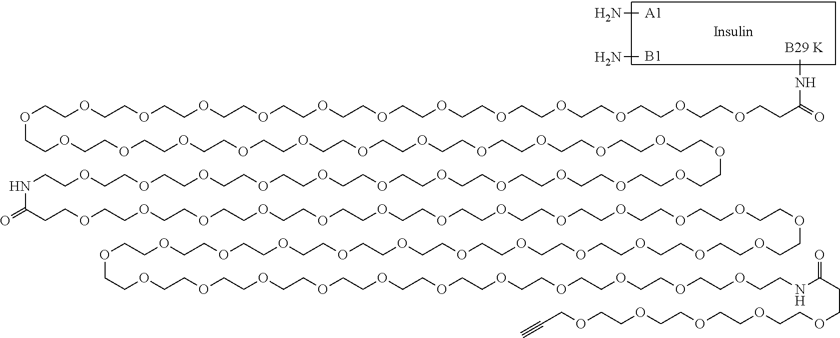 |

### Example 19

Synthesis of N^{αB1}-propargyl-C5 N^{εB29}C₁₄ RHI (INS22).

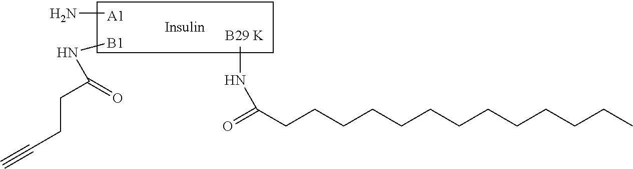

Step 1: N^{εB29}C₁₄ RHI (2.74 g, 0.472 mmol) is dissolved in DMSO (50 mL) and the mixture is stirred at RT until homogeneous (about 45 minutes). To the solution is added 1,1,3,3-tetramethylguanidine (0.19 mL, 1.179 mmol) and followed by slow addition of a solution of Boc-OSu (0.216 g, 1.005 mmol) DMSO (2.1 mL, plus 0.4 mL wash) over 30 minutes. The reaction is further stirred for 1.5 hours for N^{αA1} Boc RHI to be formed. To the reaction mixture is added a solution of 2,5-dioxopyrrolidin-1-yl pent-4-ynoate in DMSO (1 mL) using a syringe pump over 30 minutes. After stirring for two hours, the reaction mixture is dropwise added to a stirred 275 mL of IPAc to precipitate the product. The white solids are filtered, washed with IPAc and dried under N₂ and vacuum for one hour.

Step 2: TFA (15 mL, 195 mmol) is added to the above crude material (3 g, 0.493 mmol) and the reaction is stirred and sonicated until solids fully dissolved (about 30 minutes). The reaction mixture is dropwise added to water (75 mL) and the precipitated solids are filtered and dried under N₂ and vacuum. The crude material is purified using RP-HPLC to give the N^{αB1}-propargyl-C₅ N^{εB29}C₁₄ RHI.

### Example 20

Synthesis of N^{αB1}-propargyl-C₅ N^{εB29}γE-C₁₆ RHI (INS23).

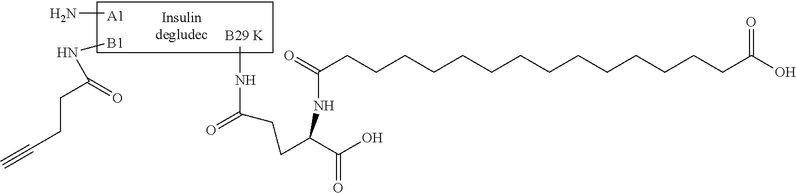

Step 1: N^{εB29}γE-C₁₆ RHI (2.74 g, 0.472 mmol) is dissolved in DMSO (50 mL) and the mixture is stirred at RT until homogeneous (about 45 minutes). To the solution is added 1,1,3,3-tetramethylguanidine (0.19 mL, 1.179 mmol) and followed by slow addition of a solution of Boc-OSu (0.216 g, 1.005 mmol) DMSO (2.1 mL, plus 0.4 mL wash) over 30 minutes. The reaction is further stirred for 1.5 hours for N^{αA1} Boc RHI to be formed. To the reaction mixture is added a solution of 2,5-dioxopyrrolidin-1-yl pent-4-ynoate in DMSO (1 mL) using a syringe pump over 30 minutes. After stirring for two hours, the reaction mixture is dropwise added to a stirred 275 mL of IPAc to precipitate the product. The white solids are filtered, washed with IPAc and dried under N₂ and vacuum for one hour.

Step 2: TFA (15 mL, 195 mmol) is added to the above crude material (3 g, 0.493 mmol) and the reaction is stirred and sonicated until solids fully dissolved (about 30 minutes). The reaction mixture is dropwise added to water (75 mL) and the precipitated solids are filtered and dried under N₂ and vacuum. The crude material is purified using RP-HPLC to give the N^{αB1}-propargyl-C₅ N^{εB29}γE-C₁₆ RHI.

### Example 21

Synthesis of N^{αB1}-propargyl-PEG₁₃ N^{εB29}γE-C₁₆ RHI (INS24).

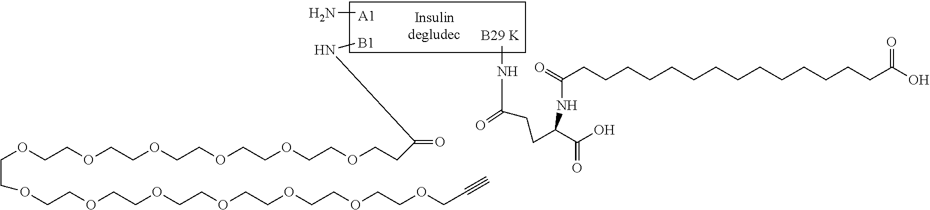

Step 1: N^{εB29}γE-C₁₆ RHI (2.74 g, 0.472 mmol) is dissolved in DMSO (50 mL) and the mixture is stirred at RT until homogeneous (about 45 minutes). To the solution is added 1,1,3,3-tetramethylguanidine (0.19 mL, 1.179 mmol) and followed by slow addition of a solution of Boc-OSu (0.216 g, 1.005 mmol) DMSO (2.1 mL, plus 0.4 mL wash) over 30 minutes. The reaction is further stirred for 1.5 hours for N^{αA1} Boc RHI to be formed. To the reaction mixture is added a solution of 2,5-dioxopyrrolidin-1-yl pent-4-ynoate in DMSO (1 mL) using a syringe pump over 30 minutes. After stirring for two hours, the reaction mixture is dropwise added to a stirred 275 mL of IPAc to precipitate the product. The white solids are filtered, washed with IPAc and dried under N₂ and vacuum for one hour.

Step 2: TFA (15 mL, 195 mmol) is added to the above crude material (3 g, 0.493 mmol) and the reaction is stirred and sonicated until solids fully dissolved (about 30 minutes). The reaction mixture is dropwise added to water (75 mL) and the precipitated solids are filtered and dried under N₂ and vacuum. The crude material is purified using RP-HPLC to give the N^{αB1}-propargyl-PEG₁₃ N^{εB29}γE-C₁₆ RHI.

### Example 21

General procedure A for insulin-incretin conjugate preparation (with TBTA ligand) using copper(I) catalyzed Azide-Alkyne Cycloaddition (CuAAC) (schematically shown in FIG. 1B):

A 10 mM Cu(II)-TBTA in 55% DMSO stock solution is prepared as follows: CuSO₄.5H₂O (50 mg) is dissolved in water (10 mL) and Tris[(1-benzyl-1H-1,2,3-triazol-4-yl)methyl]amine (TBTA, 116 mg) is dissolved in DMSO (11 mL). The two solutions are then mixed slowly at 0° C.

In a 20 mL vial, incretin peptide comprising an azido group (11 mg, 2.79 μmol) is dissolved in a pre-mixed solvent DMSO/H₂O (5 mL, 2:3). In another 50 mL centrifuge tube insulin intermediate comprising an alkyne group, e.g. N^{αB1}-propargyl-PEG₄ RHI, (17.4 mg, 2.87 μmol) is dissolved in a pre-mixed solvent DMSO/H₂O (5 ml, 2:3), and then 2 M triethylammonium acetate buffer solution (pH 7.0, 1.2 mL) is added. The above two solutions are mixed on a vortex and degassed by gently bubbling nitrogen gas. To the reaction mixture is added freshly prepared 5 mM ascorbic acid solution (1.2 ml) and the mixture vortexed. The solution is degassed by bubbling N₂ for one minute. A stock solution of 10 mM Cu(II)-TBTA in 55% DMSO (0.6 mL) is added and the mixture is flushed with N₂ for two minutes. The mixture is shaken slowly overnight. The reaction mixture is diluted with 50 mL mixed solvent (80% water, 20% ACN pH=3.0) and the pH is adjusted to 3.0. The solution is concentrated using 3K Amicon centrifuge tube to final total volume 15 mL. The residue is purified by RP-HPLC, using the separation condition (31-46%) 0.05% TFA in ACN/H₂O on Kromasil 100-10-C8 from AkzoNobel, 100 A and 10 μM 50×250 mm column; FR=85 mL/minute, ramp 25 minutes, wavelength=210 nm. The desired fractions are collected and lyophilized to give the insulin-incretin conjugate wherein the azido and alkynyl groups have formed a 1,4-disubstituted 1,2,3-triazole. MS: 1993 (M+5)/5.

### Example 22

General procedure B for CuAAC conjugate preparation (without TBTA ligand): insulin intermediate comprising an alkyne group, e.g. 1N^{εB29}-propargyl-(PEG₄)-(PEG₂₄) RHI, (33 mg, 4.60 μmol) and incretin peptide comprising an azido group (23.9 mg, 5.52 μmol) are dissolved in DMSO (3.3 mL). Under nitrogen flow, to the above solution in a water bath is added dropwise a stock solution of CuSO₄ 5H₂O in water (1.23 mL, 0.014 mmol, 2.8 mg/ml). Subsequently, a freshly prepared solution of sodium ascorbate in water (0.969 mL, 0.018 mmol, 3.76 mg/mL) is added dropwise. The reaction is stirred at RT for two hours, quenched with a mixed solvent (10 mL, 80% water, 20% ACN, pH=3.0) and pH is adjusted to 3.0. The reaction solution is purified by RP-HPLC column, using the separation condition (32-47%) 0.05% TFA in ACN/H₂O on Kromasil 100-10-C8 from AkzoNobel, 100 A and 10 μM 50×250 mm column; FR=85 mL/minute, ramp 25 minutes, wavelength=210 nm. The desired fractions are collected and lyophilized to give the insulin-incretin conjugate wherein the azido and alkynyl groups have formed a 1,4-disubstituted 1,2,3-triazole. MS: 1849 (M+6)/6.

### Example 23

Preparation of N^{αB1}-BCN-PEG₄ (endo) RHI-incretin peptide conjugate using Copper-free click chemistry.

Insulin intermediate, e.g., N^{αB1}-BCN-PEG₄ (endo), (17 mg, 2.73 μmol) is dissolved in water (3 mL) to give a clear solution. To the solution is added 0.5 mL pH 7 buffer (2 M triethylammonium acetate buffer, pH=7.0:2.78 mL Et₃N mixed with 1.14 ml acetic acid, water is added to 10 mL, and the pH is adjusted to 7.0). Incretin peptide comprising an azido group (14.8 mg, 3.41 μmol) is dissolved in a mixed solvent (3 mL, water/ACN 3:2). The two solutions are mixed together and stirred at RT for six hours. The solution is adjusted pH with 1.0 N HCl until the solution turns clear (total volume 8 mL). The reaction solution is purified by RP-HPLC column, using the separation condition (31-47%) 0.05% TFA in AcCN/H₂O on Kromasil 100-10-C8 from AkzoNobel, 100 A and 10 uM 50×250 mm column; FR=85 mL/minute, ramp 25 minutes, wavelength=210 nm. Fractions are collected and lyophilized to give the product. MS: 1691 (M+6)/6.

### Example 24

Table 4 shows various insulin-incretin conjugates (“CON”) that have been synthesized according to methods disclosed herein. Each conjugate was synthesized from a particular peptide having a linker with a terminal azido group and a particular insulin having a linker with a terminal alkynal group to produce the conjugate in which the insulin and peptide are linked via the azido and alkynal groups in a 1,4 disubstituted 1,2,3-triazol linkage, except for CON23 which is linked via a 1,4,5 disubstituted 1,2,3-triazol.

TABLE 4
Conjugate

Components

| Name | Peptide | Insulin |
|---|---|---|
| CON1 | PEP68 | INS16 |
| CON2 | PEP68 | INS2 |
| CON3 | PEP68 | INS1 |
| CON4 | PEP69 | INS2 |
| CON5 | PEP69 | INS16 |
| CON6 | PEP69 | INS1 |
| CON7 | PEP70 | INS16 |
| CON8 | PEP70 | INS1 |
| CON9 | PEP70 | INS2 |
| CON10 | PEP71 | INS16 |
| CON11 | PEP71 | INS2 |
| CON12 | PEP71 | INS1 |
| CON13 | PEP72 | INS2 |
| CON14 | PEP73 | INS2 |
| CON15 | PEP73 | INS16 |
| CON16 | PEP74 | INS2 |
| CON17 | PEP74 | INS16 |
| CON19 | PEP69 | INS3 |
| CON20 | PEP69 | INS10 |
| CON21 | PEP74 | INS3 |
| CON22 | PEP74 | INS10 |
| CON23 | PEP74 | INS6 |
| CON24 | PEP74 | INS4 |
| CON25 | PEP74 | INS5 |
| CON26 | PEP74 | INS13 |
| CON27 | PEP74 | INS7 |
| CON28 | PEP1 | INS2 |
| CON29 | PEP2 | INS2 |
| CON30 | PEP3 | INS2 |
| CON31 | PEP4 | INS2 |
| CON32 | PEP5 | INS2 |
| CON33 | PEP6 | INS2 |
| CON34 | PEP7 | INS2 |
| CON35 | PEP8 | INS2 |
| CON36 | PEP9 | INS2 |
| CON37 | PEP10 | INS2 |
| CON38 | PEP11 | INS2 |
| CON39 | PEP12 | INS2 |
| CON40 | PEP13 | INS2 |
| CON41 | PEP14 | INS2 |
| CON42 | PEP15 | INS2 |
| CON43 | PEP16 | INS2 |
| CON44 | PEP17 | INS2 |
| CON45 | PEP18 | INS2 |
| CON46 | PEP19 | INS2 |
| CON47 | PEP20 | INS2 |
| CON48 | PEP21 | INS2 |
| CON49 | PEP22 | INS2 |
| CON50 | PEP25 | INS2 |
| CON51 | PEP26 | INS2 |
| CON52 | PEP27 | INS2 |
| CON53 | PEP28 | INS2 |
| CON55 | PEP74 | INS8 |
| CON56 | PEP74 | INS9 |
| CON57 | PEP74 | INS11 |
| CON58 | PEP69 | INS22 |
| CON59 | PEP69 | INS23 |
| CON60 | PEP74 | INS24 |
| CON61 | PEP74 | INS19 |
| CON62 | PEP74 | INS12 |
| CON63 | PEP10 | INS3 |
| CON64 | PEP74 | INS18 |
| CON65 | PEP74 | INS17 |
| CON66 | PEP74 | INS14 |
| CON67 | PEP69 | INS15 |
| CON68 | PEP74 | INS15 |
| CON69 | PEP74 | INS25 |
| CON70 | PEP74 | INS20 |
| CON71 | PEP29 | INS2 |
| CON72 | PEP30 | INS2 |
| CON73 | PEP31 | INS2 |
| CON74 | PEP32 | INS2 |
| CON75 | PEP34 | INS2 |
| CON76 | PEP35 | INS2 |
| CON77 | PEP37 | INS2 |
| CON78 | PEP74 | INS21 |
| CON79 | PEP74 | INS1 |
| CON80 | PEP1 | INS3 |
| CON81 | PEP6 | INS3 |
| CON82 | PEP29 | INS3 |
| CON83 | PEP33 | INS2 |
| CON84 | PEP36 | INS2 |
| CON85 | PEP38 | INS2 |
| CON86 | PEP39 | INS2 |
| CON87 | PEP40 | INS2 |
| CON88 | PEP43 | INS2 |
| CON89 | PEP41 | INS2 |
| CON90 | PEP42 | INS2 |
| CON91 | PEP75 | INS2 |
| CON92 | PEP74 | INS25 |
| CON93 | PEP1 | INS3 |
| CON94 | PEP6 | INS3 |
| CON95 | PEP74 | INS25 |
| CON96 | PEP74 | INS26 |
| CON97 | PEP74 | INS27 |
| CON98 | PEP74 | INS28 |
| CON99 | PEP74 | INS29 |
| CON100 | PEP74 | INS30 |
| CON101 | PEP74 | INS31 |
| CON102 | PEP76 | INS2 |
| CON103 | PEP77 | INS2 |
| CON104 | PEP74 | INS19 |
| CON105 | PEP36 | INS19 |
| CON106 | PEP78 | INS0 |
| CON107 | PEP101 | INS32 |
| CON108 | PEP79 | INS32 |
| CON109 | PEP80 | INS32 |
| CON110 | PEP81 | INS32 |
| CON111 | PEP82 | INS32 |
| CON112 | PEP83 | INS32 |
| CON113 | PEP84 | INS32 |
| CON114 | PEP85 | INS32 |
| CON115 | PEP86 | INS32 |
| CON116 | PEP87 | INS32 |
| CON117 | PEP88 | INS32 |
| CON118 | PEP89 | INS32 |
| CON119 | PEP90 | INS32 |
| CON120 | PEP91 | INS32 |
| CON121 | PEP92 | INS32 |
| CON122 | PEP93 | INS32 |
| CON123 | PEP94 | INS32 |
| CON124 | PEP95 | INS32 |
| CON125 | PEP96 | INS32 |
| CON126 | PEP97 | INS32 |
| CON127 | PEP98 | INS32 |
| CON128 | PEP99 | INS32 |
| CON129 | PEP100 | INS32 |
| CON130 | PEP101 | INS32 |
| CON131 | PEP102 | INS32 |
| CON132 | PEP103 | INS32 |
| CON133 | PEP104 | INS32 |
| CON134 | PEP105 | INS32 |
| CON135 | PEP106 | INS32 |
| CON136 | PEP107 | INS32 |
| CON137 | PEP108 | INS32 |
| CON138 | PEP109 | INS32 |
| CON139 | PEP110 | INS32 |
| CON140 | PEP111 | INS32 |
| CON141 | PEP112 | INS32 |
| CON142 | PEP113 | INS32 |
| CON143 | PEP114 | INS32 |
| CON144 | PEP115 | INS32 |
| CON145 | PEP116 | INS32 |
| CON146 | PEP117 | INS32 |
| CON147 | PEP118 | INS32 |
| CON148 | PEP119 | INS32 |
| CON149 | PEP120 | INS32 |
| CON150 | PEP121 | INS32 |
| CON151 | PEP122 | INS32 |
| CON152 | PEP123 | INS32 |
| CON153 | PEP124 | INS32 |
| CON154 | PEP125 | INS32 |
| CON155 | PEP126 | INS32 |
| CON156 | PEP127 | INS32 |
| CON157 | PEP128 | INS32 |
| CON158 | PEP129 | INS32 |
| CON159 | PEP130 | INS32 |
| CON160 | PEP131 | INS32 |
| CON161 | PEP132 | INS32 |

The conjugates are shown in FIG. 2A, FIG. 2B, and FIG. 2C which show various exemplary insulin-incretin conjugates in which the Norleucine (Nle) amino acid of particular incretins is conjugated to the epsilon amino group of Lysine (Lys) at position B29 of the B-chain peptide; FIG. 3, which shows various exemplary insulin-incretin conjugates in which the Ne amino acid of particular incretins is conjugated to the amino group of the Glycine (Gly) at position A1 of the A-chain peptide; FIG. 4A, FIG. 4B, FIG. 4C, FIG. 4D, FIG. 4E, FIG. 4F, FIG. 4G, FIG. 4H, FIG. 4I, FIG. 4J, FIG. 4K, FIG. 4L, FIG. 4M, FIG. 4N, FIG. 4O, and FIG. 4P, which show exemplary insulin-incretin conjugates in which the Ne amino acid of particular incretins is conjugated to the amino group of the Phenylalanine (Phe) at position B1 of the B-chain peptide; FIG. 5, which shows an exemplary insulin-incretin conjugate in which the Nle amino acid of PEP74* is conjugated to the amino group of the Phe at position B1 of the B-chain peptide; FIG. 6A, FIG. 6B, FIG. 6C, and FIG. 6D show exemplary insulin-incretin conjugates in which the epsilon amino group of Lys of the incretin is conjugated to the amino group of the Phe at position B1 of the B-chain peptide; FIG. 9A, FIG. 9B, FIG. 9C, FIG. 9D, FIG. 9E, FIG. 9F, FIG. 9G, FIG. 9H, FIG. 9I, FIG. 9J, FIG. 9K, and FIG. 9L, which show exemplary insulin-incretin conjugates in which the Norleucine (Nle) amino acid of the incretin is conjugated to the amino group of the Phe at position B1 of the B-chain peptide; FIG. 10A, FIG. 10B, FIG. 10C, FIG. 10D, FIG. 10E, FIG. 10F, and FIG. 10G, which show exemplary insulin-incretin conjugates in which the epsilon amino group of Lys of the incretin is conjugated to the amino group of the Phenylalanine (Phe) at position B1 of the B-chain peptide; and FIG. 11, which shows exemplary insulin-incretin conjugates CON106 and CON107. The “*” shown for various peptides indicates that the incretin has the amino acid sequence and structure of the indicated incretin but wherein the azide group of the linker has formed a 1,2-disubstituted 1,2,3-triazole with the alkyne group on the insulin.

### Example 25

Activity of the incretin peptides or the conjugates at the Glucagon receptor (GCGR) and GLP-1 receptor (GLP1R) may be measured in a cAMP activity assay as follows.

Peptides are dissolved in 100% DMSO and serially diluted to generate 10 point titrations. The peptide solutions are then transferred into 384-well assay plates (150 nL/well). Assay ready frozen cells expressing human GLP1R or human GCGR are suspended in growth media consisting of DMEM medium (GIBCO), 10% FBS (GIBCO), 1×NEAA(GIBCO), 1×P/S (GIBCO), 10 μg/mL Blasticidin (GIBCO) and 200 μg/mL Hygromycin (GIBCO). Cells are then diluted in assay buffer consisting of PBS (GIBCO), 7.5% BSA (Perkin Elmer), 100 μM RO 20-1724 (Sigma), with or without 20% human serum (MP Biomedical). The cell suspensions (15 μL) are then added to the assay plates containing the peptide solutions (30,000 cells/well for human GCGR; 10,000 cells/well for human GLP1R). The cells are incubated for one hour at room temperature in the dark.

Production of cAMP may be determined using HitHunter™ cAMPXS kits (DiscoverX) following the manufacturer's protocol. The plates are incubated for overnight at room temperature in the dark. Luminescence may be measured using an EnVision Multilabel plate reader (Perkin Elmer). Native GLP-1 and Glucagon (Bachem) may be used as control peptides. EC₅₀ values may be calculated using uses a 4 parameter logistic fit based on the Levenberg-Marquardt algorithm. The results for several of the peptides are shown in Table 5.

TABLE 5

| Peptide | Azide | hGCGR_EC₅₀ | hGLP1R EC₅₀ | GCGR/GLP1R |
|---|---|---|---|---|
| Name | position | (nM) | (nM) | ratio |
| PEP1 | 21 | 0.01 | 0.03 | 0.5 |
| PEP2 | 24 | 0.11 | 0.03 | 3.7 |
| PEP3 | 24 | 0.07 | 0.02 | 4.6 |
| PEP4 | 24 | 0.41 | 0.48 | 0.9 |
| PEP5 | 30 | 0.24 | 1.04 | 0.2 |
| PEP6 | 30 | 0.07 | 0.17 | 0.4 |
| PEP7 | 24 | 0.06 | 0.38 | 0.2 |
| PEP8 | 30 | 0.77 | 0.31 | 2.5 |
| PEP9 | 24 | 0.45 | 0.15 | 3.1 |
| PEP10 | 24 | 0.02 | 0.03 | 0.7 |
| PEP11 | 24 | 0.03 | 0.06 | 0.4 |
| PEP12 | 24 | 0.02 | 0.07 | 0.3 |
| PEP13 | 24 | 0.04 | 0.10 | 0.4 |
| PEP14 | 24 | 0.05 | 0.07 | 0.8 |
| PEP15 | 24 | 0.02 | 0.03 | 0.7 |
| PEP16 | 21 | 0.05 | 0.06 | 0.8 |
| PEP17 | 21 | 0.06 | 0.05 | 1.2 |
| PEP18 | 31 | 0.21 | 0.03 | 6.7 |
| PEP19 | 31 | 0.47 | 0.34 | 1.4 |
| PEP20 | 31 | 0.10 | 0.05 | 2.0 |
| PEP21 | 31 | 0.08 | 0.07 | 1.1 |
| PEP22 | 24 | 0.13 | 0.07 | 1.9 |
| PEP25 | 24 | 0.04 | 0.05 | 0.8 |
| PEP26 | 20 | 0.12 | 0.12 | 1.0 |
| PEP27 | 21 | 0.39 | 0.06 | 6.5 |
| PEP28 | 31 | 0.17 | 0.16 | 1.1 |
| PEP29 | 24 | 3.26 | 3.28 | 1.0 |
| PEP30 | 24 | 2.27 | 0.50 | 4.5 |
| PEP31 | 24 | 4.24 | 1.38 | 3.1 |
| PEP32 | 24 | 0.06 | 0.04 | 1.5 |
| PEP34 | 24 | 0.05 | 0.05 | 1.0 |
| PEP35 | 24 | 0.05 | 0.05 | 1.0 |
| PEP37 | 24 | 0.06 | 0.04 | 1.5 |
| PEP68 | 24 | 2.7 | 0.07 | 38.6 |
| PEP69 | 24 | 0.31 | 0.16 | 1.9 |
| PEP70 | 20 | 3.47 | 0.04 | 86.8 |
| PEP71 | 20 | 0.12 | 0.06 | 2.0 |
| PEP72 | 32 | 3.90 | 0.04 | 97.5 |
| PEP73 | 30 | 4.57 | 7.58 | 0.6 |
| PEP74 | 24 | 0.02 | 0.05 | 0.4 |
| PEP75 | 24 | >5 | 0.07 | >72 |

### Example 26

The conjugate binding or affinity to the insulin receptor may be performed using the following Insulin Receptor Binding Assays.

Two competition binding assays may be utilized to determine affinity for the human insulin receptor type B (IR(B)) against the endogenous ligand, insulin, labeled with ¹²⁵[I].

Method 1 IR binding assay is a whole cell binding method using CHO cells overexpressing human IR(B). The cells are grown in F12 media containing 10% FBS and antibiotics (G418, Penicillin/Strepavidin), plated at 40,000 cells/well in a 96-well tissue culture plate for at least eight hours. The cells are then serum starved by switching to DMEM media containing 1% BSA (insulin-free) overnight. The cells are washed twice with chilled DMEM media containing 1% BSA (insulin-free) followed by the addition of conjugate at appropriate concentration in 90 μL of the same media. The cells are incubated on ice for 60 minutes. The ¹²⁵[I]-insulin (10 μL) is added at 0.015 nM final concentration and incubated on ice for four hours. The cells are then gently washed three times with chilled media and lysed with 30 μL of Cell Signaling lysis buffer (cat #9803) with shaking for 10 minutes at room temperature. The lysate is added to scintillation liquid and counted to determine ¹²⁵[I]-insulin binding to IR and the titration effects of the conjugate on this interaction.

Method 2 IR binding assay is run in a scintillation proximity assay (SPA) in 384-well format using cell membranes prepared from CHO cells overexpressing human IR(B) grown in F12 media containing 10% FBS and antibiotics (G418, Penicillin/Strepavidin). Cell membranes are prepared in 50 mM Tris buffer, pH 7.8 containing 5 mM MgCl₂. The assay buffer contains 50 mM Tris buffer, pH 7.5, 150 mM NaCl, 1 mM CaCl₂, 5 mM MgCl₂, 0.1% BSA and protease inhibitors (Complete-Mini-Roche). Cell membranes are added to WGA PVT PEI SPA beads (5 mg/mL final concentration) followed by addition of conjugate at appropriate concentrations. After 5-15 min incubation at room temperature, ¹²⁵[I]-insulin is added at 0.015 nM final concentration for a final total volume of 50 μL. The mixture is then incubated with shaking at room temperature for 1 to 12 hours followed by scintillation counting to determine ¹²⁵[I]-insulin binding to IR and the titration effects of conjugate on this interaction.

### Example 27

Conjugate agonist activity at the insulin receptor may be performed using the following Insulin Receptor Phosphorylation Assays.

Insulin receptor activation can be assessed by measuring phosphorylation of the Akt protein, a key step in the insulin receptor signaling cascade. CHO cell lines overexpressing either human, minipig or dog IR are utilized in an HTRF sandwich ELISA assay kit (Cisbio “Phospho-AKT(Ser473) and Phospho-AKT(Thr308) Cellular Assay Kits”). Cells are grown in F12 media supplemented with 10% FBS, 400 ug/ml G418 and 10 mM HEPES. Prior to assay, the cells are incubated in serum free media for 2 to 4 hours. Alternatively, the cells may be frozen and aliquoted ahead of time in media containing 20% DMSO and used in the assay upon thawing, spin down and re-suspension.

Cells are plated at 10,000 cells per well in 20 ul of the serum free F12 media in 384-well plates. Humulin and glargine controls were run on each plate of test compounds. The titrated compounds are added to the cells (2 μL per well, final concentrations=1000 nM titrated down to 0.512 μM in 1:5 fold dilutions) and incubated at 37° C. for 30 minutes. The cells are lysed with 8 μL of the prepared lysis buffer provided in the CisBio kit and incubated at 25° C. for one hour. The diluted antibody reagents (anti-AKT-d2 and anti-pAKT-Eu3/cryptate) are prepared according to the kit instructions and then 10 μL is added to each well of cell lysate followed by incubation at 25° C. for 3.5 to 5 hours. The plate may be read by in an Envision plate reader (Excitation=320 nm; Emission=665 nm) to determine the IR pAkt agonist activity with regard to both potency and maximum response for each compound. Alternatively, the compounds may be tested in the same manner in the presence of 1.6 nM of Humulin to determine how each compound was able to compete against the full agonist activity of insulin.

### Example 28

Table 6 shows the incretin and insulin agonist activity of the various insulin incretin conjugates disclosed herein. Table 7 shows the incretin agonist activity or insulin agonist activity of several control molecules. Control molecule PEP76 has the amino acid sequence HsQGTFTSDK(γEγEC₁₆)SKYLDERAAQDFVQWLLDT-NH₂ (SEQ ID NO:110) and corresponds to the amino acid sequence of PEP74 except that PEP76 has the amino acid Gln (Q) at position 24 instead of Nle(εN₃) as in PEP74.

TABLE 6

| | Incretin Receptor Assay | | | | | | |
|---|---|---|---|---|---|---|---|
| | (cAMP) EC₅₀ | | | Insulin Receptor | | Insulin Receptor | |
| | (nM) | | | Binding Assay EC₅₀ | | pAK Assay EC₅₀ | |
| | GCGR | GLP1R | Ratio | (nM) | | (nM) | |
| Conjugate | (Human) | (Human) | GCGR/GLP1R | Human | Minipig | Human | Minipig |
| CON1 | 20 | 1.42 | 14.1 | 1445 | 2500 | 4.325 | 3.276 |
| CON2 | 20 | 3.35 | 6.0 | 2.347 | 4.339 | 0.185 | 0.352 |
| CON3 | 20 | 4.93 | 4.1 | 2500 | 2500 | 24.6 | 27.98 |
| CON4 | 2.82 | 6.1 | 0.5 | 1.1 | 6 | 0.206 | 0.35 |
| CON5 | 5 | 5 | 1.0 | 22.5 | 41.9 | 0.9 | 0.74 |
| CON6 | 1.55 | 3.67 | 0.4 | 61.13 | 191.7 | 4.353 | 3.276 |
| CON7 | 20 | 2.72 | 7.4 | 372.5 | 2500 | 9.6 | 3.276 |
| CON8 | 20 | 5 | 4.0 | 2500 | 2500 | 21.1 | 21.3 |
| CON9 | 20 | 5 | 4.0 | 3.964 | 7.5 | 0.312 | 0.63 |
| CON10 | 2.56 | 2.39 | 1.1 | 142.7 | 451 | 13.17 | 13.86 |
| CON11 | 3.34 | 5 | 0.7 | 3.21 | 8.988 | 0.292 | 0.673 |
| CON12 | 5 | 5 | 1.0 | 233.6 | 234.6 | 9.97 | 6.22 |
| CON13 | 20 | 5 | 4.0 | 15.11 | 24.36 | 0.26 | 0.65 |
| CON14 | 20 | 20 | 1.0 | 1.89 | 3.43 | 0.02 | 0.07 |
| CON15 | 20 | 20 | 1.0 | 25.59 | 13.71 | 0.53 | 0.37 |
| CON16 | 0.06 | 0.17 | 0.4 | 19.53 | 13.67 | 0.37 | 0.63 |
| CON17 | 0.1 | 0.06 | 1.7 | 149.9 | 169.9 | 33 | 16.2 |
| CON19 | 5 | 20 | 0.3 | 0.77 | 2.15 | 0.05 | 0.13 |
| CON20 | 5 | 5 | 1.0 | 2.76 | 3.93 | 0.05 | 0.09 |
| CON21 | 0.09 | 0.08 | 1.1 | 12.68 | 26.58 | 0.49 | 0.96 |
| CON22 | 0.06 | 0.04 | 1.5 | 12 | 29.84 | 0.11 | 0.19 |
| CON23 | 0.12 | 0.13 | 0.9 | 10.28 | 23.59 | 0.08 | 0.18 |
| CON24 | 0.07 | 0.09 | 0.8 | 7.61 | 16.76 | 0.4 | 0.75 |
| CON25 | 0.1 | 0.1 | 1.0 | 20.26 | 28.66 | 0.08 | 0.19 |
| CON26 | 0.08 | 0.18 | 0.4 | | | 0.3 | 0.83 |
| CON27 | 0.08 | 0.11 | 0.7 | | | 0.07 | 0.27 |
| CON28 | 0.32 | 0.23 | 1.4 | | | 0.25 | 0.74 |
| CON29 | 1.47 | 0.06 | 24.5 | | | 0.61 | 0.95 |
| CON30 | 2.89 | 0.07 | 41.3 | | | 5.29 | 17.24 |
| CON31 | 2.02 | 4.42 | 0.5 | | | 0.62 | 2.04 |
| CON32 | 1.2 | 6.05 | 0.2 | | | 0.41 | 2.54 |
| CON33 | 0.25 | 0.42 | 0.6 | | | 0.21 | 0.97 |
| CON34 | 0.42 | 1.32 | 0.3 | | | 0.72 | 1.65 |
| CON35 | 2.2 | 1.88 | 1.2 | | | 0.33 | 1.35 |
| CON36 | 16.47 | 3.08 | 5.3 | | | 0.12 | 0.35 |
| CON37 | 0.34 | 0.08 | 4.3 | | | 0.14 | 0.5 |
| CON38 | 0.34 | 3.32 | 0.1 | | | 0.63 | 2.22 |
| CON39 | 0.08 | 0.34 | 0.2 | | | 2.02 | 4.55 |
| CON40 | 0.13 | 0.38 | 0.3 | | | 6.82 | 12.58 |
| CON41 | 0.18 | 0.13 | 1.4 | | | 0.58 | 1.28 |
| CON42 | 0.11 | 0.08 | 1.4 | | | 0.15 | 0.32 |
| CON43 | 0.31 | 0.11 | 2.8 | | | 0.72 | 1.71 |
| CON44 | 0.21 | 0.1 | 2.1 | | | 0.79 | 1.81 |
| CON45 | 2.42 | 0.44 | 5.5 | | | 0.69 | 1.49 |
| CON46 | 0.76 | 0.08 | 9.5 | | | 0.18 | 0.53 |
| CON47 | 0.72 | 0.07 | 10.3 | | | 0.49 | 1.25 |
| CON48 | 0.53 | 0.05 | 10.6 | | | 0.33 | 0.66 |
| CON49 | 0.65 | 0.11 | 5.9 | | | 0.47 | 0.72 |
| CON50 | 0.53 | 2.57 | 0.2 | | | 0.08 | 0.2 |
| CON51 | 1.42 | 2.83 | 0.5 | | | 0.11 | 0.3 |
| CON52 | 0.43 | 2.08 | 0.2 | | | 0.15 | 0.42 |
| CON53 | 4.95 | 1.74 | 2.8 | | | 0.94 | 2.84 |
| CON55 | 0.16 | 0.13 | 1.2 | | | 0.59 | 1.42 |
| CON56 | 0.12 | 0.05 | 2.4 | | | 0.33 | 0.59 |
| CON57 | 0.23 | 0.09 | 2.6 | | | 0.29 | 0.5 |
| CON58 | 19.8 | 19.8 | 1.0 | | | 1.51 | 2.82 |
| CON59 | 19.8 | 19.8 | 1.0 | | | 3.18 | 4.49 |
| CON60 | 1.23 | 1.08 | 1.2 | | | 43.03 | 1000 |
| CON61 | 0.25 | 0.1 | 2.5 | | | 6.47 | 6.32 |
| CON62 | 0.06 | 0.02 | 3.0 | | | 0.25 | 0.63 |
| CON63 | 0.16 | 0.05 | 3.2 | | | 0.05 | 0.28 |
| CON64 | 0.26 | 0.16 | 1.6 | | | 9.8 | 7.46 |
| CON65 | 0.23 | 0.08 | 2.9 | | | 1000 | 1000 |
| CON66 | 0.24 | 0.1 | 2.4 | | | 0.17 | 0.3 |
| CON67 | 4.95 | 4.95 | 1.0 | | | 0.18 | 0.29 |
| CON68 | 0.15 | 0.09 | 1.7 | | | 2.43 | 4.5 |
| CON69 | 0.22 | 0.17 | 1.3 | | | 1000 | 117.8 |
| CON70 | 0.0585 | 0.035 | 1.7 | | | 3.28 | 3.28 |
| CON71 | 19.8 | 19.8 | 1.0 | | | 15.01 | 22.47 |
| CON72 | 19.8 | 11.95 | 1.7 | | | 47.6 | 15.75 |
| CON73 | 19.8 | 8.69 | 2.3 | | | 14.6 | 10.02 |
| CON74 | 0.75 | 1.53 | 0.5 | | | 0.27 | 0.28 |
| CON75 | 0.39 | 2.32 | 0.2 | | | 0.96 | 1.53 |
| CON76 | 0.15 | 3.05 | 0.0 | | | 0.55 | 1.05 |
| CON77 | 0.46 | 2.17 | 0.2 | | | 1.27 | 1.01 |
| CON78 | 0.18 | 0.11 | 1.6 | | | 1.65 | 1.27 |
| CON79 | 0.13 | 0.14 | 0.9 | | | 51.23 | 27.5 |
| CON80 | 0.24 | 0.25 | 1.0 | | | 0.25 | 0.69 |
| CON81 | 0.22 | 0.46 | 0.5 | | | 0.21 | 0.58 |
| CON82 | 19.8 | 19.8 | 1.0 | | | 5.13 | 9.73 |
| CON83 | 0.57 | 0.86 | 0.7 | | | 6.1 | 4.5 |
| CON84 | 0.16 | 0.3 | 0.5 | | | 0.05 | 0.09 |
| CON85 | 0.08 | 0.68 | 0.1 | | | 0.07 | 0.19 |
| CON86 | 2.55 | 0.26 | 9.8 | | | 0.71 | 1.55 |
| CON87 | 0.51 | 1.73 | 0.3 | | | 0.51 | 1.19 |
| CON88 | 0.37 | 2.13 | 0.2 | | | 0.61 | 1.21 |
| CON89 | 0.55 | 0.15 | 3.7 | | | 0.27 | 0.61 |
| CON90 | 0.31 | 0.19 | 1.6 | | | 0.14 | 0.4 |
| CON91 | 19.8 | 0.21 | 94.3 | | | 0.15 | 0.38 |
| CON92 | 0.18 | 0.18 | 1.01 | | | 0.23 | |
| CON93 | 0.22 | 0.46 | 0.48 | | | 0.205 | |
| CON94 | 0.56 | 15.24 | 0.036 | | | 2.94 | |
| CON95 | 0.94 | 2.62 | 0.36 | | | 1.4 | |
| CON96 | 1.21 | 2.78 | 0.43 | | | 8.67 | |
| CON97 | 2.51 | 4.04 | 0.62 | | | 4.86 | |
| CON98 | 0.63 | 1.11 | 0.56 | | | 17.29 | |
| CON99 | 0.08 | 0.16 | 0.48 | | | 0.96 | |
| CON101 | 19.8 | 1.19 | 8.0916.5 | | | 0.48 | |
| CON102 | 1.81 | 0.22 | 7.74 | | | 0.569 | |
| CON103 | 19.8 | 0.31 | 62.79 | | | 0.073 | |
| CON104 | 0.08 | 0.56 | 0.14 | | | 2.57 | |
| CON105 | 0.18 | 1.62 | 0.11 | | | 13.92 | |
| CON106 | 0.21 | 1.17 | 0.17 | | | 2.16 | |
| CON107 | 0.08 | 6.02 | 0.013 | | | 0.47 | |
| CON108 | 0.57 | 8.62 | 0.066 | | | 6.102 | |
| CON109 | 0.16 | 0.30 | 0.53 | | | 0.046 | |
| CON110 | 0.09 | 0.68 | 0.13 | | | 0.070 | |
| CON111 | 2.55 | 0.26 | 9.7 | | | 0.70 | |
| CON112 | 0.51 | 1.73 | 0.29 | | | 0.51 | |
| CON113 | 0.37 | 2.126 | 0.17 | | | 0.61 | |
| CON114 | 0.31 | 0.19 | 1.63 | | | 0.13 | |
| CON115 | 0.55 | 0.15 | 3.55 | | | 0.27 | |
| CON116 | 19.8 | 0.20 | 96.39 | | | 0.15 | |
| CON117 | 0.17 | 0.28 | 0.60 | | | 0.16 | |
| CON118 | 0.22 | 0.37 | 0.59 | | | 0.36 | |
| CON119 | 0.68 | 0.26 | 2.72 | | | 0.45 | |
| CON120 | 0.16 | 0.079 | 1.42 | | | 0.37 | |
| CON121 | 2.5 | 0.28 | 9.0 | | | 1.48 | |
| CON122 | 2.42 | 0.46 | 5.28 | | | 0.92 | |
| CON123 | 14.07 | 1.1 | 12.72 | | | 0.44 | |
| CON124 | 0.22 | 6.18 | 0.035 | | | 0.3454 | |
| CON125 | 1.71 | 1.15 | 1.48 | | | 0.28 | |
| CON126 | 0.56 | 0.59 | 0.93 | | | 0.438 | |
| CON127 | 1.27 | 19.8 | 0.064 | | | 0.93 | |
| CON128 | 0.15 | 0.20 | 0.72 | | | 0.197 | |
| CON129 | 1.54 | 1.75 | 0.878 | | | 1.44 | |
| CON130 | 0.21 | 19.8 | 0.01 | | | 0.137 | |
| CON131 | 0.22 | 1.73 | 0.128 | | | 0.51 | |
| CON132 | 1.19 | 1.66 | 0.72 | | | 0.36 | |
| CON133 | 0.64 | 0.47 | 1.37 | | | 0.42 | |
| CON134 | 0.25 | 0.05 | 4.54 | | | 0.25 | |
| CON135 | 0.48 | 0.71 | 0.66 | | | 0.14 | |
| CON136 | 0.7 | 0.50 | 1.39 | | | 2.09 | |
| CON137 | 1.04 | 0.16 | 6.62 | | | 0.063 | |
| CON138 | 0.95 | 0.72 | 1.32 | | | 0.12 | |
| CON139 | 0.2 | 19.8 | 0.01 | | | 0.47 | |
| CON140 | 0.34 | 0.21 | 1.61 | | | 0.066 | |
| CON141 | 0.96 | 0.72 | 4.57 | | | 0.12 | |
| CON142 | 0.68 | 0.74 | 0.91 | | | 0.730 | |
| CON143 | 0.33 | 2.77 | 0.11 | | | 1.04 | |
| CON144 | 0.04 | 1.95 | 0.02 | | | 27.9 | |
| CON145 | 0.43 | 0.20 | 2.15 | | | 0.10 | |
| CON146 | 0.42 | 4.95 | 0.08 | | | 0.20 | |
| CON147 | 0.3555 | 0.59 | 0.59 | | | 0.63 | |
| CON148 | 0.29 | 0.05 | 5.8 | | | 0.42 | |
| CON149 | 0.22 | 0.07 | 3.14 | | | 0.6 | |
| CON150 | 0.17 | 0.09 | 1.88 | | | 1.36 | |
| CON151 | 0.27 | 0.16 | 1.68 | | | 0.6 | |
| CON152 | 0.37 | 2.89 | 0.13 | | | 0.15 | |
| CON153 | 3.47 | 2.68 | 1.29 | | | 0.18 | |
| CON154 | 1.73 | 0.46 | 2.82 | | | 0.3 | |
| CON155 | 0.32 | 1.58 | 0.20 | | | 0.14 | |
| CON156 | >20 | >20 | | | | 17.39 | |
| CON157 | >20 | >20 | | | | 9.45 | |
| CON158 | >5 | >5 | | | | 9.45 | |
| CON159 | >20 | >5 | | | | 8.52 | |
| CON160 | 1.87 | 0.05 | 37.4 | | | 1.12 | |
| CON161 | 5.99 | 0.12 | 49.9 | | | 1.22 | |

TABLE 7

| | Incretin Receptor Assay | | | | | | |
|---|---|---|---|---|---|---|---|
| | (cAMP) EC50 | | | Insulin Receptor | | Insulin Receptor | |
| | (nM) | | | Binding Assay EC50 | | pAK Assay EC50 | |
| | GCGR | GLP1R | Ratio | (nM) | | (nM) | |
| | (Human) | (Human) | GCGR/GLP1R | Human | Minipig | Human | Minipig |
| PEP76 | 0.009 | 0.007 | 1.3 | | | | |
| PEP74 | 0.02 | 0.05 | 0.4 | | | | |
| RHI | | | | 0.35 | 0.59 | 0.01 | 0.02 |
| IG | | | | 0.57 | 0.77 | 0.01 | 0.01 |
| IL | | | | | | 0.51 | 0.5 |
| ID | | | | 9.03 | 8.46 | 0.48 | 0.35 |

RHI = Recombinant human insulin
IG = Insulin glargine
IL = Insulin levemir
ID = Insulin degludec

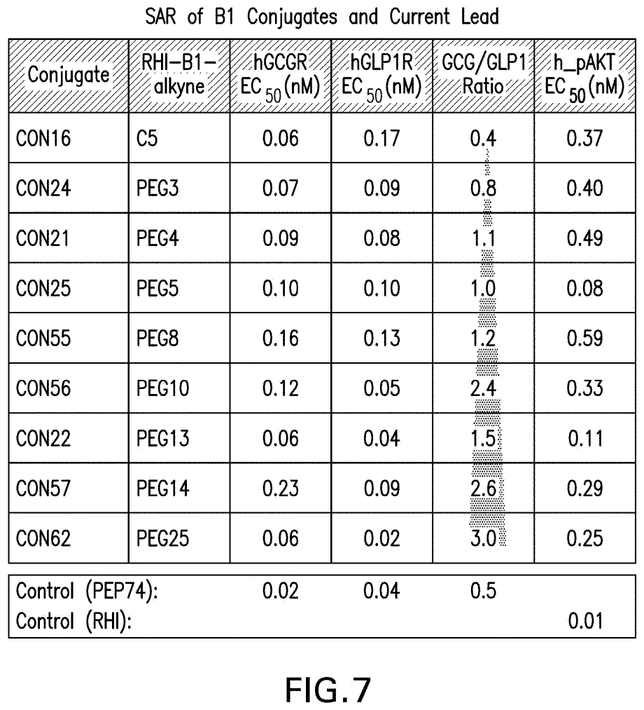

FIG. 7 shows that the length of the linking moiety at B1 has an effect on the ratio of glucagon (GCG) activity to GLP-1 activity.

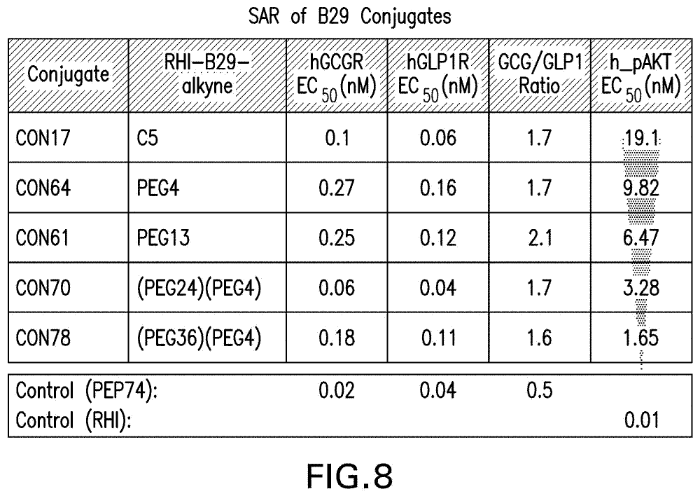

FIG. 8 shows that the length of the linking moiety at B29 has an effect on the activity of the insulin at the insulin receptor ratio of GCG activity to GLP-1 activity.

### Example 29

The following compounds were prepared following the procedure as disclosed in Example 6 in the provisional filing.

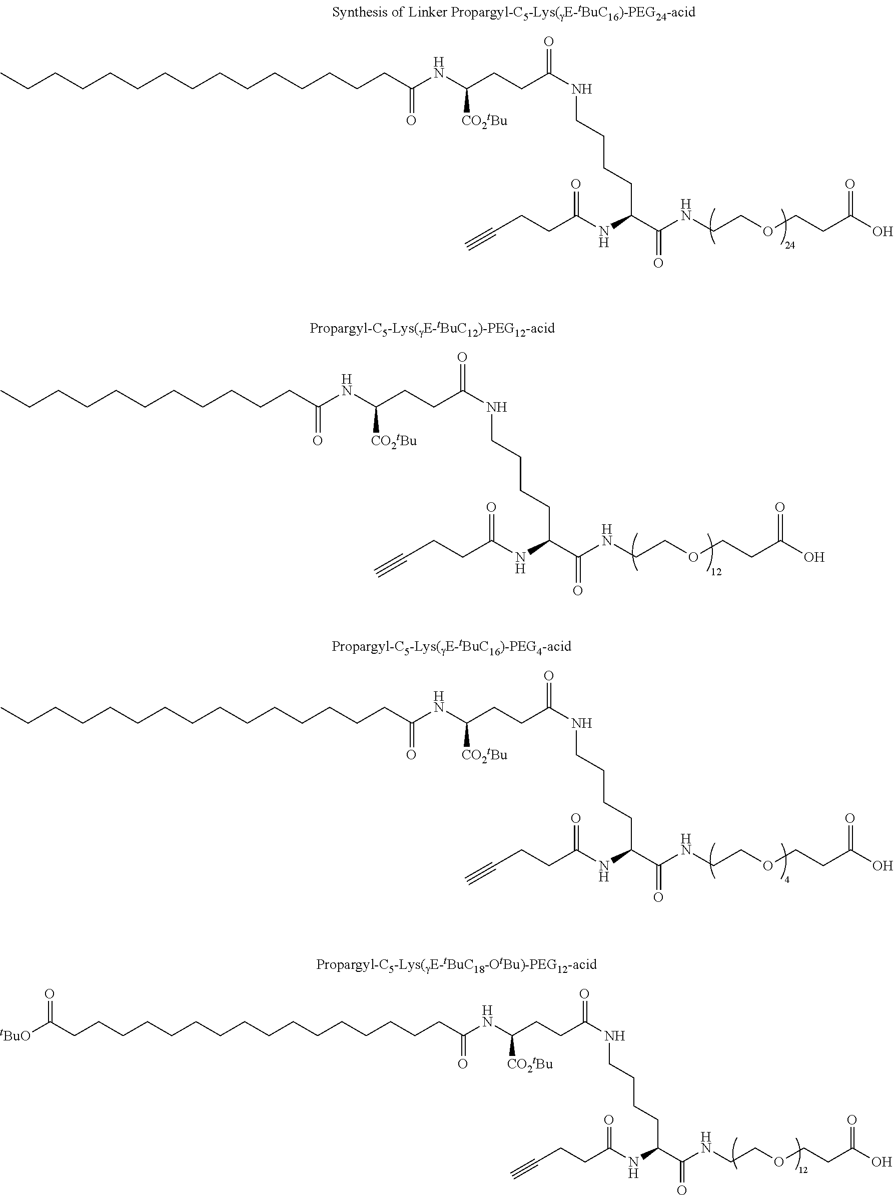

### Example 30

The insulin analogs shown below were prepared using the procedure described for INS7 (Example 14)

1. N^{αB1}-propargyl-C₅-Lys(γE-C₁₆)-PEG₂₄-RHI (INS25)

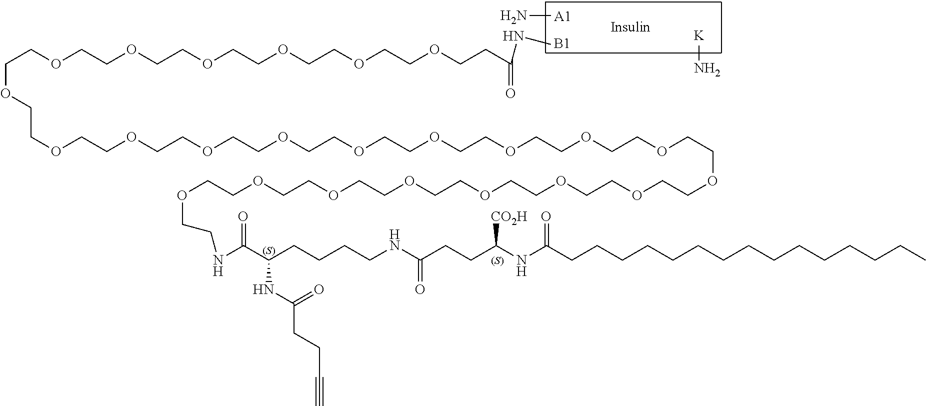

2. N^{αB1}-propargyl-C5-Lys(γE-C₁₂)-PEG₁₂-RHI (INS26)

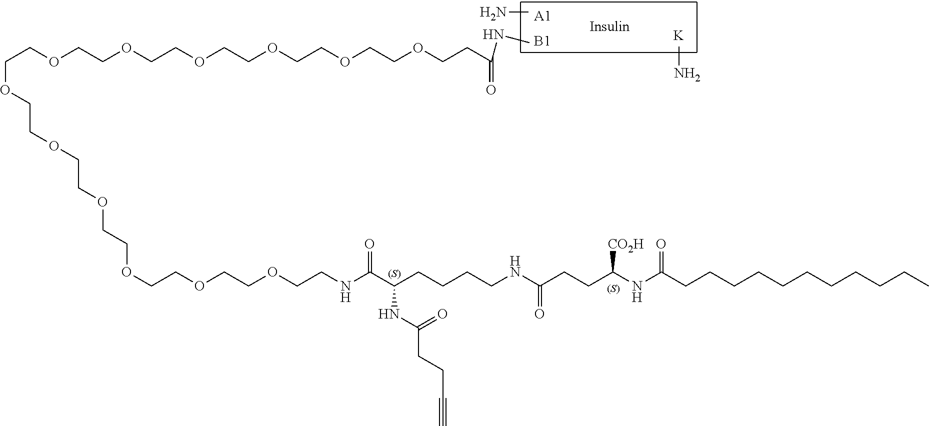

3. N^{αB1}-propargyl-C5-Lys(γE-C₁₆)-PEG₄-RHI (INS27)

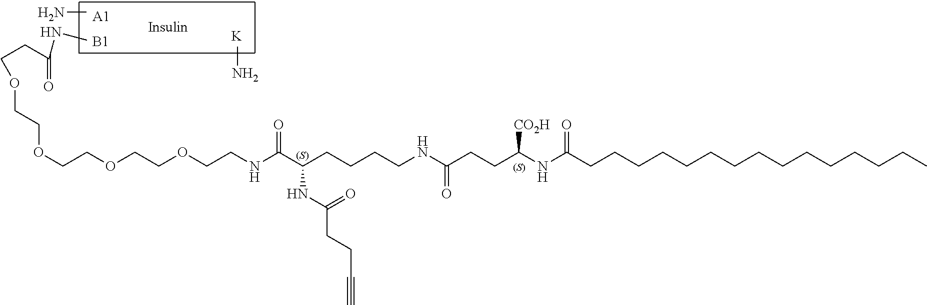

4. N^{αB1}-propargyl-C5-Lys(γE-C₁₈—OH)-PEG₁₂-RHI (INS28)

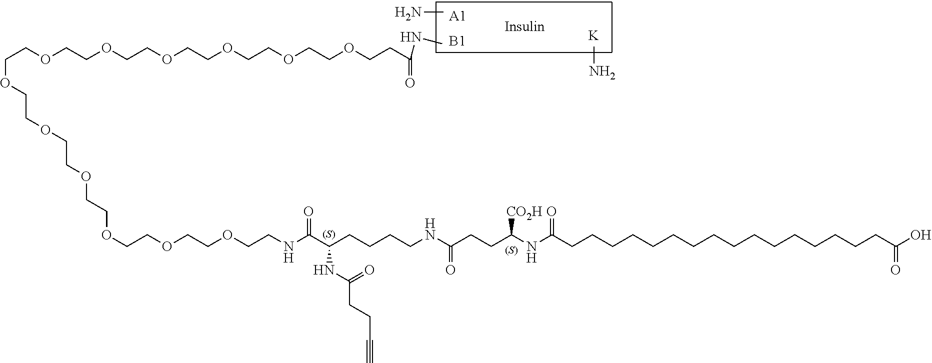

### Example 31

Synthesis of N^{εB28}-propargyl-PEG₄ Lispro (INS29)

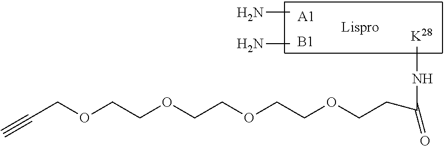

was as follows.

In a 20 mL vial, insulin Lispro (100 mg, 0.017 mmol) is dissolved in DMSO (1 mL) and aged at RT until homogeneous. 1,1,3,3-tetramethylguanidine (43 μl, 0.35 mmol) is added and followed by slow addition of alkyne-PEG₄-NHS (2,5-dioxopyrrolidin-1-yl 4,7,10,13-tetraoxahexadec-15-ynoate, 6.8 mg, 0.019 mmol) solution in DMSO (0.5 mL) via syringe pump over 30 min. The reaction is stirred at RT for one hour. The mixture is added dropwise to IPAc (20 mL) to precipitate out the product. The solids are filtered and dried under nitrogen gas and vacuum for 30 minutes. The solids are dissolved in 15 mL mix solvent (80% water, 20% ACN, pH=3.0), adjusted pH to 3. The residue is purified by RP-HPLC column, using the separation condition (28-35%) 0.05% TFA in AcCN/H₂O on Kromasil 100-10-C8 from AkzoNobel, 100 A and 10 μM 50×250 mm column; FR=85 mL/minute, ramp 25 minutes, wavelength=210 nm. The fractions are collected and lyophilized to give N^{εB28}-propargyl-PEG₄ Lispro.

### Example 32

Synthesis of N^{εB1}-propargyl-PEG₄ Lispro (INS30)

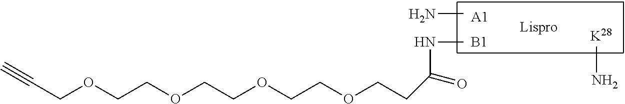

followed the procedure disclosed for INS3 (Example 13).

### Example 33

Synthesis of N^{εB29}-propargyl-PEG₄ Aspart (INS31)

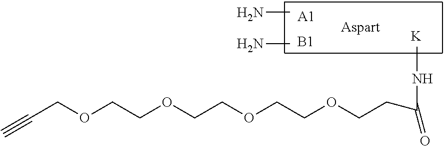

followed the procedure disclosed for INS17 (Example 16).

### Example 34

Synthesis of CON106 in which a C8 hydrocarbon linker links the lysine at position 24 of PEP78 to the B1 amino group of RHI (INS0) was as follows.

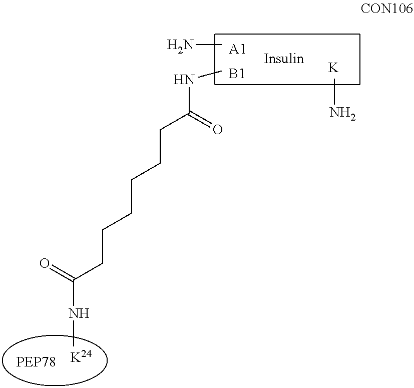

Step 1: Synthesis of PEP78 with Dde protecting groups at the N-terminus amino group and the lysine at position 12 followed the general procedure set out in Example 1.

Step 2: To a solution of bis(2,5-dioxopyrrolidin-1-yl) octanedioate (35.0 mg, 0.095 mmol) and TEA (0.026 ml, 0.190 mmol) in DMSO (2 ml) was added Pep7290-Dde (20 mg, 4.75 μmol) in DMSO (1 ml) via syringe pump over 1 h. The reaction was quenched after 2 h with 2 ml mixed solvent (ACN/water 20:80, pH 3) and adjusted to pH 2.5. The residue is purified by RP-HPLC, using the separation condition (50-70%) 0.05% TFA in ACN/H₂O on Kromasil 100-10-C8 from AkzoNobel, 100 A and 10 μM 50×250 mm column; FR=85 mL/minute, ramp 25 minutes, wavelength=210 nm. The desired fractions are collected and lyophilized to give the desired product. m/z 1489.5 [M+3]/3.

Step 3: A1,B29-bis-Boc RHI (34.4 mg, 5.73 μmol) was dissolved in DMF 0.5 ml in a 10 ml vail, to the solution was added Et₃N (4 μl, 0.029 mmol). The product from step 1 (12.8 mg, 2.87 μmol) in DMSO (400 μl and rinsed with 200 μl) was added over 30 min. The reaction mixture was stirred at rt overnight and used in the next step without purification. m/z: 1727.4 [M+6]/6.

Step 4: To the DMSO solution of crude product in step 3 was added 6 μl of hydrazine in DMF (36% in DMF). After stirring for one hour, the reaction mixture is dropwise added to a stirred 10 mL of IPAc to precipitate the product. The white solids are filtered, washed with IPAc and dried under N₂ and vacuum for one hour. m/z: 1672.2 [M+6]/6.

Step 5: TFA (0.5 mL, 6.5 mmol) is added to the above crude material (28.8 mg, 2.87 μmol) and the reaction is stirred and sonicated until solids fully dissolved (about 30 minutes). The reaction mixture is dropwise added to IPAc (10 mL) and the precipitated solids are filtered and dried under N₂ and vacuum. The crude material is purified by RP-HPLC, using the separation condition (32-46%) 0.05% TFA in ACN/H₂O on Kromasil 100-10-C8 from AkzoNobel, 100 A and 10 μM 50×250 mm column; FR=85 mL/minute, ramp 25 minutes, wavelength=210 nm. The desired fractions are collected and lyophilized to give the hydrocarbon linked conjugate CON106. m/z: 1966.4 [M+5]/5.

### Example 35

Synthesis of B1 azido RHI (INS32) was as follows.

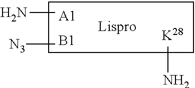

A1,B29-Bis Boc Insulin (100 mg, 0.017 mmol), sodium bicarbonate (5.6 mg, 0.067 mmol), copper(II) sulfate (0.53 mg, 3.33 μmol) was dissolved in water (0.8 mL) and MeOH (0.2 mL) was added 1H-imidazole-1-sulfonyl azide hydrochloride (5.6 mg, 0.027 mmol), maintained pH 8-9 by adding aq. sat. NaHCO₃ solution and stirred at room temperature overnight. Lyophilized the crude material and added TFA (2 mL, 0.017 mmol) for deprotection to give INS32 in which the N-terminal amino group has been converted to an azido group.

INS32 was conjugated to PEP101 following procedures disclosed herein to produce CON107 having the structure

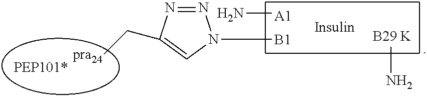

While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the claims attached herein.

1. A compound comprising:
an insulin molecule having agonist activity at the insulin receptor conjugated via a non-peptide linking moiety to a peptide having agonist activity at the glucagon-like 1 (GLP-1) receptor, the glucagon (GCG) receptor, and/or the gastric inhibitory protein (GIP) receptor, wherein the non-peptide linking moiety comprises the formula 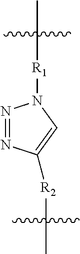
wherein R₁ and R₂ independently comprise a C₁-C₅₀ hydrocarbon chain or substituted hydrocarbon chain, a PEGₙ wherein n is 1-50, a (PEG₂)ₙ wherein n is 1-50, a (PEG₂)ₙ-(γGlu)ₚ-Cₙ wherein each n is independently 1-50 and p is 1 or 2, a (PEG₂)ₙ-Cₙ wherein each n is independently is 1-50, a (PEG)ₙ PEGₙ wherein each n is independently 1-50, a PEGₙ-(Lys-(γGlu)ₚ-Cₙ)—Cₙ wherein each n is independently 1-50 and p is 1 or 2, and a C₅-Lys(γE-Cₙ)-PEGₙ wherein each n is independently 1-50, and wherein the bond between the linking moiety and the insulin molecule and the incretin peptide are indicated by the wavy lines with the proviso that if the bond adjacent to R₁ is to insulin then the bond adjacent to R₂ is to the incretin peptide or that if the bond adjacent to R₁ is to the incretin peptide then the bond adjacent to R₂ is to insulin.

2. The compound of claim 1, wherein the peptide comprises (a) a first linker arm having a proximal end linked to an amino acid of the peptide and a distal end linked to an azide group and the insulin molecule comprises a second linker arm having a proximal end linked to an amino acid of the insulin molecule and a distal end linked to an alkyne group, wherein the azide group and the alkyne group form the 1,4-disubstituted 1,2,3-triazole; or (b) a first linker arm having a proximal end linked to an amino acid of the peptide and a distal end linked to an alkyne group and the insulin molecule comprises a second linker arm having a proximal end linked to an amino acid of the insulin molecule and a distal end linked to an azide group, wherein the azide group and the alkyne group form the 1,4-disubstituted 1,2,3-triazole in either case.

3. The compound of claim 1, wherein the peptide comprises within its amino acid sequence (a) an azido-norleucine and the insulin molecule comprises a linker arm having a proximal end linked to the amino group of an amino acid of the insulin molecule and a distal end linked to an alkynyl group, wherein the azido group and the alkynyl group form the 1,4-disubstituted 1,2,3-triazole; or (b) an alkynyl-norleucine and the insulin molecule comprises a linker arm having a proximal end linked to the amino group of an amino acid of the insulin molecule and a distal end linked to an azido group, wherein the azido group and the alkynyl group form the 1,4-disubstituted 1,2,3-triazole; or (c) a propargyl-Glycine and the insulin molecule comprises a linker arm having a proximal end linked to the amino group of an amino acid of the insulin molecule and a distal end linked to an azido group, wherein the azido group and the alkynyl group form the 1,4-disubstituted 1,2,3-triazole.

4. The compound of claim 1, wherein the insulin molecule comprises within its amino acid sequence (a) an azido-norleucine and the peptide comprises a linker arm having a proximal end linked to the amino group of an amino acid of the peptide and a distal end linked to an alkynyl group, wherein the azido group and the alkynyl group form the 1,4-disubstituted 1,2,3-triazole; or (b) an alkynyl-norleucine and the peptide comprises a linker arm having a proximal end linked to the amino group of an amino acid of the peptide and a distal end linked to an azido group, wherein the azido group and the alkynyl group form the 1,4-disubstituted 1,2,3-triazole; or (c) a propargyl-Glycine and the insulin molecule comprises a linker arm having a proximal end linked to the amino group of an amino acid of the insulin molecule and a distal end linked to an azido group, wherein the azido group and the alkynyl group form the 1,4-disubstituted 1,2,3-triazole.

5. The compound of claim 1, wherein the insulin molecule comprises an A-chain peptide and a B-chain peptide.

6. The compound of claim 5, wherein the insulin molecule is a heterodimer molecule or a single-chain insulin molecule.

7. The compound of claim 5, wherein the insulin molecule is selected from the group consisting of human insulin, insulin lispro, insulin detemir, insulin glulisine, insulin aspart, and insulin glargine.

8. The compound of claim 1, wherein the insulin molecule is conjugated to the peptide via the N-terminal amino acid of the A-chain peptide, the N-terminal amino acid of the B-chain peptide, or the amino acid at position 28 or 29 of the B-chain peptide.

9. The compound of claim 1, wherein the peptide is conjugated to the insulin molecule via an amino acid at position 20, 21, 24, 30, or 31 of the peptide.

10. The compound of claim 1, wherein the peptide is also conjugated to a fatty acid or fatty diacid.

11. The compound of claim 1, wherein the peptide comprises the amino acid sequence
(SEQ ID NO: 1)
HSQGTFTSDYSKYLDERAAQDFVQWLLDT,
which further includes at least the following modifications: (i) a substitution of the amino acid at position 2 with an amino acid that renders the peptide resistant to cleavage and inactivation by dipeptidyl peptidase IV; (ii) a lipid moiety covalently linked to the peptide at a lysine residue substituted for the tyrosine residue at position 10 or the glutamine at position 20 of the peptide; (iii) an azide group or an alkyne group conjugated to an amino acid at position 20, 21, 24, 30, or 31; (iv) 0, 1, 2, 3, 4, 5, 6, 7, 8, 9, or 10 additional amino acid substitutions in addition to the substitution at position 2; and optionally, a protecting group that is joined to the C-terminal carboxy group and/or the N-terminal amino group.

12. The compound of claim 11, wherein the peptide further includes a substitution of the amino acid at position 1 with a large aromatic amino acid, which provides the peptide with agonist activity at the GIP receptor.

13. The compound of claim 8, wherein the fatty acid or fatty diacid is conjugated to the epsilon amine of a lysine residue at amino acid position 10 or 20 of the peptide.

14. (canceled)

15. The compound of claim 1, wherein the compound comprises a peptide selected from the group consisting of peptides shown in Table 1.

16. A compound comprising a conjugate selected from the conjugates of Table 4 which are: CON1, CON2, CON3, CON4, CON5, CON6, CON7, CON8, CON9, CON10, CON11, CON12, CON13, CON14, CON15, CON16, CON17, CON19, CON20, CON21, CON22, CON24, CON25, CON26, CON27, CON28, CON29, CON30, CON31, CON32, CON33, CON34, CON35, CON36, CON37, CON38, CON39, CON40, CON41, CON42, CON43, CON44, CON45, CON46, CON47, CON48, CON49, CON50, CON51, CON52, CON53, CON55, CON56, CON57, CON58, CON59, CON60, CON61, CON62, CON63, CON64, CON65, CON66, CON67, CON68, CON69, CON70, CON71, CON72, CON73, CON74, CON75, CON76, CON77, CON78, CON79, CON80, CON81, CON82, CON83, CON84, CON85, CON86, CON87, CON88, CON89, CON90, CON91, CON92, CON93, CON94, CON95, CON96, CON97, CON98, CON99, CON100, CON101, CON102, CON103, CON104, CON105, CON106, CON107, CON108, CON109, CON110, CON111, CON112, CON113, CON114, CON115, CON116, CON117, CON118, CON119, CON120, CON121, CON122, CON123, CON124, CON125, CON126, CON127, CON128, CON129, CON130, CON131, CON132, CON133, CON134, CON135, CON136, CON137, CON138, CON139, CON140, CON141, CON142, CON143, CON144, CON145, CON146, CON147, CON148, CON149, CON150, CON151, CON152, CON153, CON154, CON155, CON156, CON157, CON158, CON159, CON160, and CON161.

17. A compound comprising the formula
A-LM-B
wherein A is human insulin molecule or human insulin analog molecule; B is a incretin molecule or glucagon peptide modified to have agonist activity at the GLP1 receptor, agonist activity at the GIP receptor, agonist the GLP1 and GCG receptors, or agonist activity at the GLP-1 and GIP receptors; LM is a linking moiety comprising
a cyclic or acyclic bisamide,
a heterocycle or a substituted heterocycle,
a C₁-C₅₀ hydrocarbon chain or substituted hydrocarbon chain,
a PEGₙ wherein n is 1-50,
a (PEG₂)ₙ wherein n is 1-50,
a (PEG₂)ₙ-(γGlu)ₚ-Cₙ wherein each n is independently 1-50 and p is 1 or 2,
a (PEG₂)ₙ-Cₙ wherein each n is independently is 1-50,
a (PEG)ₙ(PEG)ₙ wherein each n is independently 1-50,
a PEGₙ-(Lys-(γGlu)ₚ-Cₙ)—Cₙ wherein each n is independently 1-50 and p is 1 or 2, a C₅-Lys(γE-Cₙ)-PEGₙ wherein each n is independently 1-50, or
a 1,4-disubstituted 1,2,3-triazole having the formula 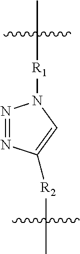
wherein R₁ and R₂ independently comprise a C₁-C₅₀ hydrocarbon chain or substituted hydrocarbon chain, a PEGₙ wherein n is 1-50, a (PEG₂)ₙ wherein n is 1-50, a (PEG₂)ₙ-(γGlu)ₚ-Cₙ wherein each n is independently 1-50 and p is 1 or 2, a (PEG₂)ₙ-Cₙ wherein each n is independently is 1-50, a (PEG)ₙ(PEG)ₙ wherein each n is independently 1-50, a PEGₙ-(Lys-(γGlu)ₚ-Cₙ)—Cₙ wherein each n is independently 1-50 and p is 1 or 2, and a C₅-Lys(γE-Cₙ)-PEGₙ wherein each n is independently 1-50, and wherein the bond between the linking moiety and the insulin molecule and the incretin peptide are indicated by the wavy lines with the proviso that if the bond adjacent to R₁ is to insulin then the bond adjacent to R₂ is to the incretin peptide or that if the bond adjacent to R₁ is to the incretin peptide then the bond adjacent to R₂ is to insulin.

18. The compound of claim 1, wherein the insulin molecule comprises an alkyne group and the peptide agonist activity is selective for the GLP-1 receptor and comprises the structure
(SEQ ID NO: 2)
HX²X³GTFTSX⁹X¹⁰SX¹²YX¹⁴DX¹⁶X¹⁷X¹⁸AX²⁰X²¹X²²V
X²⁴X²⁵X²⁶X²⁷X²⁸X²⁹X³⁰X³¹,
wherein X² is amionisobutyric acid (Aib), Gly, D-Serine (s), alpha-methyl Serine (αMS), or alpha-methyl D-Serine (αMs);
X³ is Val, Glu or Asp;
X⁹ is Asp or alpha-methyl Aspartic acid (αMD);
X¹⁰ is Tyr or Lys conjugated to a lipid moiety;
X¹² is Lysine, Leucine, or Serine;
X¹⁴ is Leu of alpha-methyl Leucine (αML);
X¹⁶ is Glutamic acid, Asparagine, Serine, Alanine, or Aib;
X¹⁷ is Arginine or Lysine;
X¹⁸ is Alanine or Arginine;
X²⁰ is Glutamine or Lysine conjugated via its epsilon amino group to a non-peptide linker comprising a terminal azide group or to a fatty acid or fatty diacid;
X²¹ is Aspartic acid, αMD, Azidonorleucine (Norleucine conjugated via its epsilon carbon to an azide group (Nle(εN₃))), or Lysine conjugated via its epsilon amino group to a non-peptide linker comprising a terminal azide group;
X²² is Phe or αMF;
X²⁴ is Glutamine, Nle(εN₃), or Lysine conjugated via its epsilon amino group to a non-peptide linker comprising a terminal azide group;
X²⁵ is Tryptophan or alpha-methyl Tryptophan (αMW);
X²⁶ is Leucine or αML;
X²⁷ is Methionine, Leucine, Methionine sulfoxide, or L-methionine sulphone (2);
X²⁸ is Aspartic acid, Alanine, Lysine, Asparagine, γGlu, Glutamine, or αMD;
X²⁹ is Threonine or Glycine;
X³⁰ is Arginine, Lysine, or Nle(εN₃), or X³⁰ is absent; and
X³¹ is Glycine, γGlu, Nle(εN₃), or Lysine conjugated via its epsilon amino group to a non-peptide linker comprising a terminal azide group, or X³¹ is absent; and
wherein the C-terminal amino acid optionally is amidated, and with the proviso that either X¹⁰ is a Lysine residue conjugated via its epsilon amino acid to the lipid moiety and one of X²⁰, X²¹, X²⁴, X³⁰, or X³¹ comprises the azide group or X²⁰ is a Lysine residue conjugated via its epsilon amino acid to the lipid moiety and one of X²¹, X²⁴, X³⁰, or X³¹ comprises the azide group.

19. The compound of claim 1, wherein the insulin molecule comprises an azide group and the peptide agonist activity is selective for the GLP-1 receptor and comprises the structure
(SEQ ID NO: 3)
HX²X³GTFTSX⁹X¹⁰SX¹²YX¹⁴DX¹⁶X¹⁷X¹⁸AX²⁰X²¹X²²V-
X²⁴X²⁵X²⁶X²⁷X²⁸X²⁹X³⁰X³¹,
wherein X² is aminoisobutyric acid (Aib), D-Serine (s), or alpha-methyl Serine (αMS);
X³ is Val, Glu, or Asp;
X⁹ is Asp or alpha-methyl Aspartic acid (αMD);
X¹⁰ is Tyr or Lys conjugated to a lipid moiety;
X¹² is Lysine, Leucine, or Serine;
X¹⁴ is Leu of alpha-methyl Leucine (αML);
X¹⁶ is Glutamic acid, Asparagine, Serine, Alanine, or Aib;
X¹⁷ is Arginine or Lysine;
X¹⁸ is Alanine or Arginine;
X²⁰ is Glutamine or Lysine conjugated via its epsilon amino group to a non-peptide linker comprising a terminal alkyne group or to a fatty acid or fatty diacid;
X²¹ is Aspartic acid, αMD, Alkynylnorleucine (Norleucine conjugated via its epsilon carbon to an alkyne group (Nle(ε-alkyne))), or Lysine conjugated via its epsilon amino group to a non-peptide linker comprising a terminal alkyne group;
X²² is Phe or alpha-methyl Phenylalanine (αMF);
X²⁴ is Glutamine, Nle(ε-alkyne), or Lysine conjugated via its epsilon amino group to a non-peptide linker comprising a terminal alkyne group;
X²⁵ is Tryptophan or alpha-methyl Tryptophan (αMW);
X²⁶ is Leucine or αML;
X²⁷ is Methionine, Leucine, methionine sulfoxide, or L-methionine sulphone (2);
X²⁸ is Aspartic acid, Alanine, Lysine, Asparagine, γGlu, Glutamine, or αMD;
X²⁹ is Threonine or Glycine;
X³⁰ is Arginine, Lysine, or Nle(ε-alkyne), or X³⁰ is absent; and
X³¹ is Glycine, γGlu, Nle(ε-alkyne), or Lysine conjugated via its epsilon amino group to a non-peptide linker comprising a terminal alkyne group, or X³¹ is absent; and
wherein the C-terminal amino acid optionally is amidated, and with the proviso that either X¹⁰ is a Lysine residue conjugated via its epsilon amino acid to the lipid moiety and one of X²⁰, X²¹, X²⁴, X³⁰, or X³¹ comprises the alkyne group or X²⁰ is a Lysine residue conjugated via its epsilon amino acid to the lipid moiety and one of X²¹, X²⁴, X³⁰, or X³¹ comprises the alkyne group.

20. The compound of claim 1, wherein the insulin molecule comprises an alkyne group and the peptide has agonist activity at the GLP-1 and GCG receptors and comprises the structure
(SEQ ID NO: 4)
HX²QGTFTSX⁹X¹⁰SX¹²YX¹⁴DX¹⁶X¹⁷X¹⁸AX²⁰X²¹X²²V-
X²⁴X²⁵X²⁶X²⁷X²⁸X²⁹X³⁰X³¹,
wherein X² is aminoisobutyric acid (Aib), D-Serine (s), or alpha-methyl Serine (αMS);
X⁹ is Asp or alpha-methyl Aspartic acid (αMD);
X¹⁰ is Tyr or Lys conjugated to a lipid moiety;
X¹² is Lysine, Leucine, or Serine;
X¹⁴ is Leu of alpha-methyl Leucine (αML);
X¹⁶ is Glutamic acid, Asparagine, Serine, Alanine, or Aib;
X¹⁷ is Arginine or Lysine;
X¹⁸ is Alanine or Arginine;
X²⁰ is Glutamine or Lysine conjugated via its epsilon amino group to a non-peptide linker comprising a terminal azide group or to a fatty acid or fatty diacid;
X²¹ is Aspartic acid, αMD, Azidonorleucine (Norleucine conjugated via its epsilon carbon to an azide group (Nle(εN₃))), or Lysine conjugated via its epsilon amino group to a non-peptide linker comprising a terminal azide group;
X²² is Phe or alpha-methyl Phenylalanine (αMF);
X²⁴ is Glutamine, Nle(εN₃), or Lysine conjugated via its epsilon amino group to a non-peptide linker comprising a terminal azide group;
X²⁵ is Tryptophan or alpha-methyl Tryptophan (αMW);
X²⁶ is Leucine or αML;
X²⁷ is Methionine, Leucine, methionine sulfoxide, or L-methionine sulphone (2);
X²⁸ is Aspartic acid, Alanine, Lysine, Asparagine, γGlu, Glutamine, or αMD;
X²⁹ is Threonine or Glycine;
X³⁰ is Arginine, Lysine, or Nle(εN₃), or X³⁰ is absent; and
X³¹ is Glycine, γGlu, Nle(εN₃), or Lysine conjugated via its epsilon amino group to a non-peptide linker comprising a terminal azide group, or X³¹ is absent; and
wherein the C-terminal amino acid optionally is amidated, and with the proviso that either X¹⁰ is a Lysine residue conjugated via its epsilon amino acid to the lipid moiety and one of X²⁰, X²¹, X²⁴, X³⁰, or X³¹ comprises the azide group or X²⁰ is a Lysine residue conjugated via its epsilon amino acid to the lipid moiety and one of X²¹, X²⁴, X³⁰, or X³¹ comprises the azide group.

21. The compound of claim 1, wherein the insulin molecule comprises an azide group and the peptide has agonist activity at the GLP-1 and GCG receptors and comprises the structure
(SEQ ID NO: 5)
HX²QGTFTSX⁹X¹⁰SX¹²YX¹⁴DX¹⁶X¹⁷X¹⁸AX²⁰X²¹X²²V-
X²⁴X²⁵X²⁶X²⁷X²⁸X²⁹X³⁰X³¹,
wherein X² is aminoisobutyric acid (Aib), D-Serine (s), or alpha-methyl Serine (αMS);
X⁹ is Asp or alpha-methyl Aspartic acid (αMD);
X¹⁰ is Tyr or Lys conjugated to a lipid moiety;
X¹² is Lysine, Leucine, or Serine;
X¹⁴ is Leu of alpha-methyl Leucine (αML);
X¹⁶ is Glutamic acid, Asparagine, Serine, Alanine, or Aib;
X¹⁷ is Arginine or Lysine;
X¹⁸ is Alanine or Arginine;
X²⁰ is Glutamine or Lysine conjugated via its epsilon amino group to a non-peptide linker comprising a terminal alkyne group or to a fatty acid or fatty diacid;
X²¹ is Aspartic acid, αMD, Alkynylnorleucine (Norleucine conjugated via its epsilon carbon to an alkyne group (Nle(ε-alkyne))), or Lysine conjugated via its epsilon amino group to a non-peptide linker comprising a terminal alkyne group;
X²² is Phe or alpha-methyl Phenylalanine (αMF);
X²⁴ is Glutamine, Nle(ε-alkyne), or Lysine conjugated via its epsilon amino group to a non-peptide linker comprising a terminal alkyne group;
X²⁵ is Tryptophan or alpha-methyl Tryptophan (αMW);
X²⁶ is Leucine or αML;
X²⁷ is Methionine, Leucine, methionine sulfoxide, or L-methionine sulphone (2);
X²⁸ is Aspartic acid, Alanine, Lysine, Asparagine, γGlu, Glutamine, or αMD;
X²⁹ is Threonine or Glycine;
X³⁰ is Arginine, Lysine, or Nle(ε-alkyne), or X³⁰ is absent; and
X³¹ is Glycine, γGlu, Nle(ε-alkyne), or Lysine conjugated via its epsilon amino group to a non-peptide linker comprising a terminal alkyne group, or X³¹ is absent; and
wherein the C-terminal amino acid optionally is amidated, and with the proviso that either X¹⁰ is a Lysine residue conjugated via its epsilon amino acid to the lipid moiety and one of X²⁰, X²¹, X²⁴, X³⁰, or X³¹ comprises the alkyne group or X²⁰ is a Lysine residue conjugated via its epsilon amino acid to the lipid moiety and one of X²¹, X²⁴, X³⁰, or X³¹ comprises the alkyne group.

22. The compound of claim 1, wherein the insulin molecule comprises an alkyne group and the peptide has agonist activity predominantly at the GLP-1 receptor and GIP receptor and comprises the structure
(SEQ ID NO: 6)
X¹X²X³GTFTSX⁹X¹⁰SX¹²YX¹⁴DX¹⁶X¹⁷X¹⁸AX²⁰X²¹X²²V-
X²⁴X²⁵X²⁶X²⁷X²⁸X²⁹X³⁰X³¹,
Wherein X¹ is Tyrosine, Phenylalanine, Tryptophan, or other amino acid with an aromatic group;
X² is amionisobutyric acid (Aib), Gly, D-Serine (s), or alpha-methyl Serine (αMS);
X³ is Val, Glu, or Asp;
X⁹ is Asp or alpha-methyl Aspartic acid (αMD);
X¹⁰ is Tyr or Lys conjugated to a lipid moiety;
X¹² is Isoleucine, Lysine, Leucine, or Serine;
X¹⁴ is Leu of alpha-methyl Leucine (αML);
X¹⁶ is Glutamic acid, Asparagine, Serine, Alanine, or Aib;
X¹⁷ is Arginine or Lysine;
X¹⁸ is Alanine or Arginine;
X²⁰ is Glutamine or Lysine conjugated via its epsilon amino group to a non-peptide linker comprising a terminal azide group or to a fatty acid or fatty diacid;
X²¹ is Aspartic acid, αMD, Azidonorleucine (Norleucine conjugated via its epsilon carbon to an azide group (Nle(εN₃))), or Lysine conjugated via its epsilon amino group to a non-peptide linker comprising a terminal azide group;
X²² is Phe or alpha-methyl Phenylalanine (αMF), X²⁴ is Glutamine, Nle(εN₃), or Lysine conjugated via its epsilon amino group to a non-peptide linker comprising a terminal azide group;
X²⁵ is Tryptophan or alpha-methyl Tryptophan (αMW);
X²⁶ is Leucine or αML;
X²⁷ is Met, Leucine, methionine sulfoxide, or L-methionine sulphone (2);
X²⁸ is Aspartic acid, Alanine, Lysine, Asparagine, γGlu, Glutamine, or αMD;
X²⁹ is Threonine or Glycine;
X³⁰ is Arginine, Lysine, or Nle(εN₃), or X³⁰ is absent; and
X³¹ is Glycine, γGlu, Nle(εN₃), or Lysine conjugated via its epsilon amino group to a non-peptide linker comprising a terminal azide group, or X³¹ is absent; and
wherein the C-terminal amino acid optionally is amidated, and with the proviso that either X¹⁰ is a Lysine residue conjugated via its epsilon amino acid to the lipid moiety and one of X²⁰, X²¹, X²⁴, X³⁰, or X³¹ comprises the azide group or X²⁰ is a Lysine residue conjugated via its epsilon amino acid to the lipid moiety and one of X²¹, X²⁴, X³⁰, or X³¹ comprises the azide group.

23. The compound of claim 1, wherein the insulin molecule comprises an azide group and the peptide has agonist activity at the GLP-1 receptor and GIP receptor and comprises the structure
(SEQ ID NO: 7)
X¹X²X³GTFTSX⁹X¹⁰SX¹²YX¹⁴DX¹⁶X¹⁷X¹⁸AX²⁰X²¹X²²V-
X²⁴X²⁵X²⁶X²⁷X²⁸X²⁹X³⁰X³¹
wherein X¹ is Tyrosine, Phenylalanine, Tryptophan, or other amino acid with an aromatic group;
X² is aminoisobutyric acid (Aib), D-Serine (s), or alpha-methyl Serine (αMS);
X³ is Val, Glu, or Asp;
X⁹ is Asp or alpha-methyl Aspartic acid (αMD);
X¹⁰ is Tyr or Lys conjugated to a lipid moiety;
X¹² is Isoleucine, Lysine, Leucine, or Serine;
X¹⁴ is Leu of alpha-methyl Leucine (αML);
X¹⁶ is Glutamic acid, Asparagine, Serine, Alanine, or Aib;
X¹⁷ is Arginine or Lysine;
X¹⁸ is Alanine or Arginine;
X²⁰ is Glutamine or Lysine conjugated via its epsilon amino group to a non-peptide linker comprising a terminal alkyne group or to a fatty acid or fatty diacid;
X²¹ is Aspartic acid, αMD, Alkynylnorleucine (Norleucine conjugated via its epsilon carbon to an alkyne group (Nle(ε-alkyne))), or Lysine conjugated via its epsilon amino group to a non-peptide linker comprising a terminal alkyne group;
X²² is Phe or alpha-methyl Phenylalanine (αMF),
X²⁴ is Glutamine, Nle(ε-alkyne), or Lysine conjugated via its epsilon amino group to a non-peptide linker comprising a terminal alkyne group;
X²⁵ is Tryptophan or alpha-methyl Tryptophan (αMW);
X²⁶ is Leucine or αML;
X²⁷ is Methionine, Leucine, methionine sulfoxide, or L-methionine sulphone (2);
X²⁸ is Aspartic acid, Alanine, Lysine, Asparagine, γGlu, Glutamine, or αMD;
X²⁹ is Threonine or Glycine;
X³⁰ is Arginine, Lysine, or Nle(ε-alkyne), or X³⁰ is absent; and
X³¹ is Glycine, γGlu, Nle(ε-alkyne), or Lysine conjugated via its epsilon amino group to a non-peptide linker comprising a terminal alkyne group, or X³¹ is absent; and
wherein the C-terminal amino acid optionally is amidated, and with the proviso that either X¹⁰ is a Lysine residue conjugated via its epsilon amino acid to the lipid moiety and one of X²⁰, X²¹, X²⁴, X³⁰, or X³¹ comprises the alkyne group or X²⁰ is a Lysine residue conjugated via its epsilon amino acid to the lipid moiety and one of X²¹, X²⁴, X³⁰, or X³¹ comprises the alkyne group.

24. The compound of claim 1, wherein the insulin molecule comprises an alkyne group and the peptide has agonist activity at the GLP-1, GIP, and GCG receptors and comprises the structure
(SEQ ID NO: 8)
X¹X²QGTFTSX⁹X¹⁰SX¹²YX¹⁴DX¹⁶X¹⁷X¹⁸AX²⁰X²¹X²²V-
X²⁴X²⁵X²⁶X²⁷X²⁸X²⁹X³⁰X³¹,
wherein X¹ is Tyrosine, Phenylalanine, Tryptophan, or other amino acid with an aromatic group;
X² is amionisobutyric acid (Aib), Gly, D-Serine (s), or alpha-methyl Serine (αMS);
X⁹ is Asp or alpha-methyl Aspartic acid (αMD);
X¹⁰ is Tyr or Lys conjugated to a lipid moiety;
X¹² is Isoleucine, Lysine, Leucine, or Serine;
X¹⁴ is Leu of alpha-methyl Leucine (αML);
X¹⁶ is Glutamic acid, Asparagine, Serine, Alanine, or Aib;
X¹⁷ is Arginine or Lysine;
X¹⁸ is Alanine or Arginine;
X²⁰ is Glutamine or Lysine conjugated via its epsilon amino group to a non-peptide linker comprising a terminal azide group or to a fatty acid or fatty diacid;
X²¹ is Aspartic acid, αMD, Azidonorleucine (Norleucine conjugated via its epsilon carbon to an azide group (Nle(εN₃))), or Lysine conjugated via its epsilon amino group to a non-peptide linker comprising a terminal azide group;
X²² is Phe or alpha-methyl Phenylalanine (αMF);
X²⁴ is Glutamine, Nle(εN₃), or Lysine conjugated via its epsilon amino group to a non-peptide linker comprising a terminal azide group;
X²⁵ is Tryptophan or alpha-methyl Tryptophan (αMW);
X²⁶ is Leucine or αML;
X²⁷ is methionine, Leucine, methionine sulfoxide, or L-methionine sulphone (2);
X²⁸ is Aspartic acid, Alanine, Lysine, Asparagine, γGlu, Glutamine, or αMD;
X²⁹ is Threonine or Glycine;
X³⁰ is Arginine, Lysine, or Nle(εN₃), or X³⁰ is absent; and
X³¹ is Glycine, γGlu, Nle(εN₃), or Lysine conjugated via its epsilon amino group to a non-peptide linker comprising a terminal azide group, or X³¹ is absent; and
wherein the C-terminal amino acid optionally is amidated, and with the proviso that either X¹⁰ is a Lysine residue conjugated via its epsilon amino acid to the lipid moiety and one of X²⁰, X²¹, X²⁴, X³⁰, or X³¹ comprises the azide group or X²⁰ is a Lysine residue conjugated via its epsilon amino acid to the lipid moiety and one of X²¹, X²⁴, X³⁰, or X³¹ comprises the azide group.

25. The compound of claim 1 wherein the insulin molecule comprises an azide group and the peptide has agonist activity at the GLP-1, GIP, and GCG receptors and comprises the structure
(SEQ ID NO: 9)
X¹X²QGTFTSX⁹X¹⁰SX¹²YX¹⁴DX¹⁶X¹⁷X¹⁸AX²⁰X²¹FV-
X²⁴X²⁵X²⁶X²⁷X²⁸X²⁹X³⁰X³¹,
wherein X¹ is Tyrosine, Phenylalanine, Tryptophan, or other amino acid with an aromatic group;
X² is aminoisobutyric acid (Aib), D-Serine (s), or alpha-methyl Serine (αMS);
X⁹ is Asp or alpha-methyl Aspartic acid (αMD);
X¹⁰ is Tyr or Lys conjugated to a lipid moiety;
X¹² is Isoleucine, Lysine, Leucine, or Serine;
X¹⁴ is Leu of alpha-methyl Leucine (αML);
X¹⁶ is Glutamic acid, Asparagine, Serine, Alanine, or Aib;
X¹⁷ is Arginine or Lysine;
X¹⁸ is Alanine or Arginine;
X²⁰ is Glutamine or Lysine conjugated via its epsilon amino group to a non-peptide linker comprising a terminal alkyne group or to a fatty acid or fatty diacid;
X²¹ is Aspartic acid, αMD, Alkynylnorleucine (Norleucine conjugated via its epsilon carbon to an alkyne group (Nle(ε-alkyne))), or Lysine conjugated via its epsilon amino group to a non-peptide linker comprising a terminal alkyne group;
X²² is Phe or alpha-methyl Phenylalanine (αMF);
X²⁴ is Glutamine, Nle(ε-alkyne), or Lysine conjugated via its epsilon amino group to a non-peptide linker comprising a terminal alkyne group;
X²⁵ is Tryptophan or alpha-methyl Tryptophan (αMW);
X²⁶ is Leucine or αML;
X²⁷ is methionine, Leucine, methionine sulfoxide, or L-methionine sulphone (2);
X²⁸ is Aspartic acid, Alanine, Lysine, Asparagine, γGlu, Glutamine, or αMD;
X²⁹ is Threonine or Glycine;
X³⁰ is Arginine, Lysine, or Nle(ε-alkyne), or X³⁰ is absent; and
X³¹ is Glycine, γGlu, Nle(ε-alkyne), or Lysine conjugated via its epsilon amino group to a non-peptide linker comprising a terminal alkyne group, or X³¹ is absent; and
wherein the C-terminal amino acid optionally is amidated, and with the proviso that either X¹⁰ is a Lysine residue conjugated via its epsilon amino acid to the lipid moiety and one of X²⁰, X²¹, X²⁴, X³⁰, or X³¹ comprises the alkyne group or X²⁰ is a Lysine residue conjugated via its epsilon amino acid to the lipid moiety and one of X²¹, X²⁴, X³⁰, or X³¹ comprises the alkyne group.

26. The compound of claim 1, wherein the peptide comprises a peptide selected from the group consisting of PEP1, PEP2, PEP3, PEP4, PEP5, PEP6, PEP7, PEP8, PEP9, PEP10, PEP11, PEP12, PEP13, PEP14, PEP15, PEP16, PEP17, PEP18, PEP19, PEP20, PEP21, PEP22, PEP23, PEP24, PEP25, PEP26, PEP27, PEP28, PEP29, PEP30, PEP31, PEP32, PEP33, PEP34, PEP35, PEP36, PEP37, PEP38, PEP39, PEP40, PEP41, PEP42, PEP43, PEP44, PEP45, PEP46, PEP47, PEP48, PEP49, PEP50, PEP51, PEP52, PEP53, PEP54, PEP55, PEP56, PEP57, PEP58, PEP59, PEP60, PEP61, PEP62, PEP63, PEP64, PEP65, PEP66, PEP67, PEP68, PEP69, PEP70, PEP71, PEP72, PEP73, PEP74, PEP75, PEP76, PEP77, PEP79, PEP80, PEP81, PEP82, PEP83, PEP84, PEP85, PEP86, PEP87, PEP88, PEP89, PEP90, PEP91, PEP92, PEP93, PEP94, PEP95, PEP96, PEP97, PEP98, PEP99, PEP100, PEP101, PEP102, PEP103, PEP104, PEP105, PEP106, PEP107, PEP108, PEP109, PEP110, PEP111, PEP112, PEP113, PEP114, PEP115, PEP116, PEP117, PEP118, PEP119, PEP120, PEP121, PEP122, PEP123, PEP124, PEP125, PEP126, PEP127, PEP128, PEP129, PEP130, PEP131, and PEP132.

27. A compound comprising an insulin analog selected from the group consisting of INS1, INS2, INS3, INS4, INS5, INS6, INS7, INS8, INS9, INS10, INS11, INS12, INS13, INS14, INS15, INS16, INS17, INS18, INS19, INS20, INS21, INS22, INS23, INS24, INS25, INS26, INS27, INS28, INS29, INS30, and INS31 conjugated to a peptide selected from the group consisting of PEP1, PEP2, PEP3, PEP4, PEP5, PEP6, PEP7, PEP8, PEP9, PEP10, PEP11, PEP12, PEP13, PEP14, PEP15, PEP16, PEP17, PEP18, PEP19, PEP20, PEP21, PEP22, PEP23, PEP24, PEP25, PEP26, PEP27, PEP28, PEP29, PEP30, PEP31, PEP32, PEP33, PEP34, PEP35, PEP36, PEP37, PEP38, PEP39, PEP40, PEP41, PEP42, PEP43, PEP44, PEP45, PEP46, PEP47, PEP48, PEP49, PEP50, PEP51, PEP52, PEP53, PEP54, PEP55, PEP56, PEP57, PEP58, PEP59, PEP60, PEP61, PEP62, PEP63, PEP64, PEP65, PEP66, PEP67, PEP68, PEP69, PEP70, PEP71, PEP72, PEP73, PEP74, PEP75, PEP76, PEP77, PEP79, PEP80, PEP81, PEP82, PEP83, PEP84, PEP85, PEP86, PEP87, PEP88, PEP89, PEP90, PEP91, PEP92, PEP93, PEP94, PEP95, PEP96, PEP97, PEP98, PEP99, PEP100, PEP101, PEP102, PEP103, PEP104, PEP105, PEP106, PEP107, PEP108, PEP109, PEP110, PEP111, PEP112, PEP113, PEP114, PEP115, PEP116, PEP117, PEP118, PEP119, PEP120, PEP121, PEP122, PEP123, PEP124, PEP125, PEP126, PEP127, PEP128, PEP129, PEP130, PEP131, and PEP132.

28. A compound comprising a conjugate selected from the group consisting of CON1, CON2, CON3, CON4, CON5, CON6, CON7, CON8, CON9, CON10, CON11, CON12, CON13, CON14, CON15, CON16, CON17, CON19, CON20, CON21, CON22, CON24, CON25, CON26, CON27, CON28, CON29, CON30, CON31, CON32, CON33, CON34, CON35, CON36, CON37, CON38, CON39, CON40, CON41, CON42, CON43, CON44, CON45, CON46, CON47, CON48, CON49, CON50, CON51, CON52, CON53, CON55, CON56, CON57, CON58, CON59, CON60, CON61, CON62, CON63, CON64, CON65, CON66, CON67, CON68, CON69, CON70, CON71, CON72, CON73, CON74, CON75, CON76, CON77, CON78, CON79, CON80, CON81, CON82, CON83, CON84, CON85, CON86, CON87, CON88, CON89, CON90, CON91, CON92, CON93, CON94, CON95, CON96, CON97, CON98, CON99, CON101, CON102, CON103, CON104, CON105, CON106, CON107, CON108, CON109, CON110, CON111, CON112, CON113, CON114, CON115, CON116, CON117, CON118, CON119, CON120, CON121, CON122, CON123, CON124, CON125, CON126, CON127, CON128, CON129, CON130, CON131, CON132, CON133, CON134, CON135, CON136, CON137, CON138, CON139, CON140, CON141, CON142, CON143, CON144, CON145, CON146, CON147, CON148, CON149, CON150, CON151, CON152, CON153, CON154, CON155, CON156, CON157, CON158, CON159, CON160, and CON161.

29. A pharmaceutical formulation comprising a compound of claim 1 and a pharmaceutically acceptable carrier.

30-35. (canceled)

36. A method for treating a metabolic disease, comprising:
administering to an individual in need an effective amount of a compound of claim 1 to treat the metabolic disease.

37. The method of claim 36, wherein the metabolic disease is diabetes, non-alcoholic fatty liver disease (NAFLD), non-alcoholic steatohepatitis (NASH), or obesity.

38. The method of claim 37, wherein the diabetes is Type I diabetes, Type II diabetes, or gestational diabetes.


SEQUENCE LISTING (166 sequences)

SEQ ID NO: 1 - protein, 29 aa, Artificial Sequence
  Glucagon-derived peptide
HSQGTFTSDYSKYLDERAAQDFVQWLLDT

SEQ ID NO: 2 - protein, 31 aa, Artificial Sequence
  Peptide
  PEPTIDE (1)..(31): wherein the C-terminal amino acid optionally is amidated
  PEPTIDE (1)..(31): with the proviso that either X10 is a Lysine residue conjugated to lipid moiety and one of X20, X21, X24, X30, or X31 comprises the azide group or X20 is a Lysine residue conjugated lipid moiety and one of X21, X24, X30, or X31 comprises the azide group
  MISC_FEATURE (2)..(2): Xaa is amionisobutyric acid (Aib), Gly, D-Serine (s), alpha-methyl Serine (?MS), or alpha-methyl D-Serine (alpha-Ms)
  MISC_FEATURE (3)..(3): Xaa is Val, Glu or Asp
  MISC_FEATURE (9)..(9): Xaa is Asp or alpha-methyl Aspartic acid (alpha-MD)
  MISC_FEATURE (10)..(10): Xaa is Tyr or Lys conjugated to a lipid moiety
  MISC_FEATURE (12)..(12): Xaa is Lysine, Leucine, or Serine
  MISC_FEATURE (14)..(14): Xaa is Leu of alpha-methyl Leucine (alpha-ML)
  MISC_FEATURE (16)..(16): Xaa is Glutamic acid, Asparagine, Serine, Alanine, or Aib
  MISC_FEATURE (17)..(17): Xaa is Arginine or Lysine
  MISC_FEATURE (18)..(18): Xaa is Alanine or Arginine
  MISC_FEATURE (20)..(20): Xaa is Glutamine or Lysine conjugated via its epsilon amino group to a non-peptide linker comprising a terminal azide group or to a fatty acid or fatty diacid
  MISC_FEATURE (21)..(21): Xaa is Aspartic acid, ?MD, Azidonorleucine (Norleucine conjugated via its epsilon carbon to an azide group (Nle(?N3))), or Lysine conjugated via its epsilon amino group to a non-peptide linker comprising a terminal azide group
  MISC_FEATURE (22)..(22): Xaa is Phe or alpha-MF
  MISC_FEATURE (24)..(24): Xaa is Glutamine, Nle(epsilon-N3), or Lysine conjugated via its epsilon amino group to a non-peptide linker comprising a terminal azide group
  MISC_FEATURE (25)..(25): Xaa is Tryptophan or alpha-methyl Tryptophan (alpha-MW);
  MISC_FEATURE (26)..(26): Xaa is Leucine or alpha-ML
  MISC_FEATURE (27)..(27): Xaa is Methionine, Leucine, Methionine sulfoxide, or L-methionine sulphone (2)
  MISC_FEATURE (28)..(28): Xaa is Aspartic acid, Alanine, Lysine, Asparagine, gamma-Glu, Glutamine, or alpha-MD
  MISC_FEATURE (29)..(29): Xaa is Threonine or Glycine
  MISC_FEATURE (30)..(30): Xaa is Arginine, Lysine, or Nle(epsilonN3), or Xaa is absent
  MISC_FEATURE (31)..(31): Xaa is Glycine, gamma-Glu, Nle(epsilon-N3), or Lysine conjugated via its epsilon amino group to a non-peptide linker comprising a terminal azide group, or Xaa is absent
HXXGTFTSXXSXYXDXXXAXXXVXXXXXXXX

SEQ ID NO: 3 - protein, 31 aa, Artificial Sequence; identical to SEQ ID NO: 2
  Peptide
  PEPTIDE (1)..(31): wherein the C-terminal amino acid optionally is amidated
  PEPTIDE (1)..(31): with proviso that either X10 is a Lysine residue conjugated the lipid moiety and one of X20, X21, X24, X30, or X31 comprises the alkyne group or X20 is a Lysine residue conjugated to the lipid moiety and one of X21, X24, X30, or X31 comprises the alkyne
  MISC_FEATURE (2)..(2): Xaa is aminoisobutyric acid (Aib), D-Serine (s), or alpha-methyl Serine (alpha-MS)
  MISC_FEATURE (3)..(3): Xaa is Val, Glu, or Asp
  MISC_FEATURE (9)..(9): Xaa is Asp or alpha-methyl Aspartic acid (alpha-MD)
  MISC_FEATURE (10)..(10): Xaa is Tyr or Lys conjugated to a lipid moiety
  MISC_FEATURE (12)..(12): Xaa is Lysine, Leucine, or Serine
  MISC_FEATURE (14)..(14): Xaa is Leu or alpha-methyl Leucine (alpha-ML)
  MISC_FEATURE (16)..(16): Xaa is Glutamic acid, Asparagine, Serine, Alanine, or Aib
  MISC_FEATURE (17)..(17): Xaa is Arginine or Lysine
  MISC_FEATURE (18)..(18): Xaa is Alanine or Arginine
  MISC_FEATURE (20)..(20): Xaa is Glutamine or Lysine conjugated via its epsilon amino group to a non-peptide linker comprising a terminal alkyne group or to a fatty acid or fatty diacid
  MISC_FEATURE (21)..(21): Xaa is Aspartic acid, alpha-MD, Alkynylnorleucine (Norleucine conjugated via its epsilon carbon to an alkyne group (Nle(epsilon-alkyne))), or Lysine conjugated via its epsilon amino group to a non-peptide linker comprising a terminal alkyne
  MISC_FEATURE (22)..(22): Xaa is Phe or alpha-methyl Phenylalanine (alpha-MF)
  MISC_FEATURE (24)..(24): Xaa is Glutamine, Nle(epsilon-alkyne), or Lysine conjugated via its epsilon amino group to a non-peptide linker comprising a terminal alkyne group
  MISC_FEATURE (25)..(25): Xaa is Tryptophan or alpha-methyl Tryptophan (alpha-MW)
  MISC_FEATURE (26)..(26): Xaa is Leucine or alpha-ML
  MISC_FEATURE (27)..(27): Xaa is Methionine, Leucine, methionine sulfoxide, or L-methionine sulphone (2)
  MISC_FEATURE (28)..(28): Xaa is Aspartic acid, Alanine, Lysine, Asparagine, gamma-Glu, Glutamine, or alpha-MD
  MISC_FEATURE (29)..(29): Xaa is Threonine or Glycine
  MISC_FEATURE (30)..(30): Xaa is Arginine, Lysine, or Nle(epsilon- alkyne), or Xaa is absent
  MISC_FEATURE (31)..(31): Xaa is Glycine, ?Glu, Nle(epsilon-alkyne), or Lysine conjugated via its epsilon amino group to a non-peptide linker comprising a terminal alkyne group, or Xaa is absent

SEQ ID NO: 4 - protein, 31 aa, Artificial Sequence
  Peptide
  PEPTIDE (1)..(31): wherein the C-terminal amino acid optionally is amidated
  PEPTIDE (1)..(31): with proviso that either X10 is a Lysine residue conjugated lipid moiety and one of X20, X21, X24, X30, or X31 comprises the azide group or X20 is a Lysine residue conjugated lipid moiety and one of X21, X24, X30, or X31 comprises the azide group
  MISC_FEATURE (2)..(2): Xaa is aminoisobutyric acid (Aib), D-Serine (s), or alpha-methyl Serine (alpha-MS)
  MISC_FEATURE (9)..(9): Xaa is Asp or alpha-methyl Aspartic acid (alpha-MD)
  MISC_FEATURE (10)..(10): Xaa is Tyr or Lys conjugated to a lipid moiety
  MISC_FEATURE (12)..(12): Xaa is Lysine, Leucine, or Serine
  MISC_FEATURE (16)..(16): Xaa is Glutamic acid, Asparagine, Serine, Alanine, or Aib
  MISC_FEATURE (17)..(17): Xaa is Arginine or Lysine
  MISC_FEATURE (18)..(18): Xaa is Alanine or Arginine
  MISC_FEATURE (20)..(20): Xaa is Glutamine or Lysine conjugated via its epsilon amino group to a non-peptide linker comprising a terminal azide group or to a fatty acid or fatty diacid
  MISC_FEATURE (21)..(21): Xaa is Aspartic acid, alpha-MD, Azidonorleucine (Norleucine conjugated via its epsilon carbon to an azide group (Nle(epsilon-N3))), or Lysine conjugated via its epsilon amino group to a non-peptide linker comprising a terminal azide group
  MISC_FEATURE (22)..(22): Xaa is Phe or alpha-methyl Phenylalanine (alpha-MF)
  MISC_FEATURE (24)..(24): Xaa is Glutamine, Nle(epsilon-N3), or Lysine conjugated via its epsilon amino group to a non-peptide linker comprising a terminal azide group
  MISC_FEATURE (25)..(25): Xaa is Tryptophan or alpha-methyl Tryptophan (alpha-MW)
  MISC_FEATURE (26)..(26): Xaa is Leucine or alpha-ML
  MISC_FEATURE (27)..(27): Xaa is Methionine, Leucine, methionine sulfoxide, or L-methionine sulphone (2)
  MISC_FEATURE (28)..(28): Xaa is Aspartic acid, Alanine, Lysine, Asparagine, ?Glu, Glutamine, or alpha-MD
  MISC_FEATURE (29)..(29): Xaa is Threonine or Glycine
  MISC_FEATURE (30)..(30): Xaa is Arginine, Lysine, or Nle(epsilon-N3), or X30 is absent
  MISC_FEATURE (31)..(31): Xaa is Glycine, gamma-Glu, Nle(epsilon-N3), or Lysine conjugated via its epsilon amino group to a non-peptide linker comprising a terminal azide group, or X31 is absent
HXQGTFTSXXSXYXDXXXAXXXVXXXXXXXX

SEQ ID NO: 5 - protein, 31 aa, Artificial Sequence; identical to SEQ ID NO: 4
  Peptide
  PEPTIDE (1)..(31): wherein the C-terminal amino acid optionally is amidated
  PEPTIDE (1)..(31): with the proviso that either X10 is a Lysine residue conjugated lipid moiety and one of X20, X21, X24, X30, or X31 comprises the alkyne group or X20 is a Lysine residue conjugated lipid moiety and one of X21, X24, X30, or X31 comprises the alkyne group
  MISC_FEATURE (2)..(2): Xaa is aminoisobutyric acid (Aib), D-Serine (s), or alpha-methyl Serine (alpha-MS)
  MISC_FEATURE (9)..(9): Xaa is Asp or alpha-methyl Aspartic acid (alpha-MD)
  MISC_FEATURE (10)..(10): Xaa is Tyr or Lys conjugated to a lipid moiety
  MISC_FEATURE (12)..(12): Xaa is Lysine, Leucine, or Serine
  MISC_FEATURE (14)..(14): Xaa is Leu of alpha-methyl Leucine (alpha-ML)
  MISC_FEATURE (16)..(16): Xaa is Glutamic acid, Asparagine, Serine, Alanine, or Aib
  MISC_FEATURE (17)..(17): Xaa is Arginine or Lysine
  MISC_FEATURE (18)..(18): Xaa is Alanine or Arginine
  MISC_FEATURE (20)..(20): Xaa is Glutamine or Lysine conjugated via its epsilon amino group to a non-peptide linker comprising a terminal alkyne group or to a fatty acid or fatty diacid
  MISC_FEATURE (21)..(21): Xaa is Aspartic acid, alpha-MD, Alkynylnorleucine (Norleucine conjugated via its epsilon carbon to an alkyne group (Nle(epsilon-alkyne))), or Lysine conjugated via its epsilon amino group to a non-peptide linker comprising a terminal alkyne
  MISC_FEATURE (22)..(22): Xaa is Phe or alpha-methyl Phenylalanine (alpha-MF)
  MISC_FEATURE (24)..(24): Xaa is Glutamine, Nle(epsilon-alkyne), or Lysine conjugated via its epsilon amino group to a non-peptide linker comprising a terminal alkyne group
  MISC_FEATURE (25)..(25): Xaa is Tryptophan or alpha-methyl Tryptophan (alpha-MW)
  MISC_FEATURE (26)..(26): Xaa is Leucine or alpha-ML
  MISC_FEATURE (27)..(27): Xaa is Methionine, Leucine, methionine sulfoxide, or L-methionine sulphone (2)
  MISC_FEATURE (28)..(28): Xaa is Aspartic acid, Alanine, Lysine, Asparagine, gamma-Glu, Glutamine, or alpha-MD
  MISC_FEATURE (29)..(29): Xaa is Threonine or Glycine
  MISC_FEATURE (30)..(30): Xaa is Arginine, Lysine, or Nle(epsilon- alkyne), or Xaa is absent and
  MISC_FEATURE (31)..(31): Xaa is Glycine, gamma-Glu, Nle(epsilon-alkyne), or Lysine conjugated via its epsilon amino group to a non-peptide linker comprising a terminal alkyne group, or Xaa is absent

SEQ ID NO: 6 - protein, 31 aa, Artificial Sequence
  Peptide
  MISC_FEATURE (1)..(1): Xaa is Tyrosine, Phenylalanine, Tryptophan, or other amino acid with an aromatic group
  PEPTIDE (1)..(31): wherein the C-terminal amino acid optionally is amidated
  PEPTIDE (1)..(31): with the proviso that either X10 is a Lysine residue conjugated lipid moiety and one of X20, X21, X24, X30, or X31 comprises the azide group or X20 is a Lysine residue conjugated lipid moiety and one of X21, X24, X30, or X31 comprises the azide group
  MISC_FEATURE (2)..(2): Xaa is amionisobutyric acid (Aib), Gly, D-Serine (s), or alpha-methyl Serine (alpha-MS)
  MISC_FEATURE (3)..(3): Xaa is Val, Glu, or Asp
  MISC_FEATURE (9)..(9): Xaa is Asp or alpha-methyl Aspartic acid (alpha-MD)
  MISC_FEATURE (10)..(10): X10 is Tyr or Lys conjugated to a lipid moiety
  MISC_FEATURE (12)..(12): Xaa is Isoleucine, Lysine, Leucine, or Serine
  MISC_FEATURE (14)..(14): Xaa is Leu of alpha-methyl Leucine (alpha-ML)
  MISC_FEATURE (16)..(16): Xaa is Glutamic acid, Asparagine, Serine, Alanine, or Aib
  MISC_FEATURE (17)..(17): Xaa is Arginine or Lysine
  MISC_FEATURE (18)..(18): Xaa is Alanine or Arginine
  MISC_FEATURE (20)..(20): Xaa is Glutamine or Lysine conjugated via its epsilon amino group to a non-peptide linker comprising a terminal azide group or to a fatty acid or fatty diacid
  MISC_FEATURE (21)..(21): Xaa is Aspartic acid, alpha-MD, Azidonorleucine (Norleucine conjugated via its epsilon carbon to an azide group (Nle(epsilon-N3))), or Lysine conjugated via its epsilon amino group to a non-peptide linker comprising a terminal azide group
  MISC_FEATURE (22)..(22): Xaa is Phe or alpha-methyl Phenylalanine (alpha-MF),
  MISC_FEATURE (24)..(24): Xaa is Glutamine, Nle(epsilon-N3), or Lysine conjugated via its epsilon amino group to a non-peptide linker comprising a terminal azide group
  MISC_FEATURE (25)..(25): Xaa is Tryptophan or alpha-methyl Tryptophan (alpha-MW)
  MISC_FEATURE (26)..(26): Xaa is Leucine or alpha-ML
  MISC_FEATURE (27)..(27): Xaa is Met, Leucine, methionine sulfoxide, or L-methionine sulphone (2)
  MISC_FEATURE (28)..(28): Xaa is Aspartic acid, Alanine, Lysine, Asparagine, gamma-Glu, Glutamine, or alpha-MD
  MISC_FEATURE (29)..(29): Xaa is Threonine or Glycine
  MISC_FEATURE (30)..(30): Xaa is Arginine, Lysine, or Nle(epsilon-N3), or Xaa is absent and
  MISC_FEATURE (31)..(31): Xaa is Glycine, gamma-Glu, Nle(epsilon-N3), or Lysine conjugated via its epsilon amino group to a non-peptide linker comprising a terminal azide group, or Xaa is absent
XXXGTFTSXXSXYXDXXXAXXXVXXXXXXXX

SEQ ID NO: 7 - protein, 31 aa, Artificial Sequence; identical to SEQ ID NO: 6
  Peptide
  MISC_FEATURE (1)..(1): Xaa is Tyrosine, Phenylalanine, Tryptophan, or other amino acid with an aromatic group
  PEPTIDE (1)..(31): wherein the C-terminal amino acid optionally is amidated
  PEPTIDE (1)..(31): with the proviso that either X10 is a Lysine residue conjugated to  lipid moiety and one of X20, X21, X24, X30, or X31 comprises the alkyne group or X20 is a Lysine residue conjugated to lipid moiety and one of X21, X24, X30, or X31 comprises the alkyne
  MISC_FEATURE (2)..(2): Xaa is aminoisobutyric acid (Aib), D-Serine (s), or alpha-methyl Serine (alpha-MS)
  MISC_FEATURE (3)..(3): Xaa is Val, Glu, or Asp
  MISC_FEATURE (9)..(9): Xaa is Asp or alpha-methyl Aspartic acid (alpha-MD)
  MISC_FEATURE (10)..(10): Xaa is Tyr or Lys conjugated to a lipid moiety
  MISC_FEATURE (12)..(12): Xaa is Isoleucine, Lysine, Leucine, or Serine
  MISC_FEATURE (14)..(14): Xaa is Leu of alpha-methyl Leucine (alpha-ML)
  MISC_FEATURE (16)..(16): Xaa is Glutamic acid, Asparagine, Serine, Alanine, or Aib
  MISC_FEATURE (17)..(17): Xaa is Arginine or Lysine
  MISC_FEATURE (18)..(18): Xaa is Alanine or Arginine
  MISC_FEATURE (20)..(20): Xaa is Glutamine or Lysine conjugated via its epsilon amino group to a non-peptide linker comprising a terminal alkyne group or to a fatty acid or fatty diacid
  MISC_FEATURE (21)..(21): Xaa is Aspartic acid, alpha-MD, Alkynylnorleucine (Norleucine conjugated via its epsilon carbon to an alkyne group (Nle(epsilon-alkyne))), or Lysine conjugated via its epsilon amino group to a non-peptide linker comprising a terminal alkyne
  MISC_FEATURE (22)..(22): Xaa is Phe or alpha-methyl Phenylalanine (alpha-MF),
  MISC_FEATURE (24)..(24): Xaa is Glutamine, Nle(epsilon-alkyne), or Lysine conjugated via its epsilon amino group to a non-peptide linker comprising a terminal alkyne group
  MISC_FEATURE (26)..(26): Xaa is Leucine or alpha-ML
  MISC_FEATURE (27)..(27): Xaa is Methionine, Leucine, methionine sulfoxide, or L-methionine sulphone (2)
  MISC_FEATURE (28)..(28): Xaa is Aspartic acid, Alanine, Lysine, Asparagine, gamma-Glu, Glutamine, or alpha-MD
  MISC_FEATURE (29)..(29): Xaa is Threonine or Glycine
  MISC_FEATURE (30)..(30): Xaa is Arginine, Lysine, or Nle(epsilon- alkyne), or Xaa is absent and
  MISC_FEATURE (31)..(31): Xaa is Glycine, gamma-Glu, Nle(epsilon-alkyne), or Lysine conjugated via its epsilon amino group to a non-peptide linker comprising a terminal alkyne group, or Xaa is absent and

SEQ ID NO: 8 - protein, 31 aa, Artificial Sequence
  Peptide
  MISC_FEATURE (1)..(1): Xaa is Tyrosine, Phenylalanine, Tryptophan, or other amino acid with an aromatic group
  PEPTIDE (1)..(31): wherein the C-terminal amino acid optionally is amidated
  PEPTIDE (1)..(31): with the proviso that either X10 is a Lysine residue conjugated to  lipid moiety and one of X20, X21, X24, X30, or X31 comprises the azide group or X20 is a Lysine residue conjugated to  lipid moiety and one of X21, X24, X30, or X31 comprises the azide group
  MISC_FEATURE (2)..(2): Xaa is amionisobutyric acid (Aib), Gly, D-Serine (s), or alpha-methyl Serine (alpha-MS)
  MISC_FEATURE (10)..(10): Xaa is Tyr or Lys conjugated to a lipid moiety
  MISC_FEATURE (12)..(12): Xaa is Isoleucine, Lysine, Leucine, or Serine
  MISC_FEATURE (14)..(14): Xaa is Leu of alpha-methyl Leucine (alpha-ML)
  MISC_FEATURE (16)..(16): Xaa is Glutamic acid, Asparagine, Serine, Alanine, or Aib
  MISC_FEATURE (17)..(17): Xaa is Glutamic acid, Asparagine, Serine, Alanine, or Aib
  MISC_FEATURE (17)..(17): Xaa is Arginine or Lysine
  MISC_FEATURE (18)..(18): Xaa is Alanine or Arginine
  MISC_FEATURE (20)..(20): Xaa is Glutamine or Lysine conjugated via its epsilon amino group to a non-peptide linker comprising a terminal azide group or to a fatty acid or fatty diacid
  MISC_FEATURE (21)..(21): Xaa is Aspartic acid, alpha-MD, Azidonorleucine (Norleucine conjugated to an azide group (Nle(epsilonN3))), or Lysine conjugated via to a non-peptide linker comprising a terminal azide group
  MISC_FEATURE (22)..(22): Xaa is Phe or alpha-methyl Phenylalanine (alpha-MF)
  MISC_FEATURE (24)..(24): Xaa is Glutamine, Nle(epsilonN3), or Lysine conjugated via its epsilon amino group to a non-peptide linker comprising a terminal azide group
  MISC_FEATURE (25)..(25): Xaa is Tryptophan or alpha-methyl Tryptophan (alpha-MW)
  MISC_FEATURE (26)..(26): Xaa is Leucine or alpha-ML
  MISC_FEATURE (27)..(27): Xaa is methionine, Leucine, methionine sulfoxide, or L-methionine sulphone (2)
  MISC_FEATURE (28)..(28): Xaa is Aspartic acid, Alanine, Lysine, Asparagine, gamma-Glu, Glutamine, or alpha-MD
  MISC_FEATURE (29)..(29): Xaa is Threonine or Glycine
  MISC_FEATURE (30)..(30): Xaa is Arginine, Lysine, or Nle(epsilonN3), or Xaa is absent and
  MISC_FEATURE (31)..(31): XAA is Glycine, gamma-Glu, Nle(epsilonN3), or Lysine conjugated via its epsilon amino group to a non-peptide linker comprising a terminal azide group, or XAA is absent and
XXQGTFTSXXSXYXDXXXAXXXVXXXXXXXX

SEQ ID NO: 9 - protein, 31 aa, Artificial Sequence
  Peptide
  MISC_FEATURE (1)..(1): Xaa is Tyrosine, Phenylalanine, Tryptophan, or other amino acid with an aromatic group
  PEPTIDE (1)..(31): wherein the C-terminal amino acid optionally is amidated
  PEPTIDE (1)..(31): with the proviso that either X10 is a Lysine residue conjugated to lipid moiety and one of X20, X21, X24, X30, or X31 comprises the alkyne group or X20 is a Lysine residue conjugated to  lipid moiety and one of X21, X24, X30, or X31 comprises the alkyne
  MISC_FEATURE (2)..(2): X2 is aminoisobutyric acid (Aib), D-Serine (s), or alpha-methyl Serine (alpha-MS)
  MISC_FEATURE (9)..(9): Xaa is Asp or alpha-methyl Aspartic acid (alpha-MD)
  MISC_FEATURE (10)..(10): X10 is Tyr or Lys conjugated to a lipid moiety
  MISC_FEATURE (12)..(12): Xaa is Isoleucine, Lysine, Leucine, or Serine
  MISC_FEATURE (14)..(14): Xaa is Leu of alpha-methyl Leucine (alpha-ML)
  MISC_FEATURE (16)..(16): Xaa is Glutamic acid, Asparagine, Serine, Alanine, or Aib
  MISC_FEATURE (17)..(17): Xaa is Arginine or Lysine
  MISC_FEATURE (18)..(18): Xaa is Alanine or Arginine
  MISC_FEATURE (20)..(20): Xaa is Glutamine or Lysine conjugated via its epsilon amino group to a non-peptide linker comprising a terminal alkyne group or to a fatty acid or fatty diacid
  MISC_FEATURE (21)..(21): Xaa is Aspartic acid, alpha-MD, Alkynylnorleucine (Norleucine conjugated via its epsilon carbon to an alkyne group (Nle(epsilon-alkyne))), or Lysine conjugated via its epsilon amino group to a non-peptide linker comprising a terminal alkyne
  MISC_FEATURE (22)..(22): Xaa is Phe or alpha-methyl Phenylalanine (alpha-MF)
  MISC_FEATURE (24)..(24): Xaa is Glutamine, Nle(epsilon-alkyne), or Lysine conjugated via its epsilon amino group to a non-peptide linker comprising a terminal alkyne group
  MISC_FEATURE (25)..(25): Xaa is Tryptophan or alpha-methyl Tryptophan (alpha-MW)
  MISC_FEATURE (26)..(26): Xaa is Leucine or alpha-ML
  MISC_FEATURE (27)..(27): Xaa is methionine, Leucine, methionine sulfoxide, or L-methionine sulphone (2)
  MISC_FEATURE (28)..(28): Xaa is Aspartic acid, Alanine, Lysine, Asparagine, gamma-Glu, Glutamine, or alpha-MD
  MISC_FEATURE (29)..(29): Xaa is Threonine or Glycine
  MISC_FEATURE (30)..(30): Xaa is Arginine, Lysine, or Nle(epsilon- alkyne), or Xaa is absent and
  MISC_FEATURE (31)..(31): Xaa is Glycine, gamma-Glu, Nle(epsilon-alkyne), or Lysine conjugated via its epsilon amino group to a non-peptide linker comprising a terminal alkyne group, or Xaa is absent
XXQGTFTSXXSXYXDXXXAXXFVXXXXXXXX

SEQ ID NO: 10 - protein, 31 aa, Artificial Sequence
  Peptide
  PEPTIDE (1)..(31): wherein the C-terminal amino acid optionally is amidated
  PEPTIDE (1)..(31): with the proviso that either X10 is a Lysine residue conjugated to a gamma-Glu-gamma-Glu-C16 and one of X20, X21, X24, X30, or X31 comprises azide or N3 group or X20 is a Lysine residue conjugated to  PEG2PEG2-gamma-Glu-C18-OH or
  PEPTIDE (1)..(31): the PEG2PEG2gamma-E-C20-OH and one of  X21, X24, X30, or X31 comprises the azide or N3 group
  MISC_FEATURE (2)..(2): Xaa is aminoisobutyric acid (Aib), D-Serine (s), or alpha-methyl Serine (alpha-MS)
  MISC_FEATURE (9)..(9): Xaa is Serine or alpha-methyl Aspartic acid (alpha-MD)
  MISC_FEATURE (10)..(10): Xaa is Tyr or Lys conjugated to gamma-Glu- gamma-Glu-C16
  MISC_FEATURE (12)..(12): Xaa is Lysine, Leucine, or Serine
  MISC_FEATURE (14)..(14): Xaa is Leu of alpha-methyl Leucine (alpha-ML)
  MISC_FEATURE (16)..(16): Xaa is Glutamic acid, Asparagine, Serine, Alanine, or Aib
  MISC_FEATURE (17)..(17): Xaa is Arginine or Lysine
  MISC_FEATURE (18)..(18): Xaa is Alanine or Arginine
  MISC_FEATURE (20)..(20): Xaa is Glutamine or Lysine conjugated via its epsilon amine group to PEG2-C5N3, PEG2PEG2-C5N3, PEG2PEG2-gamma- Glu-C16N3, PEG2PEG2-gamma-Glu-C18-OH, or PEG2PEG2gamma-E-C20-OH
  MISC_FEATURE (21)..(21): Xaa is Aspartic acid, alpha-methyl Phenylalanine (alpha-MF), alpha-MD, Azidonorleucine (Norleucine conjugated to an azide group (Nle(epsilon-N3))), or Lys conjugated via its epsilon amine group to PEG2-C5N3, PEG2PEG2-C5N3, or PEG2PEG2-gamma-Glu-C16N3
  MISC_FEATURE (24)..(24): Xaa is Glutamine, Nle(epsilon-N3), or Lysine conjugated to PEG2PEG2-gamma-Glu-C16N3
  MISC_FEATURE (25)..(25): Xaa is Tryptophan or alpha-methyl Tryptophan (alpha-MW)
  MISC_FEATURE (26)..(26): Xaa is Leucine or alpha-ML
  MISC_FEATURE (27)..(27): Xaa is Leucine or L-methionine sulphone (2)
  MISC_FEATURE (28)..(28): Xaa is Aspartic acid, Alanine, Lysine, Asparagine, gamma-Glu, Glutamine, or alpha-MD
  MISC_FEATURE (29)..(29): Xaa is Threonine or Glycine
  MISC_FEATURE (30)..(30): Xaa is Arginine, Lysine, or Nle(epsilon-N3), or Xaa is absent and
  MISC_FEATURE (31)..(31): Xaa is Glycine, gamma-Glu, Nle(epsilon-N3), or Lysine conjugated via its epsilon amine group to PEG2PEG2-gamma- Glu-C16N3, PEG2-C5N3 or PEG2PEG2-C5N3, or Xaa is absent
HXQGTFTSXXSXYXDXXXAXXFVXXXXXXXX

SEQ ID NO: 11 - protein, 31 aa, Artificial Sequence; identical to SEQ ID NO: 10
  Peptide
  PEPTIDE (1)..(31): wherein the C-terminal amino acid optionally is amidated
  PEPTIDE (1)..(31): with the proviso that either X10 is a Lysine residue conjugated to a gamma-Glu-gamma-Glu-C16 and one of X20, X21, X24, X30, or X31 comprises the -alkyne or X20 is a Lysine residue conjugated to the PEG2PEG2-gamma-Glu-C18-OH or the PEG2PEG2gamma-E-C20-OH
  PEPTIDE (1)..(31): and one of  X21, X24, X30, or X31 comprises the -alkyne
  MISC_FEATURE (2)..(2): Xaa is aminoisobutyric acid (Aib), D-Serine (s), or alpha-methyl Serine (alpha-MS)
  MISC_FEATURE (9)..(9): Xaa is Serine or alpha-methyl Aspartic acid (alpha-MD)
  MISC_FEATURE (10)..(10): Xaa is Tyr or Lys conjugated to gamma-Glu- gamma-Glu-C16
  MISC_FEATURE (12)..(12): Xaa is Lysine, Leucine, or Serine
  MISC_FEATURE (14)..(14): Xaa is Leu of alpha-methyl Leucine (alpha-ML)
  MISC_FEATURE (16)..(16): Xaa is Glutamic acid, Asparagine, Serine, Alanine, or Aib
  MISC_FEATURE (18)..(18): Xaa is Alanine or Arginine
  MISC_FEATURE (20)..(20): X20 is Glutamine or Lysine conjugated via its epsilon amine group to PEG2-C5N3, PEG2PEG2-C5-alkyne, PEG2PEG2- gamma-Glu-C16-alkyne, PEG2PEG2-gamma-Glu-C18-OH, or PEG2PEG2gamma- E-C20-OH
  MISC_FEATURE (21)..(21): Xaa is Aspartic acid, alpha-methyl Phenylalanine (alpha-MF), alpha-MD, Alkynylnorleucine (Norleucine conjugated via its epsilon carbon to an alkyne group (Nle(epsilon- alkyne))), or Lys conjugated v to PEG2-C5-alkyne, PEG2PEG2-C5- alkyne
  MISC_FEATURE (24)..(24): Xaa is Glutamine, Nle(epsilon-alkyne), or Lysine conjugated to PEG2PEG2-gamma-Glu-C16-alkyne
  MISC_FEATURE (25)..(25): Xaa is Tryptophan or alpha-methyl Tryptophan (alpha-MW)
  MISC_FEATURE (26)..(26): Xaa is Leucine or alpha-ML
  MISC_FEATURE (27)..(27): Xaa is Leucine or L-methionine sulphone (2)
  MISC_FEATURE (28)..(28): Xaa is Aspartic acid, Alanine, Lysine, Asparagine, gamma-Glu, Glutamine, or alpha-MD
  MISC_FEATURE (29)..(29): Xaa is Threonine or Glycine
  MISC_FEATURE (30)..(30): Xaa is Arginine, Lysine, or Nle(epsilon- alkyne), or Xaa is absent and
  MISC_FEATURE (31)..(31): Xaa is Glycine, gamma-Glu, Nle(epsilon-alkyne), or Lysine conjugated via its epsilon amine group to PEG2PEG2-gamma-Glu-C16-alkyne, PEG2-C5-alkyne, PEG2PEG2-C5-alkyne, or Xaa is absent

SEQ ID NO: 12 - protein, 31 aa, Artificial Sequence
  Peptide
  PEPTIDE (1)..(31): wherein the C-terminal amino acid optionally is amidated
  PEPTIDE (1)..(31): with the proviso that either X10 is a Lysine residue conjugated to a gamma-Glu-gamma-Glu-C16 and one of X20, X21, X24, X30, or X31 comprises the azide or N3 group or X20 is a Lysine residue conjugated to the PEG2PEG2-gamma-Glu-C18-OH or
  PEPTIDE (1)..(31): the PEG2PEG2gamma-E-C20-OH and one of  X21, X24, X30, or X31 comprises the azide or N3 group
  MISC_FEATURE (2)..(2): Xaa is aminoisobutyric acid (Aib), D-Serine (s), or alpha-methyl Serine (alpha-MS)
  MISC_FEATURE (3)..(3): Xaa is Glu or Asp
  MISC_FEATURE (9)..(9): Xaa is Serine or alpha-methyl Aspartic acid (alpha-MD)
  MISC_FEATURE (10)..(10): Xaa is Tyr or Lys conjugated to gamma-Glu- gamma-Glu-C16
  MISC_FEATURE (12)..(12): Xaa is Lysine, Leucine, or Serine
  MISC_FEATURE (14)..(14): Xaa is Leu of alpha-methyl Leucine (alpha-ML)
  MISC_FEATURE (16)..(16): Xaa is Glutamic acid, Asparagine, Serine, Alanine, or Aib
  MISC_FEATURE (17)..(17): Xaa is Arginine or Lysine
  MISC_FEATURE (18)..(18): Xaa is Alanine or Arginine
  MISC_FEATURE (20)..(20): Xaa is Glutamine or Lysine conjugated via its epsilon amine group to PEG2-C5N3, PEG2PEG2-C5N3, PEG2PEG2-gamma- Glu-C16N3, PEG2PEG2-gamma-Glu-C18-OH, or PEG2PEG2gamma-E-C20-OH
  MISC_FEATURE (21)..(21): X21 is Aspartic acid, alpha-methyl Phenylalanine (alpha-MF), alpha-MD, Azidonorleucine (Norleucine conjugated via its epsilon carbon to an azide group (Nle(epsilon- N3))), or Lys conjugated v to PEG2-C5N3, PEG2PEG2-C5N3, or PEG2PEG2-gamma-Glu-C16N3
  MISC_FEATURE (24)..(24): Xaa is Glutamine, Nle(epsilon-N3), or Lysine conjugated to PEG2PEG2-gamma-Glu-C16N3
  MISC_FEATURE (25)..(25): Xaa is Tryptophan or alpha-methyl Tryptophan (alpha-MW)
  MISC_FEATURE (26)..(26): Xaa is Leucine or alpha-ML
  MISC_FEATURE (27)..(27): Xaa is Leucine or L-methionine sulphone (2)
  MISC_FEATURE (28)..(28): Xaa is Aspartic acid, Alanine, Lysine, Asparagine, gamma-Glu, Glutamine, or alpha-MD
  MISC_FEATURE (30)..(30): Xaa is Arginine, Lysine, or Nle(epsilon-N3), or X30 is absent
  MISC_FEATURE (31)..(31): Xaa is Glycine, gamma-Glu, Nle(epsilon-N3), or Lysine conjugated via its epsilon amine group to PEG2PEG2-gamma- Glu-C16N3, PEG2-C5N3 or PEG2PEG2-C5N3, or Xaa is absent
HXXGTFTSXXSXYXDXXXAXXFVXXXXXXXX

SEQ ID NO: 13 - protein, 31 aa, Artificial Sequence; identical to SEQ ID NO: 12
  Pepetide
  PEPTIDE (1)..(31): wherein the C-terminal amino acid optionally is amidated
  PEPTIDE (1)..(31): with the proviso that either X10 is a Lysine residue conjugated to a gamma-Glu-gamma-Glu-C16 and one of X20, X21, X24, X30, or X31 comprises the -alkyne or X20 is a Lysine residue conjugated to the PEG2PEG2-gamma-Glu-C18-OH or
  PEPTIDE (1)..(31): the PEG2PEG2gamma-E-C20-OH and one of  X21, X24, X30, or X31 comprises the -alkyne
  MISC_FEATURE (2)..(2): Xaa is aminoisobutyric acid (Aib), D-Serine (s), or alpha-methyl Serine (alpha-MS)
  MISC_FEATURE (3)..(3): Xaa is Glu or Asp
  MISC_FEATURE (9)..(9): Xaa is Serine or alpha-methyl Aspartic acid (alpha-MD)
  MISC_FEATURE (10)..(10): Xaa is Tyr or Lys conjugated to gamma-Glu- gamma-Glu-C16
  MISC_FEATURE (12)..(12): Xaa is Lysine, Leucine, or Serine
  MISC_FEATURE (14)..(14): Xaa is Leu of alpha-methyl Leucine (alpha-ML)
  MISC_FEATURE (16)..(16): Xaa is Glutamic acid, Asparagine, Serine, Alanine, or Aib
  MISC_FEATURE (17)..(17): Xaa is Arginine or Lysine
  MISC_FEATURE (18)..(18): Xaa is Alanine or Arginine
  MISC_FEATURE (20)..(20): Xaa is Glutamine or Lysine conjugated via its epsilon amine group to PEG2-C5N3, PEG2PEG2-C5-alkyne, PEG2PEG2- gamma-Glu-C16-alkyne, PEG2PEG2-gamma-Glu-C18-OH, or PEG2PEG2gamma- E-C20-OH
  MISC_FEATURE (21)..(21): Xaa is Aspartic acid, alpha-methyl Phenylalanine (alpha-MF), alpha-MD, Alkynylnorleucine (Norleucine conjugated to an alkyne group (Nle(epsilon-alkyne))), or Lys conjugated  to PEG2-C5-alkyne, PEG2PEG2-C5-alkyne, or
  MISC_FEATURE (24)..(24): Xaa is Glutamine, Nle(epsilon-alkyne), or Lysine conjugated to PEG2PEG2-gamma-Glu-C16-alkyne
  MISC_FEATURE (25)..(25): Xaa is Tryptophan or alpha-methyl Tryptophan (alpha-MW)
  MISC_FEATURE (26)..(26): Xaa is Leucine or alpha-ML
  MISC_FEATURE (27)..(27): Xaa is Leucine or L-methionine sulphone (2)
  MISC_FEATURE (28)..(28): Xaa is Aspartic acid, Alanine, Lysine, Asparagine, gamma-Glu, Glutamine, or alpha-MD
  MISC_FEATURE (29)..(29): Xaa is Threonine or Glycine
  MISC_FEATURE (30)..(30): Xaa is Arginine, Lysine, or Nle(epsilon- alkyne), or Xaa is absent and
  MISC_FEATURE (31)..(31): Xaa is Glycine, gamma-Glu, Nle(epsilon-alkyne), or Lysine conjugated via its epsilon amine group to PEG2PEG2-gamma-Glu-C16-alkyne, PEG2-C5-alkyne, PEG2PEG2-C5-alkyne, or Xaa is absent

SEQ ID NO: 14 - protein, 31 aa, Artificial Sequence
  Peptide
  MISC_FEATURE (1)..(1): Xaa is Tyrosine, Phenylalanine, Tryptophan, or other amino acid with an aromatic group
  PEPTIDE (1)..(31): wherein the C-terminal amino acid optionally is amidated
  PEPTIDE (1)..(31): with the proviso that either X10 is a Lysine residue conjugated to a gamma-Glu-gamma-Glu-C16 and one of X20, X21, X24, X30, or X31 comprises the azide or N3 group or X20 is a Lysine residue conjugated to the PEG2PEG2-gamma-Glu-C18-OH or
  PEPTIDE (1)..(31): the PEG2PEG2gamma-E-C20-OH and one of  X21, X24, X30, or X31 comprises the azide or N3 group
  MISC_FEATURE (2)..(2): Xaa is aminoisobutyric acid (Aib), D-Serine (s), or alpha-methyl Serine (alpha-MS)
  MISC_FEATURE (3)..(3): Xaa is Glu or Asp
  MISC_FEATURE (9)..(9): Xaa is Serine or alpha-methyl Aspartic acid (alpha-MD)
  MISC_FEATURE (10)..(10): Xaa is Tyr or Lys conjugated to gamma-Glu- gamma-Glu-C16
  MISC_FEATURE (12)..(12): Xaa is Isoleucine, Lysine, Leucine, or Serine
  MISC_FEATURE (14)..(14): Xaa is Leu of alpha-methyl Leucine (alpha-ML)
  MISC_FEATURE (16)..(16): Xaa is Glutamic acid, Asparagine, Serine, Alanine, or Aib
  MISC_FEATURE (17)..(17): Xaa is Arginine or Lysine
  MISC_FEATURE (18)..(18): Xaa is Alanine or Arginine
  MISC_FEATURE (20)..(20): Xaa is Glutamine or Lysine conjugated via its epsilon amine group to PEG2-C5N3, PEG2PEG2-C5N3, PEG2PEG2-gamma- Glu-C16N3, PEG2PEG2-gamma-Glu-C18-OH, or PEG2PEG2gamma-E-C20-OH
  MISC_FEATURE (21)..(21): Xaa is Aspartic acid, alpha-methyl Phenylalanine (alpha-MF), alpha-MD, Azidonorleucine (Norleucine conjugated via its epsilon carbon to an azide group (Nle(epsilon- N3))), or Lys conjugated to PEG2-C5N3, PEG2PEG2-C5N3, or PEG2PEG2- gamma-Glu-C16N3
  MISC_FEATURE (24)..(24): Xaa is Glutamine, Nle(epsilon-N3), or Lysine conjugated to PEG2PEG2-gamma-Glu-C16N3
  MISC_FEATURE (25)..(25): Xaa is Tryptophan or alpha-methyl Tryptophan (alpha-MW)
  MISC_FEATURE (26)..(26): Xaa is Leucine or alpha-ML
  MISC_FEATURE (27)..(27): Xaa is Leucine or L-methionine sulphone (2)
  MISC_FEATURE (28)..(28): Xaa is Aspartic acid, Alanine, Lysine, Asparagine, gamma-Glu, Glutamine, or alpha-MD
  MISC_FEATURE (29)..(29): Xaa is Threonine or Glycine
  MISC_FEATURE (30)..(30): Xaa is Arginine, Lysine, or Nle(epsilon-N3), or Xaa is absent
  MISC_FEATURE (31)..(31): Xaa is Glycine, gamma-Glu, Nle(epsilon-N3), or Lysine conjugated via its epsilon amine group to PEG2PEG2-gamma- Glu-C16N3, PEG2-C5N3 or PEG2PEG2-C5N3, or Xaa is absent
XXXGTFTSXXSXYXDXXXAXXFVXXXXXXXX

SEQ ID NO: 15 - protein, 31 aa, Artificial Sequence; identical to SEQ ID NO: 14
  Peptide
  MISC_FEATURE (1)..(1): Xaa is Tyrosine, Phenylalanine, Tryptophan, or other amino acid with an aromatic group
  PEPTIDE (1)..(31): wherein the C-terminal amino acid optionally is amidated
  PEPTIDE (1)..(31): with the proviso that either X10 is a Lysine residue conjugated to a gamma-Glu-gamma-Glu-C16 and one of X20, X21, X24, X30, or X31 comprises the -alkyne or X20 is a Lysine residue conjugated to the PEG2PEG2-gamma-Glu-C18-OH or
  PEPTIDE (1)..(31): the PEG2PEG2gamma-E-C20-OH and one of  X21, X24, X30, or X31 comprises the -alkyne
  MISC_FEATURE (2)..(2): Xaa is aminoisobutyric acid (Aib), D-Serine (s), or alpha-methyl Serine (alpha-MS)
  MISC_FEATURE (3)..(3): Xaa is Glu or Asp
  MISC_FEATURE (9)..(9): Xaa is Serine or alpha-methyl Aspartic acid (alpha-MD)
  MISC_FEATURE (10)..(10): Xaa is Tyr or Lys conjugated to gamma-Glu- gamma-Glu-C16
  MISC_FEATURE (12)..(12): Xaa is Isoleucine, Lysine, Leucine, or Serine
  MISC_FEATURE (14)..(14): Xaa is Leu of alpha-methyl Leucine (alpha-ML)
  MISC_FEATURE (16)..(16): Xaa is Glutamic acid, Asparagine, Serine, Alanine, or Aib
  MISC_FEATURE (17)..(17): Xaa is Arginine or Lysine
  MISC_FEATURE (18)..(18): Xaa is Alanine or Arginine
  MISC_FEATURE (20)..(20): Xaa is Glutamine or Lysine conjugated via its epsilon amine group to PEG2-C5N3, PEG2PEG2-C5-alkyne, PEG2PEG2- gamma-Glu-C16-alkyne, PEG2PEG2-gamma-Glu-C18-OH, or PEG2PEG2gamma- E-C20-OH
  MISC_FEATURE (21)..(21): Xaa is Aspartic acid, alpha-methyl Phenylalanine (alpha-MF), alpha-MD, Alkynylnorleucine (Norleucine conjugated  to an alkyne group (Nle(epsilon-alkyne))), or Lys conjugated to PEG2-C5-alkyne, PEG2PEG2-C5-alkyne, or PEG2PEG2- gamma-Glu-C16
  MISC_FEATURE (24)..(24): Xaa is Glutamine, Nle(epsilon-alkyne), or Lysine conjugated to PEG2PEG2-gamma-Glu-C16-alkyne
  MISC_FEATURE (25)..(25): Xaa is Tryptophan or alpha-methyl Tryptophan (alpha-MW)
  MISC_FEATURE (26)..(26): Xaa is Leucine or alpha-ML
  MISC_FEATURE (27)..(27): Xaa is Leucine or L-methionine sulphone (2)
  MISC_FEATURE (28)..(28): Xaa is Aspartic acid, Alanine, Lysine, Asparagine, gamma-Glu, Glutamine, or alpha-MD
  MISC_FEATURE (29)..(29): Xaa is Threonine or Glycine
  MISC_FEATURE (30)..(30): Xaa is Arginine, Lysine, or Nle(epsilon- alkyne), or Xaa is absent and
  MISC_FEATURE (31)..(31): Xaa is Glycine, gamma-Glu, Nle(epsilon-alkyne), or Lysine conjugated via its epsilon amine group to PEG2PEG2-gamma-Glu-C16-alkyne, PEG2-C5-alkyne, PEG2PEG2-C5-alkyne, or Xaa is absent

SEQ ID NO: 16 - protein, 31 aa, Artificial Sequence; identical to SEQ ID NO: 9
  Peptide
  MISC_FEATURE (1)..(1): Xaa is Tyrosine, Phenylalanine, Tryptophan, or other amino acid with an aromatic group
  PEPTIDE (1)..(31): wherein the C-terminal amino acid optionally is amidated
  PEPTIDE (1)..(31): with the proviso that either X10 is a Lysine residue conjugated to a gamma-Glu-gamma-Glu-C16 and one of X20, X21, X24, X30, or X31 comprises the azide or N3 group or X20 is a Lysine residue conjugated to the PEG2PEG2-gamma-Glu-C18-OH or
  PEPTIDE (1)..(31): the PEG2PEG2gamma-E-C20-OH and one of  X21, X24, X30, or X31 comprises the azide or N3 group
  MISC_FEATURE (2)..(2): Xaa is aminoisobutyric acid (Aib), D-Serine (s), or alpha-methyl Serine (alpha-MS)
  MISC_FEATURE (9)..(9): Xaa is Serine or alpha-methyl Aspartic acid (alpha-MD)
  MISC_FEATURE (10)..(10): Xaa is Tyr or Lys conjugated to gamma-Glu- gamma-Glu-C16
  MISC_FEATURE (12)..(12): Xaa is Isoleucine, Lysine, Leucine, or Serine
  MISC_FEATURE (14)..(14): Xaa is Leu of alpha-methyl Leucine (alpha-ML)
  MISC_FEATURE (16)..(16): Xaa is Glutamic acid, Asparagine, Serine, Alanine, or Aib
  MISC_FEATURE (17)..(17): Xaa is Arginine or Lysine
  MISC_FEATURE (18)..(18): Xaa is Alanine or Arginine
  MISC_FEATURE (20)..(20): X20 is Glutamine or Lysine conjugated via its epsilon amine group to PEG2-C5N3, PEG2PEG2-C5N3, PEG2PEG2-gamma- Glu-C16N3, PEG2PEG2-gamma-Glu-C18-OH, or PEG2PEG2gamma-E-C20-OH
  MISC_FEATURE (21)..(21): Xaa is Aspartic acid, alpha-methyl Phenylalanine (alpha-MF), alpha-MD, Azidonorleucine (Norleucine conjugated via its epsilon carbon to an azide group (Nle(epsilon- N3))), or Lys conjugated to PEG2-C5N3, PEG2PEG2-C5N3, or PEG2PEG2- gamma-Glu-C16N3
  MISC_FEATURE (24)..(24): Xaa is Glutamine, Nle(epsilon-N3), or Lysine conjugated to PEG2PEG2-gamma-Glu-C16N3
  MISC_FEATURE (25)..(25): Xaa is Tryptophan or alpha-methyl Tryptophan (alpha-MW)
  MISC_FEATURE (26)..(26): Xaa is Leucine or alpha-ML
  MISC_FEATURE (27)..(27): Xaa is Leucine or L-methionine sulphone (2)
  MISC_FEATURE (28)..(28): Xaa is Aspartic acid, Alanine, Lysine, Asparagine, gamma-Glu, Glutamine, or alpha-MD
  MISC_FEATURE (29)..(29): Xaa is Threonine or Glycine
  MISC_FEATURE (30)..(30): Xaa is Arginine, Lysine, or Nle(epsilon-N3), or Xaa is absent
  MISC_FEATURE (31)..(31): Xaa is Glycine, gamma-Glu, Nle(epsilon-N3), or Lysine conjugated via its epsilon amine group to PEG2PEG2-gamma- Glu-C16N3, PEG2-C5N3 or PEG2PEG2-C5N3, or Xaa is absent

SEQ ID NO: 17 - protein, 31 aa, Artificial Sequence; identical to SEQ ID NO: 9
  Peptide
  MISC_FEATURE (1)..(1): Xaa is Tyrosine, Phenylalanine, Tryptophan, or other amino acid with an aromatic group
  PEPETIDE (1)..(31): wherein the C-terminal amino acid optionally is amidated
  PEPETIDE (1)..(31): with the proviso that either X10 is a Lysine residue conjugated to a gamma-Glu-gamma-Glu-C16 and one of X20, X21, X24, X30, or X31 comprises the -alkyne or X20 is a Lysine residue conjugated to the PEG2PEG2-gamma-Glu-C18-OH or
  PEPETIDE (1)..(31): the PEG2PEG2gamma-E-C20-OH and one of  X21, X24, X30, or X31
  MISC_FEATURE (2)..(2): Xaa is aminoisobutyric acid (Aib), D-Serine (s), or alpha-methyl Serine (alpha-MS)
  MISC_FEATURE (9)..(9): Xaa is Serine or alpha-methyl Aspartic acid (alpha-MD)
  MISC_FEATURE (10)..(10): Xaa is Tyr or Lys conjugated to gamma-Glu- gamma-Glu-C16
  MISC_FEATURE (12)..(12): Xaa is Isoleucine, Lysine, Leucine, or Serine
  MISC_FEATURE (14)..(14): Xaa is Leu of alpha-methyl Leucine (alpha-ML)
  MISC_FEATURE (16)..(16): Xaa is Glutamic acid, Asparagine, Serine, Alanine, or Aib
  MISC_FEATURE (17)..(17): Xaa is Arginine or Lysine
  MISC_FEATURE (18)..(18): Xaa is Alanine or Arginine
  MISC_FEATURE (20)..(20): Xaa is Glutamine or Lysine conjugated via its epsilon amine group to PEG2-C5N3, PEG2PEG2-C5-alkyne, PEG2PEG2- gamma-Glu-C16-alkyne, PEG2PEG2-gamma-Glu-C18-OH, or PEG2PEG2gamma- E-C20-OH
  MISC_FEATURE (21)..(21): Xaa is Asp, alpha-methyl Phenylalanine (alpha- MF), alpha-MD, Alkynylnorleucine (Norleucine conjugated via its epsilon carbon to an alkyne group (Nle(epsilon-alkyne))), or Lys conjugated to PEG2-C5-alkyne, PEG2PEG2-C5-alkyne, or
  MISC_FEATURE (24)..(24): Xaa is Glutamine, Nle(epsilon-alkyne), or Lysine conjugated to PEG2PEG2-gamma-Glu-C16-alkyne
  MISC_FEATURE (25)..(25): Xaa is Tryptophan or alpha-methyl Tryptophan (alpha-MW)
  MISC_FEATURE (26)..(26): Xaa is Leucine or alpha-ML
  MISC_FEATURE (27)..(27): Xaa is Leucine or L-methionine sulphone (2)
  MISC_FEATURE (28)..(28): Xaa is Aspartic acid, Alanine, Lysine, Asparagine, gamma-Glu, Glutamine, or alpha-MD
  MISC_FEATURE (29)..(29): Xaa is Threonine or Glycine
  MISC_FEATURE (30)..(30): Xaa is Arginine, Lysine, or Nle(epsilon- alkyne), or Xaa is absent
  MISC_FEATURE (31)..(31): X31 is Glycine, gamma-Glu, Nle(epsilon-alkyne), or Lysine conjugated via its epsilon amine group to PEG2PEG2-gamma-Glu-C16-alkyne, PEG2-C5-alkyne, PEG2PEG2-C5-alkyne, or Xaa is absent

SEQ ID NO: 18 - protein, 32 aa, Artificial Sequence
  Peptide
  PEPTIDE (1)..(29): with the proviso that only one of X10, X20, X21, or X28, or X29 is Nle(epsilon-N3) or Lysine conjugated via its epsilon amine group to PEG2PEG2-gamma-Glu-C16- N3, PEG2-C5- N3, PEG2PEG2-C5- N3
  MISC_FEATURE (10)..(10): Xaa is Valine, Nle(epsilon-N3), or Lysine conjugated via its epsilon amine group to PEG2PEG2-gamma-Glu-C16- N3, PEG2-C5- N3, or PEG2PEG2-C5- N3
  MISC_FEATURE (20)..(20): Xaa is Lysine, Nle(epsilon-N3), or Lysine conjugated via its epsilon amine group to PEG2PEG2-gamma-Glu-C16- N3, PEG2-C5- N3, or PEG2PEG2-C5- N3
  MISC_FEATURE (21)..(21): Xaa is glutamic acid, Nle(epsilon-N3), or Lysine conjugated via its epsilon amine group to PEG2PEG2-gamma- Glu-C16- N3, PEG2-C5- N3, or PEG2PEG2-C5- N3
  MISC_FEATURE (28)..(28): Xaa is Lysine, Nle(epsilon-N3), or Lysine conjugated via its epsilon amine group to PEG2PEG2-gamma-Glu-C16- N3, PEG2-C5- N3, or PEG2PEG2-C5- N3
  MISC_FEATURE (29)..(29): Xaa Nle(epsilon-N3), or Lysine conjugated via its epsilon amine group to PEG2PEG2-gamma-Glu-C16- N3, PEG2-C5- N3, PEG2PEG2-C5- N3 or absent
  misc_feature (32)..(32): Xaa can be any naturally occurring amino acid
HGEGTFTSDXSSYLEEQAAXXFIAWLVXGGGX

SEQ ID NO: 19 - protein, 32 aa, Artificial Sequence; identical to SEQ ID NO: 18
  Peptide
  PEPTIDE (1)..(29): with the proviso that only one of X10, X20, X21, or X28, or X29 is Nle(epsilon-N3) or Lysine conjugated via its epsilon amine group to PEG2PEG2-gamma-Glu-C16-alkyne, PEG2-C5- alkyne, PEG2PEG2-C5-alkyne
  MISC_FEATURE (10)..(10): Xaa is Valine, Nle(epsilon-alkyne), or Lysine conjugated via its epsilon amine group to PEG2PEG2-gamma-Glu-C16- alkyne, PEG2-C5-alkyne, or PEG2PEG2-C5-alkyne
  MISC_FEATURE (20)..(20): Xaa is Lysine, Nle(epsilon-N3), or Lysine conjugated via its epsilon amine group to PEG2PEG2-gamma-Glu-C16- alkyne, PEG2-C5-alkyne, or PEG2PEG2-C5-alkyne
  MISC_FEATURE (21)..(21): Xaa is glutamic acid, Nle(epsilon-alkyne), or Lysine conjugated via its epsilon amine group to PEG2PEG2-gamma- Glu-C16-alkyne, PEG2-C5-alkyne, or PEG2PEG2-C5-alkyne
  MISC_FEATURE (28)..(28): Xaa is Lysine, Nle(epsilon-alkyne), or Lysine conjugated via its epsilon amine group to PEG2PEG2-gamma-Glu-C16- alkyne, PEG2-C5-alkyne, or PEG2PEG2-C5-alkyne
  MISC_FEATURE (29)..(29): Xaa Nle(epsilon-alkyne), or Lysine conjugated via its epsilon amine group to PEG2PEG2-gamma-Glu-C16-alkyne, PEG2-C5-alkyne, PEG2PEG2-C5-alkyne or absent
  misc_feature (32)..(32): Xaa can be any naturally occurring amino acid

SEQ ID NO: 20 - protein, 21 aa, Homo sapiens
GIVEQCCTSICSLYQLENYCN

SEQ ID NO: 21 - protein, 30 aa, Homo sapiens
FVNQHLCGSHLVEALYLVCGERGFFYTPKT

SEQ ID NO: 22 - protein, 30 aa, Artificial Sequence
  Lispro B chain
FVNQHLCGSHLVEALYLVCGERGFFYTKPT

SEQ ID NO: 23 - protein, 30 aa, Artificial Sequence
  Aspart B chain
FVNQHLCGSHLVEALYLVCGERGFFYTDKT

SEQ ID NO: 24 - protein, 30 aa, Artificial Sequence
  Glulisine B chain
FVKQHLCGSHLVEALYLVCGERGFFYTPET

SEQ ID NO: 25 - protein, 21 aa, Artificial Sequence
  Glargine A chain
GIVEQCCTSICSLYQLENYCG

SEQ ID NO: 26 - protein, 32 aa, Artificial Sequence
  Glargine B chain
FVNQHLCGSHLVEALYLVCGERGFFYTPKTRR

SEQ ID NO: 27 - protein, 21 aa, Artificial Sequence
  Peptide
  MISC_FEATURE (8)..(8)
  MISC_FEATURE (21)..(21): Xaa is asparagine or glycine
GIVEQCCXSICSLYQLENYCX

SEQ ID NO: 28 - protein, 21 aa, Artificial Sequence
  Peptide
  MISC_FEATURE (1)..(1): Xaa is selected from the group consisting of histidine and threonine
  MISC_FEATURE (5)..(5): Xaa is selected from the group consisting of alanine, glycine and serine;
  MISC_FEATURE (6)..(6): Xaa is selected from the group consisting of histidine, aspartic acid, glutamic acid, homocysteic acid and cysteic acid
XLCGXXLVEALYLVCGERGFF

SEQ ID NO: 29 - protein, 29 aa, Artificial Sequence
  Peptide
  MISC_FEATURE (1)..(1): Xaa is selected from the group consisting of phenylalanine and desamino-phenylalanine.
  MISC_FEATURE (5)..(5): Xaa is selected from the group consisting of histidine and threonine;
  MISC_FEATURE (9)..(9): Xaa is selected from the group consisting of alanine, glycine and serine;
  MISC_FEATURE (10)..(10): Xaa is selected from the group consisting of histidine, aspartic acid, glutamic acid, homocysteic acid and cysteic acid
  misc_feature (28)..(28): Xaa can be any naturally occurring amino acid
  MISC_FEATURE (29)..(29): Xaa is threonine, alanine, or a threonine- arginine-arginine tripeptide
XVNQXLCGXXLVEALYLVCGERGFFYTXX

SEQ ID NO: 30 - protein, 22 aa, Artificial Sequence
  Peptide
  MISC_FEATURE (1)..(1): Xaa1 is glycine (G) or lysine (K)
  MISC_FEATURE (3)..(3): Xaa3 is valine (V), glycine (G), or lysine (K)
  MISC_FEATURE (5)..(5): Xaa5 is valine (V), glycine (G), or lysine (K)
  MISC_FEATURE (8)..(8): Xaa8 is threonine (T) or histidine (H)
  MISC_FEATURE (9)..(9): Xaa9 is serine (S) or lysine (K)
  MISC_FEATURE (10)..(10): Xaa10 is isoleucine (I) or lysine
  MISC_FEATURE (13)..(13): Xaa13 is leucine (L) or lysine (K)
  MISC_FEATURE (14)..(14): Xaa14 is tyrosine (Y) or lysine (K)
  MISC_FEATURE (15)..(15): Xaa15 is glutamine (Q) or lysine (K)
  MISC_FEATURE (18)..(18): Xaa18 is asparagine (N) or lysine (K)
  MISC_FEATURE (21)..(21): Xaa21 is asparagine (N) or glycine (G)
  MISC_FEATURE (22)..(22): Xaa22 is arginine (R), lysine (K) or absent
XIXEXCCXXXCSXXXLEXYCXX

SEQ ID NO: 31 - protein, 35 aa, Artificial Sequence
  Peptide
  MISC_FEATURE (1)..(1): Xaa1 is phenylalanine (F) or lysine (K)
  MISC_FEATURE (3)..(3): Xaa3 is asparagine (N) or lysine (K)
  MISC_FEATURE (4)..(4): Xaa4 is glutamine (Q) or lysine (K)
  MISC_FEATURE (16)..(16): Xaa16 is tyrosine (Y) or lysine (K)
  MISC_FEATURE (17)..(17): Xaa17 is leucine (L) or lysine (K)
  MISC_FEATURE (25)..(25): Xaa25 is phenylalanine (F) or lysine (K)
  MISC_FEATURE (28)..(28): Xaa28 is proline (P) or lysine (K)
  MISC_FEATURE (29)..(29): Xaa29 is lysine (K) or proline (P)
  MISC_FEATURE (30)..(30): Xaa30 is threonine (T) or absent
  MISC_FEATURE (31)..(31): Xaa31 is arginine (R) if Xaa30 is threonine (T), or absent
  MISC_FEATURE (32)..(32): Xaa32 is proline (P) if Xaa31 is arginine (R), or absent
  MISC_FEATURE (33)..(33): Xaa33 is arginine (R) if Xaa32 is proline (P), or absent
  MISC_FEATURE (34)..(34): Xaa34 is proline (P) if Xaa33 is arginine (R), or absent
  MISC_FEATURE (35)..(35): Xaa35 is arginine (R) if Xaa34 is proline (P), or absent
XVXXHLCGSHLVEALXXVCGERGFXYTXXXXXXXX

SEQ ID NO: 32 - protein, 29 aa, Artificial Sequence
  Peptide
  MISC_FEATURE (1)..(1): Xaa1 is phenylalanine (F) or lysine (K)
  MISC_FEATURE (3)..(3): Xaa3 is asparagine (N) or lysine (K)
  MISC_FEATURE (4)..(4): Xaa4 is glutamine (Q) or lysine (K)
  MISC_FEATURE (16)..(16): Xaa16 is tyrosine (Y) or lysine (K)
  MISC_FEATURE (17)..(17): Xaa17 is leucine (L) or lysine (K)
  MISC_FEATURE (25)..(25): Xaa25 is phenylalanine (F) or lysine (K)
  MISC_FEATURE (28)..(28): Xaa28 is proline (P) or lysine (K)
  MISC_FEATURE (29)..(29): Xaa29 is lysine (K) or proline (P)
XVXXHLCGSHLVEALXXVCGERGFXYTXX

SEQ ID NO: 33 - protein, 35 aa, Artificial Sequence
  Peptide
  MISC_FEATURE (1)..(1): Xaa1 is phenylalanine (F) or lysine (K)
  MISC_FEATURE (3)..(3): Xaa3 is asparagine (N) or lysine (K)
  MISC_FEATURE (4)..(4): Xaa4 is glutamine (Q) or lysine (K)
  MISC_FEATURE (16)..(16): Xaa16 is tyrosine (Y) or lysine (K)
  MISC_FEATURE (17)..(17): Xaa17 is leucine (L) or lysine (K)
  MISC_FEATURE (25)..(25): Xaa25 is phenylalanine (F) or lysine (K)
  MISC_FEATURE (28)..(28): Xaa28 is proline (P) or lysine (K)
  MISC_FEATURE (29)..(29): Xaa29 is lysine (K) or proline (P)
XVXXHLCGSHLVEALXXVCGERGFXYTXXTRPRPR

SEQ ID NO: 34 - protein, 33 aa, Artificial Sequence
  Peptide
  MISC_FEATURE (1)..(1): Xaa1 is phenylalanine (F) or lysine (K)
  MISC_FEATURE (3)..(3): Xaa3 is asparagine (N) or lysine (K)
  MISC_FEATURE (4)..(4): Xaa4 is glutamine (Q) or lysine (K)
  MISC_FEATURE (16)..(16): Xaa16 is tyrosine (Y) or lysine (K)
  MISC_FEATURE (17)..(17): Xaa17 is leucine (L) or lysine (K)
  MISC_FEATURE (25)..(25): Xaa25 is phenylalanine (F) or lysine (K)
  MISC_FEATURE (28)..(28): Xaa28 is proline (P) or lysine (K)
  MISC_FEATURE (29)..(29): Xaa29 is lysine (K) or proline (P)
XVXXHLCGSHLVEALXXVCGERGFXYTXXTRPR

SEQ ID NO: 35 - protein, 29 aa, Artificial Sequence
  Peptide
  PEPTIDE (1)..(29): C-terminal amide
  MISC_FEATURE (2)..(2): Xaa2 is D-Ser
  MISC_FEATURE (10)..(10): Lys10 conjugated to  gamma-Glu-gamma-Glu-C16
  MISC_FEATURE (21)..(21): Xaa21 is Nle(epsilon-N3)
HXQGTFTSDKSKYLDERAAQXFVQWLLDT

SEQ ID NO: 36 - protein, 31 aa, Artificial Sequence
  Peptide
  PEPTIDE (1)..(31): C-terminal amide
  MISC_FEATURE (2)..(2): Xaa2 is aib
  MISC_FEATURE (10)..(10): Lys10 conjugated to  gamma-Glu-gamma-Glu-C16
  MISC_FEATURE (24)..(24): Xaa24 is Nle(epsilon-N3)
HXQGTFTSDKSKYLDERAAQDFVXWLLDGRG

SEQ ID NO: 37 - protein, 31 aa, Artificial Sequence
  Peptide
  PEPTIDE (1)..(31)
  MISC_FEATURE (2)..(2): Xaa2 is aib
  MISC_FEATURE (10)..(10): Lys10 conjugated to  gamma-Glu-gamma-Glu-C16
  MISC_FEATURE (16)..(16): Xaa16 is aib
  MISC_FEATURE (24)..(24): Xaa24 is Nle(epsilon-N3)
  MISC_FEATURE (27)..(27): Xaa27 is methione sulfone
HXQGTFTSDKSKYLDXRAAQDFVXWLXKGRG

SEQ ID NO: 38 - protein, 31 aa, Artificial Sequence
  Peptide
  PEPTIDE (1)..(31): C-terminal amide
  MISC_FEATURE (2)..(2): Xaa2 is D-Ser
  MISC_FEATURE (10)..(10): Lys10 conjugated to  gamma-Glu-gamma-Glu-C16
  MISC_FEATURE (16)..(16): Xaa16 is aib
  MISC_FEATURE (24)..(24): Xaa24 is Nle(epsilon-N3)
  MISC_FEATURE (31)..(31): Xaa31 is gamma-Glu
HXQGTFTSDKSLYLDXRAAQDFVXWLLNTKX

SEQ ID NO: 39 - protein, 31 aa, Artificial Sequence
  Peptide
  PEPTIDE (1)..(31): C-terminal amide
  MISC_FEATURE (2)..(2): Xaa2 is D-Ser
  MISC_FEATURE (10)..(10): Lys10 conjugated to  gamma-Glu-gamma-Glu-C16
  MISC_FEATURE (16)..(16): Xaa16 is aib
  MISC_FEATURE (30)..(30): Xaa30 is Nle(epsilon-N3)
  MISC_FEATURE (31)..(31): Xaa31 is  gamma-Glu
HXQGTFTSDKSLYLDXRAAQDFVQWLLNTXX

SEQ ID NO: 40 - protein, 31 aa, Artificial Sequence
  Peptide
  PEPTIDE (1)..(31): C-terminal amide
  MISC_FEATURE (2)..(2): Xaa2 is aib
  MISC_FEATURE (10)..(10): Lys10 conjugated to  gamma-Glu-gamma-Glu-C16
  MISC_FEATURE (16)..(16): Xaa16 is aib
  MISC_FEATURE (30)..(30): Xaa30 is Nle(epsilon-N3)
  MISC_FEATURE (31)..(31): Xaa31 is  gamma-Glu
HXQGTFTSDKSKYLDXRRAQDFVQWLLDTXX

SEQ ID NO: 41 - protein, 29 aa, Artificial Sequence
  Peptide
  PEPTIDE (1)..(29): C-terminal amide
  MISC_FEATURE (2)..(2): Xaa2 is D-Ser
  MISC_FEATURE (10)..(10): Lys10 conjugated to  gamma-Glu-gamma-Glu-C16
  MISC_FEATURE (16)..(16): Xaa16 is aib
  MISC_FEATURE (24)..(24): Xaa24 is Nle(epsilon-N3)
HXQGTFTSDKSKYLDXRAAQDFVXWLLDT

SEQ ID NO: 42 - protein, 31 aa, Artificial Sequence
  Peptide
  PEPTIDE (1)..(31): C-terminal amide
  MISC_FEATURE (2)..(2): Xaa2 is D-Ser
  MISC_FEATURE (10)..(10): Lys10 conjugated to  gamma-Glu-gamma-Glu-C16
  MISC_FEATURE (16)..(16): Xaa16 is aib
  MISC_FEATURE (30)..(30): Xaa30 is Nle(epsilon-N3)
  MISC_FEATURE (31)..(31): Xaa31 is  gamma-Glu
HXQGTFTSDKSSYLDXRAAQDFVQWLLNTXX

SEQ ID NO: 43 - protein, 31 aa, Artificial Sequence
  Peptide
  PEPTIDE (1)..(31): C-terminal amide
  MISC_FEATURE (2)..(2): Xaa2 is D-Ser
  MISC_FEATURE (10)..(10): Lys10 conjugated to  gamma-Glu-gamma-Glu-C16
  MISC_FEATURE (16)..(16): Xaa16 is aib
  MISC_FEATURE (24)..(24): Xaa24 is Nle(epsilon-N3)
HXQGTFTSDKSSYLDXRAAQDFVXWLLNTKX

SEQ ID NO: 44 - protein, 29 aa, Artificial Sequence
  Peptide
  PEPTIDE (1)..(29): C-terminal amide
  MISC_FEATURE (2)..(2): Xaa2 is aib
  MISC_FEATURE (10)..(10): Lys10 conjugated to  gamma-Glu-gamma-Glu-C16
  MISC_FEATURE (16)..(16): Xaa16 is aib
  MISC_FEATURE (24)..(24): Xaa24 is Nle(epsilon-N3)
  MISC_FEATURE (27)..(27): Xaa27 is methione sulfone
HXQGTFTSDKSKYLDXRAAQDFVXWLXDT

SEQ ID NO: 45 - protein, 29 aa, Artificial Sequence
  Peptide
  PEPTIDE (1)..(29): C-terminal amide
  MISC_FEATURE (2)..(2): Xaa2 is D-Ser
  MISC_FEATURE (10)..(10): Lys10 conjugated to  gamma-Glu-gamma-Glu-C16
  MISC_FEATURE (24)..(24): Xaa24 is Nle(epsilon-N3)
HXQGTFTSDKSKYLDERAAQDFVXWLLXT

SEQ ID NO: 46 - protein, 29 aa, Artificial Sequence
  Peptide
  PEPTIDE (1)..(29): C-terminal amide
  MISC_FEATURE (2)..(2): Xaa2 is D-Ser
  MISC_FEATURE (10)..(10): Lys10 conjugated to  gamma-Glu-gamma-Glu-C16
  MISC_FEATURE (24)..(24): Xaa24 is Nle(epsilon-N3)
HXQGTFTSDKSKYLDNKRAQDFVXWLLQT

SEQ ID NO: 47 - protein, 29 aa, Artificial Sequence
  Peptide
  PEPTIDE (1)..(29): C-terminal amide
  MISC_FEATURE (2)..(2): Xaa2 is D-Ser
  MISC_FEATURE (10)..(10): Lys10 conjugated to  gamma-Glu-gamma-Glu-C16
  MISC_FEATURE (24)..(24): Xaa24 is Nle(epsilon-N3)
HXQGTFTSDKSKYLDSRRAHDFVXWLLNT

SEQ ID NO: 48 - protein, 29 aa, Artificial Sequence
  Peptide
  PEPTIDE (1)..(29): C-terminal amide
  MISC_FEATURE (2)..(2): Xaa2 is D-Ser
  MISC_FEATURE (10)..(10): Lys10 conjugated to  gamma-Glu-gamma-Glu-C16
  MISC_FEATURE (24)..(24): Lys24 is conjugated to  PEG2-C5N3
HXQGTFTSDKSKYLDERAAQDFVKWLLDT

SEQ ID NO: 49 - protein, 29 aa, Artificial Sequence
  Peptide
  PEPTIDE (1)..(29): C-terminal amide
  MISC_FEATURE (2)..(2): Xaa2 is D-Ser
  MISC_FEATURE (10)..(10): Lys10 conjugated to  gamma-Glu-gamma-Glu-C16
  MISC_FEATURE (24)..(24): Lys24 is conjugated to  PEG2PEG2-C5N3
HXQGTFTSDKSKYLDERAAQDFVKWLLDT

SEQ ID NO: 50 - protein, 29 aa, Artificial Sequence
  Peptide
  PEPTIDE (1)..(29): C-terminal amide
  MISC_FEATURE (2)..(2): Xaa2 is D-Ser
  MISC_FEATURE (10)..(10): Lys10 conjugated to  gamma-Glu-gamma-Glu-C16
  MISC_FEATURE (20)..(20): Lys20 is conjugated to  PEG2-C5N3
HXQGTFTSDKSKYLDERAAKDFVQWLLDT

SEQ ID NO: 51 - protein, 29 aa, Artificial Sequence
  Peptide
  PEPTIDE (1)..(29): C-terminal amide
  MISC_FEATURE (2)..(2): Xaa2 is D-Ser
  MISC_FEATURE (10)..(10): Lys10 conjugated to  gamma-Glu-gamma-Glu-C16
  MISC_FEATURE (20)..(20): Lys20 is conjugated to  PEG2PEG2-C5N3
HXQGTFTSDKSKYLDERAAKDFVQWLLDT

SEQ ID NO: 52 - protein, 31 aa, Artificial Sequence
  Peptide
  PEPTIDE (1)..(31): C-terminal amide
  MISC_FEATURE (2)..(2): Xaa2 is aib
  MISC_FEATURE (16)..(16): Xaa16 is aib
  MISC_FEATURE (31)..(31): Xaa31 is Nle(epsilon-N3)
HXQGTFTSDYSKYLDXRAAQDFVQWLLDTKX

SEQ ID NO: 53 - protein, 31 aa, Artificial Sequence
  Peptide
  PEPTIDE (1)..(31): C-terminal amide
  MISC_FEATURE (2)..(2): Xaa2 is aib
  MISC_FEATURE (10)..(10): Lys10 conjugated to  gamma-Glu-gamma-Glu-C16
  MISC_FEATURE (16)..(16): Xaa16 is aib
  MISC_FEATURE (31)..(31): Xaa31 is Nle(epsilon-N3)
HXQGTFTSDKSKYLDXRAAQDFVQWLLDTKX

SEQ ID NO: 54 - protein, 31 aa, Artificial Sequence
  Peptide
  PEPTIDE (1)..(31): C-terminal amide
  MISC_FEATURE (2)..(2): Xaa2 is aib
  MISC_FEATURE (10)..(10): Lys10 conjugated to  gamma-Glu-gamma-Glu-C16
  MISC_FEATURE (16)..(16): Xaa16 is aib
  MISC_FEATURE (31)..(31): K31 conjugated to  PEG2-C5N3
HXQGTFTSDKSKYLDXRAAQDFVQWLLDTKK

SEQ ID NO: 55 - protein, 31 aa, Artificial Sequence; identical to SEQ ID NO: 54
  Peptide
  PEPTIDE (1)..(31): C-terminal amide
  MISC_FEATURE (2)..(2): Xaa2 is aib
  MISC_FEATURE (10)..(10): Lys10 conjugated to  gamma-Glu-gamma-Glu-C16
  MISC_FEATURE (16)..(16): Xaa16 is aib
  MISC_FEATURE (31)..(31): Lys31 is conjugated to  PEG2PEG2-C5N3

SEQ ID NO: 56 - protein, 31 aa, Artificial Sequence
  Peptide
  PEPTIDE (1)..(31): C-terminal amide
  MISC_FEATURE (2)..(2): Xaa2 is aib
  MISC_FEATURE (10)..(10): Lys10 conjugated to  gamma-Glu-gamma-Glu-C16
  MISC_FEATURE (16)..(16): Xaa16 is aib
  MISC_FEATURE (24)..(24): Xaa24 is Nle(epsilon-N3)
  MISC_FEATURE (31)..(31): X31 is gamma-Glu
HXQGTFTSDKSKYLDXRAAQDFVXWLLDTKX

SEQ ID NO: 57 - protein, 29 aa, Artificial Sequence
  Peptide
  PEPTIDE (1)..(29): C-terminal amide
  MISC_FEATURE (2)..(2): Xaa2 is D-Ser
  MISC_FEATURE (10)..(10): Lys10 conjugated to  gamma-Glu-gamma-Glu-C16
  MISC_FEATURE (21)..(21): Xaa21 is Nle(epsilon-N3)
  MISC_FEATURE (27)..(27): Xaa27 is methione sulfone
HXQGTFTSDKSKYLDERAAQXFVQWLXDT

SEQ ID NO: 58 - protein, 29 aa, Artificial Sequence
  Peptide
  PEPTIDE (1)..(29): C-terminal amide
  MISC_FEATURE (2)..(2): Xaa2 is D-Ser
  MISC_FEATURE (10)..(10): Lys10 conjugated to  gamma-Glu-gamma-Glu-C16
  MISC_FEATURE (21)..(21): Xaa21 is Nle(epsilon-N3)
  MISC_FEATURE (27)..(27): Xaa27 is methione sulfone
  MISC_FEATURE (28)..(28): Xaa28 is alpha-MD
HXQGTFTSDKSKYLDERAAQXFVQWLXXT

SEQ ID NO: 59 - protein, 29 aa, Artificial Sequence
  Peptide
  PEPTIDE (1)..(29): C-terminal amide
  MISC_FEATURE (2)..(2): Xaa2 is D-Ser
  MISC_FEATURE (21)..(21): Xaa21 is Nle(epsilon-N3)
  MISC_FEATURE (24)..(24): Lys24 is conjugated to  PEG2PEG2-gamma-Glu- C16N3
HXQGTFTSDYSKYLDERAAQDFVKWLLDT

SEQ ID NO: 60 - protein, 29 aa, Artificial Sequence
  Peptide
  PEPTIDE (1)..(29): C-terminal amide
  MISC_FEATURE (2)..(2): Xaa2 is D-Ser
  MISC_FEATURE (20)..(20): Lys20 is conjugated to  PEG2PEG2-gamma-Glu- C16N3
HXQGTFTSDYSKYLDERAAKDFVQWLLDT

SEQ ID NO: 61 - protein, 29 aa, Artificial Sequence
  Peptide
  PEPTIDE (1)..(29): C-terminal amide
  MISC_FEATURE (2)..(2): Xaa2 is D-Ser
  MISC_FEATURE (21)..(21): Lys21 is conjugated to  PEG2PEG2gamma-Glu-C16N3
HXQGTFTSDYSKYLDERAAQKFVQWLLDT

SEQ ID NO: 62 - protein, 31 aa, Artificial Sequence
  Peptide
  PEPTIDE (1)..(31): C-terminal amide
  MISC_FEATURE (2)..(2): Xaa2 is aib
  MISC_FEATURE (16)..(16): Xaa16 is aib
  MISC_FEATURE (31)..(31): Lys31 is conjugated to  PEG2PEG2-gamma-Glu- C16N3
HXQGTFTSDYSKYLDXRAAQDFVQWLLDTKK

SEQ ID NO: 63 - protein, 29 aa, Artificial Sequence
  Peptide
  PEPTIDE (1)..(29): C-terminal amide
  MISC_FEATURE (2)..(2): Xaa2 is D-Ser
  MISC_FEATURE (10)..(10): Lys10 is conjugated to  PEG2PEG2-gamma-Glu- C18OH
  MISC_FEATURE (24)..(24): Xaa24 is Nle(epsilon-N3)
HXQGTFTSDKSKYLDERAAQDFVXWLLDT

SEQ ID NO: 64 - protein, 29 aa, Artificial Sequence
  Peptide
  PEPTIDE (1)..(29): C-terminal amide
  MISC_FEATURE (2)..(2): Xaa2 is aib
  MISC_FEATURE (20)..(20): Lys20 is cojugated to  PEG2PEG2-gamma-Glu-C18- OH
  MISC_FEATURE (24)..(24): Xaa24 is Nle(epsilon-N3)
  MISC_FEATURE (27)..(27): Xaa27 is methione sulfone
HXQGTFTSDYSKYLDARAAKDFVXWLXDT

SEQ ID NO: 65 - protein, 29 aa, Artificial Sequence
  Peptide
  PEPTIDE (1)..(29): C-terminal amide
  MISC_FEATURE (2)..(2): Xaa2 is aib
  MISC_FEATURE (20)..(20): Lys20 is conjugated to  PEG2PEG2-gamma-Glu-C20- OH
  MISC_FEATURE (24)..(24): Xaa24 is Nle(epsilon-N3)
  MISC_FEATURE (27)..(27): Xaa27 is methione sulfone
HXQGTFTSDYSKYLDARAAKDFVXWLXDT

SEQ ID NO: 66 - protein, 29 aa, Artificial Sequence
  Peptide
  PEPTIDE (1)..(29): C-terminal amide
  MISC_FEATURE (2)..(2): Xaa2 is D-Ser
  MISC_FEATURE (9)..(9): Xaa9 is alpha-MD
  MISC_FEATURE (10)..(10): Lys10 conjugated to  gamma-Glu-gamma-Glu-C16
  MISC_FEATURE (24)..(24): Xaa24 is Nle(epsilon-N3)
  MISC_FEATURE (27)..(27): Xaa27 is methione sulfone
HXQGTFTSXKSKYLDERAAQDFVXWLXDT

SEQ ID NO: 67 - protein, 29 aa, Artificial Sequence
  Peptide
  PEPTIDE (1)..(29): C-terminal amide
  MISC_FEATURE (2)..(2): Xaa2 is D-Ser
  MISC_FEATURE (10)..(10): Lys10 conjugated to  gamma-Glu-gamma-Glu-C16
  MISC_FEATURE (14)..(14): Xaa14 is alpha-ML
  MISC_FEATURE (24)..(24): Xaa24 is Nle(epsilon-N3)
  MISC_FEATURE (27)..(27): Xaa27 is methione sulfone
HXQGTFTSDKSKYXDERAAQDFVXWLXDT

SEQ ID NO: 68 - protein, 29 aa, Artificial Sequence
  Peptide
  PEPTIDE (1)..(29): C-terminal amide
  MISC_FEATURE (2)..(2): Xaa2 is D-Ser
  MISC_FEATURE (10)..(10): Lys10 conjugated to  gamma-Glu-gamma-Glu-C16
  MISC_FEATURE (21)..(21): Xaa21 is alpha-MD
  MISC_FEATURE (24)..(24): Xaa24 is Nle(epsilon-N3)
  MISC_FEATURE (27)..(27): Xaa27 is methione sulfone
HXQGTFTSDKSKYLDERAAQXFVXWLXDT

SEQ ID NO: 69 - protein, 29 aa, Artificial Sequence
  Peptide
  PEPTIDE (1)..(29): C-terminal amide
  MISC_FEATURE (2)..(2): Xaa2 is D-Ser
  MISC_FEATURE (9)..(9): Xaa9 is alpha-MF
  MISC_FEATURE (10)..(10): Lys10 conjugated to  gamma-Glu-gamma-Glu-C16
  MISC_FEATURE (24)..(24): Xaa24 is Nle(epsilon-N3)
  MISC_FEATURE (27)..(27): Xaa27 is methione sulfone
HXQGTFTSDKSKYLDERAAQDXVXWLXDT

SEQ ID NO: 70 - protein, 29 aa, Artificial Sequence
  Peptide
  PEPTIDE (1)..(29): C-terminal amide
  MISC_FEATURE (2)..(2): Xaa2 is D-Ser
  MISC_FEATURE (10)..(10): Lys10 conjugated to  gamma-Glu-gamma-Glu-C16
  MISC_FEATURE (24)..(24): Xaa24 is Nle(epsilon-N3)
  MISC_FEATURE (25)..(25): Xaa25 is alpha-MW
  MISC_FEATURE (27)..(27): Xaa27 is methione sulfone
HXQGTFTSDKSKYLDERAAQDFVXXLXDT

SEQ ID NO: 71 - protein, 29 aa, Artificial Sequence
  Peptide
  PEPTIDE (1)..(29): C-terminal amide
  MISC_FEATURE (2)..(2): Xaa2 is D-Ser
  MISC_FEATURE (10)..(10): Lys10 conjugated to  gamma-Glu-gamma-Glu-C16
  MISC_FEATURE (24)..(24): Xaa24 is Nle(epsilon-N3)
  MISC_FEATURE (26)..(26): Xaa26 is alpha-ML
  MISC_FEATURE (27)..(27): Xaa27 is methione sulfone
HXQGTFTSDKSKYLDERAAQDFVXWXXDT

SEQ ID NO: 72 - protein, 29 aa, Artificial Sequence
  Peptide
  PEPTIDE (1)..(29): C-terminal amide
  MISC_FEATURE (2)..(2): Xaa2 is D-Ser
  MISC_FEATURE (10)..(10): Lys10 conjugated to  gamma-Glu-gamma-Glu-C16
  MISC_FEATURE (24)..(24): Xaa24 is Nle(epsilon-N3)
  MISC_FEATURE (27)..(27): Xaa27 is methione sulfone
  MISC_FEATURE (28)..(28): Xaa28 is alpha-MD
HXQGTFTSDKSKYLDERAAQDFVXWLXXT

SEQ ID NO: 73 - protein, 31 aa, Artificial Sequence
  Peptide
  PEPTIDE (1)..(31): C-terminal amide
  MISC_FEATURE (2)..(2): Xaa2 is alpha-MS
  MISC_FEATURE (10)..(10): Lys10 conjugated to  gamma-Glu-gamma-Glu-C16
  MISC_FEATURE (16)..(16): Xaa16 is aib
  MISC_FEATURE (24)..(24): Xaa24 is Nle(epsilon-N3)
  MISC_FEATURE (31)..(31): Xaa31 is gamma-Glu
HXQGTFTSDKSKYLDXRAAQDFVXWLLATKX

SEQ ID NO: 74 - protein, 31 aa, Artificial Sequence; identical to SEQ ID NO: 73
  Peptide
  PEPTIDE (1)..(31): C-terminal amide
  MISC_FEATURE (2)..(2): Xaa2 is alpha-Ms
  MISC_FEATURE (10)..(10): Lys10 conjugated to  gamma-Glu-gamma-Glu-C16
  MISC_FEATURE (16)..(16): Xaa16 is aib
  MISC_FEATURE (24)..(24): Xaa24 is Nle(epsilon-N3)
  MISC_FEATURE (31)..(31): Xaa31 is gamma-Glu

SEQ ID NO: 75 - protein, 29 aa, Artificial Sequence
  Peptide
  PEPTIDE (1)..(29): C-terminal amide
  MISC_FEATURE (2)..(2): Xaa2 is alpha-MS
  MISC_FEATURE (10)..(10): Lys10 conjugated to  gamma-Glu-gamma-Glu-C16
  MISC_FEATURE (24)..(24): Xaa24 is Nle(epsilon-N3)
HXQGTFTSDKSKYLDARAAQDFVXWLLDT

SEQ ID NO: 76 - protein, 29 aa, Artificial Sequence
  Peptide
  PEPTIDE (1)..(29): C-terminal amide
  MISC_FEATURE (2)..(2): Xaa2 is alpha-Ms
  MISC_FEATURE (10)..(10): Lys10 conjugated to  gamma-Glu-gamma-Glu-C16
  MISC_FEATURE (24)..(24): Xaa24 is Nle(epsilon-N3)
HXQGTFTSDKSKYLDARAAQDFVXWLLDT

SEQ ID NO: 77 - protein, 29 aa, Artificial Sequence
  Peptide
  PEPTIDE (1)..(29): C-terminal amide
  MISC_FEATURE (2)..(2): Xaa2 is alpha-Ms
  MISC_FEATURE (10)..(10): Lys10 conjugated to  gamma-Glu-gamma-Glu-C16
  MISC_FEATURE (16)..(16): Xaa16 is aib
  MISC_FEATURE (24)..(24): Xaa24 is Nle(epsilon-N3)
  MISC_FEATURE (27)..(27): Xaa27 is methione sulfone
HXVGTFTSDKSKYLDXRAAQDFVXWLXDT

SEQ ID NO: 78 - protein, 31 aa, Artificial Sequence
  Peptide
  PEPTIDE (1)..(31): C-terminal amide
  MISC_FEATURE (2)..(2): Xaa2 is aib
  MISC_FEATURE (10)..(10): Lys10 conjugated to  gamma-Glu-gamma-Glu-C16
  MISC_FEATURE (24)..(24): Xaa24 is Nle(epsilon-N3)
  MISC_FEATURE (27)..(27): Xaa27 is methione sulfone
HXQGTFTSDKSKYLDERAAQDFVXWLXDGRG

SEQ ID NO: 79 - protein, 31 aa, Artificial Sequence
  Peptide
  PEPTIDE (1)..(31): C-terminal amide
  MISC_FEATURE (2)..(2): Xaa2 is aib
  MISC_FEATURE (10)..(10): Lys10 conjugated to  gamma-Glu-gamma-Glu-C16
  MISC_FEATURE (24)..(24): Xaa24 is Nle(epsilon-N3)
  MISC_FEATURE (27)..(27): Xaa27 is methione sulfone
  MISC_FEATURE (28)..(28): Xaa28 is alpha-MD
HXQGTFTSDKSKYLDERAAQDFVXWLXXGRG

SEQ ID NO: 80 - protein, 31 aa, Artificial Sequence
  Peptide
  PEPTIDE (1)..(31): C-terminal amide
  MISC_FEATURE (2)..(2): Xaa2 is aib
  MISC_FEATURE (10)..(10): Lys10 conjugated to  gamma-Glu-gamma-Glu-C16
  MISC_FEATURE (16)..(16): Xaa16 is aib
  MISC_FEATURE (30)..(30): Xaa30 is Nle(epsilon-N3)
  MISC_FEATURE (31)..(31): Xaa31 is  gamma-Glu
HXQGTFTSDKSKYLDXRAAQDFVQWLLDTXX

SEQ ID NO: 81 - protein, 31 aa, Artificial Sequence
  Peptide
  PEPTIDE (1)..(31): C-terminal amide
  MISC_FEATURE (2)..(2): Xaa2 is aib
  MISC_FEATURE (10)..(10): Lys10 conjugated to  gamma-Glu-gamma-Glu-C16
  MISC_FEATURE (16)..(16): Xaa16 is aib
  MISC_FEATURE (27)..(27): Xaa27 is methione sulfone
  MISC_FEATURE (30)..(30): Xaa30 is Nle(epsilon-N3)
  MISC_FEATURE (31)..(31): Xaa31 is  gamma-Glu
HXQGTFTSDKSKYLDXRAAQDFVQWLXDTXX

SEQ ID NO: 82 - protein, 31 aa, Artificial Sequence
  Peptide
  PEPTIDE (1)..(31): C-terminal amide
  MISC_FEATURE (2)..(2): Xaa2 is aib
  MISC_FEATURE (10)..(10): Lys10 conjugated to  gamma-Glu-gamma-Glu-C16
  MISC_FEATURE (16)..(16): Xaa16 is aib
  MISC_FEATURE (27)..(27): Xaa27 is methione sulfone
  MISC_FEATURE (28)..(28): Xaa28 is alpha-MD
  MISC_FEATURE (30)..(30): Xaa30 is Nle(epsilon-N3)
  MISC_FEATURE (31)..(31): Xaa31 is  gamma-Glu
HXQGTFTSDKSKYLDXRAAQDFVQWLXXTXX

SEQ ID NO: 83 - protein, 29 aa, Artificial Sequence
  Peptide
  PEPTIDE (1)..(28)
  MISC_FEATURE (2)..(2): Xaa2 is aib
  MISC_FEATURE (10)..(10): Lys10 conjugated to  gamma-Glu-gamma-Glu-C16
  MISC_FEATURE (16)..(16): Xaa16 is aib
  MISC_FEATURE (24)..(24): Xaa24 is Nle(epsilon-N3)
  MISC_FEATURE (27)..(27): Xaa27 is methione sulfone
  MISC_FEATURE (28)..(28): Xaa28 is alpha-MD
HXQGTFTSDKSKYLDXRAAQDFVXWLXXT

SEQ ID NO: 84 - protein, 29 aa, Artificial Sequence
  Peptide
  PEPTIDE (1)..(29): C-terminal amide
  MISC_FEATURE (2)..(2): Xaa2 is D-Ser
  MISC_FEATURE (10)..(10): Lys10 conjugated to  gamma-Glu-gamma-Glu-C16
  MISC_FEATURE (24)..(24): Xaa24 is Nle(epsilon-N3)
HXEGTFTSDKSKYLDERAAQDFVXWLLDT

SEQ ID NO: 85 - protein, 29 aa, Artificial Sequence
  Peptide
  PEPTIDE (1)..(29): C-terminal amide
  MISC_FEATURE (2)..(2): Xaa2 is D-Ser
  MISC_FEATURE (10)..(10): Lys10 conjugated to  gamma-Glu-gamma-Glu-C16
  MISC_FEATURE (24)..(24): Lys24 is conjugated to  PEG2PEG2-C5N3
  MISC_FEATURE (27)..(27): Xaa27 is methione sulfone
HXQGTFTSDKSKYLDERAAQDFVKWLXDT

SEQ ID NO: 86 - protein, 29 aa, Artificial Sequence
  Peptide
  PEPTIDE (1)..(29): C-terminal amide
  MISC_FEATURE (2)..(2): Xaa2 is D-Ser
  MISC_FEATURE (10)..(10): Lys10 conjugated to  gamma-Glu-gamma-Glu-C16
  MISC_FEATURE (24)..(24): Lys24 is conjugated to  PEG2PEG2-C5N3
  MISC_FEATURE (27)..(27): Xaa27 is methione sulfone
  MISC_FEATURE (28)..(28): Xaa28 is alpha-MD
HXQGTFTSDKSKYLDERAAQDFVKWLXXT

SEQ ID NO: 87 - protein, 29 aa, Artificial Sequence
  Peptide
  PEPTIDE (1)..(29): C-terminal amide
  MISC_FEATURE (2)..(2): Xaa2 is D-Ser
  MISC_FEATURE (10)..(10): Lys10 conjugated to  gamma-Glu-gamma-Glu-C16
  MISC_FEATURE (24)..(24): Lys24 is conjugated to  PEG2-C10N3
HXQGTFTSDKSKYLDERAAQDFVKWLLDT

SEQ ID NO: 88 - protein, 29 aa, Artificial Sequence
  Peptide
  PEPTIDE (1)..(29): C-terminal amide
  MISC_FEATURE (2)..(2): Xaa2 is D-Ser
  MISC_FEATURE (10)..(10): Lys10 conjugated to  gamma-Glu-gamma-Glu-C16
  MISC_FEATURE (24)..(24): Lys24 is conjugated to  PEG2-C16N3
HXQGTFTSDKSKYLDERAAQDFVKWLLDT

SEQ ID NO: 89 - protein, 29 aa, Artificial Sequence
  Peptide
  PEPTIDE (1)..(29): C-terminal amide
  MISC_FEATURE (2)..(2): Xaa2 is aib
  MISC_FEATURE (10)..(10): Lys10 conjugated to  gamma-Glu-gamma-Glu-C16
  MISC_FEATURE (16)..(16): Xaa16 is aib
  MISC_FEATURE (24)..(24): Lys24 is conjugated to PEG2PEG2- C5N3
  MISC_FEATURE (27)..(27): Xaa27 is methione sulfone
HXQGTFTSDKSKYLDXRAAQDFVKWLXDT

SEQ ID NO: 90 - protein, 29 aa, Artificial Sequence
  Peptide
  PEPTIDE (1)..(29): C-terminal amide
  MISC_FEATURE (2)..(2): Xaa2 is aib
  MISC_FEATURE (10)..(10): Lys10 conjugated to  gamma-Glu-gamma-Glu-C16
  MISC_FEATURE (16)..(16): Xaa16 is aib
  MISC_FEATURE (24)..(24): Lys24 is conjugated to PEG2PEG2- C5N3
  MISC_FEATURE (27)..(27): Xaa27 is methione sulfone
HXQGTFTSDKSKYLDXRAAQDFVKWLXDT

SEQ ID NO: 91 - protein, 29 aa, Artificial Sequence
  Peptide
  PEPTIDE (1)..(29): C-terminal amide
  MISC_FEATURE (2)..(2): Xaa2 is D-Ser
  MISC_FEATURE (10)..(10): Lys10 conjugated to  gamma-Glu-gamma-Glu-C16
  MISC_FEATURE (20)..(20): Lys20 is conjugated to PEG2PEG2-gamma-Glu-C10N3
HXQGTFTSDKSKYLDERAAKDFVQWLLDT

SEQ ID NO: 92 - protein, 29 aa, Artificial Sequence
  Peptide
  PEPTIDE (1)..(29): C-terminal amide
  MISC_FEATURE (2)..(2): Xaa2 is D-Ser
  MISC_FEATURE (10)..(10): Lys10 conjugated to  gamma-Glu-gamma-Glu-C16
  MISC_FEATURE (20)..(20): Lys20 is conjugated to PEG2PEG2-gamma-Glu-C16N3
HXQGTFTSDKSKYLDERAAKDFVQWLLDT

SEQ ID NO: 93 - protein, 31 aa, Artificial Sequence; identical to SEQ ID NO: 53
  Peptide
  PEPTIDE (1)..(31): C-terminal amide
  MISC_FEATURE (2)..(2): Xaa2 is D-Ser
  MISC_FEATURE (10)..(10): Lys10 conjugated to  gamma-Glu-gamma-Glu-C16
  MISC_FEATURE (16)..(16): Xaa16 is aib
  MISC_FEATURE (30)..(30): Xaa30 is Nle(epsilon-N3)
  MISC_FEATURE (31)..(31): Xaa31 is  gamma-Glu

SEQ ID NO: 94 - protein, 31 aa, Artificial Sequence
  Peptide
  PEPTIDE (1)..(31): C-terminal amide
  MISC_FEATURE (2)..(2): Xaa2 is D-Ser
  MISC_FEATURE (10)..(10): Lys10 conjugated to  gamma-Glu-gamma-Glu-C16
  MISC_FEATURE (30)..(30): Xaa30 is Nle(epsilon-N3)
  MISC_FEATURE (31)..(31): Xaa31 is  gamma-Glu
HXQGTFTSDKSKYLDERAAQDFVQWLLDTKX

SEQ ID NO: 95 - protein, 31 aa, Artificial Sequence; identical to SEQ ID NO: 54
  Peptide
  PEPTIDE (1)..(31): C-terminal amide
  MISC_FEATURE (2)..(2): Xaa2 is D-Ser
  MISC_FEATURE (10)..(10): Lys10 conjugated to  gamma-Glu-gamma-Glu-C16
  MISC_FEATURE (16)..(16): Xaa16 is aib
  MISC_FEATURE (31)..(31): Xaa31 is conjugated to  PEG2PEG2-C5N3

SEQ ID NO: 96 - protein, 31 aa, Artificial Sequence
  Peptide
  PEPTIDE (1)..(31): C-terminal amide
  MISC_FEATURE (2)..(2): Xaa2 is D-Ser
  MISC_FEATURE (10)..(10): Lys10 conjugated to  gamma-Glu-gamma-Glu-C16
  MISC_FEATURE (31)..(31): Xaa31 is conjugated to  PEG2PEG2-C5N3
HXQGTFTSDKSKYLDERAAQDFVQWLLDTKK

SEQ ID NO: 97 - protein, 31 aa, Artificial Sequence; identical to SEQ ID NO: 56
  Peptide
  PEPTIDE (1)..(31): C-terminal amide
  MISC_FEATURE (2)..(2): Xaa2 is D-Ser
  MISC_FEATURE (10)..(10): Lys10 conjugated to  gamma-Glu-gamma-Glu-C16
  MISC_FEATURE (16)..(16): Xaa16 is aib
  MISC_FEATURE (24)..(24): Xaa24 is Nle(epsilon-N3)
  MISC_FEATURE (31)..(31): Xaa31 is gamma-Glu

SEQ ID NO: 98 - protein, 31 aa, Artificial Sequence
  Peptide
  PEPTIDE (1)..(31): C-terminal amide
  MISC_FEATURE (2)..(2): Xaa2 is aib
  MISC_FEATURE (10)..(10): Lys10 conjugated to  gamma-Glu-gamma-Glu-C16
  MISC_FEATURE (16)..(16): Xaa16 is aib
  MISC_FEATURE (24)..(24): Lys24 is conjugated to PEG2-C5N3
  MISC_FEATURE (31)..(31): Xaa31 is gamma-Glu
HXQGTFTSDKSKYLDXRAAQDFVKWLLDTKX

SEQ ID NO: 99 - protein, 31 aa, Artificial Sequence; identical to SEQ ID NO: 98
  Peptide
  PEPTIDE (1)..(31): C-terminal amide
  MISC_FEATURE (2)..(2): Xaa2 is aib
  MISC_FEATURE (10)..(10): Lys10 conjugated to  gamma-Glu-gamma-Glu-C16
  MISC_FEATURE (16)..(16): Xaa16 is aib
  MISC_FEATURE (24)..(24): Lys24 is conjugated to PEG2PEG2-C5N3
  MISC_FEATURE (31)..(31): Xaa31 is gamma-Glu

SEQ ID NO: 100 - protein, 29 aa, Artificial Sequence
  Peptide
  PEPTIDE (1)..(29): C-terminal amide
  MISC_FEATURE (2)..(2): Xaa2 is D-Ser
  MISC_FEATURE (10)..(10): Lys10 conjugated to  gamma-Glu-gamma-Glu-C16
  MISC_FEATURE (21)..(21): Lys21 is conjugated to  PEG2PEG2-gamma-GluC16N3
HXQGTFTSDKSKYLDERAAQKFVQWLLDT

SEQ ID NO: 101 - protein, 29 aa, Artificial Sequence
  Peptide
  PEPTIDE (1)..(29): C-terminal amide
  MISC_FEATURE (2)..(2): Xaa2 is D-Ser
  MISC_FEATURE (10)..(10): Lys10 conjugated to  gamma-Glu-gamma-Glu-C16
  MISC_FEATURE (24)..(24): Lys24 is conjugated to PEG2PEG2-gamma-glu-C16N3
HXQGTFTSDKSKYLDERAAQDFVKWLLDT

SEQ ID NO: 102 - protein, 31 aa, Artificial Sequence
  Peptide
  PEPTIDE (1)..(31): C-terminal amide
  MISC_FEATURE (2)..(2): Xaa2 is aib
  MISC_FEATURE (16)..(16): Xaa16 is aib
  MISC_FEATURE (24)..(24): Xaa24 is Nle(epsilon-N3)
HXQGTFTSDKSKYLDXRAAQDFVXWLMNTKQ

SEQ ID NO: 103 - protein, 29 aa, Artificial Sequence
  Peptide
  PEPTIDE (1)..(29): C-terminal amide
  MISC_FEATURE (2)..(2): Xaa2 is D-Ser
  MISC_FEATURE (24)..(24): Xaa24 is Nle(epsilon-N3)
HXQGTFTSDKSKYLDERAAQDFVXWLLDT

SEQ ID NO: 104 - protein, 31 aa, Artificial Sequence
  Peptide
  PEPTIDE (1)..(31): C-terminal amide
  MISC_FEATURE (2)..(2): Xaa2 is aib
  MISC_FEATURE (16)..(16): Xaa16 is aib
  MISC_FEATURE (20)..(20): Xaa20 is Nle(epsilon-N3)
HXQGTFTSDKSKYLDXRAAXDFVQWLMNTKQ

SEQ ID NO: 105 - protein, 29 aa, Artificial Sequence
  Peptide
  PEPTIDE (1)..(29): C-terminal amide
  MISC_FEATURE (2)..(2): Xaa2 is D-Ser
  MISC_FEATURE (20)..(20): Xaa20 is Nle(epsilon-N3)
HXQGTFTSDKSKYLDERAAXDFVQWLLDT

SEQ ID NO: 106 - protein, 32 aa, Artificial Sequence
  Peptide
  PEPTIDE (1)..(32): C-terminal amide
  MISC_FEATURE (2)..(2): Xaa2 is aib
  MISC_FEATURE (16)..(16): Xaa16 is aib
  MISC_FEATURE (32)..(32): Xaa32 is Nle(epsilon-N3)
HXQGTFTSDKSKYLDXRAAQDFVQWLMNTKQX

SEQ ID NO: 107 - protein, 30 aa, Artificial Sequence
  Peptide
  PEPTIDE (1)..(30): C-terminal amide
  MISC_FEATURE (2)..(2): Xaa2 is D-Ser
  MISC_FEATURE (30)..(30): Xaa30 is Nle(epsilon-N3)
HXQGTFTSDKSKYLDERAAQDFVQWLLDTX

SEQ ID NO: 108 - protein, 29 aa, Artificial Sequence
  Peptide
  PEPTIDE (1)..(29): C-terminal amide
  MISC_FEATURE (2)..(2): Xaa2 is D-Ser
  MISC_FEATURE (10)..(10): Lys10 conjugated to  gamma-Glu-gamma-Glu-C16
  MISC_FEATURE (24)..(24): Lys10 conjugated to  gamma-Glu-gamma-Glu-C16
HXQGTFTSDKSKYLDERAAQDFVXWLLDT

SEQ ID NO: 109 - protein, 29 aa, Artificial Sequence
  Peptide
  PEPTIDE (1)..(29): C-terminal amide
  MISC_FEATURE (2)..(2): Xaa2 is D-Ser
  MISC_FEATURE (10)..(10): Lys10 conjugated to  gamma-Glu-gamma-Glu-C16
  MISC_FEATURE (24)..(24): Xaa24 is Nle(epsilon-N3)
HXEGTFTSDKSKYLDERAAQDFVXWLLDT

SEQ ID NO: 110 - protein, 39 aa, Artificial Sequence
  Peptide
  PEPTIDE (1)..(29): C-terminal amide
  MISC_FEATURE (2)..(2): Xaa2 is aib
  MISC_FEATURE (10)..(10): Lys10 conjugated to  gamma-Glu-C16
  MISC_FEATURE (24)..(24): Xaa24 is Nle(epsilon-N3)
HXQGTFTSDKSKYLDERAAQDFVXWLLDGGPSSGAPPPS

SEQ ID NO: 111 - protein, 39 aa, Artificial Sequence
  Peptide
  PEPTIDE (1)..(39): C-terminal amide
  MISC_FEATURE (24)..(24): Xaa24 is Nle(epsilon-N3)
HGEGTFTSDLSKQMEEEAVRLFIXWLKNGGPSSGAPPPS

SEQ ID NO: 112 - protein, 29 aa, Artificial Sequence
  Peptide
  PEPTIDE (1)..(29): C-terminal amide
  MISC_FEATURE (2)..(2): Xaa2 is D-Ser
  MISC_FEATURE (10)..(10): Lys10 conjugated to  gamma-Glu-gamma-Glu-C16
HXQGTFTSDKSKYLDERAAQDFVKWLLDT

SEQ ID NO: 113 - protein, 29 aa, Artificial Sequence
  Peptide
  PEPTIDE (1)..(29): C-terminal amide
  MISC_FEATURE (2)..(2): Xaa2 is D-Ser
  MISC_FEATURE (10)..(10): Lys10 conjugated to  gamma-Glu-gamma-Glu-C16
  MISC_FEATURE (14)..(14): Xaa14 is alpha-ML
  MISC_FEATURE (24)..(24): Xaa24 is Nle(epsilon-N3)
  MISC_FEATURE (27)..(27): Xaa27 is methione sulfone
HXQGTFTSDKSKYXDERAAQDFVXWLXDT

SEQ ID NO: 114 - protein, 29 aa, Artificial Sequence
  Peptide
  PEPTIDE (1)..(29): C-terminal amide
  MISC_FEATURE (2)..(2): Xaa2 is D-Ser
  MISC_FEATURE (10)..(10): Lys10 conjugated to  gamma-Glu-gamma-Glu-C16
  MISC_FEATURE (24)..(24): Xaa24 is Nle(epsilon-N3)
  MISC_FEATURE (25)..(25): Xaa25 is alpha-MW
  MISC_FEATURE (27)..(27): Xaa27 is methione sulfone
HXQGTFTSDKSKYLDERAAQDFVXXLXDT

SEQ ID NO: 115 - protein, 29 aa, Artificial Sequence
  Peptide
  PEPTIDE (1)..(29): C-terminal amide
  MISC_FEATURE (2)..(2): Xaa2 is D-Ser
  MISC_FEATURE (10)..(10): Lys10 conjugated to  gamma-Glu-gamma-Glu-C16
  MISC_FEATURE (24)..(24): Xaa24 is Nle(epsilon-N3)
  MISC_FEATURE (27)..(27): Xaa27 is methione sulfone
  MISC_FEATURE (28)..(28): Xaa28 is alpha-MD
HXQGTFTSDKSKYLDERAAQDFVXWLXXT

SEQ ID NO: 116 - protein, 31 aa, Artificial Sequence; identical to SEQ ID NO: 73
  Peptide
  PEPTIDE (1)..(31): C-terminal amide
  MISC_FEATURE (2)..(2): Xaa2 is alpha-MS
  MISC_FEATURE (10)..(10): Lys10 conjugated to  gamma-Glu-gamma-Glu-C16
  MISC_FEATURE (16)..(16): Xaa16 is aib
  MISC_FEATURE (24)..(24): Xaa24 is Nle(epsilon-N3)
  MISC_FEATURE (31)..(31): Xaa31 is gamma-Glu

SEQ ID NO: 117 - protein, 31 aa, Artificial Sequence; identical to SEQ ID NO: 73
  Peptide
  PEPTIDE (1)..(31): C-terminal amide
  MISC_FEATURE (2)..(2): Xaa2 is alpha-Ms
  MISC_FEATURE (10)..(10): Lys10 conjugated to  gamma-Glu-gamma-Glu-C16
  MISC_FEATURE (16)..(16): Xaa16 is aib
  MISC_FEATURE (24)..(24): Xaa24 is Nle(epsilon-N3)
  MISC_FEATURE (31)..(31): Xaa31 is gamma-Glu

SEQ ID NO: 118 - protein, 29 aa, Artificial Sequence
  Peptide
  PEPTIDE (1)..(29): C-terminal amide
  MISC_FEATURE (2)..(2): Xaa2 is alpha-Ms
  MISC_FEATURE (10)..(10): Lys10 conjugated to  gamma-Glu-gamma-Glu-C16
  MISC_FEATURE (16)..(16): Xaa16 is aib
  MISC_FEATURE (24)..(24): Xaa24 is Nle(epsilon-N3)
  MISC_FEATURE (27)..(27): Xaa27 is methione sulfone
HXVGTFTSDKSKYLDXRAAQDFVXWLXDT

SEQ ID NO: 119 - protein, 29 aa, Artificial Sequence
  Peptide
  PEPTIDE (1)..(29): C-terminal amide
  MISC_FEATURE (2)..(2): Xaa2 is alpha-Ms
  MISC_FEATURE (10)..(10): Lys10 conjugated to  gamma-Glu-gamma-Glu-C16
  MISC_FEATURE (24)..(24): Xaa24 is Nle(epsilon-N3)
HXQGTFTSDKSKYLDARAAQDFVXWLLDT

SEQ ID NO: 120 - protein, 29 aa, Artificial Sequence
  Peptide
  PEPTIDE (1)..(29): C-terminal amide
  MISC_FEATURE (2)..(2): Xaa2 is alpha-MS
  MISC_FEATURE (10)..(10): Lys10 conjugated to  gamma-Glu-gamma-Glu-C16
  MISC_FEATURE (24)..(24): Xaa24 is Nle(epsilon-N3)
HXQGTFTSDKSKYLDARAAQDFVXWLLDT

SEQ ID NO: 121 - protein, 29 aa, Artificial Sequence
  Peptide
  PEPTIDE (1)..(29): C-terminal amide
  MISC_FEATURE (2)..(2): Xaa2 is D-Ser
  MISC_FEATURE (10)..(10): Lys10 conjugated to  gamma-Glu-gamma-Glu-C16
  MISC_FEATURE (24)..(24): Xaa24 is Nle(epsilon-N3)
HXEGTFTSDKSKYLDERAAQDFVXWLLDT

SEQ ID NO: 122 - protein, 29 aa, Artificial Sequence
  Peptide
  PEPTIDE (1)..(29): C-terminal amide
  MISC_FEATURE (2)..(2): Xaa2 is D-Ser
  MISC_FEATURE (10)..(10): Lys10 conjugated to  gamma-Glu-gamma-Glu-C16
  MISC_FEATURE (21)..(21): Xaa21 is Nle(epsilon-N3)
  MISC_FEATURE (27)..(27): Xaa27 is methione sulfone
HXQGTFTSDKSKYLDERAAQXFVQWLXDT

SEQ ID NO: 123 - protein, 29 aa, Artificial Sequence
  Peptide
  PEPTIDE (1)..(29): C-terminal amide
  MISC_FEATURE (2)..(2): Xaa2 is D-Ser
  MISC_FEATURE (10)..(10): Lys10 conjugated to  gamma-Glu-gamma-Glu-C16
  MISC_FEATURE (21)..(21): Xaa21 is Nle(epsilon-N3)
  MISC_FEATURE (27)..(27): Xaa27 is methione sulfone
  MISC_FEATURE (28)..(28): Xaa28 is alpha-MD
HXQGTFTSDKSKYLDERAAQXFVQWLXXT

SEQ ID NO: 124 - protein, 31 aa, Artificial Sequence; identical to SEQ ID NO: 82
  Peptide
  PEPTIDE (1)..(31): C-terminal amide
  MISC_FEATURE (2)..(2): Xaa2 is aib
  MISC_FEATURE (10)..(10): Lys10 conjugated to  gamma-Glu-gamma-Glu-C16
  MISC_FEATURE (16)..(16): Xaa16 is aib
  MISC_FEATURE (27)..(27): Xaa27 is methione sulfone
  MISC_FEATURE (28)..(28): Xaa28 is alph-MD
  MISC_FEATURE (30)..(30): Xaa30 is Nle(epsilon-N3)
  MISC_FEATURE (31)..(31): Xaa31 is  gamma-Glu

SEQ ID NO: 125 - protein, 29 aa, Artificial Sequence
  Peptide
  PEPTIDE (1)..(29): C-terminal amide
  MISC_FEATURE (2)..(2): Xaa2 is aib
  MISC_FEATURE (10)..(10): Lys10 conjugated to  gamma-Glu-gamma-Glu-C16
  MISC_FEATURE (16)..(16): Xaa16 is aib
  MISC_FEATURE (24)..(24): Xaa24 is Nle(epsilon-N3)
  MISC_FEATURE (27)..(27): Xaa27 is methione sulfone
  MISC_FEATURE (28)..(28): Xaa28 is alpha-MD
HXQGTFTSDKSKYLDXRAAQDFVXWLXXT

SEQ ID NO: 126 - protein, 31 aa, Artificial Sequence; identical to SEQ ID NO: 78
  Peptide
  PEPTIDE (1)..(31): C-terminal amide
  MISC_FEATURE (2)..(2): Xaa2 is aib
  MISC_FEATURE (10)..(10): Lys10 conjugated to  gamma-Glu-gamma-Glu-C16
  MISC_FEATURE (24)..(24): Xaa24 is Nle(epsilon-N3)
  MISC_FEATURE (27)..(27): Xaa27 is methione sulfone

SEQ ID NO: 127 - protein, 31 aa, Artificial Sequence; identical to SEQ ID NO: 80
  Peptide
  PEPTIDE (1)..(31): C-terminal amide
  MISC_FEATURE (2)..(2): Xaa2 is aib
  MISC_FEATURE (10)..(10): Lys10 conjugated to  gamma-Glu-gamma-Glu-C16
  MISC_FEATURE (16)..(16): Xaa16 is aib
  MISC_FEATURE (30)..(30): Xaa30 is Nle(epsilon-N3)
  MISC_FEATURE (31)..(31): Xaa31 is  gamma-Glu

SEQ ID NO: 128 - protein, 31 aa, Artificial Sequence; identical to SEQ ID NO: 81
  Peptide
  PEPTIDE (1)..(31): C-terminal amide
  MISC_FEATURE (2)..(2): Xaa2 is aib
  MISC_FEATURE (10)..(10): Lys10 conjugated to  gamma-Glu-gamma-Glu-C16
  MISC_FEATURE (16)..(16): Xaa16 is aib
  MISC_FEATURE (27)..(27): Xaa27 is methione sulfone
  MISC_FEATURE (30)..(30): Xaa30 is Nle(epsilon-N3)
  MISC_FEATURE (31)..(31): Xaa31 is  gamma-Glu

SEQ ID NO: 129 - protein, 29 aa, Artificial Sequence
  Peptide
  PEPTIDE (1)..(29): C-terminal amide
  MISC_FEATURE (2)..(2): Xaa2 is D-Ser
  MISC_FEATURE (10)..(10): Lys10 conjugated to  gamma-Glu-gamma-Glu-C16
  MISC_FEATURE (24)..(24): Lys 24 is conjugated to  PEG2PEG2-C5N3
  MISC_FEATURE (27)..(27): Xaa27 is methione sulfone
HXQGTFTSDKSKYLDERAAQDFVKWLXDT

SEQ ID NO: 130 - protein, 29 aa, Artificial Sequence
  Peptide
  PEPTIDE (1)..(29): C-terminal amide
  MISC_FEATURE (2)..(2): Xaa2 is D-Ser
  MISC_FEATURE (10)..(10): Lys10 conjugated to  gamma-Glu-gamma-Glu-C16
  MISC_FEATURE (24)..(24): Lys 24 is conjugated to  PEG2PEG2-C5N3
  MISC_FEATURE (27)..(27): Xaa27 is methione sulfone
  MISC_FEATURE (28)..(28): Xaa28 is alpha-MD
HXQGTFTSDKSKYLDERAAQDFVKWLXXT

SEQ ID NO: 131 - protein, 29 aa, Artificial Sequence
  Peptide
  PEPTIDE (1)..(29): C-terminal amide
  MISC_FEATURE (2)..(2): Xaa2 is D-Ser
  MISC_FEATURE (10)..(10): Lys10 conjugated to  gamma-Glu-gamma-Glu-C16
  MISC_FEATURE (24)..(24): Lys 24 is conjugated to  PEG2-C10N3
HXQGTFTSDKSKYLDERAAQDFVKWLLDT

SEQ ID NO: 132 - protein, 29 aa, Artificial Sequence
  Peptide
  PEPTIDE (1)..(29): C-terminal amide
  MISC_FEATURE (2)..(2): Xaa2 is D-Ser
  MISC_FEATURE (10)..(10): Lys10 conjugated to  gamma-Glu-gamma-Glu-C16
  MISC_FEATURE (24)..(24): Lys 24 is conjugated to  PEG2-C16N3
HXQGTFTSDKSKYLDERAAQDFVKWLLDT

SEQ ID NO: 133 - protein, 29 aa, Artificial Sequence
  Peptide
  PEPTIDE (1)..(29): C-terminal amide
  MISC_FEATURE (2)..(2): Xaa2 is D-Ser
  MISC_FEATURE (10)..(10): Lys10 conjugated to  gamma-Glu-gamma-Glu-C16
  MISC_FEATURE (20)..(20): Lys20 is conjugated to  PEG2PEG2-gamma-Glu- C10N3
HXQGTFTSDKSKYLDERAAKDFVQWLLDT

SEQ ID NO: 134 - protein, 29 aa, Artificial Sequence
  Peptide
  PEPTIDE (1)..(29): C-terminal amide
  MISC_FEATURE (2)..(2): Xaa2 is D-Ser
  MISC_FEATURE (10)..(10): Lys10 conjugated to  gamma-Glu-gamma-Glu-C16
  MISC_FEATURE (20)..(20): Lys20 is conjugated to  PEG2PEG2-gamma-Glu- C16N3
HXQGTFTSDKSKYLDERAAKDFVQWLLDT

SEQ ID NO: 135 - protein, 29 aa, Artificial Sequence
  Peptide
  PEPTIDE (1)..(29): C-terminal amide
  MISC_FEATURE (2)..(2): Xaa2 is D-Ser
  MISC_FEATURE (10)..(10): Lys10 conjugated to  gamma-Glu-gamma-Glu-C16
  MISC_FEATURE (24)..(24): Xaa 24 is pra
HXQGTFTSDKSKYLDERAAQDFVXWLLDT

SEQ ID NO: 136 - protein, 31 aa, Artificial Sequence; identical to SEQ ID NO: 53
  Peptide
  PEPTIDE (1)..(31): C-terminal amide
  MISC_FEATURE (2)..(2): Xaa2 is D-Ser
  MISC_FEATURE (10)..(10): Lys10 conjugated to  gamma-Glu-gamma-Glu-C16
  MISC_FEATURE (16)..(16): Xaa16 is aib
  MISC_FEATURE (30)..(30): Xaa30 is Nle(epsilon-N3)
  MISC_FEATURE (31)..(31): Xaa31 is  gamma-Glu

SEQ ID NO: 137 - protein, 31 aa, Artificial Sequence; identical to SEQ ID NO: 54
  Peptide
  PEPTIDE (1)..(31): C-terminal amide
  MISC_FEATURE (2)..(2): Xaa2 is D-Ser
  MISC_FEATURE (10)..(10): Lys10 conjugated to  gamma-Glu-gamma-Glu-C16
  MISC_FEATURE (16)..(16): Xaa16 is aib
  MISC_FEATURE (31)..(31): Lys31 conjugated to  PEG2PEG2-C5N3

SEQ ID NO: 138 - protein, 29 aa, Artificial Sequence
  Peptide
  PEPTIDE (1)..(29): C-terminal amide
  MISC_FEATURE (2)..(2): Xaa2 is D-Ser
  MISC_FEATURE (10)..(10): Lys10 conjugated to  gamma-Glu-gamma-Glu-C16
  MISC_FEATURE (21)..(21): Lys21 is conjugated to  PEG2PEG2-gamma-Glu- C16N3
HXQGTFTSDKSKYLDERAAQKFVQWLLDT

SEQ ID NO: 139 - protein, 29 aa, Artificial Sequence
  Peptide
  PEPTIDE (1)..(29): C-terminal amide
  MISC_FEATURE (2)..(2): Xaa2 is aib
  MISC_FEATURE (10)..(10): Lys10 conjugated to  gamma-Glu-gamma-Glu-C16
  MISC_FEATURE (16)..(16): Xaa16 is aib
  MISC_FEATURE (24)..(24): Lys24 conjugated to  PEG2PEG2-C5N3
  MISC_FEATURE (27)..(27): Xaa27 is methione sulfone
HXQGTFTSDKSKYLDXRAAQDFVKWLXDT

SEQ ID NO: 140 - protein, 31 aa, Artificial Sequence; identical to SEQ ID NO: 94
  Peptide
  PEPTIDE (1)..(31): C-terminal amide
  MISC_FEATURE (2)..(2): Xaa2 is D-Ser
  MISC_FEATURE (10)..(10): Lys10 conjugated to  gamma-Glu-gamma-Glu-C16
  MISC_FEATURE (30)..(30): Xaa30 is Nle(epsilon-N3)
  MISC_FEATURE (31)..(31): Xaa31 is  gamma-Glu

SEQ ID NO: 141 - protein, 29 aa, Artificial Sequence
  Peptide
  PEPTIDE (1)..(29): C-terminal amide
  MISC_FEATURE (2)..(2): Xaa2 is D-Ser
  MISC_FEATURE (10)..(10): Lys10 conjugated to  gamma-Glu-gamma-Glu-C16
  MISC_FEATURE (24)..(24): Lys24 is conjugated to PEG2PEG2-gamma-Glu- EC16N3
HXQGTFTSDKSKYLDERAAQDFVKWLLDT

SEQ ID NO: 142 - protein, 29 aa, Artificial Sequence
  Peptide
  PEPTIDE (1)..(29): C-terminal amide
  MISC_FEATURE (2)..(2): Xaa2 is D-Ser
  MISC_FEATURE (10)..(10): Lys10 conjugated to  gamma-Glu-gamma-Glu-C16
  MISC_FEATURE (16)..(16): Xaa16 is aib
  MISC_FEATURE (24)..(24): Lys24 is conjugated to  PEG2PEG2-C5N3
  MISC_FEATURE (27)..(27): Xaa27 is methione sulfone
HXQGTFTSDKSKYLDXRAAQDFVKWLXDT

SEQ ID NO: 143 - protein, 31 aa, Artificial Sequence; identical to SEQ ID NO: 96
  Peptide
  PEPTIDE (1)..(31): C-terminal amide
  MISC_FEATURE (2)..(2): Xaa2 is D-Ser
  MISC_FEATURE (10)..(10): Lys10 conjugated to  gamma-Glu-gamma-Glu-C16
  MISC_FEATURE (31)..(31): Lys31 is conjugated to  PEG2PEG2-C5N3

SEQ ID NO: 144 - protein, 31 aa, Artificial Sequence; identical to SEQ ID NO: 56
  Peptide
  PEPTIDE (1)..(31): C-terminal amide
  MISC_FEATURE (2)..(2): Xaa2 is D-Ser
  MISC_FEATURE (10)..(10): Lys10 conjugated to  gamma-Glu-gamma-Glu-C16
  MISC_FEATURE (16)..(16): Xaa16 is aib
  MISC_FEATURE (24)..(24): Xaa24 is Nle(epsilon-N3)
  MISC_FEATURE (31)..(31): Xaa31 is gamma-Glu

SEQ ID NO: 145 - protein, 31 aa, Artificial Sequence; identical to SEQ ID NO: 98
  Peptide
  PEPTIDE (1)..(31): C-terminal amide
  MISC_FEATURE (2)..(2): Xaa2 is aib
  MISC_FEATURE (16)..(16): Xaa16 is aib
  MISC_FEATURE (24)..(24): Lys24 conjugated to   PEG2PEG2-C5N3
  MISC_FEATURE (31)..(31): Xaa31 is gamma-Glu

SEQ ID NO: 146 - protein, 31 aa, Artificial Sequence; identical to SEQ ID NO: 98
  Peptide
  PEPTIDE (1)..(31): C-terminal amide
  MISC_FEATURE (2)..(2): Xaa2 is aib
  MISC_FEATURE (16)..(16): Xaa16 is aib
  MISC_FEATURE (24)..(24): Lys24 conjugated to   PEG2PEG2-C5N3
  MISC_FEATURE (31)..(31): Xaa31 is gamma-Glu

SEQ ID NO: 147 - protein, 29 aa, Artificial Sequence
  Peptide
  PEPTIDE (1)..(29): C-terminal amide
  MISC_FEATURE (2)..(2): Xaa2 is D-Ser
  MISC_FEATURE (24)..(24): Xaa24 is Nle(epsilon-N3)
HXQGTFTSDKSKYLDSRAAQDFVXWLLDT

SEQ ID NO: 148 - protein, 29 aa, Artificial Sequence
  Peptide
  PEPTIDE (1)..(29): C-terminal amide
  MISC_FEATURE (2)..(2): Xaa2 is D-Ser
  MISC_FEATURE (24)..(24): Xaa24 is Nle(epsilon-N3)
  MISC_FEATURE (28)..(28): Xaa28 is gamma-Glu
HXQGTFTSDKSKYLDERAAQDFVXWLLXT

SEQ ID NO: 149 - protein, 29 aa, Artificial Sequence
  Peptide
  PEPTIDE (1)..(29): C-terminal amide
  MISC_FEATURE (2)..(2): Xaa2 is D-Ser
  MISC_FEATURE (10)..(10): Lys10 conjugated to gamma-Glu-gamma-Glu-C16
  MISC_FEATURE (24)..(24): Xaa24 is Nle(epsilon-N3)
HXQGTFTSDKSKYLDNKRAQDFVXWLMQT

SEQ ID NO: 150 - protein, 29 aa, Artificial Sequence
  Peptide
  PEPTIDE (1)..(29): C-terminal amide
  MISC_FEATURE (2)..(2): Xaa2 is aib
  MISC_FEATURE (10)..(10): Lys10 conjugated to  gamma-Glu-gamma-Glu-C16
  MISC_FEATURE (16)..(16): Xaa16 is aib
  MISC_FEATURE (24)..(24): Xaa24 is Nle(epsilon-N3)
  MISC_FEATURE (27)..(27): Xaa27 is methione sulfone
  MISC_FEATURE (28)..(28): Xaa28 is gamma-Glu
HXQGTFTSDKSKYLDXRAAQDFVXWLXXT

SEQ ID NO: 151 - protein, 29 aa, Artificial Sequence
  Peptide
  PEPTIDE (1)..(29): C-terminal amide
  MISC_FEATURE (2)..(2): Xaa2 is aib
  MISC_FEATURE (10)..(10): Lys10 conjugated to  gamma-Glu-gamma-Glu-C16
  MISC_FEATURE (16)..(16): Xaa16 is aib
  MISC_FEATURE (24)..(24): Xaa24 is Nle(epsilon-N3)
  MISC_FEATURE (27)..(27): Xaa27 is methione sulfone
HXQGTFVSDKSKYLDXRAAQDFVXWLXET

SEQ ID NO: 152 - protein, 31 aa, Artificial Sequence
  Peptide
  PEPTIDE (1)..(31): C-terminal amide
  MISC_FEATURE (2)..(2): Xaa2 is aib
  MISC_FEATURE (10)..(10): Lys10 conjugated to  gamma-Glu-gamma-Glu-C16
  MISC_FEATURE (16)..(16): Xaa16 is aib
  MISC_FEATURE (24)..(24): Xaa24 is Nle(epsilon-N3)
  MISC_FEATURE (28)..(28): Xaa28 is D-Asp
  MISC_FEATURE (31)..(31): Xaa31 is gamma-Glu
HXQGTFTSDKSKYLDXRAAQDFVXWLLXTKX

SEQ ID NO: 153 - protein, 29 aa, Artificial Sequence
  Peptide
  PEPTIDE (1)..(29): C-terminal amide
  MISC_FEATURE (2)..(2): Xaa2 is aib
  MISC_FEATURE (10)..(10): Lys10 conjugated to  gamma-Glu-gamma-Glu-C16
  MISC_FEATURE (24)..(24): Xaa24 is Nle(epsilon-N3)
  MISC_FEATURE (25)..(25): Xaa25 is alpha-MW
  MISC_FEATURE (27)..(27): Xaa27 is methione sulfone
HXQGTFTSDKSKYLDERAAQDFVXXLXDT

SEQ ID NO: 154 - protein, 29 aa, Artificial Sequence
  Peptide
  PEPTIDE (1)..(29): C-terminal amide
  MISC_FEATURE (2)..(2): Xaa2 is aib
  MISC_FEATURE (10)..(10): Lys10 conjugated to  gamma-Glu-gamma-Glu-C16
  MISC_FEATURE (24)..(24): Xaa24 is Nle(epsilon-N3)
  MISC_FEATURE (27)..(27): Xaa27 is methione sulfone
  MISC_FEATURE (28)..(28): Xaa28 is alpha-MD
HXQGTFTSDKSKYLDERAAQDFVXWLXXT

SEQ ID NO: 155 - protein, 29 aa, Artificial Sequence
  Peptide
  PEPTIDE (1)..(29): C-terminal amide
  MISC_FEATURE (2)..(2): Xaa2 is aib
  MISC_FEATURE (10)..(10): Lys10 conjugated to  gamma-Glu-gamma-Glu-C16
  MISC_FEATURE (16)..(16): Xaa16 is aib
  MISC_FEATURE (24)..(24): Xaa24 is Nle(epsilon-N3)
  MISC_FEATURE (25)..(25): Xaa25 is alpha-MW
  MISC_FEATURE (27)..(27): Xaa27 is methione sulfone
HXQGTFTSDKSKYLDXRAAQDFVXXLXDT

SEQ ID NO: 156 - protein, 29 aa, Artificial Sequence
  Peptide
  PEPTIDE (1)..(29): C-terminal amide
  MISC_FEATURE (2)..(2): Xaa2 is aib
  MISC_FEATURE (10)..(10): Lys10 conjugated to  gamma-Glu-gamma-Glu-C16
  MISC_FEATURE (16)..(16): Xaa16 is aib
  MISC_FEATURE (24)..(24): Xaa24 is Nle(epsilon-N3)
  MISC_FEATURE (27)..(27): Xaa27 is methione sulfone
  MISC_FEATURE (28)..(28): Xaa28 is alpha-MD
HXQGTFTSDKSKYLDXRAAQDFVXWLXXT

SEQ ID NO: 157 - protein, 31 aa, Artificial Sequence
  Peptide
  PEPTIDE (1)..(31): C-terminal amide
  MISC_FEATURE (2)..(2): Xaa2 is D-Ser
  MISC_FEATURE (10)..(10): Lys10 conjugated to  gamma-Glu-gamma-Glu-C16
  MISC_FEATURE (27)..(27): Xaa27 is methione sulfone
  MISC_FEATURE (28)..(28): Xaa28 is alpha-MD
  MISC_FEATURE (31)..(31): Xaa31 is Nle(epsilon-N3)
HXQGTFTSDKSKYLDERAAQDFVQWLXXTKX

SEQ ID NO: 158 - protein, 31 aa, Artificial Sequence
  Peptide
  PEPTIDE (1)..(31): C-terminal amide
  MISC_FEATURE (2)..(2): Xaa2 is D-Ser
  MISC_FEATURE (10)..(10): Lys10 conjugated to  gamma-Glu-gamma-Glu-C16
  MISC_FEATURE (25)..(25): Xaa25 is alpha-MW
  MISC_FEATURE (27)..(27): Xaa27 is methione sulfone
  MISC_FEATURE (31)..(31): Xaa31 is Nle(epsilon-N3)
HXQGTFTSDKSKYLDERAAQDFVQXLXDTKX

SEQ ID NO: 159 - protein, 31 aa, Artificial Sequence
  Peptide
  PEPTIDE (1)..(31): C-terminal amide
  MISC_FEATURE (2)..(2): Xaa2 is D-Ser
  MISC_FEATURE (10)..(10): Lys10 conjugated to  gamma-Glu-gamma-Glu-C16
  MISC_FEATURE (25)..(25): Xaa25 is alpha-MW
  MISC_FEATURE (27)..(27): Xaa27 is methione sulfone
  MISC_FEATURE (31)..(31): K31 conjugated to  PEG2PEG2C5N3
HXQGTFTSDKSKYLDERAAQDFVQXLXDTKK

SEQ ID NO: 160 - protein, 31 aa, Artificial Sequence
  Peptide
  PEPTIDE (1)..(31): C-terminal amide
  MISC_FEATURE (2)..(2): Xaa2 is D-Ser
  MISC_FEATURE (10)..(10): Lys10 conjugated to  gamma-Glu-gamma-Glu-C16
  MISC_FEATURE (27)..(27): Xaa27 is methione sulfone
  MISC_FEATURE (28)..(28): Xaa28 is alpha-MD
  MISC_FEATURE (31)..(31): K31 conjugated to  PEG2PEG2C5N3
HXQGTFTSDKSKYLDERAAQDFVQWLXXTKK

SEQ ID NO: 161 - protein, 31 aa, Artificial Sequence
  Peptide
  PEPTIDE (1)..(31): C-terminal amide
  MISC_FEATURE (2)..(2): Xaa2 is aib
  MISC_FEATURE (20)..(20): K20 is conjugated to  PEG2PEG2-gamma-Glu-C18OH
  MISC_FEATURE (27)..(27): Xaa27 is methione sulfone
  MISC_FEATURE (31)..(31): Xaa31 is Nle(epsilon-N3)
HXQGTFTSDYSKYLDARAAKDFVQWLXDTKX

SEQ ID NO: 162 - protein, 31 aa, Artificial Sequence; identical to SEQ ID NO: 94
  Peptide
  PEPTIDE (1)..(31): C-terminal amide
  MISC_FEATURE (2)..(2): Xaa2 is D-Ser
  MISC_FEATURE (10)..(10): Lys10 conjugated to  PEG2PEG2gamma-Glu-C18OH
  MISC_FEATURE (31)..(31): Xaa31 is Nle(epsilon-N3)

SEQ ID NO: 163 - protein, 29 aa, Artificial Sequence
  Peptide
  PEPTIDE (1)..(29): C-terminal amide
  MISC_FEATURE (2)..(2): Xaa2 is aib
  MISC_FEATURE (20)..(20): K20 conjugated to (PEG2PEG2-Gamma-Glu-C18OH)
  MISC_FEATURE (21)..(21): Xaa21 is Nle(epsilon-N3)
  MISC_FEATURE (27)..(27): Xaa27 is methione sulfone
  MISC_FEATURE (28)..(28): Xaa28 is alpha-MD
HXQGTFTSDYSKYLDARAAKDFVXWLXXT

SEQ ID NO: 164 - protein, 29 aa, Artificial Sequence
  Peptide
  PEPTIDE (1)..(29): C-terminal amide
  MISC_FEATURE (2)..(2): Xaa2 is aib
  MISC_FEATURE (20)..(20): K20 conjugated to (PEG2PEG2-gamma-GluC18OH)
  MISC_FEATURE (24)..(24): K24 is conjugated to (PEG2PEG2C16N3)
  MISC_FEATURE (27)..(27): Xaa27 is methione sulfone
  MISC_FEATURE (28)..(28): Xaa28 is alpha-MD
HXQGTFTSDYSKYLDARAAKDFVKWLXXT

SEQ ID NO: 165 - protein, 29 aa, Artificial Sequence
  Peptide
  PEPTIDE (1)..(29): C-terminal amide
  MISC_FEATURE (2)..(2): Xaa2 is aib
  MISC_FEATURE (9)..(9): Xaa9 is alpha-MD
  MISC_FEATURE (10)..(10): Lys10 conjugated to  gamma-Glu-gamma-Glu-C16
  MISC_FEATURE (24)..(24): Xaa24 is Nle(epsilon-N3)
  MISC_FEATURE (27)..(27): Xaa27 is methione sulfone
HXQGTFTSXKSKYLDERAAQDFVXWLXDT

SEQ ID NO: 166 - protein, 29 aa, Artificial Sequence
  Peptide
  PEPTIDE (1)..(29): C-terminal amide
  MISC_FEATURE (2)..(2): Xaa2 is aib
  MISC_FEATURE (9)..(9): Xaa9 is alpha-MD
  MISC_FEATURE (10)..(10): Lys10 conjugated to  gamma-Glu-gamma-Glu-C16
  MISC_FEATURE (24)..(24): K24 is conjugated to (PEG2PEG2C16N3)
  MISC_FEATURE (27)..(27): Xaa27 is methione sulfone
HXQGTFTSXKSKYLDERAAQDFVKWLXDT